(19) United States
(12) Patent Application Publication
(10) Pub. No.: US 2006/0247303 A1
(43) Pub. Date: Nov. 2, 2006

(54) 7H-dibenzo[b,g][1,5]dioxocin-5-one derivatives and use thereof

(75) Inventors: Hilmar Bischoff, Leverkusen (DE); Frank-Thorsten Hafner, Leverkusen (DE); Carsten Schmeck, Leverkusen (DE); Joachim Telser, Leverkusen (DE); Alexandros Vakalopoulos, Leverkusen (DE); Gabriele Wirtz, Leverkusen (DE); Marcus Bauser, Leverkusen (DE); David Bruckner, Leverkusen (DE); Martina Wuttke, Leverkusen (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, LEVERKUSEN (DE)

(21) Appl. No.: 10/531,881

(22) PCT Filed: Oct. 21, 2003
(86) PCT No.: PCT/EP03/11619

§ 371 Date: May 15, 2006

(30) Foreign Application Priority Data
Oct. 31, 2002 (DE) 102 50 687.6

(51) Int. Cl. A61K 31/365; C07D 321/02

(52) U.S. Cl. 514/450; 549/267

(57) ABSTRACT

The present invention relates to substituted 7H-dibenzo[b,g][1,5]dioxocin-5-one-derivatives, to processes for their preparation and to their use in medicaments, in particular as inhibitors of the cholesterol ester transfer protein (CETP) for the treatment and/or prevention of cardiovascular disorders, in particular hypolipoproteinaemia, dyslipidaemias, hypertriglyceridaemias, hyperlipidaemias and arteriosclerosis.

The present application relates to substituted 7H-dibenzo[b,g][1,5]dioxocin-5-one derivatives, to processes for their preparation and to their use in medicaments, in particular as inhibitors of the cholesterol ester transfer protein (CETP) for the treatment and/or prevention of cardiovascular disorders, in particular hypolipoproteinaemia, dyslipidaemias, hypertriglyceridaemias, hyperlipidaemias and arteriosclerosis.

Coronary heart disease caused by arteriosclerosis is one of the main causes of death in modern society. In a large number of studies, it was shown that low plasma concentrations of HDL cholesterol are an important risk factor for the development of arteriosclerosis [Barter and Rye, Atherosclerosis 121, 1-12 (1996)]. HDL (high density lipoprotein), in addition to LDL (low density lipoprotein) and VLDL (very low density lipoprotein), is a class of lipoproteins whose most important function is the transport of lipids, such as, for example, cholesterol, cholesterol esters, triglycerides, fatty acids or phospholipids, in the blood. High LDL cholesterol concentrations (>180 mg/dl) and low HDL cholesterol concentrations (<35 mg/dl) contribute substantially to the development of arteriosclerosis. In addition to coronary heart disease, unfavourable HDL/LDL ratios also promote the development of peripheral vascular disorders and stroke. Accordingly, novel methods for elevating HDL cholesterol in the plasma are a therapeutically useful advance in prevention and treatment of arteriosclerosis and the disorders associated therewith.

Cholesterol ester transfer protein (CETP) mediates the exchange of cholesterol esters and triglycerides between the different lipoproteins in the blood [Tall, J. Lipid Res. 34, 1255-74 (1993)]. Of particular importance here is the transfer of cholesterol esters from HDL to LDL which results in a reduction of the HDL cholesterol plasma concentration. Accordingly, inhibition of CETP should result in elevated HDL cholesterol plasma concentrations and a reduction of the LDL cholesterol plasma concentrations and thus in a therapeutically useful effect on the lipid profile in the plasma [McCarthy, Medicinal Res. Revs. 1, 139-59 (1993); Sitori, Pharmac. Ther. 67, 443-47 (1995); Swenson, J. Biol. Chem. 264, 14318 (1989)].

7H-Dibeno[b,g][1,5]dioxocin-5-ones, their preparation and their use, based on ANP release, as antihypertensives, cardiotherapeutics and coronary therapeutics are described in EP-A-411 268. An action of the natural product penicillide [11-hydroxy-3-[(1S)-1-hydroxy-3-methylbutyl]-4-methoxy-9-methyl-5H,7H-dibenzo[b,g][1,5]dioxocin-5-one; Tetrahedron Lett. 45, 3941-2 (1974)] and some derivatives as oxytocin antagonists is described in U.S. Pat. No. 5,198,463 and in Bioorg. Med. Chem. Lett. 3, 337-340 (1993). An ACAT-inhibitory action of penicillide and the 1′-O-acetyl derivative purpactin A is reported in J. Antibiot. 44, 136-143, 144-151, 152-159 (1991), ibid 47, 16-22 (1994) and in JP-A-03052884. WO 94/12175 claims a cholesterol- and lipid-lowering action of penicillide.

It has now been found that compounds of the general formula (I) 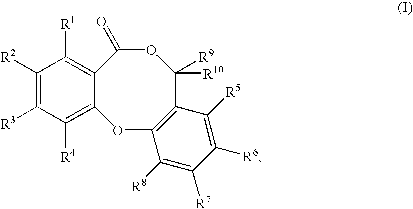
in which
R¹ represents hydrogen, halogen, cyano, (C₁-C₄)-alkyl, (C₁-C₄)-alkoxy, mono- or di-(C₁-C₄)-alkylamino, trifluoromethyl, trifluoromethoxy, hydroxy, vinyl or ethynyl,
R² represents a group of the formula 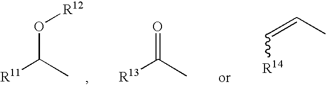
   where
   R¹¹ represents (C₁-C₆)-alkyl or (C₂-C₆)-alkenyl, each of which may be mono- or polysubstituted by substituents selected from the group consisting of (C₃-C₆)-cycloalkyl, phenyl, (C₁-C₄)-alkoxy and fluorine, or represents (C₆-C₁₀)-aryl which may be mono- or disubstituted by identical or different substituents from the group consisting of halogen, (C₁-C₄)-alkyl, (C₁-C₄)-alkoxy, trifluoromethyl and trifluoromethoxy,
   R¹² represents hydrogen or formyl,
   R¹³ and R¹⁴ each represent (C₁-C₆)-alkyl,
R³ and R⁴ independently of one another represent hydrogen, halogen, trifluoromethyl, trifluoromethoxy, (C₁-C₄)-alkyl, (C₁-C₄)-alkoxy, (C₂-C₄)-alkenyl or (C₃-C₆)-cycloalkyl,
R⁵, R⁶ and R⁷ independently of one another represent hydrogen, halogen, cyano, nitro, hydroxy, trifluoromethoxy, formyl, (C₁-C₄)-alkoxy, (C₂-C₄)-alkenyl, (C₃-C₆)-cycloalkyl or represent (C₁-C₄)-alkyl which may be substituted by hydroxy, trifluoromethoxy, (C₁-C₄)-alkoxy or up to three times by fluorine,
R⁸ represents (C₁-C₈)-alkyl, (C₂-C₈)-alkenyl or (C₂-C₈)-alkynyl, each of which may be substituted by (C₃-C₈)-cycloalkyl, (C₁-C₄)-alkoxy, pyrrolyl, imidazolyl, triazolyl, tetrazolyl or phenyl which for its part is optionally substituted by (C₁-C₄)-alkyl,
   represents (C₆-C₁₀)-aryl which may be mono- or disubstituted by identical or different substituents from the group consisting of halogen, (C₁-C₄)-alkyl, (C₁-C₄)-alkoxy, trifluoromethyl, trifluoromethoxy, cyano and nitro,
   represents (C₁-C₈)-alkoxy or (C₂-C₈)-alkenyloxy, each of which may be substituted by (C₃-C₈)-cycloalkyl, (C₃-C₈)-cycloalkenyl or phenyl, (which for its part is optionally substituted by halogen, nitro or cyano) or up to five times by fluorine and/or chlorine,
   represents (C₃-C₈)-cycloalkoxy or represents (C₆-C₁₀)-aryloxy which may be substituted by halogen, nitro or cyano,
   represents mono- or di-(C₁-C₈)-alkylamino, (C₁-C₈)-alkylsulphonylamino or N-[(C₁-C₈)-alkyl]-(C₁-C₈)-alkylsulphonylamino, or
   represents a group of the formula —O—SO₂—R¹⁵, —O—C(O)—R¹⁶, —O—C(O)—NR¹⁷R¹⁸, —C(O)—OR¹⁹, —NR²⁰—C(O)—R²¹ or —NR²²—C(O)—NR²³R²⁴, where
   R¹⁵ represents (C₁-C₈)-alkyl which may be substituted up to five times by fluorine, represents (C₃-C₈)-cycloalkyl or represents phenyl which may be substituted by halogen or (C₁-C₄)-alkyl,
   R¹⁶ represents (C₁-C₁₀)-alkyl which may be substituted by phenyl or phenoxy (which for their part may each be mono- or disubstituted by halogen), by (C₃-C₈)-cycloalkyl, (C₃-C₈)-cycloalkenyl, (C₁-C₆) alkoxy, (C₁-C₆)-alkylthio, (C₂-C₆)-alkenylthio or up to six times by fluorine,
      represents (C₃-C₁₂)-cycloalkyl which may be mono- or polysubstituted by substituents selected from the group consisting of phenyl, (C₂-C₆) alkenyl, trifluoromethyl, (C₁-C₆)-alkyl, cyano and fluorine, where phenyl for its part may be mono- or disubstituted by identical or different substituents from the group consisting of halogen, (C₁-C₄)-alkyl and (C₁-C₄)-alkoxy,
      represents (C₃-C₁₂)cycloalkenyl which may be substituted up to three times by (C₁-C₄)-alkyl, trifluoromethyl or fluorine,
      represents a 5- to 7-membered mono- or bicyclic saturated or partially unsaturated heterocycle which has up to two heteroatoms from the group consisting of N, O and S and which may be substituted up to two times by (C₁-C₄)-alkyl, or
      represents (C₆-C₁₀)-aryl which may be mono- or disubstituted by identical or different substituents from the group consisting of halogen, nitro, cyano, trifluoromethyl, trifluoromethoxy, (C₁-C₄)-alkyl and (C₁-C₄)-alkoxy,
   R¹⁷ and R¹⁸ independently of one another represent hydrogen, (C₁-C₆)-alkyl which may be substituted by (C₁-C₄)-alkoxycarbonyl or up to three times by fluorine, represent (C₂-C₆)-alkenyl, (C₃-C₈)-cycloalkyl, (C₁-C₄)-alkylsulphonyl or represent phenyl which may be mono- or disubstituted by identical or different substituents from the group consisting of halogen and trifluoromethyl, or
   together with the nitrogen atom to which they are attached form a 4 to 12-membered mono-, bi- or tricyclic saturated or partially unsaturated heterocycle which may contain up to two further heteroatoms from the group consisting of N, O and S and which may be substituted by phenyl or up to four times by (C₁-C₄)-alkyl,
R¹⁹ represents (C₁-C₆)-alkyl which may be substituted by (C₃-C₈)-cycloalkyl, represents (C₃-C₁₀)-cycloalkyl which may be substituted up to two times by (C₁-C₄)-alkyl or represents (C₂-C₆)-alkenyl,
R²⁰ represents hydrogen or (C₁-C₆)-alkyl,
R²¹ represents (C₁-C₈)-alkoxy, (C₁-C₈)-alkyl, (C₆-C₁₀)-aryl or represents (C₃-C₁₀)-cycloalkyl which may be substituted up to two times by (C₁-C₄)-alkyl,
R²² represents hydrogen or (C₁-C₆)-alkyl, and
R²³ and R²⁴ independently of one another represent hydrogen, (C₁-C₆)-alkyl or (C₃-C₁₀)-cycloalkyl, and
R⁹ and R¹⁰ independently of one another represent hydrogen or (C₁-C₄)-alkyl,
   and their pharmaceutically acceptable salts, solvates and solvates of the salts,
   have a CETP-inhibitory action and can be used as medicaments or for the preparation of medicament formulations for the treatment and/or prevention of cardiovascular disorders, in particular of hypolipoproteinaemia, dislipidaemias, hypertriglyceridaemias, hyperlipidaemias and arteriosclerosis.

Unless defined in more detail below, the following definitions apply to the meanings of the substituents and radicals in the general formulae given:

In the context of the invention, (C₁-C₁₀)-alkyl, (C₁-C₈)-alkyl, (C₁-C₆)-alkyl, (C₁-C₄)-alkyl represent a straight-chain or branched alkyl radical having 1 to 10, 1 to 8, 1 to 6 and 1 to 4 carbon atoms, respectively. Preference is given to a straight-chain or branched alkyl radical having 1 to 6, particularly preferably 1 to 4, carbon atoms. The following radicals may be mentioned by way of example and by way of preference: methyl, ethyl, n-propyl, isopropyl, n-butyl, tert-butyl, n-pentyl and n-hexyl.

In the context of the invention, (C₂-C₈)-alkenyl, (C₂-C₆)alkenyl and (C₂-C₄)-alkenyl alkyl represent a straight-chain or branched alkenyl radical having 2 to 8, 2 to 6 and 2 to 4 carbon atoms, respectively. Preference is given to a straight-chain or branched alkenyl radical having 2 to 6, particularly preferably 2 to 4, carbon atoms. The following radicals may be mentioned by way of example and by way of preference: vinyl, allyl, isopropenyl and n-but-2-en-1-yl.

In the context of the invention, (C₂-C₈)-alkynyl represents a straight-chain or branched alkynyl radical having 2 to 8 carbon atoms. Preference is given to a straight-chain or branched alkynyl radical having 2 to 6 carbon atoms. The following radicals may be mentioned by way of example and by way of preference: ethynyl, n-prop-2-yn-1-yl and n-but-2-yn-1-yl.

In the context of the invention, (C₃-C₁₂)-cycloalkyl, (C₃-C₁₀)-cycloalkyl, (C₃-C₈)-cycloalkyl and (C₃-C₆)-cycloalkyl represent a monocyclic or optionally bi- or tricyclic cycloalkyl group having 3 to 12, 3 to 10, 3 to 8 and 3 to 6 carbon atoms, respectively. Preference is given to a mono- or bicyclic cycloalkyl group having 3 to 10, particularly preferably 3 to 8, carbon atoms. The following radicals may be mentioned by way of example and by way of preference: cyclopropyl, cyclobutyl, cyclopentyl, cyclohexyl cycloheptyl, cyclooctyl, cyclononyl bicyclo[2.1.1]hexyl, bicyclo[2.2.1]heptyl, bicyclo[3.2.1]octyl, bicyclo[2.2.2]octyl bicyclo[3.2.2]nonyl, bicyclo[3.3.1]nonyl, bicyclo[3.3.2]decyl, bicyclo[4.3.1]decyl and adamantyl.

In the context of the invention, (C₃-C₁₂)-cycloalkenyl and (C₃-C₈)-cycloalkenyl represent a monocyclic or optionally bi- or tricyclic cycloalkyl group having 3 to 12 and 3 to 8 carbon atoms, respectively, which contains one or optionally two double bonds. Preference is given to a mono- or bicyclic cycloalkenyl group having 5 to 10, particularly preferably 5 to 7, carbon atoms which contains one double bond. The following radicals may be mentioned by way of example and by way of preference: cyclobutenyl cyclopentenyl, cyclohexenyl, cycloheptenyl, cyclooctenyl, cyclononenyl, bicyclo[2.2.1]heptenyl, bicyclo[2.2.2]octenyl and bicyclo[3.2.2]nonenyl.

In the context of the invention, (C₆-C₁₀)-aryl represents an aromatic hydrocarbon radical having preferably 6 to 10 carbon atoms. Preferred aryl radicals are phenyl and naphthyl.

In the context of the invention, (C₁-C₈)-alkoxy, (C₁-C₆)-alkoxy and (C₁-C₄)alkoxy represent a straight-chain or branched alkoxy radical having 1 to 8, 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy, n-butoxy, tert-butoxy, n-pentoxy and n-hexoxy.

In the context of the invention, (C₂-C₈)-alkenyloxy represents a straight-chain or branched alkenyl radical having 2 to 8 carbon atoms which is attached via an oxygen atom. Preference is given to a straight-chain or branched alkenyloxy radical having 2 to 6, particularly preferably 2 to 4, carbon atoms. The following radicals may be mentioned by way of example and by way of preference: allyloxy, but-2-en-1-oxy, pent-3-en-1-oxy and hex-2-en-1-oxy.

In the context of the invention, (C₃-C₈)-cycloalkoxy represents a monocyclic or optionally bicyclic cycloalkyl group having 3 to 8 carbon atoms which is attached via an oxygen atom. Preference is given to a monocyclic cycloalkoxy group having 5 to 7 carbon atoms. The following radicals may be mentioned by way of example and by way of preference: cyclobutoxy, cyclopentoxy, cyclohexoxy, cycloheptoxy and cyclooctoxy.

In the context of the invention, (C₆-C₁₀)-aryloxy represents an aryl radical having preferably 6 to 10 carbon atoms which is attached by way of an oxygen atom. Preferred aryloxy radicals are phenoxy and naphthoxy.

In the context of the invention, (C₁-C₄)-alkoxycarbonyl represents a straight-chain or branched alkoxy radical having 1 to 4 carbon atoms which is attached via a carbonyl group. Preference is given to an alkoxycarbonyl radical having 1 or 2 carbon atoms.

The following radicals may be mentioned by way of example and by way of preference: methoxycarbonyl, ethoxycarbonyl, n-propoxycarbonyl, isopropoxycarbonyl and tert-butoxycarbonyl.

In the context of the invention, mono-(C₁-C₈)-alkylamino and mono-(C₁-C₄)-alkylamino represent an amino group having a straight-chain or branched alkyl substituent which has 1 to 8 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino, n-butylamino, tert-butylamino, n-pentylamino and n-hexylamino.

In the context of the invention, di-(C₁-C₈)-alkylamino and di-(C₁-C₄)-alkylamino represent an amino group having two identical or different straight-chain or branched alkyl substituents each having 1 to 8 and 1 to 4 carbon atoms, respectively. Preference is given to a straight-chain or branched dialkylamino radical having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methyl-amino, N-ethyl-N-n-pentylamino and N-n-hexyl-N-methylamino.

In the context of the invention, (C₁-C₈)-alkylsulphonyl represents a straight-chain or branched alkylsulphonyl radical having 1 to 8 carbon atoms. Preference is given to a straight-chain or branched alkylsulphonyl radical having 1 to 4 carbon atoms. The following radicals may be mentioned by way of example and by way of preference: methylsulphonyl, ethylsulphonyl, n-propylsulphonyl, isopropylsulphonyl, tert-butylsulphonyl, n-pentylsulphonyl and n-hexylsulphonyl.

In the context of the invention, (C₁-C₈)-alkylsulphonylamino represents an amino group having a straight-chain or branched alkylsulphonyl substituent which has 1 to 8 carbon atoms and is attached via the sulphonyl group. Preference is given to a straight-chain or branched alkylsulphonylamino radical having 1 to 4 carbon atoms. The following radicals may be mentioned by way of example and by way of preference: methyl-sulphonylamino, ethylsulphonylamino, n-propylsulphonylamino, isopropylsulphonyl-amino, tert-butylsulphonylamino, n-pentylsulphonylamino and n-hexylsulphonylamino.

In the context of the invention, N-[(C₁-C₈)-alkyl]-(C₁-C₈)-alkylsulphonylamino represents an amino group having a straight-chain or branched alkyl substituent and a straight-chain or branched alkylsulphonyl substituent each having 1 to 8 carbon atoms. Preference is given to a straight-chain or branched N-(alkyl)-alkyl-sulphonylamino radical having in each case 1 to 4 carbon atoms. The following radicals may be mentioned by way of example and by way of preference: N-methyl-methyl-sulphonylamino, N-ethyl-methylsulphonylamino, N-n-propyl-methylsulphonylamino, N-n-butyl-methylsulphonylamino, N-tert-butyl-methylsulphonylamino, N-methyl-ethyl-sulphonylamino, N-methyl-n-propylsulphonylamino, N-methyl-isopropylsulphonyl-amino, N-methyl-tert-butylsulphonylamino, N-methyl-n-pentylsulphonylamino and N-methyl-n-hexylsulphonylamino.

In the context of the invention, (C₁-C₆)-alkylthio represents a straight-chain or branched alkylthio radical having 1 to 6 carbon atoms. Preference is given to a straight-chain or branched alkylthio radical having 1 to 4 carbon atoms. The following radicals may be mentioned by way of example and by way of preference: methylthio, ethylthio, n-propylthio, isopropylthio, tert-butylthio, n-pentylthio and n-hexylthio.

In the context of the invention, (C₂-C₆)-alkenylthio represents a straight-chain or branched alkenyl radical having 2 to 6 carbon atoms which is attached via a sulphur atom. Preference is given to a straight-chain or branched alkenylthio radical having 2 to 4 carbon atoms. The following radicals may be mentioned by way of example and by way of preference: allylthio, but-2-en-1-ylthio, pent-3-en-1-ylthio and hex-2-en-1-yl-thio.

In the context of the invention, a 5- to 7-membered heterocycle represents a mono- or bicyclic saturated or partially unsaturated heterocycle which has up to two heteroatoms from the group consisting of N, O and S and which is attached via a ring carbon atom of the heterocycle. The following radicals may be mentioned by way of example and by way of preference: tetrahydrofuryl, dihydrofuryl, thiolanyl, dioxolanyl, pyrrolidinyl, pyrrolinyl, tetrahydropyranyl, dihydropyranyl, piperidinyl, piperazinyl, morpholinyl, thiomorpholinyl, 7-oxabicyclo[2.2.1]heptanyl and 7-oxa-bicyclo[2.2.1]hept-5-enyl.

In the context of the invention, a 4- to 12-membered heterocycle having at least one ring nitrogen atom represents a saturated or partially unsaturated monocyclic or optionally bi- or tricyclic heterocycle which may contain up to two further heteroatoms from the group consisting of N, O and S and which is attached via a ring nitrogen atom of the heterocycle. Preference is given to a 4- to 10-membered saturated mono- or bicyclic N-heterocycle which may contain a second nitrogen atom or an oxygen atom as further heteroatom. The following radicals may be mentioned by way of example and by way of preference: pyrrolidinyl, pyrrolinyl, oxazolidinyl, thiazolidinyl, piperidinyl, piperazinyl, morpholinyl, thiomorpholinyl, hexahydro-azepinyl, hexahydro-1,4-diazepinyl, octahydroazocinyl, 7-azabicyclo[2.2.1]heptanyl, 3-azabicyclo[3.2.0]heptanyl, 3-azabicyclo[3.2.1]octanyl, 8-oxa-3-azabicyclo[3.2.1]-octanyl and 5-azatricyclo[5.2.1.0^{3,8}]decanyl.

In the context of the invention, halogen includes fluorine, chlorine, bromine and iodine. Preference is given to chlorine or fluorine.

Hereinbelow, the compounds of the formula (I) which can be used or are used according to the invention and the novel compounds of the formulae (I), (I-A) and (I-B) are referred to as compounds according to the invention or active compounds according to the invention.

Depending on the substitution pattern, the compounds according to the invention can exist in stereoisomeric forms which are either like image and mirror image (enantiomers) or which are not like image and mirror image (diastereomers). The invention relates both to the enantiomers or diastereomers and to their respective mixtures. The racemates, like the diastereomers, can be separated in a known manner into the stereoisomerically uniform components.

Furthermore, certain compounds can be present in tautomeric forms. This is known to the person skilled in the art, and such compounds are also embraced by the scope of the invention.

The compounds according to the invention can also be present as salts. In the context of the invention, preference is given to physiologically acceptable salts.

Physiologically acceptable salts can be salts of the compounds according to the invention with inorganic or organic acids. Preference is given to salts with inorganic acids such as, for example, hydrochloric acid, hydrobromic acid, phosphoric acid or sulphuric acid or salts with organic carboxylic or sulphonic acids such as, for example, acetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalene-disulphonic acid.

Physiologically acceptable salts can also be salts of the compounds according to the invention with bases, such as, for example, metal or ammonium salts. Preferred examples are alkali metal salts (for example sodium or potassium salts), alkaline earth metal salts (for example magnesium or calcium salts), and also ammonium salts which are derived from ammonia or organic amines such as, for example, ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The compounds according to the invention can also be present in the form of their solvates, in particular in the form of their hydrates.

Preference is given to using compounds of the general formula (I) in which
R¹ represents fluorine, chlorine, cyano, methyl, ethyl, methoxy, ethoxy, trifluoromethyl, trifluoromethoxy or hydroxy,
R² represents a group of the formula 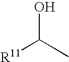
   where
   R¹¹ represents (C₁-C₆)-alkyl or (C₂-C₆)-alkenyl, each of which may be mono- or polysubstituted by substituents selected from the group consisting of (C₃-C₆)-cycloalkyl, methoxy and fluorine, or represents phenyl which may be mono- or disubstituted by identical or different substituents from the group consisting of fluorine, chlorine, methyl, methoxy, trifluoromethyl and trifluoromethoxy,
R³ and R⁴ independently of one another represent hydrogen, fluorine, chlorine, trifluoromethyl, (C₁-C₄)-alkyl, cyclobutyl or cyclopentyl,
R⁵, R⁶ and R⁷ independently of one another represent hydrogen, fluorine, chlorine, bromine, cyano, nitro, trifluoromethoxy, methoxy, vinyl, alkyl, cyclopropyl, cyclobutyl or represent (C₁-C₄)-alkyl which may be substituted by (C₁-C₄)-alkoxy or up to three times by fluorine,
R⁸ represents (C₃-C₈)-alkyl, (C₃-C₈)-alkenyl or (C₃-C₈)-alkynyl, each of which may be substituted by (C₃-C₆)-cycloalkyl or (C₁-C₄)-alkoxy,
   represents (C₃-C₈)-alkoxy or (C₃-C₈)-alkenyloxy, each of which may be substituted by (C₃-C₆)-cycloalkyl, (C₃-C₆)-cycloalkenyl or up to three times by fluorine, or represents (C₃-C₆)-cycloalkoxy, or
   represents a group of the formula —O—SO₂—R¹⁵, —O—C(O)—R¹⁶ or —O—C(O)—NR¹⁷R¹⁸ where
   R¹⁵ represents (C₁-C₆)-alkyl which may be substituted up to five times by fluorine or represents (C₃-C₆)-cycloalkyl,
   R¹⁶ represents (C₁-C₁₀)-alkyl which may be substituted by phenyl, (C₃-C₆)-cycloalkyl, (C₃-C₆)-cycloalkenyl, (C₁-C₄)-alkoxy or up to three times by fluorine,
      represents (C₃-C₁₀)-cycloalkyl which may be mono- or polysubstituted by substituents selected from the group consisting of trifluoromethyl, (C₁-C₄)-alkyl, cyano and fluorine,
      represents (C₅-C₁₀)-cycloalkenyl which may be substituted up to two times by (C₁-C₄)-alkyl, or
      represents a 5- to 7-membered saturated or partially unsaturated mono- or bicyclic heterocycle which has a ring oxygen atom and which may be substituted up to two times by (C₁-C₄)-alkyl,
   R¹⁷ and R¹⁸ independently of one another represent hydrogen, (C₁-C₆)-alkyl which may be substituted up to three times by fluorine, represent (C₃-C₆)-alkenyl or represent (C₃-C₆)-cycloalkyl, or
      together with the nitrogen atom to which they are attached form a 4 to 10-membered mono-, bi- or tricyclic saturated or partially unsaturated heterocycle which may contain an oxygen atom as further heteroatom and which may be substituted up to four times by (C₁-C₃)-alkyl,
R⁹ represents hydrogen, and
R¹⁰ represents hydrogen or (C₁-C₄)-alkyl.

Particular preference is given to using compounds of the general formula (I) in which
R¹ represents cyano, methoxy or ethoxy,
R² represents a group of the formula 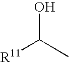
   where
   R¹¹ represents (C₁-C₆)-alkyl or (C₂-C₆)-alkenyl each of which may be mono- or polysubstituted by substituents selected from the group consisting of cyclopropyl, cyclobutyl, methoxy and fluorine,
R³ and R⁴ each represent hydrogen,
R⁵, R⁶ and R⁷ independently of one another represent hydrogen, fluorine, chlorine, bromine, cyano or represent (C₁-C₄)-alkyl which may be substituted by methoxy or up to three times by fluorine,
R⁸ represents (C₃-C₇)-alkyl, (C₃-C₇)-alkenyl or (C₃-C₇)-alkynyl, each of which may be substituted by cyclopentyl, cyclohexyl or methoxy,
   represents (C₃-C₇)-alkoxy which may be substituted by cyclopentyl, cyclohexyl or up to three times by fluorine, or represents (C₄-C₆)-cycloalkoxy, or
   represents a group of the formula —O—C(O)—R¹⁶ or —O—C(O)—NR¹⁷R¹⁸, where
   R¹⁶ represents (C₁-C₈)-alkyl which may be substituted by phenyl, cyclopentyl, cyclohexyl, (C₁-C₄)-alkoxy or up to three times by fluorine,
      represents (C₃-C₁₀)-cycloalkyl which may be mono- or polysubstituted by substituents selected from the group consisting of trifluoromethyl, (C₁-C₃)-alkyl, cyano and fluorine,
      represents (C₅-C₇)-cycloalkenyl which may be substituted up to two times by (C₁-C₃)-alkyl, or
      represents 7-oxabicyclo[2.2.1]heptanyl or 7-oxabicyclo[2.2.1]hept-5-enyl, each of which may be substituted up to two times by methyl or ethyl,
   R¹⁷ and R¹⁸ independently of one another represent (C₁-C₆)-alkyl which may be substituted up to three times by fluorine, represent (C₃-C₆)-alkenyl or represent (C₃-C₆)-cycloalkyl, or
      together with the nitrogen atom to which they are attached form a 4- to 10-membered saturated mono- or bicyclic heterocycle which may contain an oxygen atom as further heteroatom and which may be substituted up to four times by methyl or ethyl,
R⁹ represents hydrogen, and
R¹⁰ represents hydrogen, methyl or ethyl.

Very particular preference is given to using compounds of the general formula (I), in which
R¹ represents cyano, methoxy or ethoxy,
R¹ represents a group of the formula 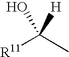
   in which
   R¹¹ represents (C₁-C₆)-alkyl or (C₂-C₆)-alkenyl, each of which may be mono- or polysubstituted by substituents selected from the group consisting of cyclopropyl, cyclobutyl, methoxy and fluorine,
R³ and R⁴ each represent hydrogen,
R⁹ represents hydrogen,
R¹⁰ represents hydrogen, methyl and ethyl,
   and R⁵, R⁶, R⁷ and R⁸ are each as defined above.

The present invention also provides novel compounds of the general formula (I) in which
R⁸ represents a group of the formula —O—C(O)—R¹⁶ where
   R¹⁶ represents (C₁-C₁₀)-alkyl which may be substituted by phenyl or phenoxy (which for their part may each be mono- or disubstituted by halogen), by (C₃-C₈)-cycloalkyl, (C₃-C₈)-cycloalkenyl, (C₁-C₆)-alkoxy, (C₁-C₆)-alkylthio, (C₂-C₆)-alkenylthio or up to six times by fluorine,
      represents (C₃-C₁₂)-cycloalkyl which may be mono- or polysubstituted by substituents selected from the group consisting of phenyl, (C₂-C₆)-alkenyl, trifluoromethyl, (C₁-C₆)-alkyl, cyano and fluorine, where phenyl for its part may be mono- or disubstituted by identical or different substituents from the group consisting of halogen, (C₁-C₄)-alkyl and (C₁-C₄)-alkoxy,
      represents (C₃-C₁₂)-cycloalkenyl which may be substituted up to three times by (C₁-C₄)-alkyl, trifluoromethyl or fluorine,
      represents a 5- to 7-membered mono- or bicyclic saturated or partially unsaturated heterocycle which has up to two heteroatoms from the group consisting of N, O and S and which may be substituted up to two times by (C₁-C₄)-alkyl, or
      represents (C₆-C₁₀)-aryl which may be mono- or disubstituted by identical or different substituents from the group consisting of halogen, nitro, cyano, trifluoromethyl, trifluoromethoxy, (C₁-C₄)-alkyl and (C₁-C₄)-alkoxy,
         and R¹, R², R³, R⁴, R⁵, R⁶, R⁷, R⁹ and R¹⁰ are each as defined above.

The present invention furthermore provides novel compounds of the general formula (I) in which
R⁸ represents a group of the formula —O—C(O)—NR¹⁷R¹⁸ where
   R¹⁷ and R¹⁸ independently of one another represent hydrogen, (C₁-C₆)-alkyl which may be substituted by (C₁-C₄)-alkoxycarbonyl or up to three times by fluorine, represent (C₂-C₆)-alkenyl, (C₃-C₈)-cycloalkyl, (C₁-C₄)-alkylsulphonyl or represent phenyl which may be mono- or disubstituted by identical or different substituents from the group consisting of halogen and trifluoromethyl or
   together with the nitrogen atom to which they are attached form a 4- to 12-membered mono-, bi- or tricyclic saturated or partially unsaturated heterocycle which may contain up to two further heteroatoms from the group consisting of N, O and S and which may be substituted by phenyl or up to four times by (C₁-C₄)-alkyl,
      and R¹, R², R³, R⁴, R⁵, R⁶, R⁷, R⁹ and R¹⁰ are each as defined above.

The invention furthermore provides, novel compounds of the general formula (I), in which
R⁸ represents a group of the formula —C(O)—OR¹⁹ where
   R¹⁹ represents (C₁-C₆)-alkyl which is substituted by (C₃-C₈)-cycloalkyl or represents (C₃-C₁₀)-cycloalkyl which may be substituted up to two times by (C₁-C₄)-alkyl,
      and R¹, R², R³, R⁴, R⁵, R⁶, R⁷, R⁹ and R¹⁰ are each as defined above.

The invention furthermore provides novel compounds of the general formula (I) in which
R⁸ represents a group of the formula —NR²⁰—C(O)—R²¹ where
   R²⁰ represents hydrogen or (C₁-C₆)-alkyl, and
   R²¹ represents (C₁-C₈)-alkoxy, (C₁-C₈)-alkyl, (C₆-C₁₀)-aryl or represents (C₃-C₁₀)-cycloalkyl which may be substituted up to two times by (C₁-C₄)-alkyl,
      and R¹, R², R³, R⁴, R⁵, R⁶, R⁷, R⁹ and R¹⁰ are each as defined above.

The invention furthermore provides novel compounds of the general formula (I) in which
R⁸ represents a group of the formula —NR²²—C(O)—NR²³R²⁴ where
   R²² represents hydrogen or (C₁-C₆)-alkyl, and
   R²³ and R²⁴ independently of one another represent hydrogen, (C₁-C₆)-alkyl or (C₃-C₁₀)-cycloalkyl,
      and R¹, R², R³, R⁴, R⁵, R⁶, R⁷, R⁹ and R¹⁰ are each as defined above.

Preference is given to compounds of the general formula (I) in which
R⁸ represents a group of the formula —O—C(O)—R¹⁶ where
   R¹⁶ represents (C₁-C₁₀)-alkyl which is substituted by phenyl, (C₃-C₆)-cycloalkyl, (C₃-C₆)-cycloalkenyl, (C₁-C₄)-alkoxy or up to three times by fluorine,
      represents (C₃-C₁₀)-cycloalkyl which may be mono- or polysubstituted by substituents selected from the group consisting of trifluoromethyl, (C₁-C₄)-alkyl, cyano and fluorine,
      represents (C₅-C₁₀)-cycloalkenyl which may be substituted up to two times by (C₁-C₄)-alkyl, or
      represents a 5- to 7-membered saturated or partially unsaturated mono- or bicyclic heterocycle which has a ring oxygen atom and may be substituted up to two times by (C₁-C₄)-alkyl,
         and R¹, R², R³, R⁴, R⁵, R⁶, R⁷, R⁹ and R¹⁰ are each as defined above.

Preference is also given to compounds of the general formula (I) in which
R⁸ represents a group of the formula —O—C(O)—NR¹⁷R⁸ where
   R¹⁷ and R¹⁸ independently of one another represent hydrogen, (C₁-C₆)-alkyl which may be substituted up to three times by fluorine, represent (C₃-C₆)-alkenyl or represent (C₃-C₆)-cycloalkyl, or
      together with the nitrogen atom to which they are attached represent a 4- to 10-membered mono-, bi- or tricyclic saturated or partially unsaturated heterocycle which may contain an oxygen atom as further heteroatom and which may be substituted up to four times by (C₁-C₃)-alkyl,
         and R¹, R², R³, R⁴, R⁵, R⁶, R⁷, R⁹ and R¹⁰ are each as defined above.

Particular preference is given to compounds of the general formula (I), in which
R⁸ represents a group of the formula —O—C(O)—R¹⁶ where
   R¹⁶ represents (C₁-C₈)-alkyl which is substituted by phenyl, cyclopentyl, cyclohexyl, (C₁-C₄)-alkoxy or up to three times by fluorine,
      represents (C₃-C₁₀)-cycloalkyl which may be mono- or polysubstituted by substituents selected from the group consisting of trifluoromethyl, (C₁-C₃)-alkyl, cyano and fluorine,
      represents (C₅-C₇)-cycloalkenyl which may be substituted up to two times by (C₁-C₃)-alkyl, or
      represents 7-oxabicyclo[2.2.1]heptanyl or 7-oxabicyclo[2.2.1]hept-5-enyl, each of which may be substituted up to two times by methyl or ethyl,
         and R¹, R², R³, R⁴, R⁵, R⁶, R⁷, R⁹ and R¹⁰ are each as defined above.

Particular preference is also given to compounds of the general formula (I) in which
R⁸ represents a group of the formula —O—C(O)—NR¹⁷R¹⁸ where
   R¹⁷ and R¹⁸ independently of one another represent (C₁-C₆)-alkyl which may be substituted up to three times by fluorine, represent (C₃-C₆)-alkenyl or represent (C₃-C₆)-cycloalkyl, or
      together with the nitrogen atom to which they are attached form a 4 to 10-membered saturated mono- or bicyclic heterocycle which may contain an oxygen atom as further heteroatom and which may be substituted up to four times by methyl or ethyl,
         and R¹, R², R³, R⁴, R⁵, R⁶, R⁷, R⁹ and R¹⁰ are each as defined above.

The general or preferred radical definitions given above apply both to the end products of the formulae (I), (I-A) and (I-B) and, correspondingly, to the starting materials and intermediates required in each case for the preparation.

Very particular preference is given to combinations of two or more of the preferred ranges mentioned above.

Of particular importance are compounds of the formula (I-A) 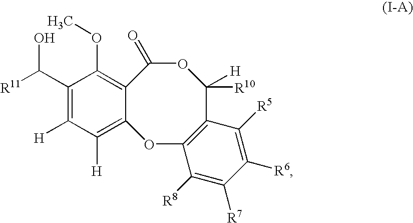
in which
R⁵, R⁶ and R⁷ independently of one another represent hydrogen, fluorine, chlorine, bromine, cyano or represent methyl or ethyl which may be substituted by methoxy or up to three times by fluorine,
R⁸ represents a group of the formula 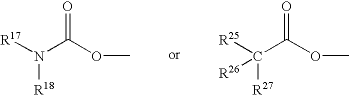
   where
   R¹⁷ and R¹⁸ independently of one another represent hydrogen, (C₁-C₆)-alkyl which may be substituted up to three times by fluorine, represent (C₃-C₆)-alkenyl or represent (C₃-C₆)-cycloalkyl, or
      together with the nitrogen atom to which they are attached form a 4- to 10-membered mono-, bi- or tricyclic saturated or partially unsaturated heterocycle which may contain an oxygen atom as further heteroatom and which may be substituted up to four times by methyl,
   R²⁵ and R²⁶ together with the carbon atom to which they are attached represent (C₃-C₁₀)-cycloalkyl which may be substituted up to four times by substituents selected from the group consisting of fluorine, methyl and trifluoromethyl, represent (C₅-C₁₀)-cycloalkenyl which may be substituted up to two times by methyl or represent a 5- to 7-membered saturated or partially saturated mono- or bicyclic heterocycle having a ring oxygen atom, and
   R²⁷ represents hydrogen, (C₁-C₄)-alkyl, cyano or trifluoromethyl,
R¹⁰ represents hydrogen, methyl or ethyl, and
   R¹¹ represents (C₁-C₆)-alkyl or (C₂-C₆)-alkenyl, each of which may be mono- to trisubstituted by substituents selected from, the group consisting of cyclopropyl, cyclobutyl, methoxy and fluorine.

Of very particular importance are compounds of the formula (I-B) 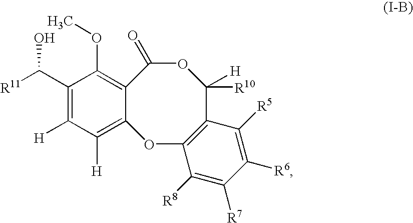
in which
R⁵, R⁶ and R⁷ independently of one another represent hydrogen, fluorine, chlorine, bromine, cyano or represent methyl or ethyl which may be substituted by methoxy or up to three times by fluorine,
R⁸ represents a group of the formula 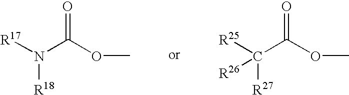
   where
   R¹⁷ and R¹⁸ independently of one another represent (C₁-C₆)-alkyl which may be substituted up to three times by fluorine, represent (C₃-C₆)-alkenyl or represent (C₃-C₆)-cycloalkyl, or
      together with the nitrogen atom to which they are attached form a 4- to 10-membered saturated mono- or bicyclic heterocycle which may contain an oxygen atom as further heteroatom and which may be substituted up to two times by methyl,
   R²⁵ and R²⁶ together with the carbon atom to which they are attached represent (C₃-C₁₀)-cycloalkyl which may be substituted up to four times by substituents selected from the group consisting of fluorine, methyl and trifluoromethyl, represent (C₅-C₇)-cycloalkenyl, 7-oxabicyclo[2.2.1]heptanyl or represent 7-oxabicyclo[2.2.1]hept-5-enyl, and
   R²⁷ represents methyl, ethyl, propyl, cyano or trifluoromethyl,
R¹⁰ represents hydrogen, methyl or ethyl and
R¹¹ represents (C₁-C₆)alkyl or (C₂-C₆)-alkenyl, each of which may be mono- to trisubstituted by substituents selected from the group consisting of cyclopropyl, cyclobutyl, methoxy and fluorine.

The known and the novel compounds of the general formulae (I), (I-A) and (I-B) can be prepared by processes described in EP-A-411 268. The contents of EP-A-411 268, in particular pages 9-17, are explicitly incorporated into the disclosure by way of reference. The natural product penicillide, which in some process variants serves as starting material, can be obtained by the method described in EP-A-411 268 [Compound (Ib)] from the strain Penicillium funiculosom Thorn. A culture of this strain was deposited on 8 Mar. 1989 at the Deutsche Sammlung für Mikroorganismen [German Collection of Microorganisms] in Brunswick under the number DSM 5249; this deposit was extended.

The compounds of the general formulae (I), (I-A) and (I-B) are obtained according to EP-A-411 268 by
[A] condensing, in inert solvents, compounds of the general formula (II) 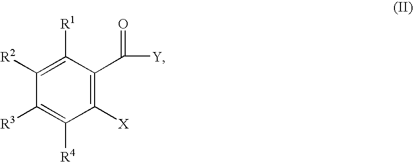
   in which
   R¹, R², R³ and R⁴ are each as defined above,
   X represents fluorine, chlorine, bromine or iodine and
   Y represents (C₁-C₆)-alkoxy or aryloxy having 6 to 10 carbon atoms,
   with compounds of the general formula (III) 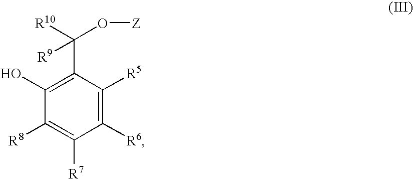
   in which
   R⁵, R⁶, R⁷, R⁸, R⁹ and R¹⁰ are each as defined above, and
   Z represents a typical hydroxyl protective group, such as, for example, tetrahydropyranyl,
   or compounds of the general formula (IIa) 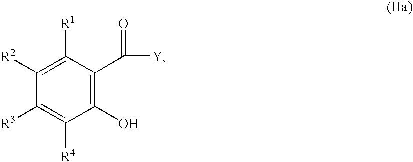
   in which
   R¹, R², R³, R⁴ and Y are each as defined above
   with compounds of the general formula (IIIa) 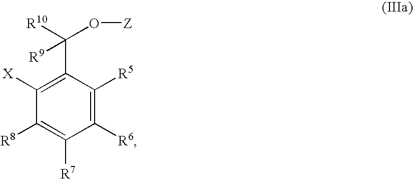
   in which
   R⁵, R⁶, R⁷, R⁸, R⁹, R¹⁰, X and Z are each as defined above
   with elimination of hydrogen halide, such as, for example, hydrogen bromide, to give compounds of the general formula (IV) 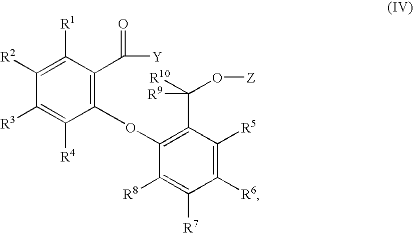
   in which
   R¹, R², R³, R⁴, R⁵, R⁶, R⁷, R⁸, R⁹, R¹⁰, Y and Z are each as defined above,
   followed by deblocking of the hydroxyl group by a customary method and by cyclization with elimination of water,
   where the substituents R¹, R², R³, R⁴, R⁵, R⁶, R⁷, R⁸, R⁹ and R¹⁰ can be introduced either (i) prior to the condensation into the compounds of the general formulae (II), (IIa), (III) and (IIIa), (ii) after the condensation into the compounds of the general formula (IV) or (iii) after the cyclization, according to known methods, such as, for example, by an alkylation, an acylation, a substitution, an addition, an elimination, a rearrangement, an oxidation, a free-radical reaction or a reduction, and then, if desired, be converted into other functional groups, or by
[B] introducing, into the natural product penicillide of the formula (Ib) 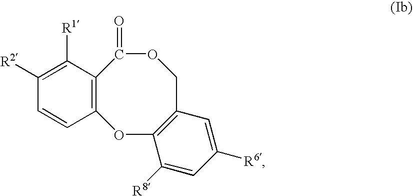
   in which
   R^{1′} represents methoxy,
   R^{2′} represents the group 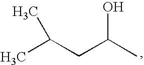
   R^{6′} represents methyl and
   R^{8′} represents hydroxy,
   using the methods mentioned under process [A], listed below by way of example, such as, for example, rearrangement, alkylation, acylation, addition, elimination, oxidation, free-radical reaction or reduction, in inert solvents, if appropriate in the presence of auxiliaries, such as, for example, bases, acids, catalysts or activating reagents, the substituents R¹, R², R³, R⁴, R⁵, R⁶, R⁷, R⁸, R⁹ and R¹⁰ and converting these, and also the substituents R^{1′}, R^{2′}, R^{6′} and R^{8′}, into other functional groups.

By way of example, the following reactions may be mentioned as suitable customary methods:
a) the natural product of the formula (Ib) or suitable derivatives prepared by the method described in process [A] are reacted once or more than once with compounds of the general formula (V)
   R-D (V),
   in which
   R corresponds to the meaning of one of the substituents R¹ to R¹⁰ listed above, but does not represent hydrogen, and
   D represents a leaving group, such as, for example, chlorine, bromine, iodine, —SO₂—CH₃ or —SO₂—(C₆H₅)-p-CH₃,
   in inert solvents, if appropriate in the presence of auxiliaries, such as, for example, bases, acids or catalysts, or
b) compounds of the formula (Ib) or suitable derivatives are reacted, for example, with amines, hydrazoic acid and diethyl azodicarboxylate, acetic acid, acetic anhydride, 3,4-dhydro-2H-pyran, thionyl chloride, methane-sulphonyl chloride, 2-pyrrolidinone-5-carboxylic acid or hydroxylamine in inert solvents, if appropriate in the presence of auxiliaries, such as bases or catalysts, or
c) are reacted with Grignard reagents of the general formula (VI)
   R—Mg—Br (VI),
   in which
   R is as defined above,
   in inert solvents, or
d) are halogenated with compounds of the general formula (VII)
   E-Hal (VII),
   in which
   Hal represents fluorine, chlorine, bromine or iodine and
   E represents one of the substituents R¹ to R¹⁰ listed above having the meaning fluorine, chlorine, bromine, iodine, or represents the radical —CH₂—NO₂,
   in inert solvents, and double bonds are then, if desired, introduced by elimination according to a known method, an epoxidation is carried out, if desired, and this is followed, if desired, by a reduction, oxidation or hydrolysis according to a customary method,
      thus introducing the substituents R¹ to R¹⁰ into the compounds of the general formula (Ib) and suitable derivatives, or converting the substituents R^{1′}, R^{2′}, R^{6′} and R^{8′} into other functional groups.

Depending on the nature of the starting materials used, the synthesis variations for the compounds according to the invention can be represented by the formula schemes below: 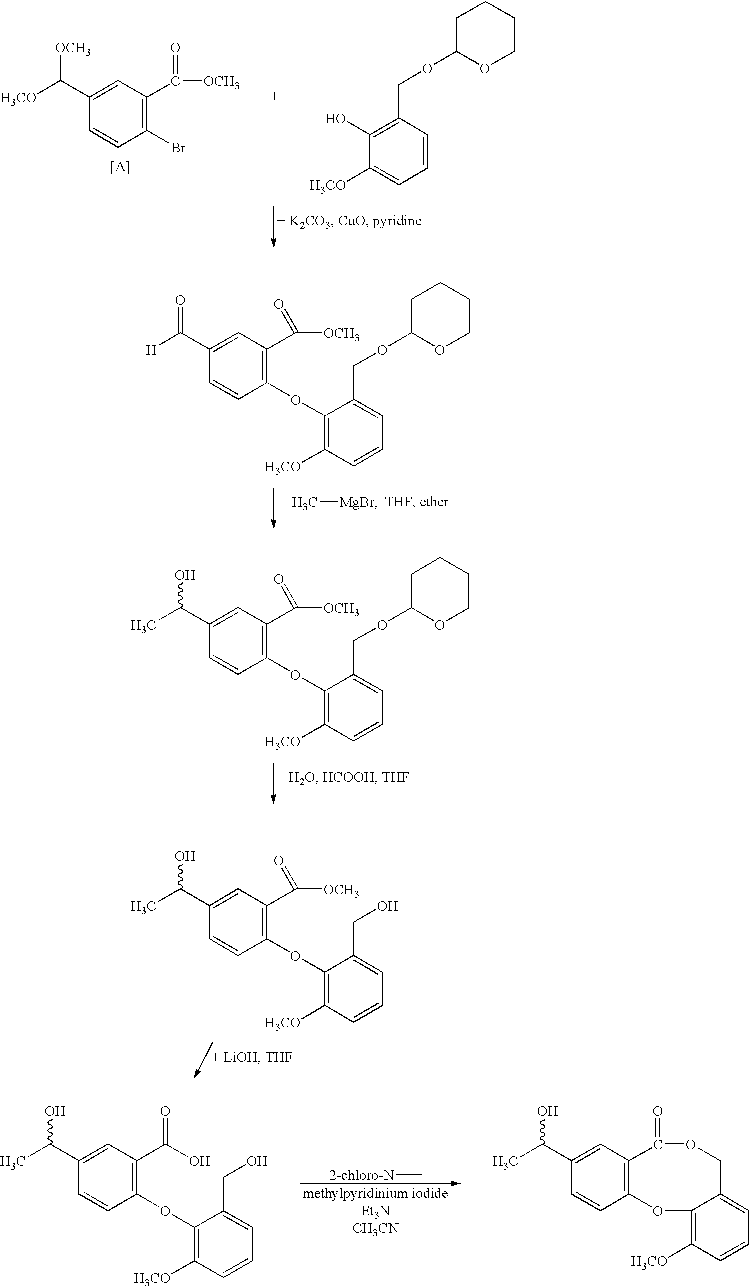 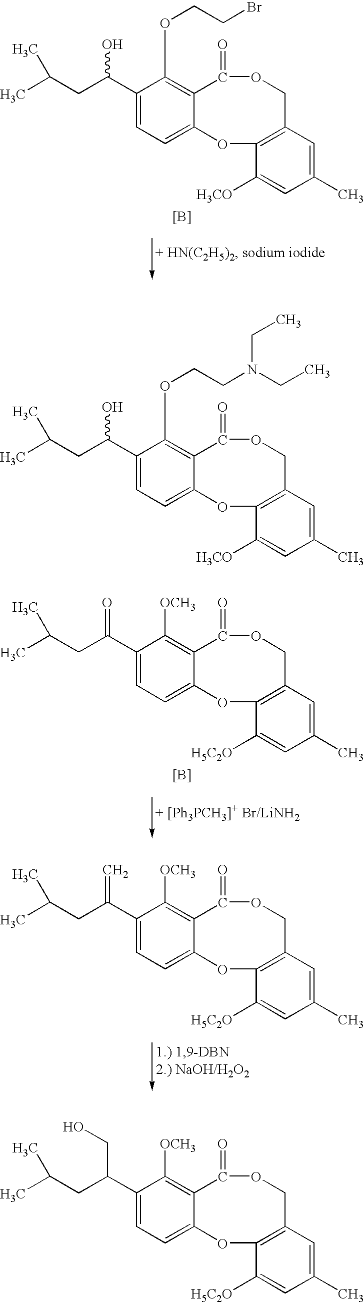 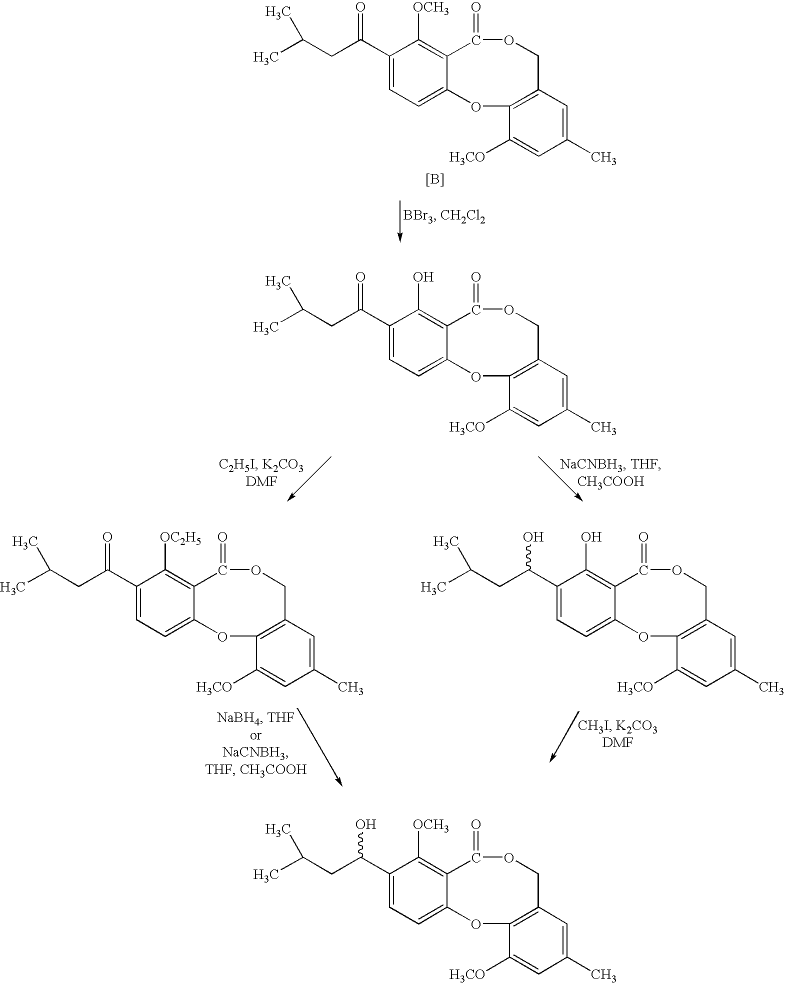
Processes [A] and [B]

Suitable for use as solvents for processes [A] and [B] are the customary organic solvents which do not change under the reaction conditions. These preferably include alcohols, such as methanol, ethanol, propanol or isopropanol, or ethers, such as diethyl ether, dioxane, tetrahydrofuran, glycol dimethyl ether or butyl methyl ether, or ketones, such as acetone or butanone, or amides, such as dimethylformamide or hexamethylphosphoric triamide, or carboxylic acids, such as acetic acid or propionic acid, or dimethyl sulphoxide, acetonitrile, ethyl acetate, or halogenated hydrocarbons, such as methylene chloride, chloroform or carbon tetrachloride, or pyridine, picoline or N-methylpiperidine. It is also possible to use mixtures of the solvents mentioned.

In all processes, the reaction temperatures can be varied within a relatively wide range. In general, the reactions are carried out between −20° C. and +200° C., preferably between +20° C. and +100° C., in particular at the boiling point of the solvent in question.

The reactions can be carried out under atmospheric pressure or else under elevated or reduced pressure. In general, the reactions are carried out under atmospheric pressure.

When carrying out process variants [A] and [B], any ratio of the substances involved in the reaction may be used. However, in general, the reactants are used in molar amounts. Isolation and purification of the substances according to the invention is preferably carried out by removing the solvent by distillation under reduced pressure and recrystallizing the residue, which may be obtained in crystalline form only after cooling with ice, from a suitable solvent. In some cases, it may be necessary to purify the compounds of the formula (I) by chromatography.

Suitable bases are the customary inorganic or organic bases. These preferably include alkali metal hydroxides, such as, for example, sodium hydroxide, lithium hydroxide or potassium hydroxide, or alkali metal carbonates, such as sodium carbonate or potassium carbonate, or alkali metal alkoxides, such as, for example, sodium methoxide or potassium methoxide, or sodium ethoxide or potassium ethoxide, or organic amines, such as triethylamine, picoline or N-methylpiperidine, or amides, such as sodium amide, lithium amide, lithium isopropylamide, or organometallic compounds, such as butyl lithium or phenyl lithium.

Suitable for use as catalysts in particular process variants are, for example, copper salts or oxides, preferably copper oxide and copper(II) acetate, or alkali metal iodides, such as sodium iodide or potassium iodide, which are added to the reaction mixture in an amount of from 0.5 to 150 mol, preferably from 5 to 50 mol.

Suitable for use as activating reagents are, for example, azodicarboxylic esters and triphenylphosphine, in molar ratios or in excess.

The condensation described in process [A] is carried out in one of the inert solvents mentioned above, with action of a base, preferably in pyridine with potassium carbonate, whereas for the cyclization, preference is given to using acetonitrile, triethylamine and 2-chloro-N-methylpyridinium iodide.

The auxiliaries used are preferably condensing agents, in particular when the carboxyl group is present in activated form as an anhydride. Preference is given here to the customary condensing agents, such as carbodiimides, for example N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride or 2-chloro-N-methyl-pyridinium iodide.

Introduction and removal of the hydroxyl protective group is carried out by known methods [Th. Greene, “Protective Groups in Organic Synthesis”, 1st edition, J. Wiley & Sons, New York, 1981]. The protective groups can be removed, for example, by acid or basic hydrolysis or by hydrogenolysis.

The alkylation is carried out in one of the inert solvents listed above, preferably in dimethylformamide in the presence of potassium carbonate.

The reduction is generally carried out using metal hydrides or borohydrides; preference is given to sodium borohydride and sodium cyanoborohydride in inert solvents such as ethers, preferably in tetrahydrofuran, diethyl ether or dioxane, in a temperature range of from −20° C. to +100° C., preferably from 0° C. to +50° C., at atmospheric pressure.

It is also possible to carry out the reduction by hydrogenation in inert solvents, such as alcohols, for example methanol, ethanol, propanol or isopropanol, in the presence of a noble metal catalyst, such as platinum, palladium, palladium on activated carbon or Raney nickel, in a temperature range of from 0° C. to +150° C., preferably from room temperature to +100° C., under atmospheric pressure or superatmospheric pressure.

The reduction of carbonyl groups to hydrocarbons is generally carried out using reducing agents such as zinc amalgam and acids such as hydrochloric acid, or using hydrazine hydrate and bases such as sodium hydroxide or potassium hydroxide, in the solvents listed above, preferably in ethers, such as tetrahydrofuran or diethyl ether. Aldoximes and ketoximes are generally reduced to the corresponding amines using the metal hydrides listed above, preferably with lithium aluminium hydride, or using zinc and acetic acid, boron hydride, sodium and alcohols, or by the catalytic hydrogenation mentioned above.

The reduction of alkoxycarbonyl groups to alcohol groups is generally carried out using hydrides, preferably with lithium aluminium hydride, in inert solvents, such as ethers or hydrocarbons or mixtures thereof, preferably in ethers, such as, for example, diethyl ether, tetrahydrofuran or dioxane, in a temperature range of from 0° C. to +150° C., preferably from +20° C. to +100° C., at atmospheric pressure.

The oxidation of alcohols to aldehydes and ketones is generally carried out using oxidizing agents, such as dichromate, potassium permanganate, bromine, manganese dioxide, pyridinium dichromate, dimethylpyrazole CrO₃ complex, silver carbonate on Celite, iodosobenzene, lead tetraacetate pyridine, pyridinium chlorochromate or Jones reagent, preferably with pyridinium chlorochromate, in the solvents listed above, preferably in a temperature range of from −20° C. to +100° C., preferably from 0° C. to +50° C., under atmospheric pressure.

The Wittig reactions are generally carried out by reaction with tetraalkyl- or tetraaryl-substituted phosphonium halides, preferably with triphenylmethylphosphonium bromide, in inert solvents, such as ethers, preferably in tetrahydrofuran, in the presence of a base, preferably lithium amide, in a temperature range of from −10° C. to +100° C., preferably at room temperature and atmospheric pressure.

The substitution reactions are generally carried out in the inert solvents listed above or in water, preferably in water, formic acid, methanol, ethanol, dimethylformamide or mixtures thereof, if appropriate in the presence of one of the bases or catalysts listed above, in a temperature range of from −60° C. to +200° C., preferably from 0° C. to +100° C., under atmospheric pressure.

The halogenation is carried out in one of the inert solvents listed above, preferably in dimethylformamide, in a temperature range of from −10° C. to +150° C., preferably from +25° C. to +80° C., under atmospheric pressure.

The reactions, which are not specifically mentioned, for introducing the substituents R¹ to R¹⁰, such as, for example, acylations, nucleophilic or electrophilic substitutions, free-radical reactions, eliminations and rearrangements, are carried out according to methods known from the literature [cf, for example, C. Ferr, Reaktionen der organischen Synthese [Reactions of organic synthesis], Georg Thieme Verlag, Stuttgart 1978; J. March, Advanced Organic Chemistry, Second edition, McGraw Hill].

The compounds of the general formulae (II), (IIa), (III), (IIIa) and (IV) are known per se and can be prepared by a customary method [cf. for example, Tietze and Eicher, Reaktionen und Synthesen im organisch-chemischen Praktikum [Reactions and syntheses in the organochemical laboratory course], Georg Thieme Verlag, Stuttgart, New York, 1981; W. Fuerer, H. W. Gschwend, J. Org. Chem. 44, 1133-1136 (1979); F. W. Vierhapper, E. Trengler, K. Kratzl, Monatshefte für Chemie 106, 1191-1201 (1975); J. A. Elix, V. Jayanthi, Aus. J. Chem. 40, 1841-1850 (1987)].

The compounds of the formula (Ib) can be isolated by customary methods from the strain Penicillium funiculosum Thorn [cf. Bodenwaschtechnik zur Isolierung von Boden- und Rhizosphärenpilzen, Methoden des mykologischen Laboratoriums [Techniques for washing soil for isolating soil and rhizosphere fungi, methods for the mycology laboratory], H. Kreisel, F. Schauer, Gustav Fischer Verlag, Stuttgart, New York, 1987]. One culture of the strain was deposited on Aug. 3, 1989 at the Deutsche Sammlung für Mikroorganismen in Brunswick under DSM 5249.

The compounds of the general formulae (V), (VI) and (VII) are known or can be prepared by known methods [cf. J. March, Advanced Organic Chemistry, Second edition, McGraw Hill].

Further novel synthesis variations for compounds according to the invention are, in an exemplary manner, shown in the reaction schemes below:
1. Starting Materials: 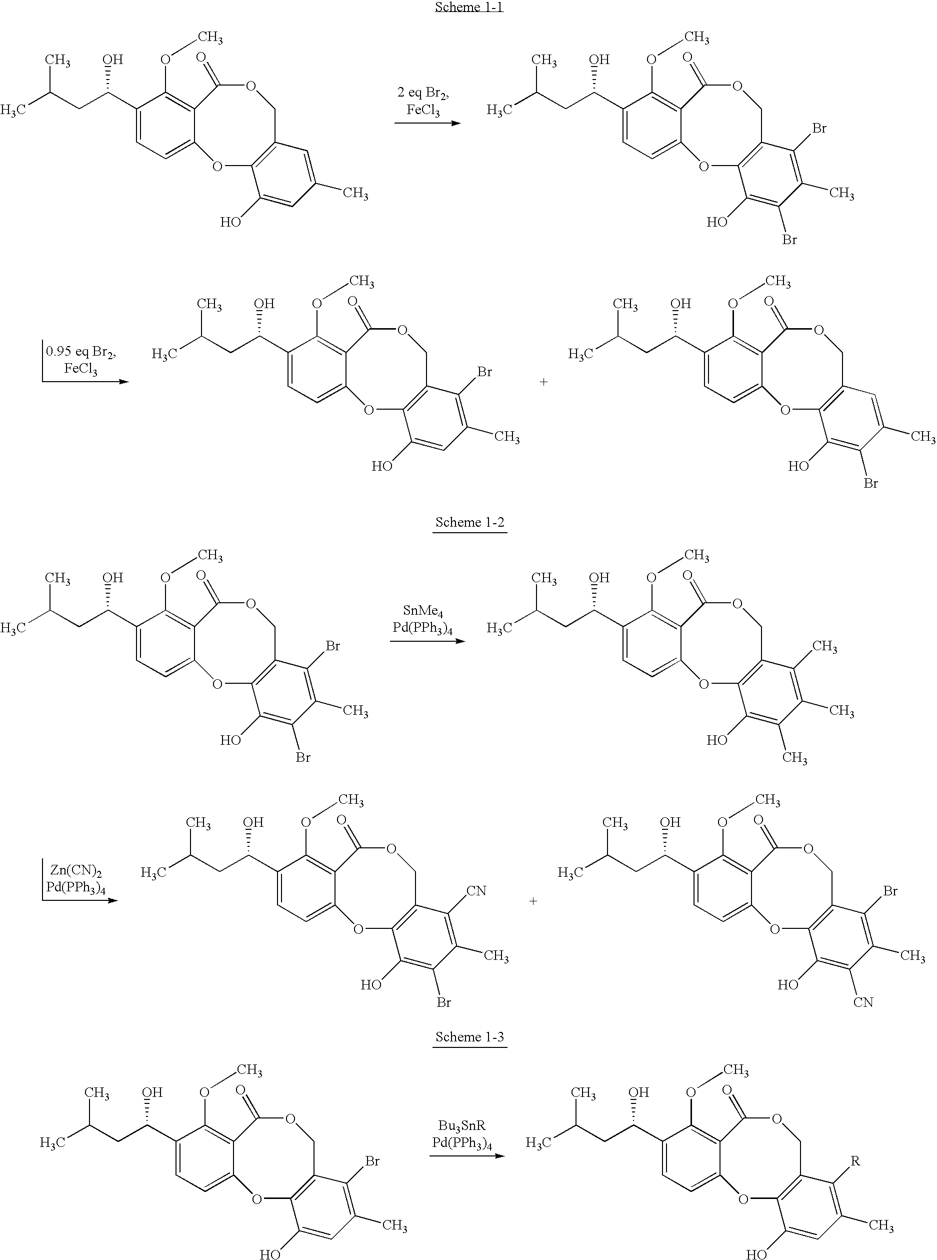 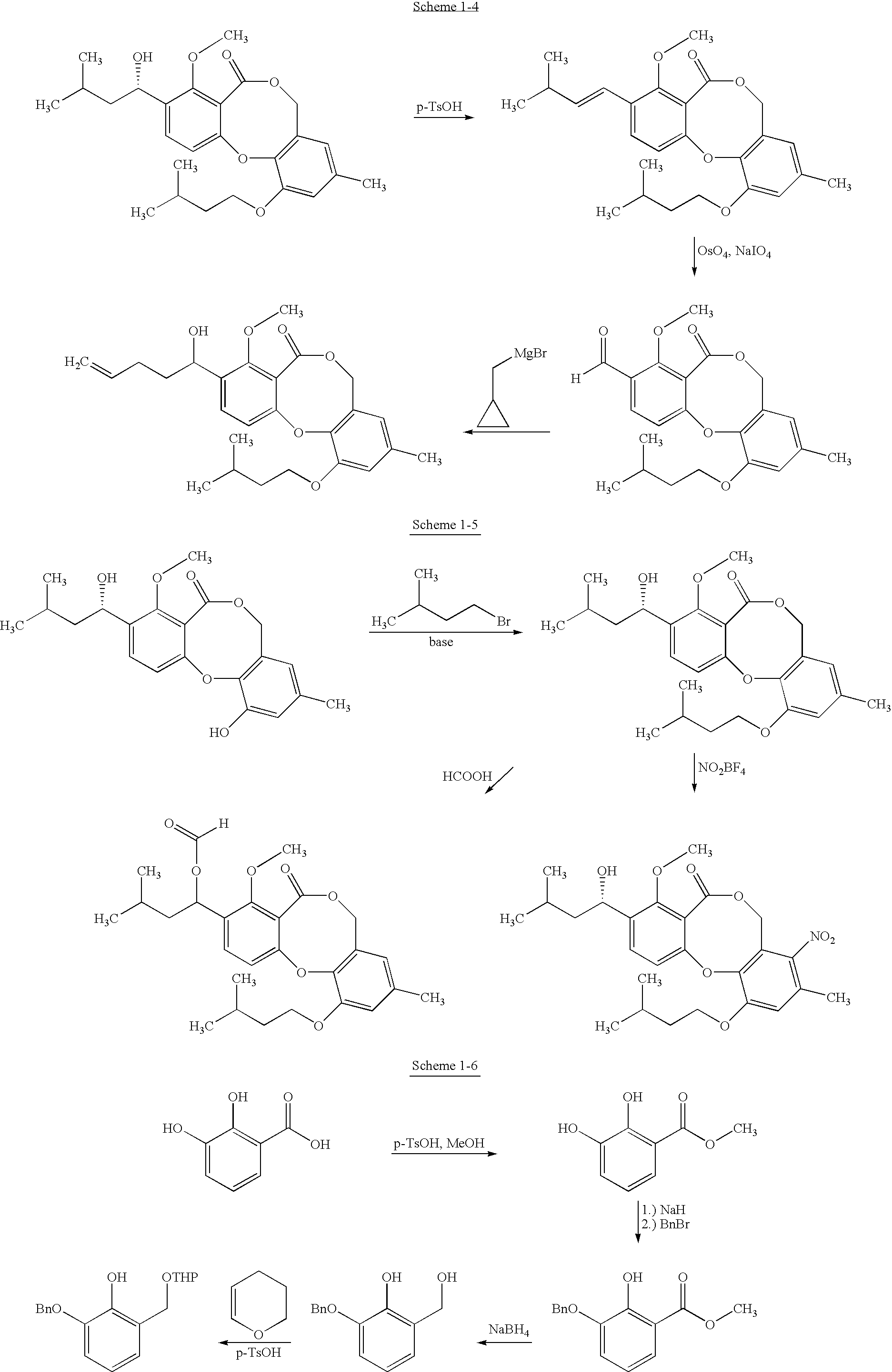 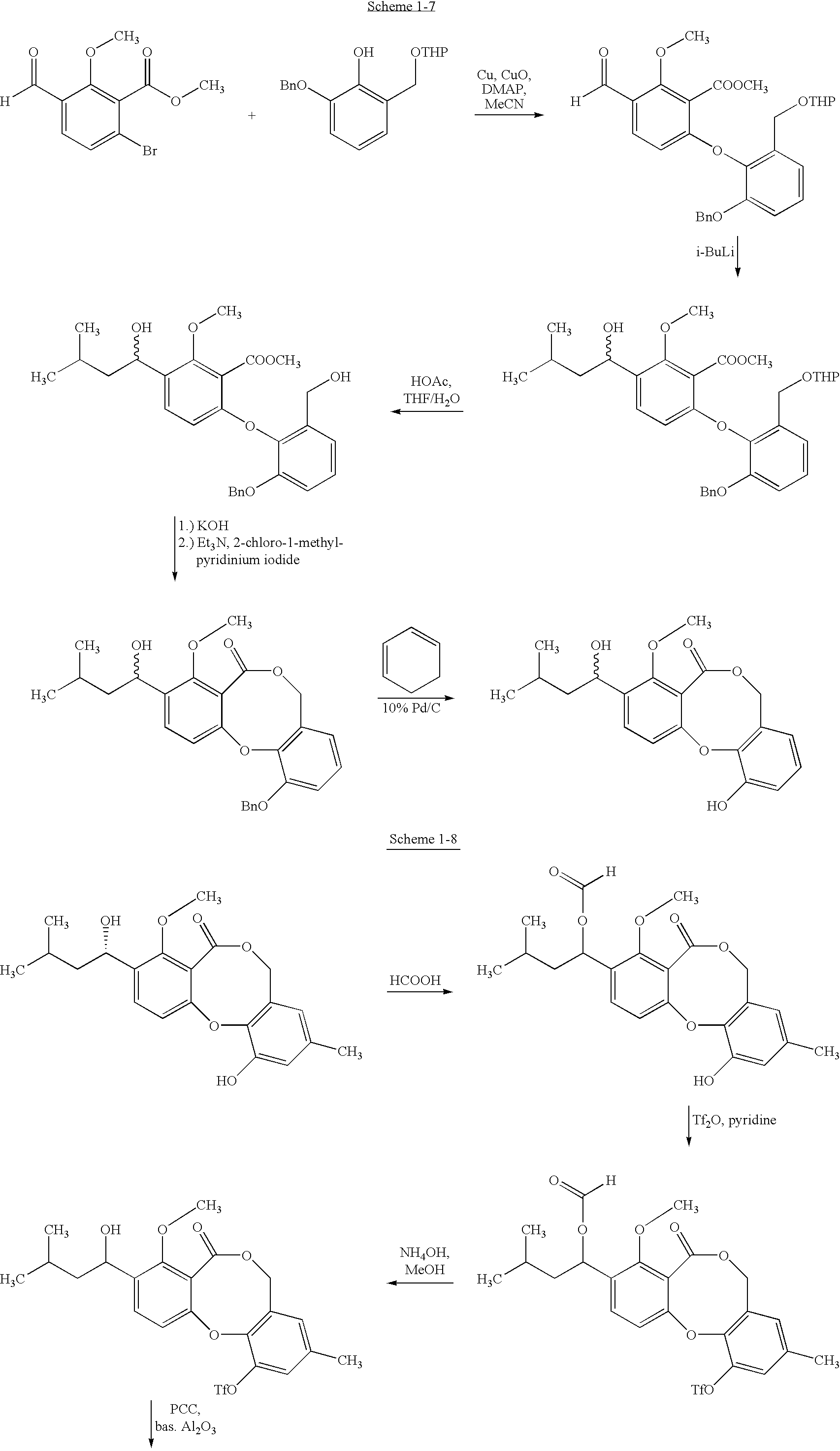 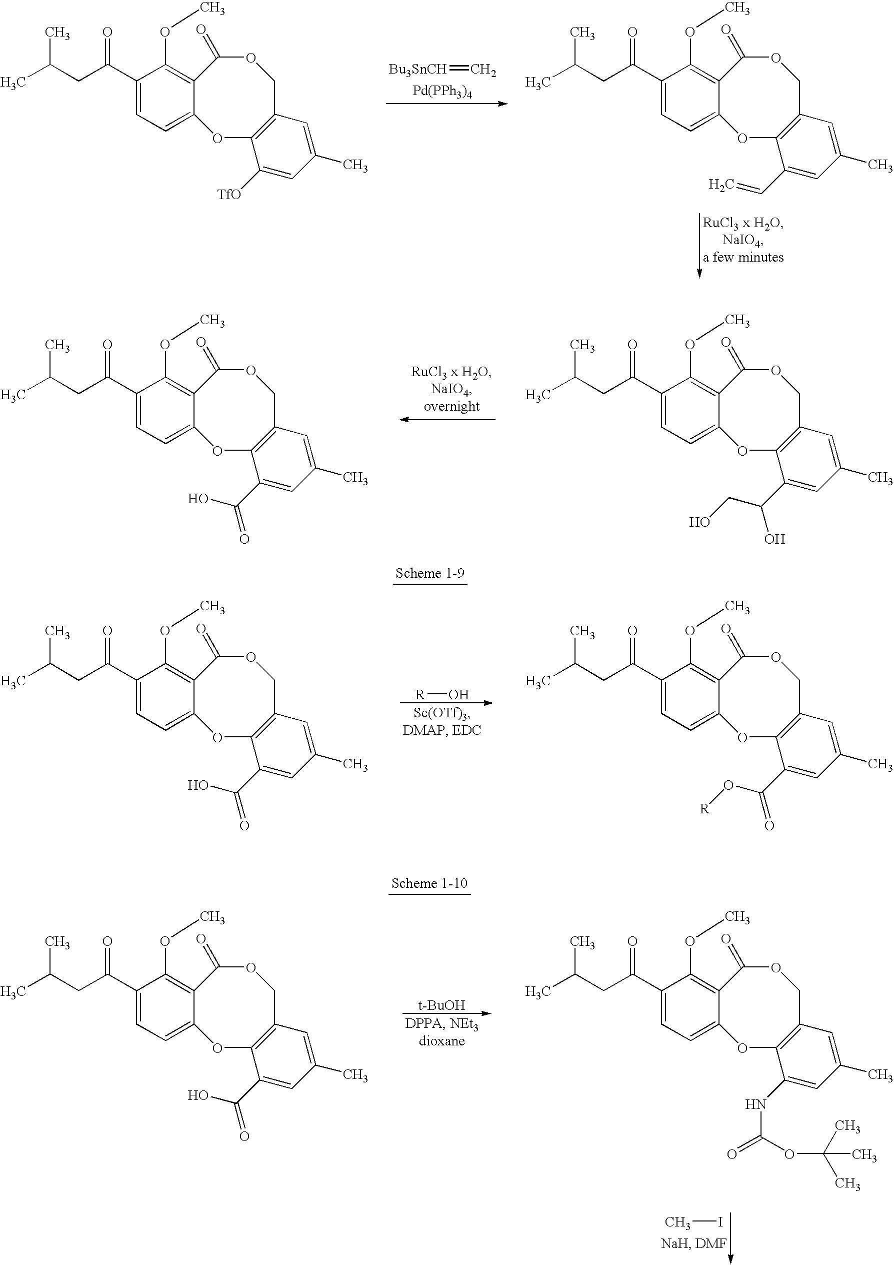 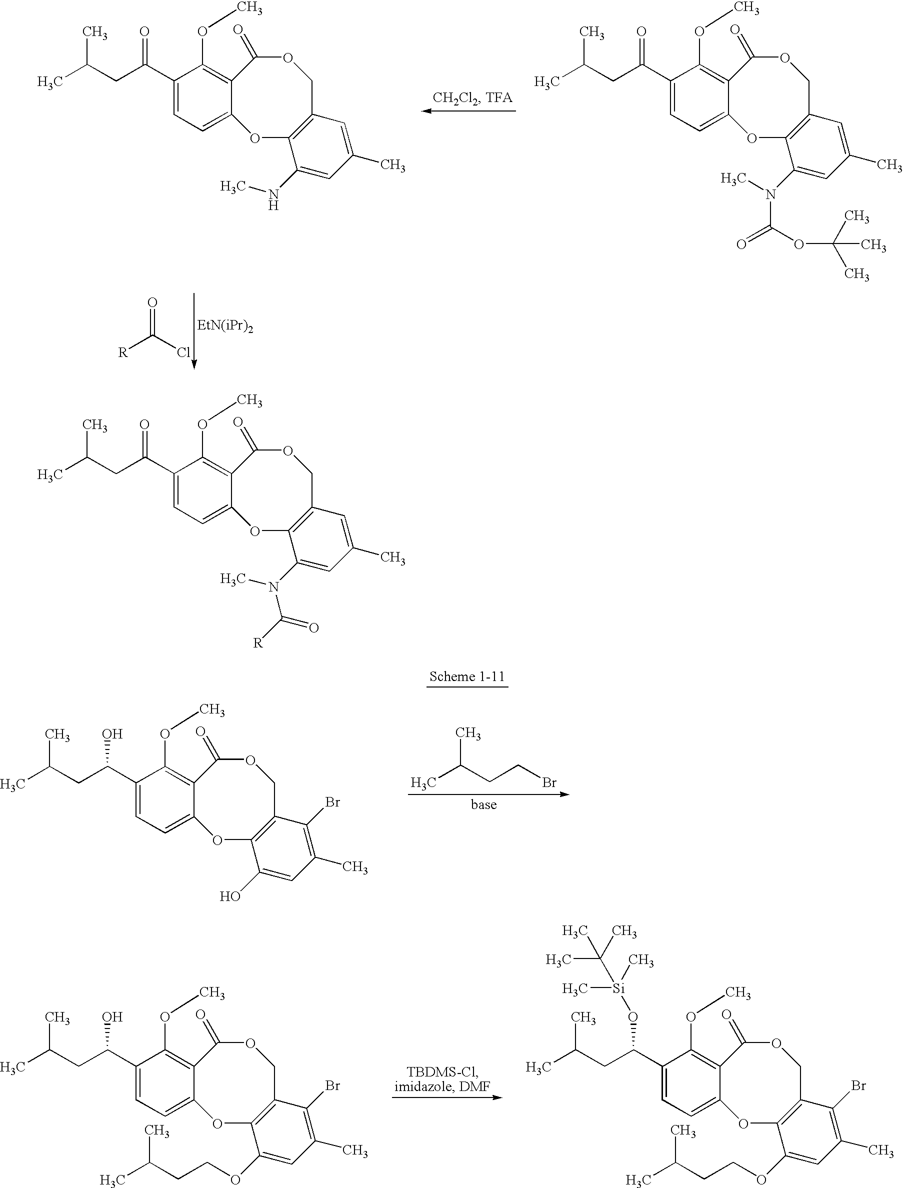 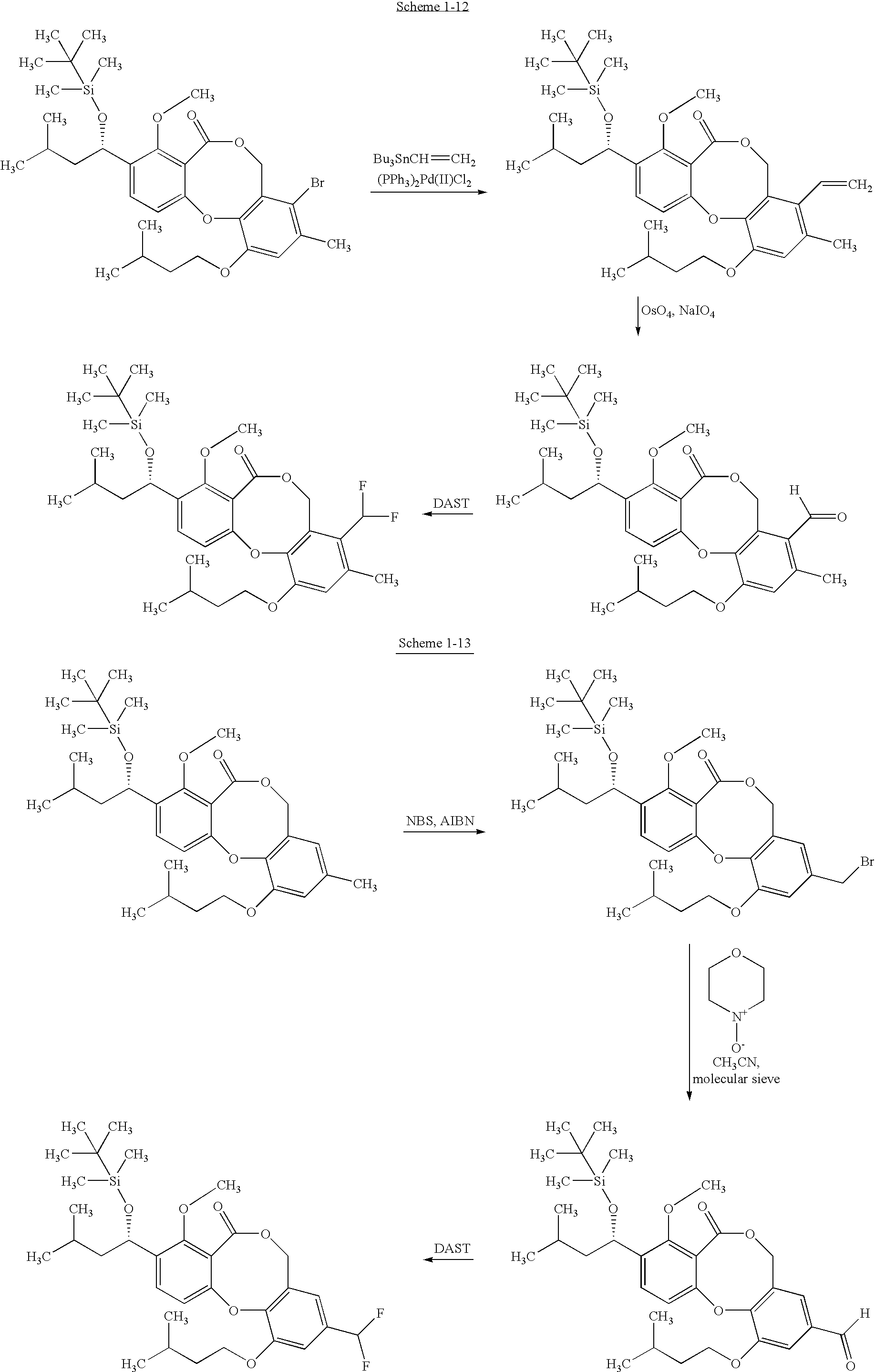 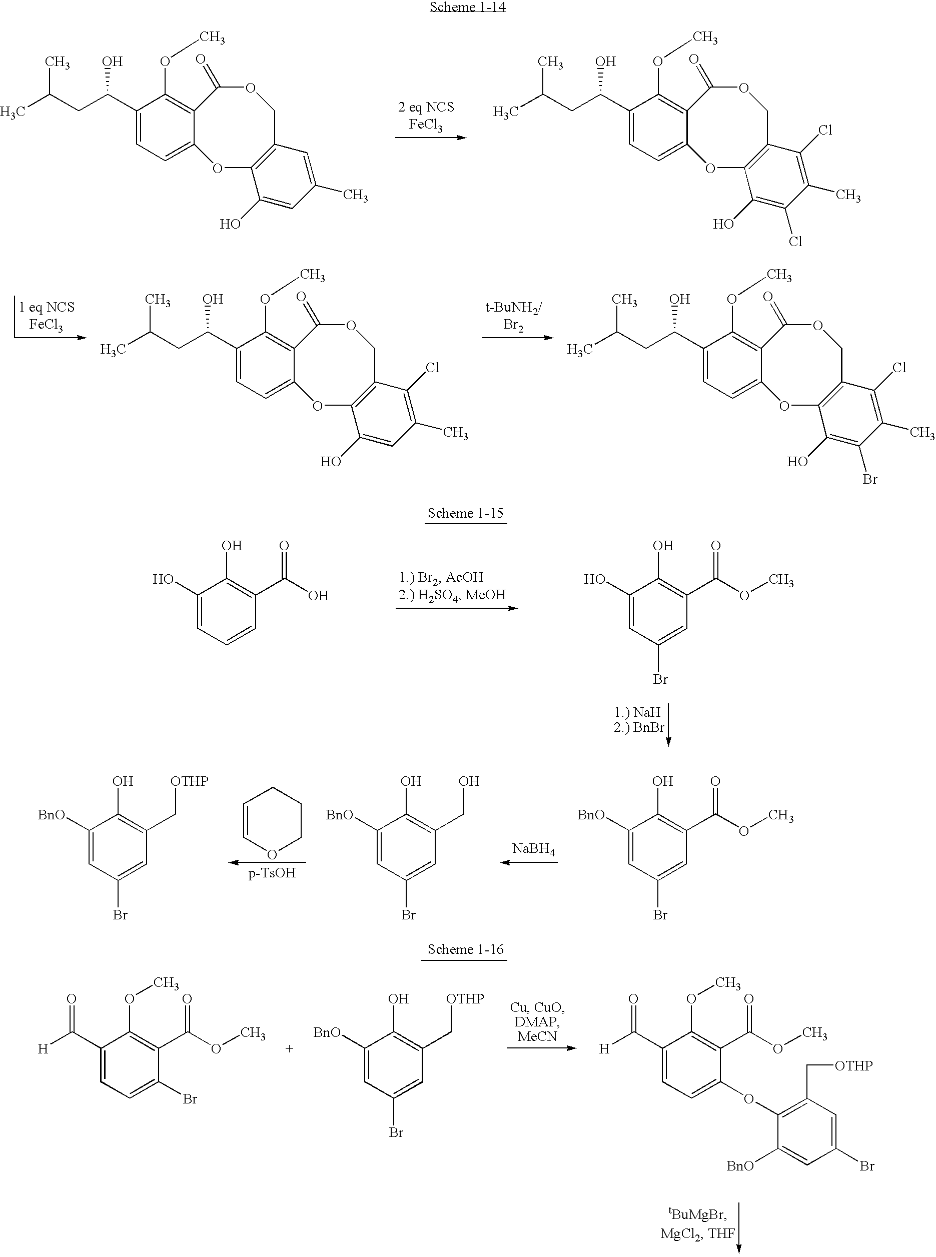 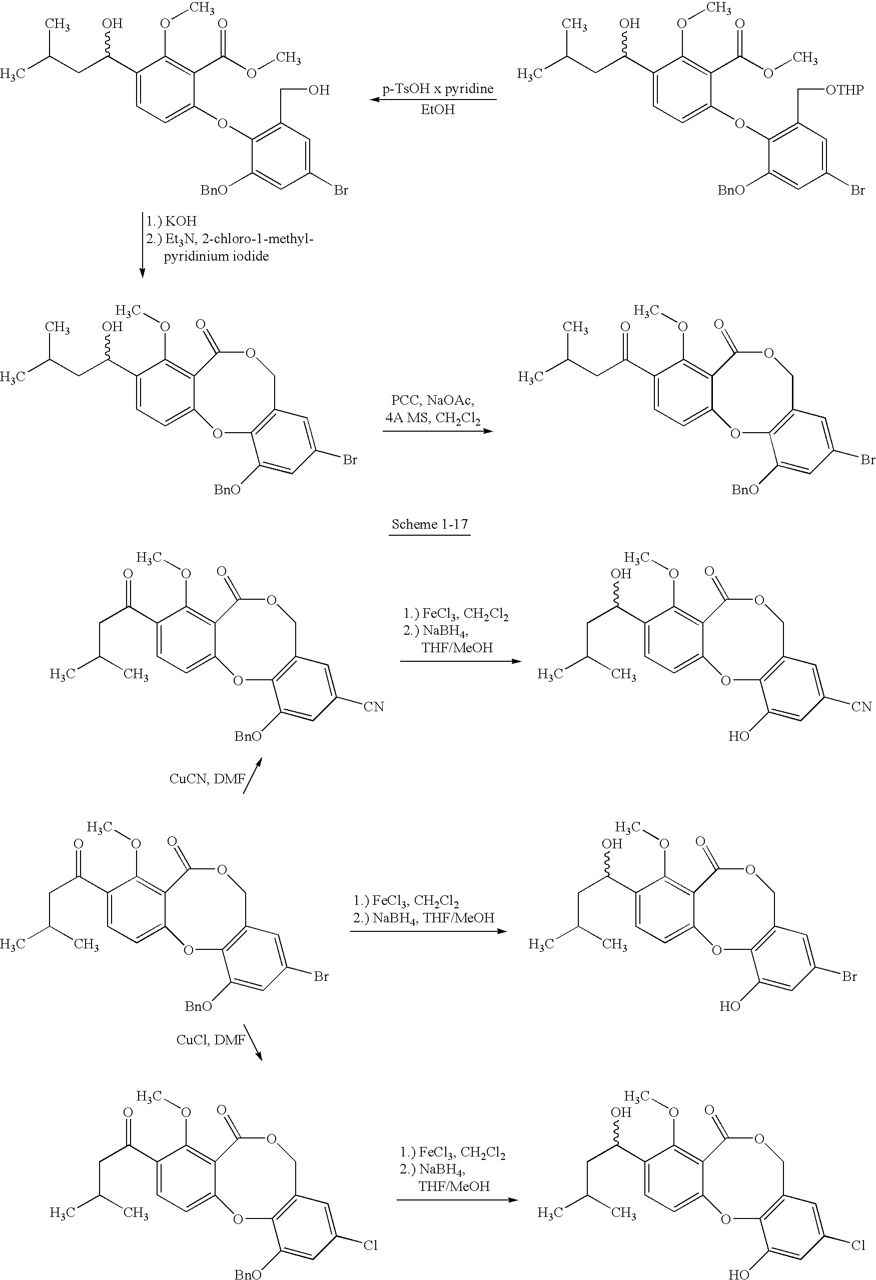 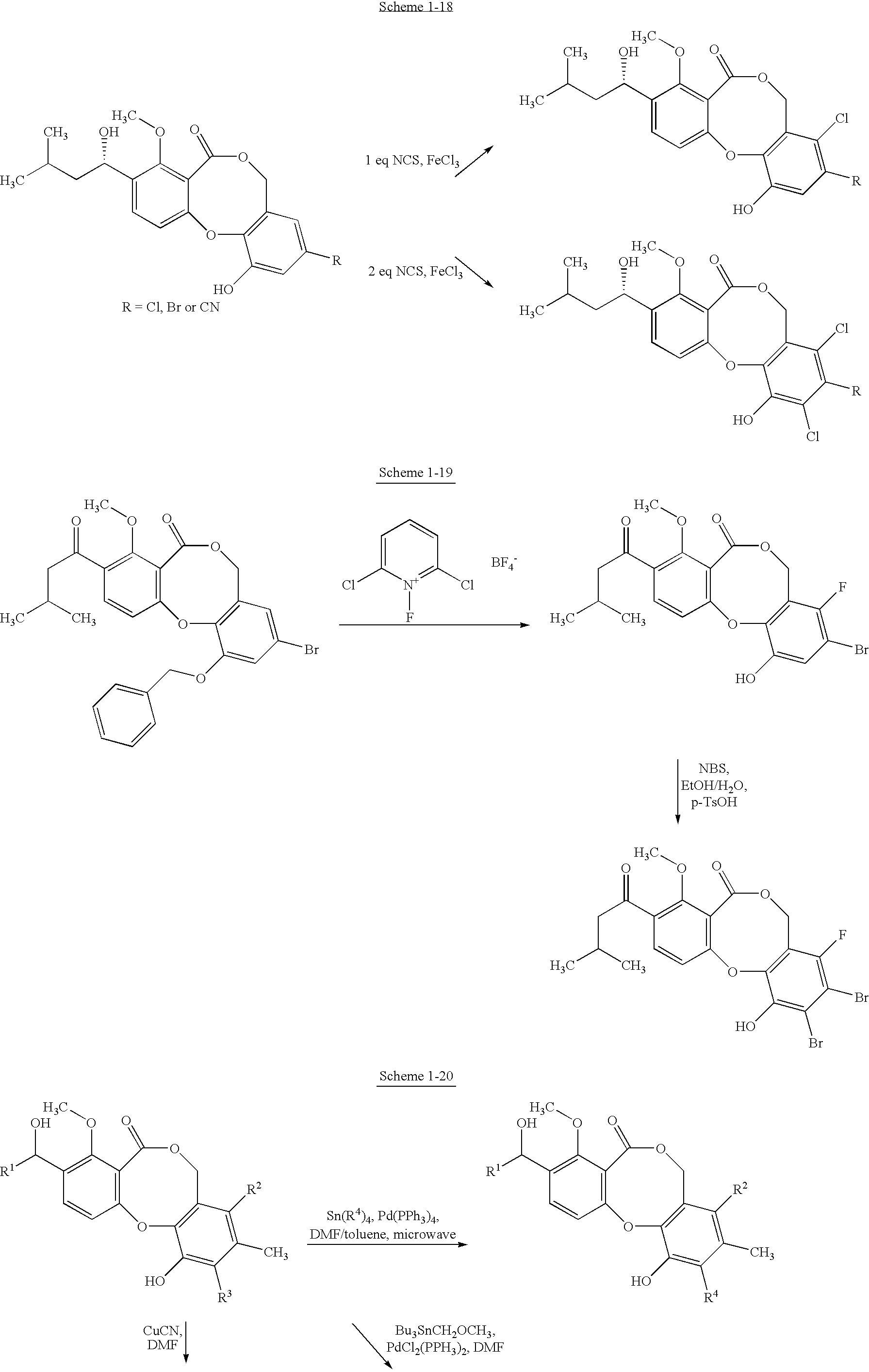 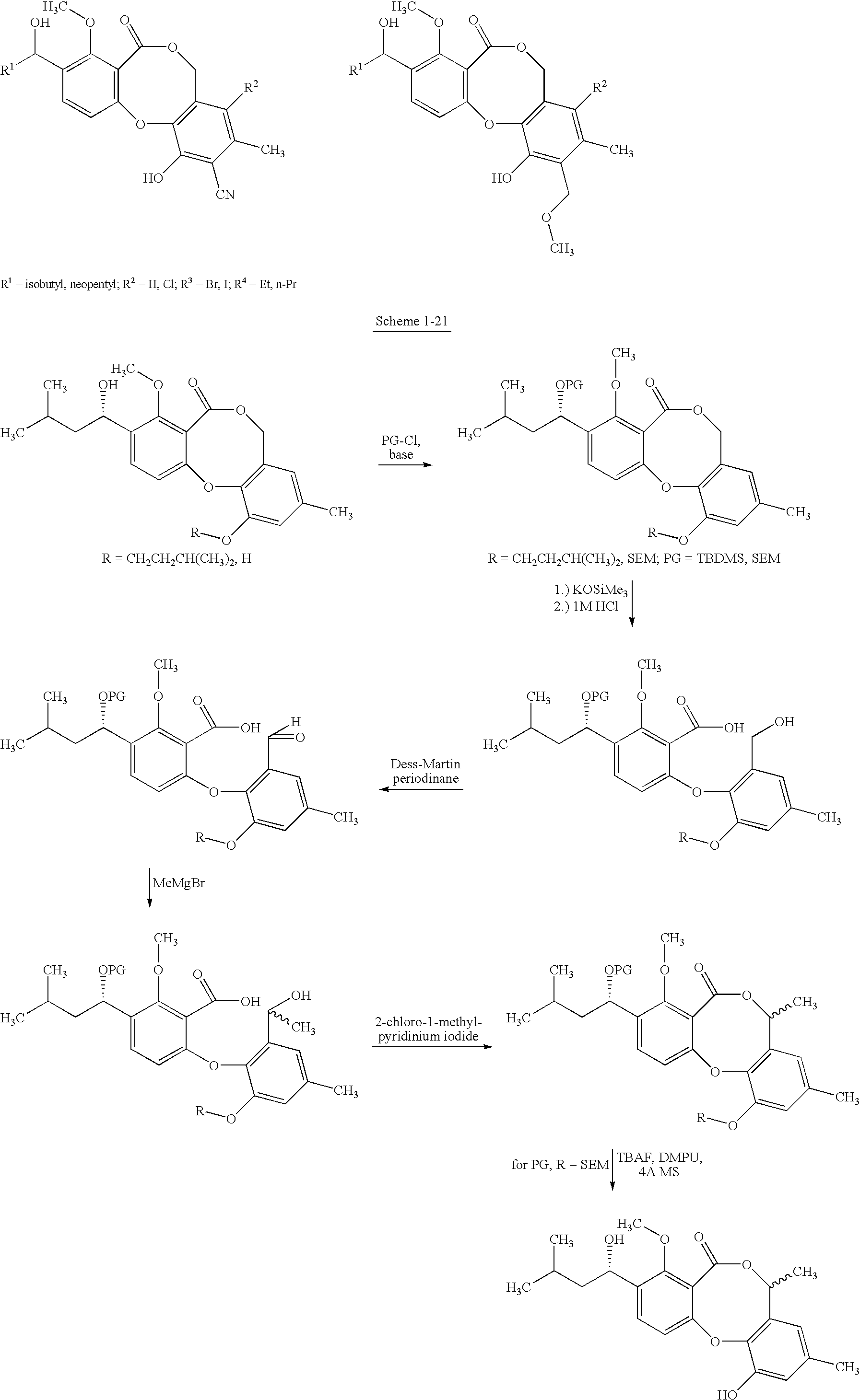
2. Preparation Examples: 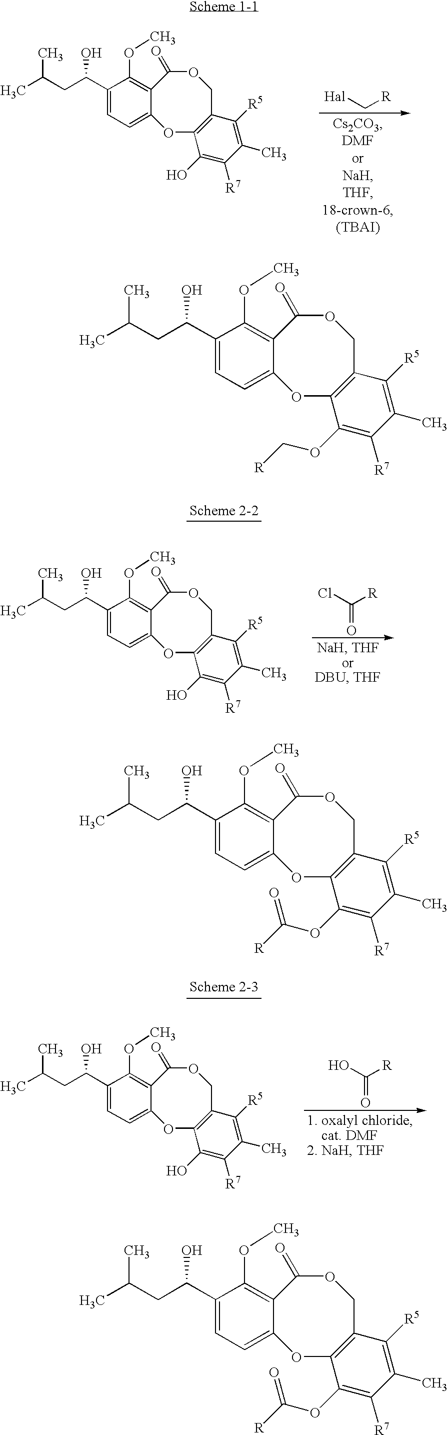 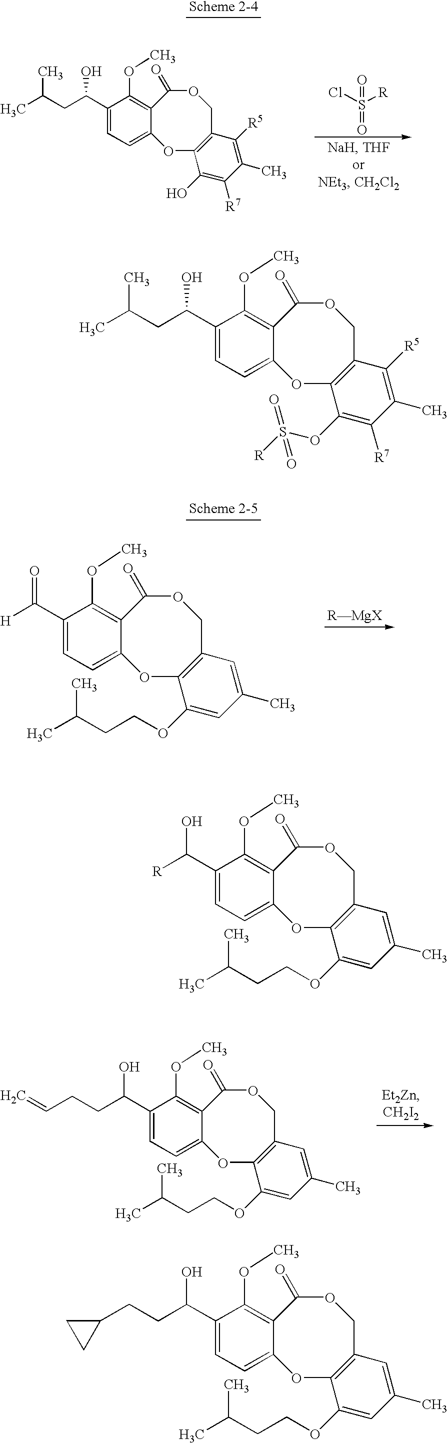 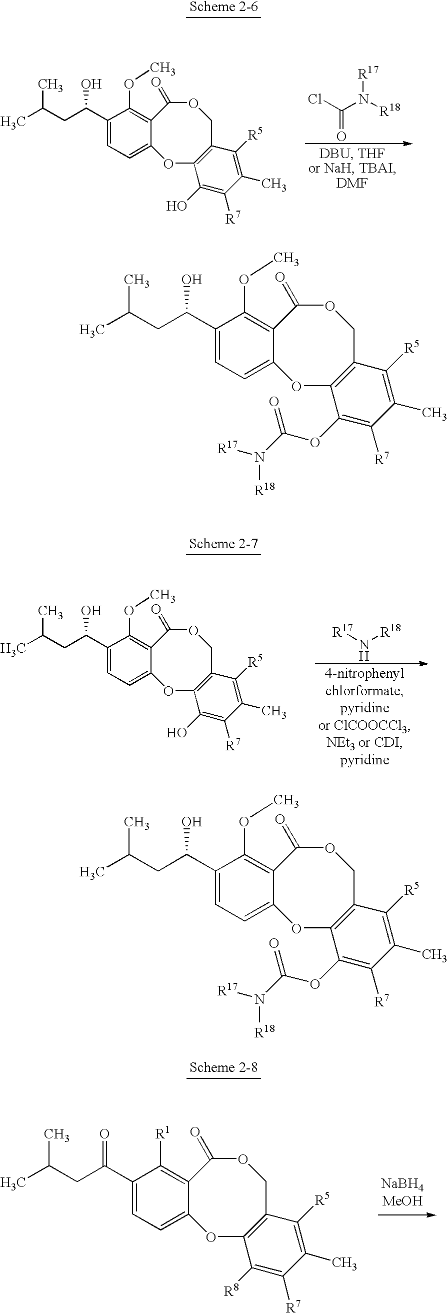 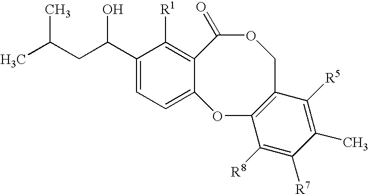
[Abbreviations:

Ac=acetyl; AIBN=α,α′-azobis(isobutyronitrile); Bn=benzyl; Bu=butyl; ⁱBu=isobutyl; cat.=catalytic; CDI=N,N′-carbonyldiimidazole; DAST=diethylamino-sulphur trifluoride; DBU=1,8-diazabicyclo[5.4.0]undec-7-ene; DMAP=4-N,N-dimethylaminopyridine; DMF=N,N-dimethylformamide; DMPU=N,N′-dimethyl-propyleneurea; DPPA=diphenylphosphoryl azide; EDC=N′-(3-dimethylamino-propyl)-N-ethylcarbodiimide hydrochloride; eq.=equivalent(s); Et=ethyl; Hal=halogen; HOAc=acetic acid; Me=methyl; 4A MS=4 Å molecular sieve; NBS=N-bromosuccinimide; NCS=N-chlorosuccinimide; PCC=pyridinium chlorochromate; PG=protecting group; Ph=phenyl; Pr=propyl; SEM=2-(trimethylsilyl)-ethoxymethyl; TBAF=tetrabutylammonium fluoride; TBAI=tetrabutylammonium iodide; TBDMS=tert-butyldimethylsilyl; TFA=trifluoroacetic acid; Tf=trifluoromethanesulphonyl; Tf₂O=trifluoromethanesulphonic anhydride; THF=tetrahydrofuran; THP=tetrahydropyranyl; p-TsOH=para-toluenesulphonic acid].

The compounds according to the invention have valuable pharmacological properties and can be used for the prevention and treatment of diseases. In particular, the compounds according to the invention are highly active inhibitors of the cholesterol ester transfer protein (CETP) and stimulate reverse cholesterol transport. The active compounds according to the invention cause a lowering of the LDL cholesterol level (low density lipoprotein) in the blood together with a simultaneous increase in the HDL cholesterol level (high density lipoprotein). They can therefore be employed for the treatment and prevention of hypolipoproteinaemia, dyslipidaemias, hyper-triglyceridaemias, hyperlipidaemias or arteriosclerosis. The active compounds according to the invention can moreover also be employed for the treatment and prevention of adiposity and obesity. The active compounds according to the invention are furthermore suitable for the treatment and prevention of stroke and of Alzheimer's disease.

The active compounds according to the invention open up a further treatment alternative and represent an enrichment of pharmacy. In comparison to the known and previously employed preparations, the compounds according to the invention show an improved spectrum of action. They are preferably distinguished by great specificity, good tolerability and fewer side-effects, in particular in the cardiovascular area.

The pharmacological action can be detected by means of known CETP inhibition tests.

The novel active compounds can be administered on their own and, if needed, also in combination with other active compounds, preferably from the group consisting of antidiabetics, antioxidants, cytostatics, calcium antagonists, hypotensive agents, thyromimetics, inhibitors of HMG-CoA reductase, inhibitors of HMG-CoA reductase gene expression, squalene synthesis inhibitors, ACAT inhibitors, circulation-promoting agents, platelet aggregation inhibitors, anticoagulants, angiotensin II receptor antagonists, cholesterol absorption inhibitors, MTP inhibitors, aldose reductase inhibitors, fibrates, niacin, anorectics, lipase inhibitors and PPAR agonists.

The combination of the compounds of the general formula (I) according to the invention with a glucosidase and/or amylase inhibitor for the treatment of familial hyperlipidaemias, adiposity and diabetes mellitus are preferred. Glucosidase and/or amylase inhibitors in the context of the invention are, for example, acarbose, adiposine, voglibose, miglitol, emiglitate, MDL-25637, camiglibose (MDL-73945), tendamistate, AI-3688, trestatin, pradimicin-Q and salbostatin.

The combination of acarbose, miglitol, emiglitate or voglibose with one of the above-mentioned compounds of the general formula (I) according to the invention is preferred.

The combination of the compounds according to the invention with cholesterol-lowering statins, HDL-raising principles, bile acid absorption blockers, cholesterol absorption blockers, vasoactive principles or ApoB-lowering principles in order to treat dyslipidaemias, combined hyperlipidaemias, hypercholesterolaemias or hyper-triglyceridaemias are furthermore preferred.

The combinations mentioned can also be employed for the primary or secondary prevention of coronary heart diseases (e.g. myocardial infarct).

Statins in the context of the invention are, for example, lovastatin, simvastatin, pravastatin, fluvastatin, atorvastatin, rosuvastatin and cerivastatin. ApoB-lowering agents are, for example, MTP inhibitors, vasoactive principles can be, for example—but not exclusively—adhesion inhibitors, chemokine receptor antagonists, cell proliferation inhibitors or substances having dilatory activity.

The combination of statins or ApoB inhibitors with one of the abovementioned compounds of the general formula (I) according to the invention is preferred.

The active compounds can act systemically and/or locally. For this purpose, they can be administered in a suitable manner, such as, for example, orally, parenterally, pulmonarily, nasally, sublingually, lingually, buccally, rectally, transdermally, conjunctivally, otically or as an implant.

For this administration route, the active compound can be administered in suitable administration forms.

For oral administration, known administration forms delivering the active compound rapidly and/or in modified form, such as, for example, tablets (uncoated and coated tablets, e.g. tablets provided with enteric coatings or film-coated tablets), capsules, sugar-coated tablets, granules, pellets, powders, emulsions, suspensions and solutions, are suitable.

Parenteral administration can be carried out with avoidance of an absorption step (intravenous, intra-arterial, intracardiac, intraspinal or intralumbal) or with involvement of an absorption (intramuscular, subcutaneous, intracutaneous, percutaneous, or intraperitoneal). Suitable administration forms for parental administration are, inter alia, injection and infusion preparations in the form of solutions, suspensions, emulsions, lyophilisates and sterile powders.

For the other administration routes, for example, pharmaceutical forms for inhalation (inter alia powder inhalers, nebulizers), nasal drops/solutions, sprays; tablets or capsules to be administered lingually, sublingually or buccally or capsules, suppositories, aural and ophthalmic preparations, vaginal capsules, aqueous suspensions (lotions, shake mixtures), lipophilic suspensions, ointments, creams, milk, pastes, dusting powder or implants are suitable.

The novel active compounds of the invention are used for the production of medicaments, in particular for the production of medicaments for the prevention and treatment of the abovementioned diseases.

Medicaments are prepared in a known manner by converting the compounds according to the invention into the customary formulations, such as tablets, coated tablets, pills, granules, aerosols, syrups, emulsions, suspensions and solutions. This is carried out using inert non-toxic, pharmaceutically suitable excipients. These include, inter alia, vehicles (e.g. microcrystalline cellulose), solvents (e.g. liquid polyethylene glycols), emulsifiers (e.g. sodium dodecyl sulphate), dispersing agents (e.g. polyvinylpyrrolidone), synthetic and natural biopolymers (e.g. albumin), stabilizers (e.g. antioxidants such as ascorbic acid), colorants (e.g. inorganic pigments such as iron oxides) or taste and/or odour corrigents. In this connection, the therapeutically active compound should in each case be present in a concentration of approximately 0.5 to 90% by weight of the total mixture, i.e. in amounts which are sufficient in order to achieve the dosage range indicated.

The formulations are prepared, for example, by extending the active compounds using solvents and/or vehicles, if appropriate using emulsifiers and/or dispersing agents, where, for example, if water is used as a diluent, organic solvents can optionally be used as auxiliary solvents.

Intravenous, parenteral, perlingual and in particular oral administration are preferred.

In the case of parenteral administration, solutions of the active compound using suitable liquid vehicles can be employed.

In general, it has proved advantageous in the case of intravenous administration to administer amounts of approximately 0.001 to 1 mg/kg, preferably approximately 0.01 to 0.5 mg/kg of body weight, to achieve efficaceous results, and in the case of oral administration the dose is approximately 0.01 to 100 mg/kg, preferably 0.01 to 20 mg/kg and very particularly preferably 0.1 to 10 mg/kg of body weight.

In spite of this, if appropriate it may be necessary to depart from the amounts mentioned, namely depending on the body weight or the type of administration route, on individual behaviour towards the medicament, the manner of its formulation and the time or interval at which administration takes place. Thus in some cases it may be sufficient to manage with less than the abovementioned minimum amount, while in other cases the upper limit mentioned has to be exceeded. In the case of the administration of relatively large amounts, it may be advisable to divide these into a number of individual doses over the course of the day.

The following examples serve to illustrate the invention. The invention is not thereby restricted to the examples.

Abbreviations:

TLC Thin-layer chromatography

DCI Direct chemical ionization (MS)

DMF N,N-Dimethylformamide

DMPU N,N′-Dimethylpropyleneurea

eq. equivalent(s)

ESI Electrospray ionization (MS)

m.p. Melting point

GC-MS Gas-chromatography-coupled mass spectroscopy

HPLC High-pressure, high-performance liquid chromatography

LC-MS Liquid-chromatography-coupled mass spectroscopy

MS Mass spectroscopy

NMR Nuclear magnetic resonance spectroscopy

R_{f} Retention index (TLC)

RP reverse phase (HPLC)

RT Room temperature

Rₜ Retention time (HPLC)

THF Tetrahydrofuran

Analytical Methods:

Method 1:

Instrument: HP 1100 with DAD detection; column: Kromasil RP-18, 60 mm×2 mm, 3.5 μm; mobile phase A=5 ml HClO₄/l H₂O, mobile phase B=acetonitrile; gradient: 0 min 2% B, 0.5 min 2% B, 4.5 min 90% B, 6.5 min 90% B; flow rate: 0.75 ml/min; temp.: 30° C.; UV detection: 210 nm.

Method 2:

Instrument: HP 1100 with DAD detection; column: Kromasil RP-18, 60 mm×2 mm, 3.5 μm; mobile phase A=5 ml HClO₄/l H₂O, mobile phase B=acetonitrile; gradient: 0 min 2% B, 0.5 min 2% B, 4.5 min 90% B, 9 min 90% B; flow rate: 0.75 ml/min; temp.: 30° C.; UV detection: 210 nm.

Method 3:

Instrument: Micromass TOF-MUX-Interface 4-fold parallel injection, Waters 600; column: YMC-ODS-AQ, 50 mm×2.1 mm, 3.0 μm; mobile phase A=water+0.05% formic acid, mobile phase B=acetonitrile+0.05% formic acid; gradient: 0.0 min 100% A→0.2 min 100% A→2.9 min 30% A→3.1 min 10% A→4.5 min 10% A; oven: room temperature; flow rate: 0.8 ml/min; UV detection: 210 nm.

Method 4:

Instrument: Finnigan MAT 900S, TSP: P4000, AS3000, UV3000HR; column: symmetry C18, 150 mm×2.1 mm, 5.0 μm; mobile phase C=water, mobile phase B=water+0.3 g/l 35% strength HCl, mobile phase A=acetonitrile; gradient: 0.0 min 2% A→2.5 min 95% A→5 min 95% A; oven: 70° C.; flow rate: 1.2 ml/min; UV detection: 210 nm.

Method 5:

Instrument: Micromass Platform LCZ, HP1100; column: symmetry C18, 50 mm×2.1 mm, 3.5 μm; mobile phase A=acetonitrile+0.1% formic acid, mobile phase B=water+0.1% formic acid; gradient: 0.0 min 10% A→4.0 min 90% A→6.0 min 90% A; oven: 40° C.; flow rate: 0.5 ml/min; UV detection: 208-400 nm.

Method 6:

Instrument: Micromass Quattro LCZ, HP1100; column: symmetry C18, 50 mm×2.1 mm, 3.5 μm; mobile phase A=acetonitrile+0.1% formic acid, mobile phase B=water+0.1% formic acid; gradient: 0.0 min 10% A→4.0 min 90% A→6.0 min 90% A; oven: 40° C.; flow rate: 0.5 ml/min; WV detection: 208-400 nm.

Method 7:

Instrument: Finnigan MAT 900S, TSP: P4000, AS3000, UV3000HR; column: symmetry C18, 150 mm×2.1 mm, 5.0 μm; mobile phase C=water, mobile phase B=water+0.6 g/l 35% strength HCl, mobile phase A=acetonitrile; gradient: 0.0 min 10% A→3 min 90% A→6 min 90% A; oven: 70° C.; flow rate: 1.2 ml/min; UV detection: 210 nm.

Method 8:

Instrument: HP 1100 with DAD detection; column: Kromasil RP-18, 60 mm×2 mm, 3.5 μm; mobile phase A=5 ml HClO₄/l H₂O, mobile phase B=acetonitrile; gradient: 0 min 2% B, 0.5 min 2% B, 4.5 min 90% B, 15 min 90% B; flow rate: 0.75 ml/min; temp.: 30° C.; UV detection: 210 nm.

Method 9:

Instrument: Micromass GCT, GC6890; column: Restek RTX-35MS, 30 m×250 μm×0.25 μm; constant flow rate with helium: 0.88 ml/min; oven: 60° C.; inlet: 250° C.; gradient: 60° C. (maintained for 0.30 min), 50° C./min→120° C., 16° C./min→250° C., 30° C./min→300° C. (maintained for 1.7 min).

Method 10:

MS Instrument: Micromass ZQ; HPLC instrument: Waters Alliance 2795; column: Phenomenex Synergi 2μ Hydro-RP Mercury 20×4 mm; mobile phase A: 11 water+0.5 ml 50% strength formic acid, mobile phase B: 11 acetonitrile+0.5 ml 50% strength formic acid; gradient: 0.0 min 90% A, flow rate 1 ml/min→2.5 min 30% A, flow rate 2 ml/min→3.0 min 5% A, flow rate 2 ml/min→4.5 min 5% A, flow rate 2 ml/min; oven: 50° C.; UV detection: 210 nm.

General Method for Preparative HPLC:

Column: Kromasil C18, 250 mm×20, 25, 30 or 40 mm; mobile phase A=water+1% formic acid, mobile phase B=acetonitrile; gradient: 90-95% A→100% B; flow rate: 10-50 ml/min; temp.: room temperature; UV detection: 210-254 nm.

Part A:

Starting Materials:

### EXAMPLE A-I

### 8,10-Dibromo-11-hydroxy-3-[(1S)-1-hydroxy-3-methylbutyl]-4-methoxy-9-methyl-5H,7H-dibenzo[b,g][1,5]dioxocin-5-one

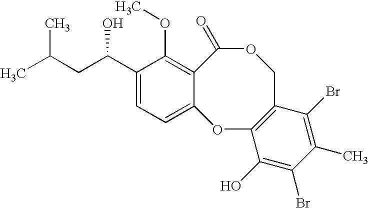

1 g of (2.69 mmol) of penicillide [T. Sassa et al., Agr. Biol. Chem. 37, 1221 (1973), Tetrahedron Lett., 2333 (1973), Tetrahedron Lett., 3941 (1974); Compound (Ib) in EP-A-411 268] is dissolved in 15 ml of ethanol. 436 mg (2.69 mmol) of iron trichloride are dissolved in 5 ml of water and added dropwise to the reaction solution. 277 μl (5.37 mmol) of bromine are then added, and the mixture is stirred at room temperature overnight. The reaction mixture is diluted with dichloromethane and washed once with 10% strength potassium iodide solution, once with water, once with 10% bisulphite solution and once with water. The organic phase is dried over sodium sulphate and concentrated under reduced pressure. This gives 1.33 g (93% of theory) of product.

¹H-NMR (300 MHz, CDCl₃): δ=0.98 (t, 6H), 1.18-1.31 (m, 1H), 1.43-1.52 (m, 1H), 1.63-1.72 (m, 1H), 1.75-1.85 (m, 1H), 2.58 (s, 3H), 3.99 (s, 3H), 5.09 (dd, 1H), 5.43 (q, 2H), 6.52 (br. s, 1H), 6.87 (d, 1H), 7.60 (d, 1H) ppm.

MS (DCI): m/z=548 (M+NH₄)⁺

HPLC (Method 1): Rₜ=5.21 min.

### EXAMPLE A-II AND EXAMPLE A-III

### 8-Bromo-11-hydroxy-3-[(1S)-1-hydroxy-3-methylbutyl]-4-methoxy-9-methyl-5H,7H-dibenzo[b,g][1,5]dioxocin-5-one (Example A-II) and 10-Bromo-11-hydroxy-3-[(1S)-1-hydroxy-3-methylbutyl]-4-methoxy-9-methyl-5H,7H-dibenzo[b,g][1,5]dioxocin-5-one (Example A-III)

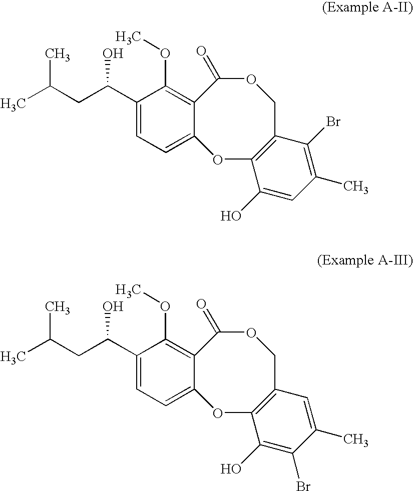

At 0° C., 1 g (2.69 mmol) of penicillide is dissolved in 15 ml of ethanol, and 436 mg (2.69 mmol) of iron trichloride, dissolved in 5 ml of water, are added. 131 μl (2.55 mmol) of bromine, dissolved in 2 ml of ethanol, are then added dropwise over a period of 30 minutes, and the mixture is stirred at room temperature for 10 hours. The reaction solution is diluted with dichloromethane and washed once with 10% strength potassium iodide solution, once with water, once with 10% bisulphite solution and once with water. The organic phase is dried over sodium sulphate and concentrated under reduced pressure. The residue is purified by preparative HPLC. This gives 431 mg (36% of theory) of the compound of Example A-II and 52 mg (4% of theory) of the compound of Example A-III.

### EXAMPLE A-II

¹H-NMR (300 MHz, CDCl₃): δ=0.98 (t, 6H), 1.43-1.52 (m, 1H), 1.63-1.72 (m, 1H), 1.75-1.85 (m, 1H), 1.95 (d, 1H), 2.34 (s, 3H), 3.99 (s, 3H), 5.09 (quintet, 1H), 5.44 (q, 2H), 6.02 (s, 1H), 6.84 (d, 1H), 6.98 (s, 1H), 7.60 (d, 1H) ppm.

MS (DCI): m/z=468/470 (M+NH₄)⁺

HPLC (Method 1): Rₜ=4.87 min

### EXAMPLE A-III:

¹H-NMR (300 MHz, CDCl₃): δ=0.98 (t, 6H), 1.43-1.52 (m, 1H), 1.63-1.86 (m, 2H), 1.94 (d, 1H), 2.33 (s, 3H), 3.98 (s, 3H), 5.01-5.13 (m, 3H), 6.30 (s, 1H), 6.51 (s, 1H), 6.93 (d, 1H), 7.60 (d, 1H) ppm.

MS (DCI): m/z=468/470 (M+NH₄)⁺

HPLC (Method 1): Rₜ=4.78 min.

### EXAMPLE A-IV

### 8,10-Dimethyl-11-hydroxy-3-[(1S)-1-hydroxy-3-methylbutyl]-4-methoxy-9-methyl-5H,7H-dibenzo[b,g][1,5]dioxocin-5-one

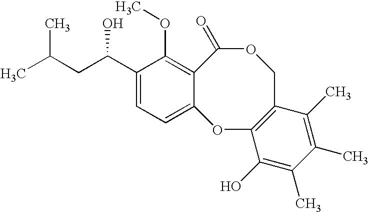

500 mg (0.94 mmol) of the compound from Example A-I are dissolved in 20 ml of dimethylformamide, and 3.92 ml (28.3 mmol) of tetramethyltin and 251 mg (0.22 mmol) of tetrakis(triphenylphosphine)palladium(0) are added under argon. The reaction vessel is closed and heated at 120° C. for 1 hour whilst being irradiated with microwaves (power 200 watt) in a microwave oven (MLS Ethos 1600). The reaction mixture is then cooled to room temperature, 20 ml of water are added and the reaction mixture is extracted a total of four times with in each case 10 ml of ethyl acetate. The combined organic phases are filtered through a 2 g Extrelut/silica gel cartridge (1:1) and the solvent is then removed under reduced pressure. The residue is purified chromatographically on silica gel (mobile phase: cyclohexane/ethyl acetate 100:0→10:90). This gives 339 mg (90% of theory) of product.

¹H-NMR (200 MHz, CDCl₃): δ=0.98 (dd, 6H), 1.40-1.85 (m, 3H), 2.00 (d, 1H), 2.08 (s, 3H), 2.15 (s, 3H), 2.26 (s, 3H), 4.00 (s, 3H), 4.91 (dd, 1H), 5.09 (quintet, 1H), 5.19-5.38 (m, 2H), 6.09 (s, 1H), 6.86 (d, 1H), 7.57 (d, 1H) ppm.

MS (DCI): m/z=418 (M+NH₄)⁺

HPLC (Method 1): Rₜ=4.87 min.

### EXAMPLE A-V AND EXAMPLE A-VI

### 8-Cyano-10-bromo-11-hydroxy-3-[(1S)-1-hydroxy-3-methylbutyl]-4-methoxy-9-methyl-5H,7H-dibenzo[b,g][1,5]dioxocin-5-one (Example A-V) and 8-Bromo-10-cyano-11-hydroxy-3-[(1S)-1-hydroxy-3-methylbutyl]-4-methoxy-9-methyl-5H,7H-dibenzo[b,g][1,5]dioxocin-5-one (Example A-VI)

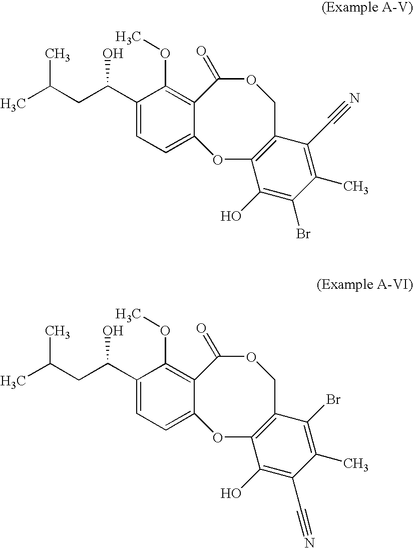

500 mg (0.94 mmol) of the compound from Example A-I are dissolved in 20 ml of dimethylformamide, and 554 mg (4.72 mmol) of zinc cyanide and 109 mg (0.09 mmol) of tetrakis(triphenylphosphine)palladium(0) are added under argon. The reaction vessel is closed and heated twice, in each case for 1 hour, with an interruption of 30 minutes, at 160° C. whilst being irradiated with microwaves (power 200 watt) in a microwave oven (MLS Ethos 1600). The reaction mixture is then cooled to room temperature, 20 ml of diethyl ether are added and the reaction mixture is washed in each case once with in each case 10 ml of saturated ammonium chloride solution and with water. The organic phase is filtered through a 2 g Extrelut/silica gel cartridge (1:1), and the cartridge is eluted with 10 ml of diethyl ether. The solvent is then removed under reduced pressure. The residue is purified chromatographically on silica gel (mobile phase: cyclohexane/ethyl acetate 100:0→10:90). This gives 104 mg (23% of theory) of the compound of Example A-V and 56 mg (12% of theory) of the compound of Example A-VI.

### EXAMPLE A-V

¹H-NMR (400 MHz, CDCl₃): δ=0.98 (t, 6H), 1.43-1.52 (m, 1H), 1.55-1.74 (m, 2H), 2.48 (br. s, 1H), 2.60 (s, 3H), 3.98 (s, 3H), 5.05 (m, 1H), 5.48 (q, 2H), 6.81 (d, 1H), 7.52 (d, 1H), 7.95 (br. s, 1H) ppm.

MS (DCI): m/z=493/495 (M+NH₄)⁺

HPLC (Method 2): Rₜ=4.82 min

### EXAMPLE A-VI

¹H-NMR (300 MHz, CDCl₃): δ=0.99 (t, 6H), 1.40-1.74 (m, 3H), 2.15 (br. s, 1H), 2.58 (s, 3H), 3.98 (s, 3H), 5.01-5.15 (m, 1H), 5.44 (q, 2H), 6.82 (d, 1H), 7.30 (br. s, 1H), 7.60 (d, 1H) ppm.

MS (DCI): m/z=493/495 (M+NH₄)⁺

HPLC (Method 1): Rₜ=4.65 min.

### EXAMPLE A-VII

### 11-Hydroxy-3-[(1S)-1-hydroxy-3-methylbutyl]-4-methoxy-9-methyl-8-vinyl-5H,7H-dibenzo[b,g][1,5]dioxocin-5-one

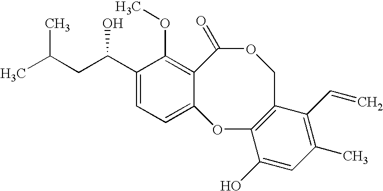

Under argon, 417 mg (0.924 mmol) of the compound from Example A-II are dissolved in 16 ml of toluene, and 36 mg (0.031 mmol) of tetrakis(triphenylphosphine)palladium(0) and 0.54 ml (1.85 mmol) of tributylvinyltin are added. The reaction vessel is closed immediately and the mixture is stirred at 100° C. overnight. After cooling, the reaction mixture is concentrated under reduced pressure. The residue is purified chromatographically on silica gel (mobile phase: cyclohexane/ethyl acetate 100:0→40:60). This gives 180 mg (49% of theory) of product.

¹H-NMR (300 MHz, CDCl₃): δ=0.98 (t, 6H), 1.43-1.85 (m, 3H), 1.97 (d, 1H), 2.18 (s, 3H), 3.99 (s, 3H), 4.91 (dd, 1H), 5.09 (quintet, 1H), 5.30 (br. s, 2H), 5.52 (dd, 1H), 5.97 (s, 1H), 6.56 (dd, 1H), 6.87 (d, 1H), 6.88 (s, 1H), 7.60 (d, 1H) ppm.

MS (ESIpos): m/z=421 (M+Na)⁺

HPLC (Method 1): Rₜ=4.76 min.

### EXAMPLE A-VIII

### (SCCA-4332-2) BAY 676253

### 8-Allyl-11-hydroxy-3-[(1S)-1-hydroxy-3-methylbutyl]-4-methoxy-9-methyl-5H,7H-dibenzo[b,g][1,5]dioxocin-5-one

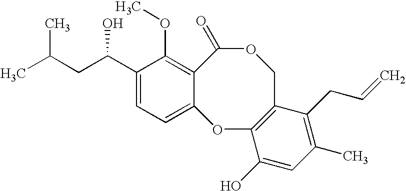

The preparation is carried out analogously to Example A-VII from 310 mg (0.687 mmol) of the compound from Example A-II. This gives 155 mg (55% of theory) of product.

¹H-NMR (300 MHz, CDCl₃): δ=0.97 (t, 6H), 1.43-1.85 (m, 3H), 1.97 (d, 1H), 2.20 (s, 3H), 3.22-3.24 (m, 2H), 3.98 (s, 3H), 4.75 (dd, 1H), 5.00 (dd, 1H), 5.09 (quintet, 1H), 5.17 (br. s, 2H), 5.77-5.92 (m, 1H), 6.04 (s, 1H), 6.87 (d, 1H), 6.88 (s, 1H), 7.58 (d, 1H) ppm.

MS (ESIpos): m/z=435 (M+Na)⁺

HPLC (Method 1): Rₜ=4.81 min.

### EXAMPLE A-IX

### 8-Bromo-3-[(1S)-1-hydroxy-3-methylbutyl]-11-(isopentyloxy)-4-methoxy-9-methyl-5H,7H-dibenzo[b,g][1,5]dioxocin-5-one

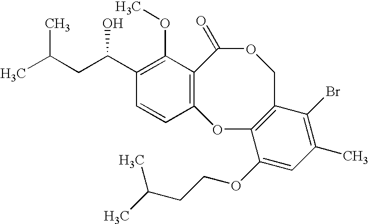

Under argon, 0.94 g (2.08 mmol) of the compound from Example A-II are dissolved in 20 ml of tetrahydrofuran, the mixture is cooled to 0° C. and 87 mg (2.187 mmol) of 60% sodium hydride are added a little at a time. After 5 minutes, 77 mg (0.208 mmol) of tetra-n-butylammonium iodide and 1.25 ml (10.42 mmol) of 3-methylbutyl bromide are added, and the mixture is stirred at 60° C. overnight. After a short while, a precipitate is formed. After cooling, water is added to the reaction mixture and the mixture is extracted twice with ethyl acetate. The organic phase is washed once with saturated sodium chloride solution, dried over sodium sulphate and concentrated under reduced pressure. The residue is purified chromatographically on silica gel (mobile phase: cyclohexane/ethyl acetate 10:1→2:1). This gives 711 mg (65% of theory) of product.

¹H-NMR (400 MHz, CDCl₃): δ=0.96-1.00 (m, 12H), 1.42-1.52 (m, 2H), 1.65-1.95 (m, 5H), 2.36 (s, 3H), 3.98 (s, 3H), 4.08 (t, 2H), 5.08 (quintet, 1H), 5.44 (q, 2H), 6.88 (d, 1H), 6.89 (s, 1H), 7.57 (d, 1H) ppm.

MS (DCI): m/z=540 (M+NH₄)⁺.

### EXAMPLE A-X

### 8,10-Dibromo-3-[(1S)-1-hydroxy-3-methylbutyl]-11-(isopentyloxy)-4-methoxy-9-methyl-5H,7H-dibenzo[b,g][1,5]dioxocin-5-one

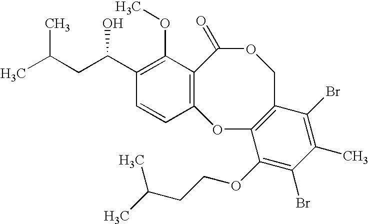

The preparation is carried out analogously to Example A-IX from 570 mg (1.08 mmol) of the compound from Example A-I. This gives 135 mg (21% of theory) of product.

¹H-NMR (200 MHz, CDCl₃): δ=0.94-1.00 (m, 12H), 1.42-1.95 (m, 7H), 2.59 (s, 3H), 3.99 (s, 3H), 4.16 (t, 2H), 5.09 (quintet, 1H), 5.43 (s, 2H), 6.97 (d, 1H), 7.59 (d, 1H) ppm.

MS (ESIpos): m/z=623 (M+Na)⁺.

### EXAMPLE A-XI

### 10-Bromo-8-chloro-11-hydroxy-3-[(1S)-1-hydroxy-3-methylbutyl]-4-methoxy-9-methyl-5H,7H-dibenzo[b,g][1,5]dioxocin-5-one

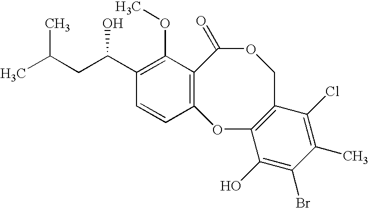

320 mg (0.47 ml, 4.42 mmol) of tert-butylamine are dissolved in 20 ml of toluene, and this solution is cooled to −30° C. Over a period of 5 minutes, a solution of 1.41 g (0.46 ml, 8.85 mmol) of bromine in 25 ml of dichloromethane is slowly added dropwise. The mixture is then cooled to −78° C., and a solution of the compound from Example A-XLIII (1.5 g, 3.68 mmol) in 25 ml of dichloromethane is added. With vigorous stirring, the mixture is warmed to room temperature and allowed to stand for 4-5 hours. The mixture is washed with 1 M hydrochloric acid and then with water. The organic phase is dried over sodium sulphate, filtered and concentrated.

The residue is separated chromatographically (silica gel, cyclohexane/ethyl acetate 5:1→3:1) and then purified further by preparative HPLC. This gives 791 mg (purity 88%, 39% of theory) of product.

R_{f}=0.35 (cyclohexane/ethyl acetate 2:1)

¹H-NMR (300 MHz, CDCl₃): δ=0.99 (m, 6H), 1.43 (m, 1H), 1.62 (m, 2H), 1.80 (m, 1H), 1.95 (m, 1H), 2.52 (s, 3H), 3.99 (s, 3H), 5.09 (dd, 1H), 5.40 (m, 2H), 6.38 (s, 1H), 6.87 (d, 1H), 7.62 (d, 1H) ppm.

MS (DCI): m/z=503 (M+NH₄)⁺

HPLC (Method 1): Rₜ 4.97 min.

### EXAMPLE A-XII

### 11-(Isopentyloxy)-4-methoxy-9-methyl-3-[(1E)-3-methyl-1-butenyl]-5H,7H-dibenzo[b,g][1,5]dioxocin-5-one

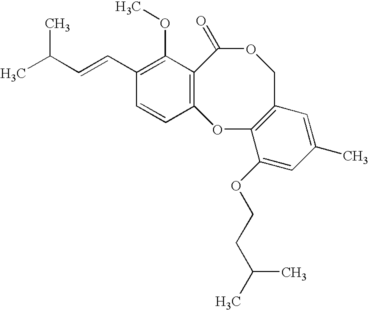

100 mg (0.23 mmol) of the compound from Example A-XXVIII are initially charged in 1.5 ml of toluene, 20 mg of molecular sieve (4 Å) and a catalytic amount of p-toluenesulphonic acid are added and the mixture is heated at 100° C. for 2 hours. The reaction mixture is cooled, three times its volume of diethyl ether is added and the mixture is stirred at room temperature for 2 hours. The mixture is then washed with saturated sodium bicarbonate solution, dried over sodium sulphate and concentrated under reduced pressure. The residue is purified chromatographically on 7 g of silica gel (mobile phase: ethyl acetate/cyclohexane 1:7). This gives 62 mg (65% of theory) of a white solid.

¹H-NMR (300 MHz, CDCl₃): δ=1.00 (d, 6H), 1.10 (d, 6H), 1.77 (q, 2H), 1.92 (sep., 1H), 2.26 (s, 3H), 2.49 (sextet, 1H), 3.91 (s, 3H), 4.09 (t, 2H), 5.04 (s, 2H), 6.13-6.21 (m, 1H), 6.40 (s, 1H), 6.55 (d, 1H), 6.78 (s, 1H), 6.88 (d, 1H), 7.54 (d, 1H) ppm.

HPLC (Method 1): Rₜ=5.88 min.

### EXAMPLE A-XIII

### 11-(Isopentyloxy)-4-methoxy-9-methyl-5-oxo-5H,7H-dibenzo[b,g][1,5]dioxocin-3-carbaldehyde

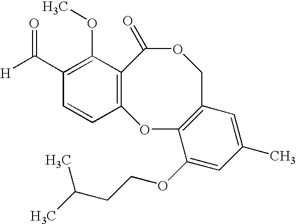

1.11 g (2.62 mmol) of the compound from Example A-XII are initially charged in 52 ml of dioxane, and 3.3 ml of osmium tetroxide (2.5% by weight strength solution in tert-butanol) are added. After 5 minutes, a solution of 2.8 g (13.07 mmol) of sodium periodate in 26 ml of water is added. A colourless suspension is formed. After 90 minutes, the mixture is filtered, the filtercake is washed with dichloromethane and the filtrate is partitioned between dichloromethane and water. The phases are separated and the aqueous phase is extracted two more times with dichloromethane. The combined organic phases are washed with saturated sodium chloride solution, dried over magnesium sulphate and concentrated under reduced pressure. The dark oily residue is purified chromatographically on silica gel (mobile phase: cyclohexane/ethyl acetate 100:0→4:1). This gives 671 mg (67% of theory) of a greenish-grey solid.

¹H-NMR (200 MHz, CDCl₃): δ 1.00 (d, 6H), 1.78 (q, 2H), 1.91 (sep., 1H), 2.28 (s, 3H), 4.07-4.12 (m, 5H), 5.11 (s, 2H), 6.43 (s, 1H), 6.81 (s, 1H), 7.03 (d, 1H), 8.00 (d, 1H), 10.35 (s, 1H) ppm.

MS (DCI): m/z=402 (M+NH₄)⁺

HPLC (Method 2): Rₜ=5.23 nm.

### EXAMPLE A-XIV

### 1-(11-Hydroxy-4-methoxy-9-methyl-5-oxo-5H,7H-dibenzo[b,g][1,5]dioxocin-3-yl)-3-methylbutyl formate

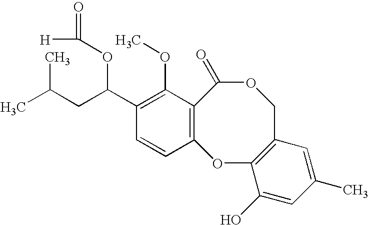

In 80 ml of formic acid, 15 g (40.3 mmol) of penicillide are warmed to 40° C. After one hour, the reaction solution is cooled and concentrated under reduced pressure. The residue is purified chromatographically on silica gel (mobile phase: cyclohexane/ethyl acetate 10:1→15:1). This gives 14.6 g (91% of theory) of product.

¹H-NMR (200 MHz, CDCl₃): δ=0.96 (d, 6H), 1.52-1.90 (m, 3H), 2.24 (s, 3H), 4.03 (s, 3H), 5.03 (q, 2H), 6.00 (s, 1H), 6.27 (dd, 1H), 6.38 (br. s, 1H), 6.86 (s, 1H), 6.88 (d, 1H), 7.48 (d, 1H), 8.07 (s, 1H) ppm.

MS (ESIpos): m/z=423 (M+Na)⁺

### EXAMPLE A-XV

### 9-[1-(Formyloxy)-3-methylbutyl]-8-methoxy-3-methyl-7-oxo-5H,7H-dibenzo[b,g]-[1,5]dioxocin-1-yl trifluoromethanesulphonate

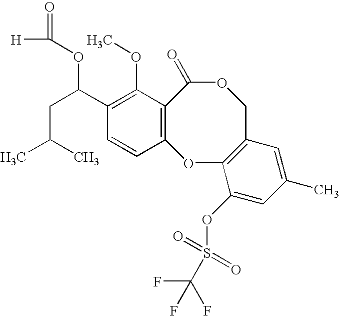

14 g (34.96 mmol) of the compound from Example A-XIV are dissolved in 160 ml of dichloromethane, the mixture is cooled to 0° C. and 20 ml (245 mmol) of pyridine are added. This is followed by addition of 24 ml (140 mmol) of trifluoromethane-sulphonic anhyhdride. A dark solution is formed. After 3 hours at room temperature, the reaction mixture is poured into ice-water and extracted twice with dichloromethane. The organic phase is washed once with saturated ammonium chloride solution, dried over sodium sulphate and concentrated under reduced pressure. The residue is purified chromatographically on silica gel (mobile phase: cyclohexane/ethyl acetate 20:1→5:1). This gives 18 g (97% of theory) of product.

¹H-NMR (300 MHz, CDCl₃): δ=0.96 (dd, 6H), 1.51-1.89 (m, 3H), 2.33 (s, 3H), 4.04 (s, 3H), 5.11 (q, 2H), 6.27 (dd, 1H), 6.90 (s, 1H), 7.10 (d, 1H), 7.14 (d, 1H), 7.52 (d, 1H), 8.06 (s, 1H).

LC-MS (Method 6): Rₜ=5.27 min.

MS (ESIpos): m/z=555 (M+Na)⁺

### EXAMPLE A-XVI

### 9-[1-Hydroxy-3-methylbutyl]-8-methoxy-3-methyl-7-oxo-5H,7H-dibenzo[b,g][1,5]-dioxocin-1-yl trifluoromethanesulphonate

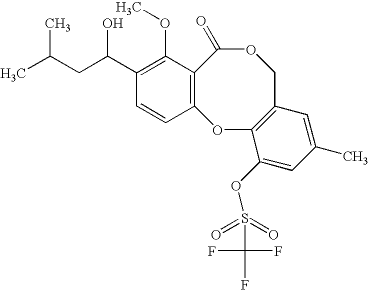

1.97 g (3.7 mmol) of the compound from Example A-XV are dissolved in 40 ml of methanol, and 1.33 ml (8.5 mmol) of 26% strength ammonia solution are added. After one hour at room temperature, the solvent is removed under reduced pressure and the residue is dried under high vacuum. This give 1.83 g (98% of theory) of product.

¹H-NMR (300 MHz, CDCl₃): δ=0.98 (t, 6H), 1.43-1.50 (m, 1H), 1.65-1.86 (m, 2H), 1.93 (d, 1H), 2.33 (s, 3H), 3.98 (s, 3H), 5.08-5.10 (m, 3H), 6.89 (s, 1H), 7.10 (d, 1H), −7.14 (s, 1H), 7.63 (d, 1H) ppm.

MS (ESIpos): m/z=527 (M+Na)⁺

### EXAMPLE A-XVII

### 8-Methoxy-3-methyl-9-(3-methylbutanoyl)-7-oxo-5H,7H-dibenzo[b,g][1,5]dioxocin-1-yl trifluoromethanesulphonate

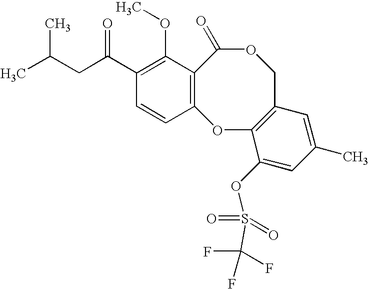

100 mg (198 μmol) of the compound from Example A-XVI are dissolved in 4 ml of dichloromethane, 40 mg (396 μmol) of basic alumina and 85 mg (396 μmol) of pyridinium chlorochromate are added and the mixture is stirred at room temperature. After a short while, the colour of the solution turns to black. After one hour, the reaction mixture is filtered through silica gel and the product is eluted with 50 ml of dichloromethane. This gives 92 mg (92% of theory) of product.

¹H-NMR (200 MHz, CDCl₃): δ=0.96 (d, 6H), 2.15-2.29 (m, 1H), 2.35 (s, 3H), 2.84 (d, 2H), 3.96 (s, 3H), 5.16 (br. s, 2H), 6.93 (s, 1H), 7.15 (d, 1H), 7.16 (s, 1H), 7.72 (d, 1H) ppm.

MS (ESIpos): m/z=503 (M+H)⁺

### EXAMPLE A-XVIII

### 4-Methoxy-9-methyl-3-(3-methylbutanoyl)-11-[3-methyl-3-(1H-pyrrol-1-yl)-1-butynyl]-5H,7H-dibenzo[b,g][1,5]dioxocin-5-one

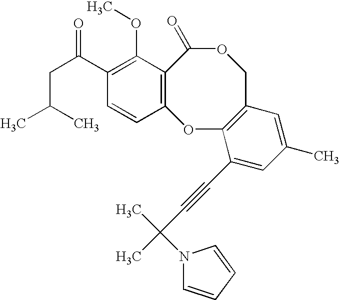

Under argon, 100 mg (200 μmol) of the compound from Example A-XVII are dissolved in 5 ml of a mixture of dimethylformamide and triethylamine (5:1), and 14 mg (20 μmol) of bis(triphenylphosphine)palladium(II) chloride, 11.4 mg (60 μmol) of copper(I) iodide and 222 mg (600 μmol) of tetra-n-butylammonium iodide are added at room temperature. After 5 minutes, 91 μl (800 μmol) of 1-(1,1-dimethyl-2-propynyl)-1H-pyrrole are added, and the mixture is stirred at 70° C. overnight. After cooling, the reaction solution is concentrated under high vacuum. Phosphate buffer (pH 7) is added to the residue, and the mixture is extracted four times with dichloromethane. The organic phase is filtered through a 1.1 g Extrelut/silica gel cartridge and eluted with dichloromethane. The filtrate is concentrated under reduced pressure. The residue is purified on a 10 g silica gel cartridge (mobile phase: cyclohexane/ethyl acetate 100:0→55:45). This gives 89 mg (91% of theory) of product.

¹H-NMR (300 MHz, CDCl₃): δ=0.96 (d, 6H), 1.90 (s, 6H), 2.17-2.24 (m, 1H), 2.27 (s, 3H), 2.83 (d, 2H), 3.97 (s, 3H), 5.11 (br. s, 2H), 6.17-6.19 (m, 2H), 6.84 (s, 1H), 7.00 (d, 1H), 7.03-7.05 (m, 2H), 7.30 (s, 1H), 7.7 (d, 1H) ppm.

MS (ESIpos): m/z=486 (M+H)⁺

HPLC (Method 1): Rₜ=5.47 min.

The examples listed in the table below are prepared analogously to the procedures described above, from the corresponding starting materials:

| Example | | |
|---|---|---|
| A- | Structure | Analytical data |
| XIX | 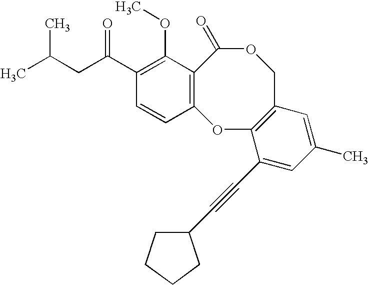 | ¹H-NMR (300 MHz, CDCl₃): δ = 0.96 (d, 6 H), 1.57-1.68 (m, 2 H), 1.70-1.85 (m, 4 H), 1.95-2.07 (m, 2 H), 2.22 (quintet, 1 H), 2.25 (s, 3 H), 2.84 (d, 2 H), 2.90 (quintet, 1 H), 3.96 (s, 3 H), 5.09 (br. s, 2 H), 6.75 (s, 1 H), 7.10 (d, 1 H), 7.26 (s, 1 H), 7.69 (d, 1 H) ppm. MS (ESIpos): m/z = 447 (M + H)⁺HPLC |
| #(Method 1): Rₜ = 5.77 min. | | |
| XX | 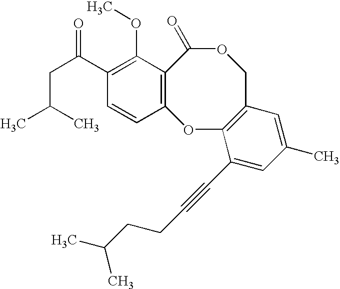 | ¹H-NMR (300 MHz, CDCl₃): δ = 0.94 (t, 12 H), 1.52-1.57 (m, 2 H), 1.79(quintet, 1 H), 2.22 (quintet, 1 H), 2.25 (s, 3 H), 2.48 (t, 2 H), 2.84 (d, 2 H), 3.96 (s, 3 H), 5.09 (br. s, 2 H), 6.76 (s, 1 H), 7.09 (d, 1 H), 7.26 (s, 1 H), 7.68 (d, 1 H) ppm. MS (ESIpos): m/z = 449 (M + H)⁺ |
| #HPLC (Method 1): Rₜ = 5.93 min. | | |
| XXI | 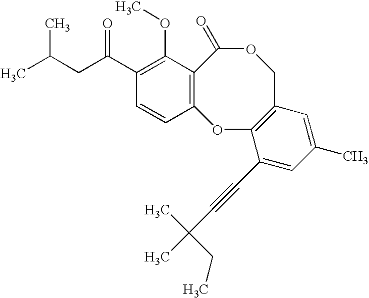 | ¹H-NMR (300 MHz, CDCl₃): δ = 0.96 (d, 6 H), 1.06 (t, 3 H), 1.25 (s, 3 H), 1.30 (s, 3 H), 1.43-1.58 (m, 3 H), 2.25 (s, 3 H), 2.84 (d, 2 H), 3.96 (s, 3 H), 5.09 (br. s, 2 H), 6.76 (s, 1 H), 7.09 (d, 1 H), 7.32 (s, 1 H), 7.69 (d, 1 H), ppm. MS (ESIpos): m/z = 449 (M + H)⁺HPLC (Method 1): Rₜ = 5.93 min. |
| XXII | 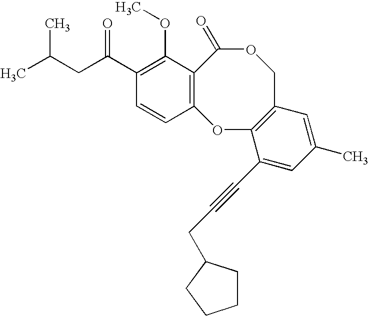 | ¹H-NMR (300 MHz, CDCl₃): δ = 0.96 (d, 6 H), 1.30-1.43 (m, 2 H), 1.48-1.72 (m, 5 H), 1.79-1.90 (m, 2H), 2.19 (sep., 1 H), 2.25 (s, 3 H), 2.49 (d, 2 H), 2.84 (d, 2 H), 3.96 (s, 3 H), 5.09 (br. s, 2 H), 6.76 (s, 1 H), 7.10 (d, 1 H), 7.26 (s, 1 H), 7.68 (d, 1 H) ppm. MS (DCI): m/z = 478 (M + NH₄)⁺HPLC |
| #(Method 1): Rₜ = 6.03 min. | | |
| XXIII | 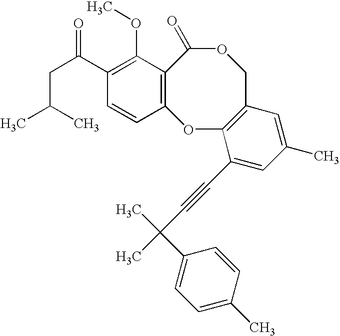 | ¹H-NMR (300 MHz, CDCl₃): δ = 0.96 (d, 6 H), 1.69 (s, 6 H), 2.17-2.24 (m, 1 H), 2.26 (s, 3 H), 2.32 (s, 3 H), 2.83 (d, 2 H), 3.97 (s, 3 H), 5.11 (br. s, 2 H), 6.79 (s, 1 H), 7.05 (d, 1 H), 7.11 (d, 2 H), 7.30 (s, 1 H), 7.53 (d, 2 H), 7.61 (d, 1 H) ppm. MS (DCI): m/z = 528 (M + NH₄)⁺HPLC |
| #(Method 1): Rₜ = 6.07 min. | | |
| XXIV | 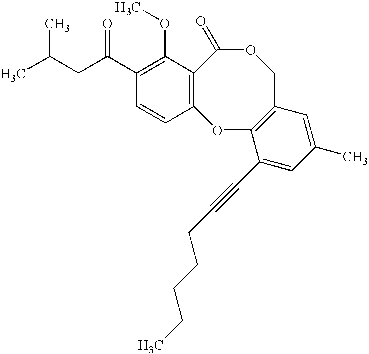 | ¹H-NMR (300 MHz, CDCl₃): δ = 0.89 (t, 3 H), 0.96 (d, 6 H), 1.32-1.50 (m, 4 H), 1.64 (quintet, 2 H), 2.19 (quintet, 1 H), 2.25 (s, 3 H), 2.47 (t, 2 H), 2.84 (d, 2 H), 3.96 (s, 3 H), 5.09 (br. s, 2 H), 6.76 (s, 1 H), 7.09 (d, 1 H), 7.26 (s, 1 H), 7.68 (d, 1 H) ppm. MS (ESIpos): m/z = 449 (M + H)⁺ |
| XXV | 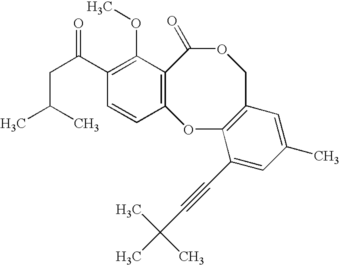 | ¹H-NMR (300 MHz, CDCl₃): δ = 0.97 (d, 6 H), 1.36 (s, 9 H), 2.22 (m, 1 H), 2.25 (s, 3 H), 2.84 (d, 2 H), 3.97 (s, 3 H), 5.10 (br. s, 2 H), 6.77 (s, 1 H), 7.10 (d, 1 H), 7.69 (d, 1 H) MS (ESIpos): m/z = 435 (M + H)⁺HPLC (Method 1): Rₜ = 5.91 min. |
| XXVI | 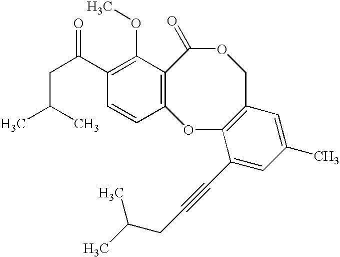 | ¹H-NMR (300 MHz, CDCl₃): δ = 0.97 (d, 6 H), 1.06 (d, 6 H), 1.95 (m, 1 H), 2.22 (m, 1 H), 2.25 (s, 3 H), 2.88 (d, 2 H), 2.84 (d, 2 H), 3.97 (s, 3 H), 5.10 (br. s, 2 H), 6.77 (s, 1 H), 7.10 (d, 1 H), 7.69 (d, 1 H) ppm. MS (ESIpos): m/z 435 (M + H)⁺HPLC (Method 1): Rₜ = 5.91 min. |
| XXVII | 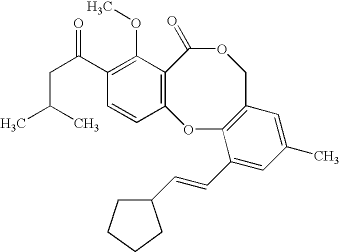 | Isomer mixture; MS (ESIpos): m/z = 449 (M + H)⁺HPLC (Method 1): Rₜ = 6.13, 6.21 and 6.29 min. |

### EXAMPLE A-XXVIII

### 3-[(1S)-1-Hydroxy-3-methylbutyl]-11-(isopentyloxy)-4-methoxy-9-methyl-5H,7H-dibenzo[b,g][1,5]dioxocin-5-one

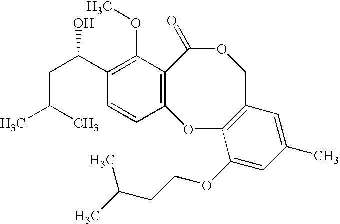

Under argon, 54 g (145 mmol) of penicillide are dissolved in 200 ml of tetrahydrofuran, and 6.09 g (152 mmol) of 60% strength sodium hydride are added a little at a time at 0° C. After 5 minutes, 5.35 g (14.5 mmol) of tetra-n-butylammonium iodide and 34.7 ml (290 mmol) of 3-methylbutyl bromide are added to the reaction solution, which is then heated at 60° C. overnight. For work-up, the reaction mixture is cooled, water is added and the mixture is extracted with ethyl acetate. The organic phase is washed once with water, dried over sodium sulphate and concentrated under reduced pressure. The residue is triturated with pentane, filtered off with suction and dried under high vacuum at 40° C. This gives 50 g (76% of theory) of product.

¹H-NMR (300 MHz, CDCl₃): δ=0.95-1.01 (m, 12H), 1.44-1.52 (m, 1H), 1.64-1.80 (m, 4H), 1.90 (quintet, 1H), 1.97 (d, 1H), 2.27 (s, 3H), 3.96 (s, 3H), 4.11 (t, 2H), 5.04-5.10 (m, 3H), 6.41 (s, 1H), 6.79 (s, 1H), 6.94 (d, 1H), 7.55 (d, 1H) ppm.

MS (DCI): m/z=460 (M+NH₄)⁺

HPLC (Method 1): Rₜ=5.33 min.

### EXAMPLE A-XXIX

### 11-(Isopentyloxy)-4-methoxy-9-methyl-3-(3-methylbutanoyl)-5H,7H-dibenzo[b,g]-[1,5]dioxocin-5-one

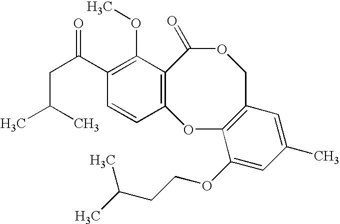

Under argon, 100 mg (226 μmol) of the compound from Example A-XXVIII are dissolved in 0.5 ml of dichloromethane, and 73 mg (339 μmol) of pyridinium chlorochromate and a little silica gel are added, and the mixture is stirred at room temperature overnight. For work-up, the reaction mixture is diluted with dichloromethane and filtered off through kieselguhr. The filtrate is concentrated under reduced pressure and the residue is purified chromatographically on silica gel (mobile phase: ethyl acetate/cyclohexane 1:15). This gives 74 mg (74% of theory) of product.

¹H-NMR (300 MHz, CDCl₃): δ=0.98 (dd, 12H), 1.77 (q, 2H), 1.92 (quintet, 1H), 2.21 (quintet, 1H), 2.28 (s, 3H), 2.83 (d, 2H), 3.96 (s, 3H), 4.09 (t, 2H), 5.10 (br. s, 2H), 6.42 (s, 1H), 6.80 (s, 1H), 6.97 (d, 1H), 7.67 (d, 1H) ppm.

MS (ESIpos): m/z=441 (M+H)⁺

HPLC (Method 1): Rₜ=5.51 min.

### EXAMPLE A-XXX

### 11-(Isopentyloxy)-4-methoxy-9-methyl-3-(3-methylbutanoyl)-8-nitro-5H,7H-dibenzo[b,g][1,5]dioxocin-5-one

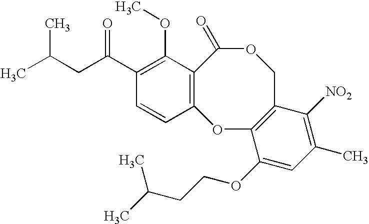

Under argon, 1.8 g (4.09 mmol) of the compound from Example A-XXX are dissolved in 50 ml of dichloromethane, and at −78° C., 597 mg (4.5 mmol) of nitronium tetrafluoroborate are added. The mixture is warmed to 0° C. After 2 hours, the reaction solution is poured into ice-water and extracted twice with dichloromethane. The organic phase is washed once with saturated sodium bicarbonate solution, dried over sodium sulphate and concentrated under reduced pressure. The residue is purified on silica gel (mobile phase: cyclohexane/ethyl acetate 20:1→5:1). This gives 1.39 g (70% of theory) of product.

¹H-NMR (300 MHz, CDCl₃): δ=0.99 (dd, 12H), 1.80 (q, 2H), 1.87-1.96 (m, 1H), 2.16-2.30 (m, 1H), 2.34 (s, 3H), 2.84 (d, 2H), 3.97 (s, 3H), 4.15 (t, 2H), 5.14 (br. s, 2H), 6.84 (s, 1H), 6.92 (d, 1H), 7.71 (d, 1H) ppm.

MS (ESIpos): m/z=486 (M+H)⁺

### EXAMPLE A-XXXI

### 11-(Isopentyloxy)-9-methyl-3-(3-methylbutanoyl)-5-oxo-5H,7H-dibenzo[b,g][1,5]-dioxocin-4-yl trifluoromethanesulphonate

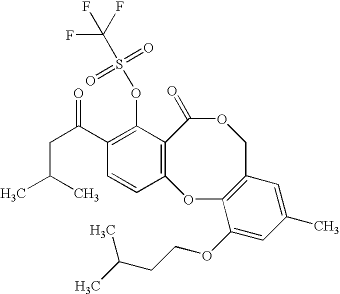

Under argon, 500 mg (1.17 mmol) of the compound from Example A-XVII are dissolved in dichloromethane, and 29 mg (234 μmol) of 4-dimethylaminopyridine and 306 μl (1.76 mmol) of N,N-diisopropylethylamine are added. At 0° C., 218 μl (1.29 mmol) of trifluoromethansulphonic anhydride are added dropwise over a period of 10 minutes. After 30 minutes, water is added to the reaction mixture and the mixture is diluted with dichloromethane and then washed three times with 1 N hydrochloric acid. The organic phase is washed two more times with water, dried over sodium sulphate and concentrated under reduced pressure. The residue is purified chromatographically on silica gel (mobile phase: cyclohexane/ethyl acetate 10:1). This gives 620 mg (95% of theory) of product.

R_{f}=0.57 (cyclohexane/ethyl acetate 5:1)

¹H-NMR (300 MHz, CDCl₃): δ=0.99 (dd, 12H), 1.77 (q, 2H), 1.91 (sep., 1H), 2.22 (sep., 1H), 2.29 (s, 3H), 2.80 (d, 2H), 4.10 (t, 2H), 5.15 (br. s, 2H), 6.45 (s, 1H), 6.82 (s, 1H), 7.34 (d, 1H), 7.78 (d, 1H) ppm.

MS (ESIpos): m/z=559 (M+H)⁺

### EXAMPLE A-XXXII

### 11-(Isopentyloxy)-9-methyl-3-(3-methylbutanoyl)-5-oxo-5H,7H-dibenzo[b,g][1,5]-dioxocin-4-carbonitrile

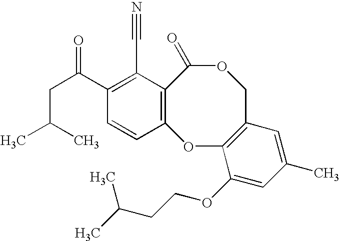

Under argon, 100 mg (179 μmol) of the compound from Example A-XXXI, 32 mg (269 μmol) of zinc cyanide and 8 mg (7 μmol) of tetrakis(triphenylphosphine)-palladium(0) are added to 2 ml of dimethylformamide in a flask which had been dried by heating and evacuation. The flask is evacuated once more, vented with argon and then heated at 100° C. overnight. After cooling, 1 ml of water and 5 ml of ethyl acetate are added to the reaction mixture and the mixture is filtered through an Extrelut cartridge and eluted with about 40 ml of ethyl acetate. The filtrate is concentrated under reduced pressure. The residue is purified by preparative HPLC.

This gives 28 mg (36% of theory) of product.

R_{f}=0.37 (cyclohexane/ethyl acetate 2:1)

¹H-NMR (200 MHz, CDCl₃): δ=1.01 (d, 12H), 1.71-1.95 (m, 3H), 2.22-2.40 (m, 4H), 2.89 (d, 2H), 4.10 (t, 2H), 5.08 (br. s, 2H), 6.46 (s, 1H), 6.83 (s, 1H), 7.50 (d, 1H), 7.96 (d, 1H) ppm.

MS (ESIpos): m/z=436 (M+H)⁺

### EXAMPLE A-XXXIII

### 1-[11-(Cyclopentylethynyl)-4-methoxy-9-methyl-5-oxo-5H,7H-dibenzo[b,g][1,5]-dioxocin-3-yl]-3-methylbutyl formate

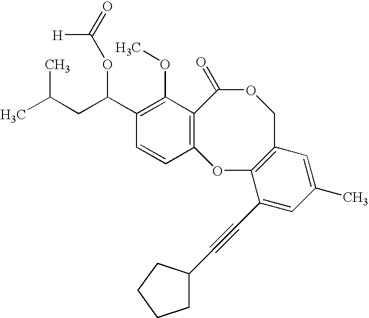

The preparation is carried out analogously to Example A-XVIII from 100 mg (188 μmol) of the compound from Example A-XV. This gives 89 mg (100% of theory) of product.

¹H-NMR (300 MHz, CDCl₃): δ=0.96 (d, 6H), 1.54-1.88 (m, 9H), 1.97-2.02 (m, 2H), 2.23 (s, 3H), 2.89 (quintet, 1H), 4.03 (s, 3H), 5.05 (q, 2H), 6.28 (dd, 1H), 6.73 (s, 1H), 7.05 (d, 1H), 7.22 (s, 1H), 7.47 (d, 1H), 8.06 (s, 1H) ppm.

LC-MS (Method 7): Rₜ=4.05 min.

MS (ESIpos): m/z=449 (M+H)⁺

The examples listed in the table below are prepared analogously to the procedures described above, from the corresponding starting materials:

| Example | | |
|---|---|---|
| A- | Structure | Analytical data |
| XXXIV | 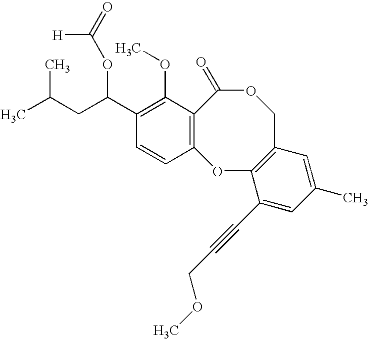 | ¹H-NMR (300 MHz, CDCl₃): δ = 0.96 (d, 6 H), 1.50-1.70 (m, 2 H), 1.78-1.88 (m, 1 H), 2.26 (s, 3 H), 3.46 (s, 3 H), 4.03 (s, 3 H), 4.38 (s, 3 H), 5.06 (q, 2 H), 6.27 (dd, 1 H), 6.81 (s, 1 H), 7.05 (d, 1 H), 7.28 (s, 1 H), 7.47 (d, 1 H), 8.06 (s, 1 H) ppm. LC-MS (Method 7): Rₜ = 3.46 min. |
| #MS (ESIpos): m/z = 425 (M + H)⁺ | | |
| XXXV | 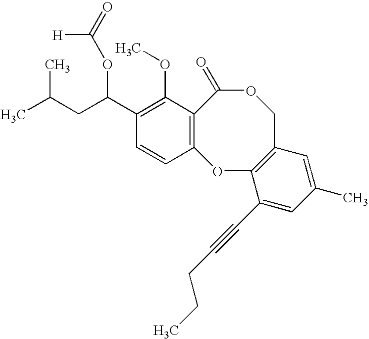 | ¹H-NMR (300 MHz, CDCl₃): δ = 0.96 (d, 6 H), 1.06 (t, 3 H), 1.52-1.70 (m, 2 H), 1.64 (q, 2 H), 1.79-1.88 (m, 1 H), 2.24 (s, 3 H), 2.46 (t, 2 H), 4.02 (s, 3 H), 5.05 (q, 2 H), 6.28 (dd, 1 H), 6.75 (s, 1 H), 7.05 (d, 1 H), 7.26 (s, 1 H), 7.47 (d, 1 H), 8.06 (s, 1 H) ppm. LC-MS (Method 7): Rₜ = 3.88 min. |
| #MS (ESIpos): m/z = 423 (M + H)⁺ | | |

### EXAMPLE A-XXXVI

### 1-(11-Heptyl-4-methoxy-9-methyl-5-oxo-5H,7H-dibenzo[b,g][1,5]dioxocin-3-yl)-3-methylbutyl formate

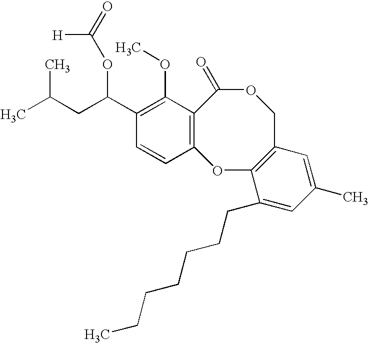

Under argon, 1.5 ml (751 μmol) of a 0.5 M solution of 9-borabicyclo[3.3.1]nonane are initially charged in tetrahydrofuran and, at 0° C. (turbid solution), 106 μl (751 μmol) of 1-heptene are added. After 4 hours at room temperature, 2 ml of dioxane, 5.4 mg (4.7 μmol) of tetrakis(triphenylphosphine)palladium(0), 60 mg (282 μmol) of potassium phosphate and 100 mg (188 μmol) of the compound from Example A-XV are added, and the mixture is heated at 85° C. overnight. After cooling, the reaction mixture is poured into water and extracted twice with ethyl acetate. The organic phase is dried over sodium sulphate and concentrated under reduced pressure. The residue is purified chromatographically on silica gel (mobile phase: cyclohexane/ethyl acetate 20:1→2:1). This gives 63 mg (69% of theory) of product.

¹H-NMR (400 MHz, CDCl₃): δ=0.88 (t, 3H), 0.96 (d, 6H), 1.23-1.90 (m, 13H), 2.25 (s, 3H), 3.80 (t, 2H), 4.03 (s, 3H), 5.05 (q, 2H), 6.27 (dd, 1H), 6.67 (s, 1H), 6.86 (d, 1H), 7.03 (s, 1H), 7.46 (d, 1H), 8.06 (s, 1H) ppm.

MS (ESIpos): m/z=483 (M+H)⁺

### EXAMPLE A-XXXVII

### 4-Hydroxy-11-(isopentyloxy)-9-methyl-3-(3-methylbutanoyl)-5H,7H-dibenzo[b,g]-[1,5]dioxocin-5-one

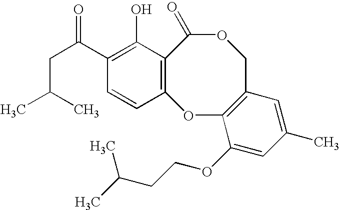

Under argon, 10 g (22.7 mmol) of the compound from Example A-XXIX are dissolved in 250 ml of dichloromethane, and 20.43 ml (20.43 mmol) of a 1 M solution of boron tribromide in tetrahydrofuran are added at −78° C. After two hours at −78° C., 30 ml of methanol are added to the reaction solution and the mixture is stirred at −78° C. for 30 minutes and then allowed to warm to room temperature. The mixture is diluted with a little dichloromethane and washed twice with saturated sodium bicarbonate solution and twice with water. The organic is dried over magnesium sulphate and concentrated under reduced pressure. The residue is taken up in diethyl ether and stirred and the solid is filtered off with suction and dried under high vacuum. This gives 7.7 g (80% of theory) of product.

¹H-NMR (300 MHz, CDCl₃): δ=1.00 (dd, 12H), 1.77 (q, 2H), 1.92 (sep., 1H), 2.22-2.40 (m, 4H), 2.80 (d, 2H), 4.09 (t, 2H), 5.10 (br. s, 2H), 6.44 (s, 1H), 6.73 (d, 1H), −6.81 (s, 1H), 7.89 (d, 1H), 13.20 (s, 1H) ppm.

MS (ESIpos): m/z=427 (M+H)⁺

HPLC (Method 1): Rₜ=5.39 min.

### EXAMPLE A-XXXVIII

### 4-Methoxy-8-fluoro-11-(isopentyloxy)-9-methyl-3-(3-methylbutanoyl)-5H,7H-dibenzo[b,g][1,5]dioxocin-5-one

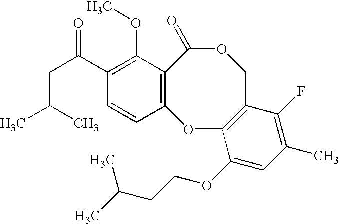

Under argon, 252 mg (0.57 mmol) of the compound from Example A-XXXVII are dissolved in 2 ml of dry acetonitrile, and 811 mg (1.26 mmol) of 1-fluoro-4-hydroxy-1,4-diazoniabicyclo[2.2.2]octane bistetrafluoroborate (50% on Al₂O₃) are added at room temperature. The mixture is stirred at 80° C. for 2 hours. After cooling to room temperature, in each case 10 ml of ethyl acetate and water are added. The phases are separated and the aqueous phase is then extracted two more times with ethyl acetate, and the combined organic phases are washed with saturated sodium chloride solution and dried over magnesium sulphate. The solvent is removed under reduced pressure and the residue is purified chromatographically on silica gel (mobile phase: cylohexane→cyclohexane/ethyl acetate 1:1→ethyl acetate). This gives 14 mg (5% of theory) of product.

¹H-NMR (300 MHz, CDCl₃): δ 0.97 (dd, 12H), 1.73 (q, 2H), 1.88 (sep., 1H), 2.12-2.28 (m, 4H), 2.81 (d, 2H), 3.96 (t, 3H), 4.05 (t, 2H), 5.26 (s, 2H), 6.78 (d, 1H), 6.95 (d, 1H), 7.67 (d, 1H) ppm.

MS (ESIpos): m/z=459 (M+H)⁺

### EXAMPLE A-XXXIX

### 4-Ethoxy-1-(isopentyloxy)-9-methyl-3-(3-methylbutanoyl)-5H,7H-dibenzo[b,g]-[1,5]dioxocin-5-one

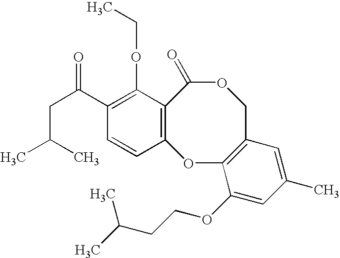

Under argon, 180 mg (422 μmol) of the compound from Example A-XVII are dissolved in 2 ml of tetrahydrofuran, 116 mg (844 mmol) of potassium carbonate and 270 μl (3.38 mmol) of iodoethane are added and the mixture is stirred at 40° C. for 16 hours. Since the reaction is still incomplete, another 135 μl (1.69 mmol) of iodoethane are added and the temperature is increased to 60° C. After 8 hours, the reaction is cooled and concentrated under reduced pressure. The residue is taken up in ethyl acetate and washed with 1 N hydrochloric acid. The aqueous phase is extracted once with ethyl acetate. The organic phase is dried over sodium sulphate and concentrated under reduced pressure. The residue is purified by preparative HPLC. This gives 132 mg (69% of theory) of product.

¹H-NMR (300 MHz, CDCl₃): δ=0.98 (dd, 12H), 1.40 (t, 3H), 1.77 (q, 2H), 1.92 (sep., 1H), 2.20 (heptet, 1H), 2.27 (s, 3H), 2.85 (d, 2H), 4.09 (t, 2H), 4.13 (q, 2H), 5.09 (br. s, 2H), 6.42 (s, 1H), 6.80 (s, 1H), 6.97 (d, 1H), 7.65 (d, 1H) ppm.

MS (DCI): m/z 455 (M+H)⁺

HPLC (Method 1): Rₜ=5.84 min.

### EXAMPLE A-XL

### 11-Hydroxy-4-methoxy-9-methyl-3-(3-methylbutanoyl)-5H,7H-dibenzo[b,g][1,5]-dioxocin-5-one

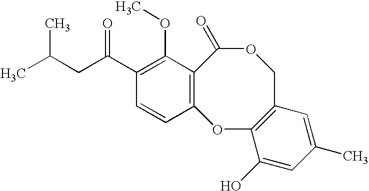

1.43 g (3.84 mmol) of penicillide are dissolved in 38 ml of dichloromethane, and 783 mg (7.68 mmol) of basic alumina and 1.65 g (7.68 mmol) of pyridinium chlorochromate are added. After one hour at room temperature, the reaction mixture is filtered through silica gel (mobile phase: cyclohexane/ethyl acetate 10:1). This gives 390 mg (27% of theory) of product.

R_{f}=0.56 (cyclohexane/ethyl acetate 2:1)

¹H-NMR (400 MHz, CDCl₃): δ=0.96 (d, 6H), 2.21 (sep., 1H), 2.25 (s, 3H), 2.84 (d, 2H), 3.97 (s, 3H), 5.12 (br. s, 2H), 5.95 (s, 1 μl), 6.40 (s, 1H), 6.87 (s, 1H), 6.94 (d, 1H), 7.68 (d, 1H) ppm.

MS (ESIpos): m/z=371 (M+H)⁺

HPLC (Method 1): Rₜ=4.69 min.

### EXAMPLE A-XLI

### 8-Methoxy-3-methyl-9-(3-methylbutanoyl)-7-oxo-5H,7H-dibenzo[b,g][1,5]dioxocin-1-yl chloromethanesulphonate

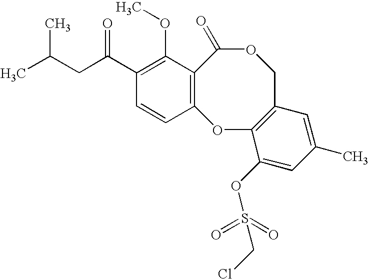

800 mg (2.16 mmol) of the compound from Example A-XL are dissolved in 20 ml dichloromethane and cooled to 0° C. 602 μl (4.32 mmol) of triethylamine and a solution of 386 mg (2.59 mmol) of chloromethanesulphonyl chloride in 5 ml of dichloromethane are then added dropwise. After 2 hours at room temperature, the reaction solution is washed with 1 M hydrochloric acid and with saturated sodium bicarbonate solution. The organic phase is dried over sodium sulphate and concentrated under reduced pressure. The residue is purified chromatographically on silica gel (mobile phase: cyclohexane/ethyl acetate 5:1). This gives 749 mg (72% of theory) of product.

R_{f}=0.43 (cyclohexane/ethyl acetate 2:1)

¹H-NMR (200 MHz, CDCl₃): δ=0.96 (d, 6H), 2.22 (sep., 1H), 2.34 (s, 3H), 2.84 (d, 2H), 3.96 (s, 3H), 4.87 (s, 2H), 5.16 (br. s, 2H), 6.88 (br. s, 1H), 7.20 (d, 1H), 7.26 (br. s, 1H), 7.28 (s, 1H), 7.71 (d, 1H) ppm.

MS (ESIpos): m/z=483 (M+H)⁺

HPLC (Method 2): Rₜ=5.01 min.

### EXAMPLE A-XLII

### 8,10-Dichloro-11-hydroxy-3-[(1S)-1-hydroxy-3-methylbutyl]-4-methoxy-9-methyl-5H,7H-dibenzo[b,g][1,5]dioxocin-5-one

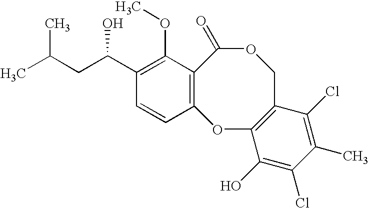

1.5 g (4.03 mmol) of penicillide are initially charged in 30 ml of ethanol/water (1:1), 1.18 g (8.86 mmol) of N-chlorosuccinimide and 1.83 g (7.81 mmol) of iron(III) chloride hexahydrate are added and the mixture is stirred at room temperature over the weekend. For work-up, the the reaction mixture is diluted with ethyl acetate and washed with water. The organic phase is dried over sodium sulphate- and concentrated under reduced pressure. The residue is purified chromatographically on silica gel (mobile phase: cyclohexane/ethyl acetate 10:1→5:1). This gives 1.23 g (69% of theory) of product.

¹H-NMR (300 MHz, CDCl₃): δ=0.98 (t, 6H), 1.44-1.86 (m, 3H), 2.00 (br. s, 1H), 2.44 (s, 3H), 3.99 (s, 3H), 5.05-5.12 (m, 1H), 5.41 (q, 2H), 6.40 (s, 1H), 6.87 (d, 1H), 7.59 (d, 1H) ppm.

MS (ESIpos): m/z=464 (M+Na)⁺

HPLC (Method 1): Rₜ=5.07 min.

### EXAMPLE A-XLIII

### 8-Chloro-11-hydroxy-3-[(1S)-1-hydroxy-3-methylbutyl]-4-methoxy-9-methyl-5H,7H-dibenzo[b,g][1,5]dioxocin-5-one

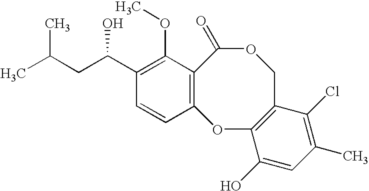

2.25 g (6.04 mmol) of penicillide are initially charged in 45 ml of ethanol/water (1:1), 847 mg (6.34 mmol) of N-chlorosuccinimide and 1.58 g (5.86 mmol) of iron(III) chloride hexahydrate are added and the mixture is stirred at room temperature over the weekend. For work-up, the reaction mixture is diluted with 100 ml of ethyl acetate and washed with water. The organic phase is washed once with saturated sodium chloride solution, dried over sodium sulphate and concentrated under reduced pressure. The residue is purified chromatographically on silica gel (mobile phase: cyclohexane/ethyl acetate 5:1→3:1). This gives 2.21 g (75% of theory) of product.

¹H-NMR (300 MHz, CDCl₃): δ=0.98 (t, 6H), 1.42-1.86 (m, 3H), 2.04 (br. s, 1H), 2.29 (s, 3H), 3.99 (s, 3H), 5.09 (dd, 1H), 5.35-5.48 (m, 2H), 6.05 (br. s, 1H), 6.84 (d, 1H), 6.94 (br. s, 1H), 7.59 (d, 1H) ppm.

MS (ESIpos): m/z=429 (M+Na)⁺

HPLC (Method 2): Rₜ=4.86 min.

### EXAMPLE A-XLIV

### 3-((1S)-1-{[tert-Butyl(dimethyl)silyl]oxy}-3-methylbutyl)-11-(isopentyloxy)-4 methoxy-9-methyl-5H,7H-dibenzo[b,g][1,5]dioxocin-5-one

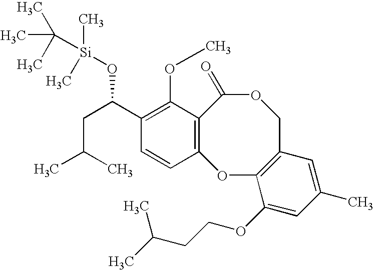

1.50 g (3.40 mmol) of the compound from Example A-XXVIII and 0.51 g (7.46 mmol) of imidazole are dissolved in 7.5 ml of DMF, the mixture is cooled to 0° C. and 0.92 g (6.1 mmol) of tert-butyldimethylchlorosilane is added. The mixture is stirred at room temperature overnight. Water is then added, the mixture is extracted with 5 portions of diethyl ether and the combined organic phases are subsequently washed with 2 portions of water, dried over sodium sulphate and concentrated under reduced pressure. This gives 1.72 g (91% of theory) of the product.

¹H-NMR (300 MHz, CDCl₃): δ=−0.21 (s, 3H), 0.02 (s, 3H), 0.81 (s, 9H), 0.88 (d, 3H), 0.95 (d, 3H), 0.99 (d, 6H), 1.30 (m, 2H), 1.60 (m, 1H), 1.75 (m, 2H), 1.92 (m, 1H), 2.22 (s, 3H), 3.90 (s, 3H), 4.06 (t, 2H), 4.98 (s, 2H), 5.05 (dd, 1H), 6.39 (br. s, 1H), 6.77 (br. s, 1H), 6.89 (d, 1H), 7.57 (d, 1H) ppm.

MS (DCI: m/z=574 (M+NH₄)⁺

HPLC (Method 1): Rₜ=9.2 min.

### EXAMPLE A-XLV

### 3-((1S)-1-{[tert-Butyl(dimethyl)silyl]oxy}-3-methylbutyl)-6-[2-hydroxymethyl)-6-(isopentyloxy)-4-methylphenoxy]-2-methoxybenzoic acid

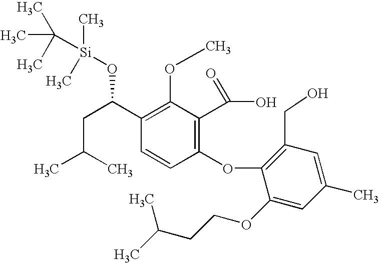

1.71 g (3.07 mmol) of Example A-XLIV are dissolved in 25 ml of dichloromethane, and 0.79 g (6.14 mmol) of potassium trimethylsilanolate is then added. The reaction mixture is stirred vigorously at room temperature for 5-6 hours. The solvent is then distilled off and the residue is initially acidified with 1 M hydrochloric acid and then immediately extracted with 2 portions of ethyl acetate. The combined organic phases are dried over sodium sulphate and concentrated. The residue is sufficiently pure for further reaction. This gives 1.72 g (98% of theory) of product.

¹H-NMR (300 MHz, CDCl₃): δ=−0.21 (s, 3H), 0.02 (s, 3H), 0.79 (m, 9H), 0.85 (m, 6H), 0.88 (d, 3H), 0.95 (d, 3H), 1.30 (m, 2H), 1.46 (m, 2H), 1.55 (m, 1H), 1.80 (m, 1H), 2.36 (s, 3H), 3.90 (m, 5H), 4.62 (s, 2H), 5.02 (dd, 1H), 6.39 (d, 1H), 6.71 (br. s, 1H), 6.87 (br. s, 1H), 7.38 (d, 1H) ppm.

MS (ESIpos): m/z=597 (M+Na)⁺

HPLC (Method 1): Rₜ=7.0 min.

### EXAMPLE A-XLVI

### 3-((1S)-1-{[tert-Butyl(dimethyl)silyl]oxy}-3-methylbutyl)-6-[2-formyl-6-(isopentyl-oxy)-4-methylphenoxy]-2-methoxybenzoic acid

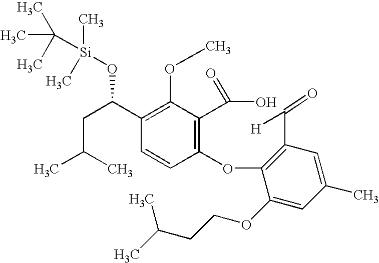

1.72 g (3.00 mmol) of Example A-XLV are dissolved in 30 ml of dichloromethane, and 2.55 g (6.00 mmol) of Dess-Martin periodinane are added. At room temperature, the solution is stirred for about 1 hour. With vigorous stirring, 1 M aqueous sodium hydroxide solution is then added dropwise until the solution is colourless, and the pH is then adjusted to 3-4 using 1 M hydrochloric acid. Immediately afterwards, the mixture is extracted with ethyl acetate and the organic phase is dried over sodium sulphate and concentrated. The crude product (2.30 g, purity 60%, 80% of theory) is reacted further without further purification.

¹H-NMR (200 MHz, CDCl₃): δ=−0.21 (s, 3H), 0.02 (s, 3H), 0.70-1.00 (m, 21H), 1.20-1.90 (m, 6H), 2.40 (m, 3H), 3.90 (m, 5H), 5.02 (m, 1H), 6.30 (m, 2H), 7.01 (m, 1H), 7.35 (m, 1H), 10.20 (s, 1H) ppm.

MS (ESIpos): m/z=595 (M+Na)⁺

HPLC (Method 1): Rₜ=7.4 min.

### EXAMPLE A-XLVII

### 3-((1S)-1-{[tert-Butyl(dimethyl)silyl]oxy}-3-methylbutyl)-6-[2-[(1R,S)-1-hydroxyethyl]-6-(isopentyloxy)-4-methylphenoxy]-2-methoxybenzoic acid

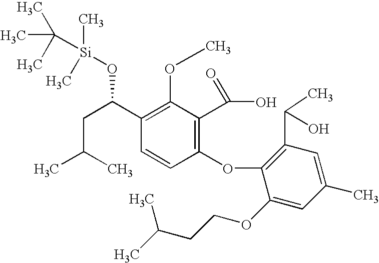

2.2 g (purity: 60%, 2.30 mmol) of Example A-XLVI are dissolved in 100 ml of tetrahydrohydrofuran, and the solution is cooled to −78° C. 1.5 ml of a 3 M solution of methylmagnesium bromide in THF (4.6 mmol) are slowly added dropwise. The mixture is then stirred at room temperature for about another 15 minutes. The reaction is quenched by addition of concentrated ammonium chloride solution, and the pH is adjusted to about 34 using 1 M hydrochloric acid. The mixture is then extracted three times with dichloromethane and the combined organic phases are dried over sodium sulphate and concentrated. The residue (2.2 g, purity 62%, 100% of theory) is reacted further as crude product.

¹H-NMR (200 MHz, CDCl₃): δ=−0.21 (s, 3H), 0.03 (s, 3H), 0.70-1.00 (m, 21H), 1.20-1.90 (m, 9H), 2.36 (s, 3H), 3.90 (m, 6H), 4.62 (s, 1H), 5.02 (m, 1H), 6.41 (d, 1H), 6.73 (br. s, 1H), 6.88 (br. s, 1H), 7.38 (d, 1H) ppm.

MS (DCI): m/z=606 (M+NH₄)⁺

HPLC (Method 1): Rₜ=7.1 min.

### EXAMPLE A-XLVIII

### 7(R,S)-3-((1S)-1-{[tert-Butyl(dimethyl)silyl]oxy}-3-methylbutyl)-11-(isopentyloxy)-4-methoxy-7,9-dimethyl-5H,7H-dibenzo[b,g][1,5]dioxocin-5-one

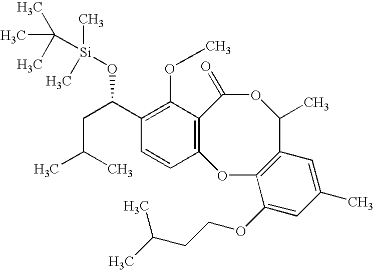

Under argon, 2.2 g (purity. 60%, 2.3 mmol) of Example A-XLVII are dissolved in 10 ml of acetonitrile, and 3.90 ml (28.01 mmol) of triethylamine are added. Over a period of 10 h, this solution is added dropwise to a mixture, kept at 80° C. under reflux, of 3.59 g (14.05 mmol) of 2-chloro-1-methylpyridinium iodide in 15 ml of acetonitrile. The solvent is then distilled off, the residue is taken up in dichloromethane and washed with water and the organic phase is dried and concentrated. The residue is purified chromatographically (silica gel, mobile phase: cyclohexane/ethyl acetate 9:1). This gives 0.79 g (37% of theory) of product as a mixture of the epimers.

R_{f}=0.73 (cyclohexane/ethyl-acetate 5:1)

¹H-NMR (300 MHz, CDCl₃): δ=−0.21-0.02 (m, 6H), 0.81-1.00 (m, 21H), 1.20-1.98 (m, 9H), 2.30 (m, 3H), 3.92 (s, 3H), 4.06 (m, 2H), 5.10 (m, 1H), 5.45 (m, 1H), 6.58 (br. s, 1H), 6.77 (br. s, 1H), 6.93 (m, 1H), 7.57 (m, 1H) ppm.

LC-MS (Method 6): R=5.40 min.

MS (ESIpos): m/z=593 (M+Na)⁺.

### WORKING EXAMPLES

### EXAMPLE A-1

### 3-[(1S)-1-Hydroxy-3-methylbutyl]-4-methoxy-9-methyl-11-(2-methylbutoxy)-5H,7H-dibenzo[b,g][1,5]dioxocin-5-one

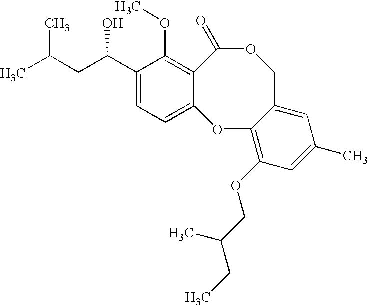

100 mg (0.27 mmol) of penicillide are dissolved in 0.5 ml of 1,3-dimethyltetrahydro-2(1H)-pyrimidinone, and 11.3 mg (0.28 mmol) of sodium hydride are added at 0° C. The mixture is stirred at room temperature for 15 minutes. A solution of 60.8 mg of 1-bromo-2-methylbutane and 10.1 mg (0.07 mmol) of sodium iodide in 0.5 ml of 1,3-dimethyltetrahydro-2(1H)-pyrimidinone is then added, and the mixture is heated at 80° C. for 2 hours. After cooling, water is added to the reaction solution and the mixture is extracted with ethyl acetate. The organic phase is washed twice with water and once with saturated sodium chloride solution, dried over sodium sulphate and concentrated under reduced pressure. The residue is purified chromatographically on silica gel (cyclohexane/ethyl acetate gradient). This gives 66 mg (56% of theory) of product.

¹H-NMR (300 MHz, CDCl₃): δ=0.95-1.00 (m, 9H), 1.09 (d, 3H), 1.32-2.00 (m, 7H), 2.26 (s, 3H), 3.80-3.95 (m, 2H), 3.97 (s, 3H), 5.04-5.14 (m, 3H), 6.52 (br. s, 1H), 6.79 (br. s, 1H), 6.93 (d, 1H), 7.52 (d, 1H) ppm.

MS (ESIpos): m/z=465 (M+Na)⁺

HPLC (Method 2): Rₜ=5.48 min.

The examples listed in the table below are prepared analogously to the procedures described above, from the corresponding starting materials:

| Example | | |
|---|---|---|
| A- | Structure | Analytical data |
| 2 | 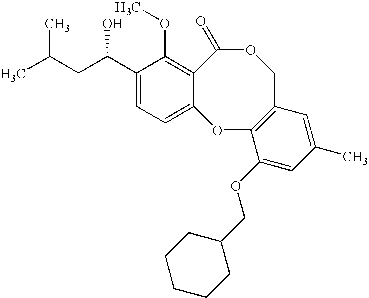 | ¹H-NMR (300 MHz, CDCl₃): δ = 0.97 (m, 6 H), 1.10-1.99(m, 15 H), 2.26 (s, 3 H), 3.84 (d, 2 H), 3.97 (s, 3 H), 5.04-5.14 (m, 3 H), 6.71 (br. s, 1 H), 6.92 (br. s, 1 H), 6.95 (d, 1 H), 7.56 (d, 1 H) ppm. MS (ESIpos): m/z = 491 (M + Na)⁺HPLC (Method 1): Rₜ = 5.94 min. |
| 3 | 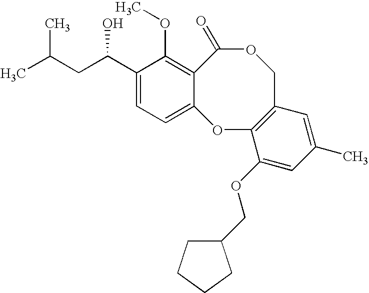 | ¹H-NMR (200 MHz, CDCl₃): δ = 0.97 (q, 6 H), 1.35-2.05 (m, 12 H), 2.26 (s, 3 H), 2.38-2.54 (m, 1 H), 3.93 (d, 2 H), 3.97 (s, 3 H), 5.04-5.14 (m, 3 H), 6.71 (br. s, 1 H), 6.92 (br. s, 1 H), 6.95 (d, 1 H), 7.56 (d, 1 H) ppm. MS (ESIpos): m/z = 477 (M + Na)⁺HPLC (Method 1): Rₜ = 5.67 min. |
| 4 | 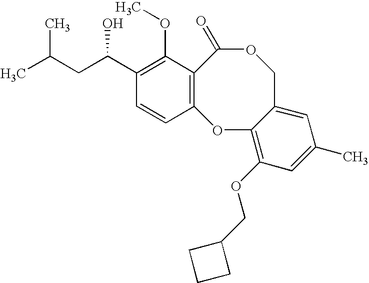 | ¹H-NMR (300 MHz, CDCl₃): δ = 0.97 (t, 6 H), 1.44-2.12 (m, 8 H), 2.13-2.24 (m, 2 H), 2.26 (s, 3 H), 2.81-2.93 (m, 1 H), 3.97 (s, 3 H), 4.03 (d, 2 H), 5.04-5.14 (m, 3 H), 6.41 (br. s, 1 H), 6.78 (br. s, 1 H), 6.94 (d, 1 H), 7.55 (d, 1 H) ppm. MS (ESIpos): m/z = 463 (M + Na)⁺HPLC (Method 1): |
| #Rₜ = 5.54 min. | | |
| 5 | 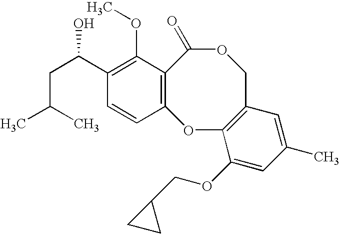 | ¹H-NMR (200 MHz, CDCl₃): δ = 0.39 (dd, 2 H), 0.67 (dd, 2 H), 0.97 (q, 6 H), 1.42-1.88 (m, 4 H), 1.96 (d, 1 H), 2.26 (s, 3 H), 3.93 (d, 2 H), 3.97 (s, 3 H), 5.04-5.14 (m, 3 H), 6.42 (br. s, 1 H), 6.78 (br. s, 1 H), 7.00 (d, 1 H), 7.56 (d, 1 H) ppm. MS (DCI): m/z = 444 (M + NH₄)⁺HPLC (Method 1): |
| #Rₜ = 5.13 min. | | |

### EXAMPLE A-6

### 3-[(1S)-1-Hydroxy-3-methylbutyl]-4-methoxy-9-methyl-11-[(4-methylpentyl)oxy]-5H,7H-dibenzo[b,g][1,5]dioxocin-5-one

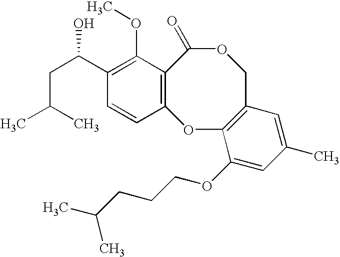

Under argon, 100 mg (0.27 mmol) of penicillide are dissolved in 1 ml of tetrahydrofuran, and 11.3 mg (0.28 mmol) of sodium hydride are added at 0° C. After 5 minutes, 66.5 mg (0.40 mmol) of 1-bromo-4-methylpentane and 0.1 mg (0.21 mmol) of tetra-n-butylammonium iodide are added to the reaction solution, and the mixture is heated at 60° C. overnight. After cooling, 1.5 ml of water are added to the mixture, and the mixture is diluted with dichloromethane. The mixture is filtered through an Extrelut NT 3 cartridge and the cartridge is washed three times with in each case 5 ml of dichloromethane. The filtrate is concentrated under reduced pressure. The residue is purified by preparative HPLC. This gives 62.5 mg (51% of theory) of product.

R_{f} value=0.43 (ethyl acetate/cyclohexane 1:2)

¹H-NMR (300 MHz, CDCl₃): δ=0.92-0.99 (m, 12H), 1.37-1.93 (m, 8H), 1.96 (d, 1H), 2.27 (s, 3H), 3.97 (s, 3H), 4.04 (t, 2H), 5.04-5.12 (m, 3H), 6.41 (s, 1H), 6.78 (s, 1H), 6.95 (d, 1H), 7.53 (d, 1H) ppm.

MS (DCI): m/z=474 (M+NH₄)⁺

HPLC (Method 1): Rₜ=5.32 min.

The examples listed in the table below are prepared analogously to the procedures described above, from the corresponding starting materials:

| Example | | |
|---|---|---|
| A- | Structure | Analytical data |
| 7 | 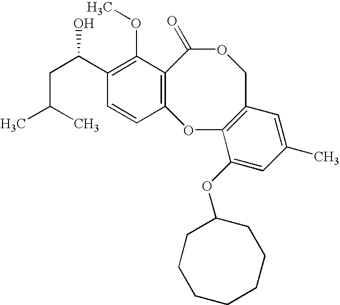 | ¹H-NMR (300 MHz, CDCl₃): δ = 0.97 (t, 6 H), 1.43-2.03 (m, 18 H), 2.26 (s, 3 H), 3.97 (s, 3 H), 4.42-4.52 (m, 1 H), 5.04-5.12 (m, 3 H), 6.40 (s, 1 H), 6.79 (s, 1 H), 6.90 (d, 1 H), 7.58 (d, 1 H) ppm. LC-MS (Method 5): Rₜ = 5.55 min. MS (ESIpos): m/z = 483 (M + H)⁺ |
| 8 | 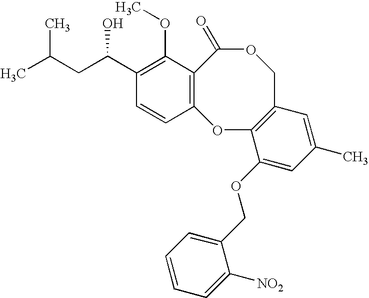 | ¹H-NMR (300 MHz, CDCl₃): δ = 0.97 (t, 6 H), 1.45-1.92 (m, 4 H), 2.28 (s, 3 H), 3.99 (s, 3 H), 5.05-5.12 (m, 3 H), 5.58 (s, 2 H), 6.50 (s, 1 H), 6.90 (s, 1 H), 6.98 (d, 1 H), 7.47-7.62 (m, 2 H), 7.72 (t, 1 H), 8.10 (d, 1 H), 8.22 (dd, 1 H) ppm. MS (ESIpos): m/z = 530 (M + Na)⁺HPLC (Method 1): Rₜ = 5.00 min. |
| 9 | 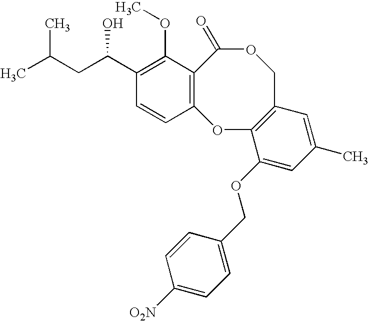 | ¹H-NMR (300 MHz, CDCl₃): δ = 0.97 (t, 6 H), 1.44-1.78 (m, 3 H), 1.95 (d, 1 H), 2.26 (s, 3 H), 3.98 (s, 3 H), 5.04-5.12 (m, 3 H), 5.28 (s, 2 H), 6.55 (s, 1 H), 6.81 (s, 1 H), 6.94 (d, 1 H), 7.58 (d, 1 H), 7.68 (d, 2 H), 8.28 (d, 2 H) ppm. LC-MS (Method 5): Rₜ = 4.76 min. MS (ESIpos): m/z = 530 (M + Na)⁺ |
| 10 | 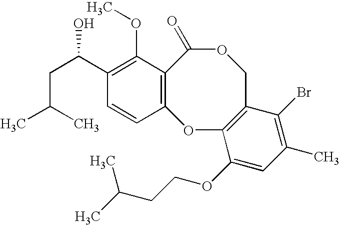 | ¹H-NMR (300 MHz, CDCl₃): δ = 0.96-1.00 (m, 12 H), 1.42-1.52 (m, 1 H), 1.65-1.95 (m, 6 H), 2.36 (s, 3 H), 3.98 (s, 3 H), 4.08 (t, 2 H), 5.08 (quintet, 1 H), 5.39-5.50 (m, 2 H), 6.89 (d, 1 H), 6.90 (s, 1 H), 7.57 (d, 1 H) ppm. LC-MS (Method 5): Rₜ = 5.54 min. MS (ESIpos): m/z = 566 (M + HCOOH)⁺ |
| 11 | 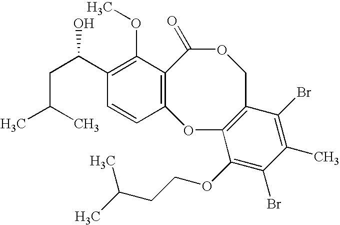 | ¹H-NMR (200 MHz, CDCl₃): δ = 0.94-1.01 (m, 12 H), 1.47-1.95 (m, 7 H), 2.25 (s, 3 H), 3.99 (s, 3 H), 4.16 (t, 2 H), 5.09 (quintet, 1 H), 5.43 (s, 2 H), 6.97 (d, 1 H), 7.59 (d, 1 H) ppm. MS (ESIpos): m/z = 623 (M + Na)⁺ |
| 12 | 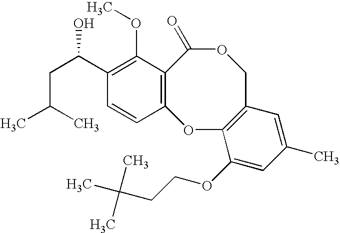 | ¹H-NMR (300 MHz, CDCl₃): δ = 0.97 (t, 6 H), 1.03 (s, 9 H), 1.44-1.85 (m, 3 H), 1.83 (t, 2 H), 1.93 (d, 1 H), 2.27 (s, 3 H), 3.97 (s, 3 H), 4.11 (t, 2 H), 5.04-5.12 (m, 3 H), 6.40 (s, 1 H), 6.80 (s, 1 H), 6.92 (d, 1 H), 7.55 (d, 1 H) ppm. MS (DCI): m/z = 474 (M + NH₄)⁺ |
| 13 | 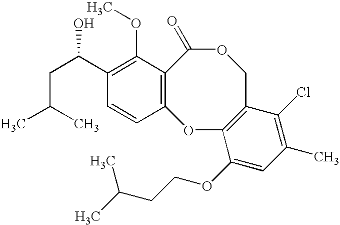 | ¹H-NMR (400 MHz, CDCl₃): δ = 0.89 (m, 6 H), 0.98 (m, 6 H), 1.49 (m, 1 H), 1.69 (m, 1 H), 1.71-1.99 (m, 5 H) 2.31 (s, 3 H), 3.99 (s, 3 H), 4.09 (t, 2 H), 5.08 (m, 1 H), 5.39 (d, 1 H), 5.43 (d, 1 H), 6.89 (br. s, 1 H), 6.90 (d, 1 H), 7.59 (m, 1 H) ppm. MS (DCI): m/z = 494 (M + NH₄)⁺HPLC |
| #(Method 1): Rₜ = 5.9 min. | | |

### EXAMPLE A-14

### 8,10-Dibromo-3-[(1S)-1-hydroxy-3-methylbutyl]-4-methoxy-9-methyl-11-[(2-methyl-2-propenyl)oxy]-5H,7H-dibenzo[b,g][1,5]dioxocin-5-one

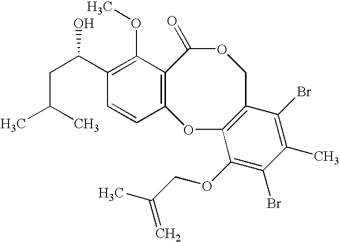

Under argon and in a closed vessel, 1 g (2 mmol) of the compound from Example A-I is dissolved in 14 ml of dimethylformamide, 0.8 g (2.4 mmol) of caesium carbonate and 0.3 g (2.4 mmol) of 3-bromo-2-methylpropene are added and the mixture is stirred at 60° C. for 4 hours. After cooling, the reaction solution is stirred into ice-cold 0.15 N hydrochloric acid. The resulting precipitated product is filtered off with suction and dried under high vacuum. The solid is purified chromatographically on a silica gel column (mobile phase: cyclohexane/ethyl acetate 20:1→6:1). This gives 915 mg (78% of theory) of product.

¹H-NMR (300 MHz, CDCl₃): δ=0.98 (t, 6H), 1.42-1.85 (m, 3H), 1.92 (s, 4H), 2.60 (s, 3H), 3.99 (s, 3H), 4.55 (s, 2H), 5.01 (s, 1H), 5.08 (quintet, 1H), 5.19 (s, 1H), 5.37-5.48 (m, 2H), 6.97 (d, 1H), 7.59 (d, 1H) ppm.

MS (ESIpos): m/z=606 (M+Na)⁺

HPLC (Method 1:): Rₜ=5.82 min.

### EXAMPLE A-15

### 8,10-Dichloro-3-[(1S)-1-hydroxy-3-methylbutyl]-11-(isopentyloxy)-4-methoxy-9-methyl-5H,7H-dibenzo[b,g][1,5]dioxocin-5-one

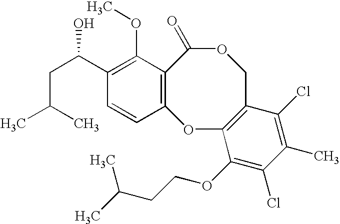

The preparation is carried out analogously to the preparation described in Example A-14 using 50 mg (0.11 mmol) of the compound from Example A-XLII. This gives 31 mg (54% of theory) of product.

¹H-NMR (300 MHz, CDCl₃): δ=0.95-1.00 (m, 12H), 1.25-1.93 (m, 7H), 2.44 (s, 3H), 3.99 (s, 3H), 4.16 (t, 2H), 5.07-5.14 (m, 1H), 5.40-5.42 (m, 2H), 6.96 (d, 1H), 7.59 (d, 1H) ppm.

### EXAMPLE A-16

### 3-[(1S)-1-Hydroxy-3-methylbutyl]-4-methoxy-9-methyl-11-(neopentyloxy)-5H,7H-dibenzo[b,g][1,5]dioxocin-5-one

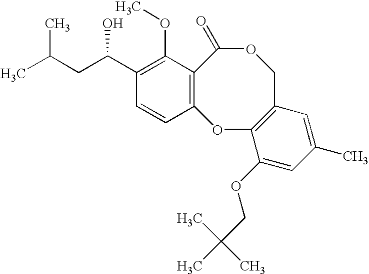

Under argon, 100 mg (0.27 mmol) of penicillide are dissolved in 0.5 ml of dimethylformamide, and 11.3 mg (0.28 mmol) of sodium hydride are added at 0° C.

After 5 minutes, the reaction solution is diluted with 0.5 ml of 1,3-dimethyltetrahydro-2(1H)-pyrimidinone, 159 mg (0.81 mmol) of neopentyl iodide and 68 mg (0.30 mmol) of silver(I) oxide are added and the mixture is heated at 100° C. overnight. After cooling, water is added to the mixture and the mixture is extracted with ethyl acetate. The organic phase is washed once with water, dried over magnesium sulphate and concentrated under reduced pressure. The residue is purified chromatographically on silica gel (mobile phase: ethyl acetate/cyclohexane 1:3). This gives 13.5 mg (11% of theory) of product.

¹H-NMR (300 MHz, CDCl₃): δ=0.97 (q, 6H); 1.10 (s, 9H), 1.45-1.83 (m, 3H), 1.99 (d, 1H), 2.26 (s, 3H), 3.67 (s, 2H), 3.97 (s, 3H), 5.04-5.12 (m, 3H), 6.40 (s, 1H), 6.78 (s, 1H), 6.95 (d, 1H), 7.56 (d, 1H) ppm.

MS (DCI): m/z=460 (M+NH₄)⁺

HPLC (Method 1): Rₜ=5.55 min.

### EXAMPLE A-17

### 3-({9-[(1S)-1-Hydroxy-3-methylbutyl]-8-methoxy-3-methyl-7-oxo-5H,7H-dibenzo[,b,g][1,5]dioxocin-1-yl}oxy)benzonitrile

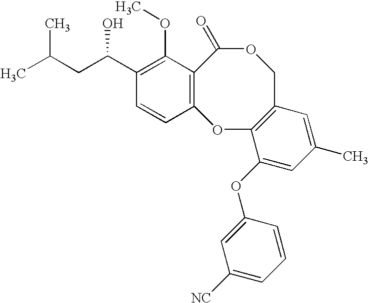

70 mg (0.19 mmol) of penicillide, together with 55.2 mg of 3-cyanophenylboronic acid, 51.2 mg (0.28 mmol) of copper(II) acetate and molecular sieve (4 Å), are suspended in 10 ml of dichloromethane. At room temperature, 130 μl (0.94 mmol) of triethylamine and 80 μl (0.94 mmol) of pyridine are simultaneously added to the reaction mixture, and the mixture is stirred overnight. The R_{f} value of the product corresponds to that of the starting material. For work-up, silica gel is added to the mixture, and the mixture is concentrated to dryness under reduced pressure. The crude product is purified on a 20 g silica gel cartridge (mobile phase: cyclohexane/ethyl acetate 90:10→50:50). This gives 53 mg (60% of theory) of product.

LC-MS (Method 3): Rₜ=3.25 min.

MS (ESIpos): m/z=474 (M+H)⁺

### EXAMPLE A-18

### 4-({9-[(1S)-1-Hydroxy-3-methylbutyl]-8-methoxy-3-methyl-7-oxo-5H,7H-dibenzo[b,g][1,5]dioxocin-1-yl}oxy)benzonitrile

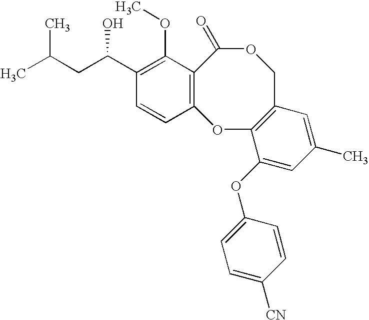

The preparation is carried out analogously to the procedure described in Example A-17 using 70 mg (0.19 mmol) of penicillide. This gives 71 mg (79% of theory) of product.

LC-MS (Method 3): Rₜ=3.27 min.

MS (ESIpos): m/z=474 (M+H)⁺

### EXAMPLE A-19

### 9-[(1S)-1-Hydroxy-3-methylbutyl]-8-methoxy-3-methyl-7-oxo-5H,7H-dibenzo[b,g]-[1,5]dioxocin-1-yl pivalate

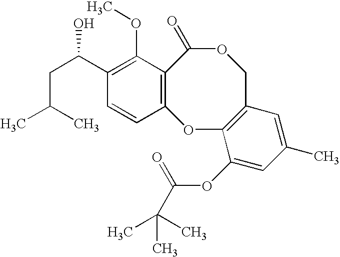

Under argon, 100 mg (0.27 mmol) of penicillide are dissolved in 1 ml of tetrahydrofuran, and 11.3 mg (0.28 mmol) of sodium hydride are added at 0° C. After 5 minutes, 30 μl (0.28 mmol) of 2,2-dimethylpropionyl chloride are added dropwise to the reaction solution, and the mixture is stirred at room temperature for one hour. For work-up, water is added to the reaction mixture and the mixture is extracted twice with ethyl acetate. The organic phase is washed with saturated sodium chloride solution, dried over sodium sulphate and concentrated under reduced pressure. The residue is purified chromatographically on silica gel (mobile phase: ethyl acetate/cyclohexane 1:4→1:1). This gives 106 mg (87% of theory) of product.

¹H-NMR (400 MHz, CDCl₃): δ=0.97 (t, 6H), 1.39 (s, 9H), 1.45-1.53 (m, 1H), 1.66-1.85 (m, 2H), 1.92 (d, 1H), 2.28 (s, 3H), 3.97 (s, 3H), 5.05-5.13 (m, 3H), 6.71 (s, 1H), 6.91 (s, 1H), 6.94 (d, 1H), 7.59 (d, 1H) ppm.

MS (ESIpos): m/z=479 (M+Na)⁺

HPLC (Method 1): Rₜ=5.26 min.

The examples listed in the table below are prepared analogously to the procedures described above, from the corresponding starting materials:

| Example | | |
|---|---|---|
| A- | Structure | Analytical data |
| 20 | 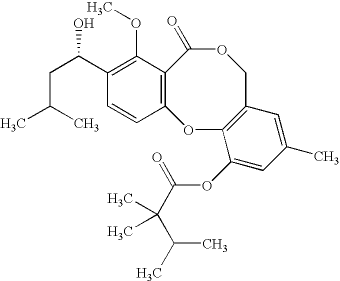 | ¹H-NMR (200 MHz, CDCl₃): δ = 0.92-1.13 (m, 12 H), 1.28 (s, 6 H), 1.24-1.88 (m, 4 H), 1.93-2.02 (m, 1 H), 2.28 (s, 3 H), 3.97 (s, 3 H), 5.03-5.12 (m, 3 H), 6.71 (d, 1 H), 6.88 (d, 1 H), 6.97 (d, 1 H), 7.59 (d, 1 H) ppm. LC-MS (Method 5): Rₜ = 5.18 min. MS (DAD): m/z = 484 (M)⁺ |
| 21 | 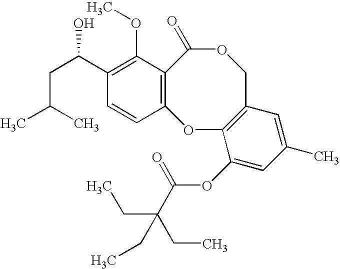 | ¹H-NMR (200 MHz, CDCl₃): δ = 0.83-1.06 (m, 15 H), 1.40-1.90 (m, 10 H), 2.28 (s, 3 H), 3.98 (s, 3 H), 5.00-5.15 (m, 3 H), 6.71 (d, 1 H), 6.85 (d, 1 H), 6.99 (d, 1 H), 7.59 (d, 1 H) ppm. LC-MS (Method 5): Rₜ = 5.34 min. MS (DAD): m/z = 498 (M)⁺ |
| 22 | 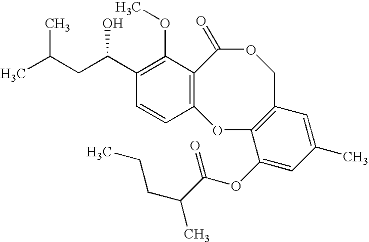 | ¹H-NMR (200 MHz, DMSO-d₆): δ = 0.82-0.94 (m, 9 H), 1.02-1.85 (m, 8 H), 1.26 (d, 3 H), 2.26 (s, 3 H), 2.75-2.90 (m, 1 H), 3.82 (s, 3 H), 4.89 (quintet, 1 H), 5.18 (s, 2 H), 6.85 (d, 1 H), 6.97 (br. s, 1 H), 7.10 (br. s, 1 H), 7.59 (d, 1 H) ppm. MS (ESIpos): m/z = 493 (M + Na)⁺HPLC (Method 2): Rₜ = 5.33 min. |
| 23 | 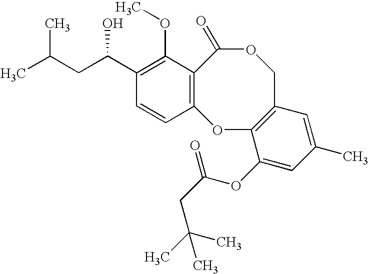 | ¹H-NMR (300 MHz, CDCl₃): δ = 0.96 (m, 6 H), 1.15 (s, 9 H), 1.44-1.82 (m, 3 H), 1.91 (d, 1 H), 2.28 (s, 3 H), 2.52 (s, 2 H), 3.97 (s, 3 H), 5.02-5.12 (m, 3 H), 6.71 (br. s, 1 H), 6.92 (br. s, 1 H), 6.95 (d, 1 H), 7.56 (d, 1 H) ppm. MS (ESIpos): m/z = 471 (M + H)⁺HPLC (Method 1): Rₜ = 5.07 min. |
| 24 | 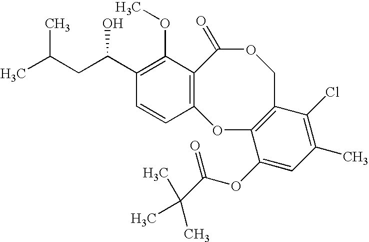 | ¹H-NMR (400 MHz, CDCl₃): δ = 0.98 (m, 6 H), 1.39 (s, 9 H), 1.50 (m, 1 H), 1.69 (m, 1 H), 1.80 (m, 1 H), 1.95 (d, 1 H), 2.31 (s, 3 H), 3.99 (s, 3 H), 5.08 (m, 1 H), 5.39 (d, 1 H), 5.43 (d, 1 H), 6.89 (d, 1 H), 7.01 (br. s, 1 H), 7.59 (d, 1 H) ppm. MS (DCI): m/z = 508 (M + NH₄)⁺HPLC (Method 1): Rₜ = 5.4 min. |
| 25 | 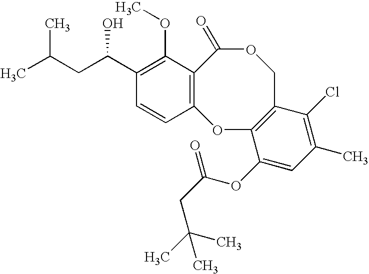 | ¹H-NMR (200 MHz, CDCl₃): δ = 0.98 (m, 6 H), 1.13 (s, 9 H), 1.42-1.80 (m, 3 H), 1.93 (d, 1 H), 2.31 (s, 3 H), 2.52 (s, 2 H), 3.99 (s, 3 H), 5.08 (m, 1 H), 5.39 (d, 1 H), 5.43 (d, 1 H), 6.98 (d, 1 H), 7.01 (br. s, 1 H), 7.59 (m, 1 H) ppm. MS (ESIpos): m/z = 527 (M + Na)⁺HPLC (Method 1): Rₜ = 5.5 min. |
| 26 | 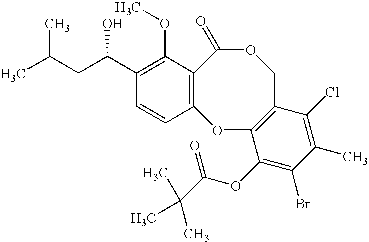 | ¹H-NMR (200 MHz, CDCl₃): δ = 0.98 (m, 6 H), 1.40 (s, 9 H), 1.42-1.88 (m, 4 H), 2.52 (s, 3 H), 3.99 (s, 3 H), 5.08 (m, 1 H), 5.40 (m, 2 H), 6.89 (d, 1 H), 7.59 (d, 1 H) ppm. MS (DCI): m/z = 586 (M + NH₄)⁺ |
| 27 | 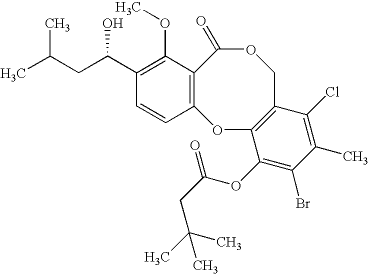 | ¹H-NMR (300 MHz, CDCl₃): δ = 0.98 (m, 6 H), 1.16 (s, 9 H), 1.42 (m, 1 H), 1.62-1.90 (m, 2 H), 1.90 (d, 1 H), 2.53 (s, 3 H), 2.61 (s, 2 H), 3.99 (s, 3 H), 5.08 (m, 1 H), 5.39 (m, 2 H), 6.95 (d, 1 H), 7.59 (d, 1 H) ppm. MS (DCI): m/z = 600 (M + NH₄)⁺HPLC (Method 1): Rₜ = 6.1 min. |

### EXAMPLE A-28

### 9-[(1S)-1-Hydroxy-3-methylbutyl]-8-methoxy-3-methyl-7-oxo-4-vinyl-5H,7H-dibenzo[b,g][1,5]dioxocin-1-yl pivalate

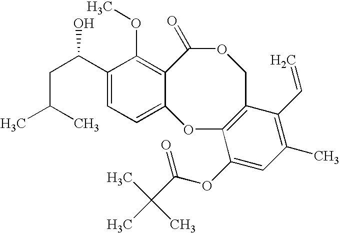

Under argon, 12.3 mg (0.10 mmol) of 2,2-dimethylpropionyl chloride are initially charged, and a solution of 27 mg (0.07 mmol) of the compound from Example A-VII in 150 μl of tetrahydrofuran are added. A solution of 20 μl (0.10 mmol) of 1,8-diazabicyclo[5.4.0]undec-7-ene in 100 μl of tetrahydrofuran is then added, and the mixture is stirred at room temperature overnight. For work-up, 0.8 ml of water, 3 drops of 1 N hydrochloric acid and 3 ml of ethyl acetate are added to the reaction mixture, and the mixture is filtered through a 1.1 g Extrelut/silica gel cartridge. The cartridge is washed with 12 ml of ethyl acetate and the filtrate is concentrated under reduced pressure. The crude product is purified by preparative HPLC. This gives 24 mg (73% of theory) of product.

¹H-NMR (300 MHz, CDCl₃): δ=0.98 (t, 6H), 1.4 (s, 9H), 1.66-1.81 (m, 2H), 1.95 (d, 1H), 2.2 (s, 3H), 2.92 (d, 1H), 3.98 (s, 3H), 4.95 (d, 1H), 5.08 (quintet, 1H), 5.22-5.33 (m, 2H), 5.58 (d, 1H), 6.58 (dd, 1H), 6.92 (s, 1H), 6.94 (d, 1H), 7.58 (d, 1H) ppm.

MS (ESIpos): m/z=505 (M+Na)⁺

The examples listed in the table below are prepared analogously to the procedures described above, from the corresponding starting materials:

| Example | | |
|---|---|---|
| A- | Structure | Analytical data |
| 29 | 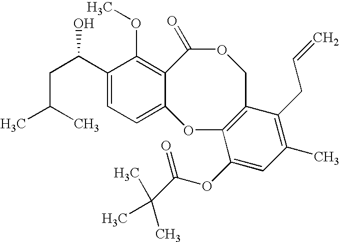 | ¹H-NMR (300 MHz, CDCl₃): δ = 0.97 (t, 6 H), 1.4 (s, 9 H), 1.49-1.93 (m, 4 H), 2.23 (s, 3 H), 3.28 (m, 2 H), 3.98 (s, 3 H), 4.77 (dd, 1 H), 5.01-5.05 (m, 2 H), 5.15 (s, 2 H), 5.79-5.88 (m, 1 H), 6.92 (d, 1 H), 6.93 (s, 1 H), 7.57 (d, 1 H) ppm. MS (ESIpos): m/z = 518 (M + Na)⁺ |
| 30 | 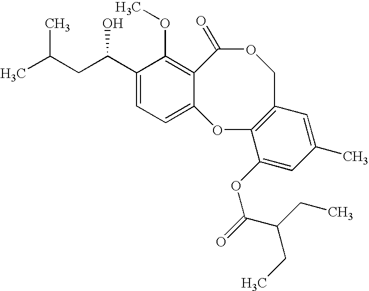 | LC-MS (Method 5): Rₜ = 5.13 min. MS (ESIpos): m/z = 471 (M + H)⁺ |
| 31 | 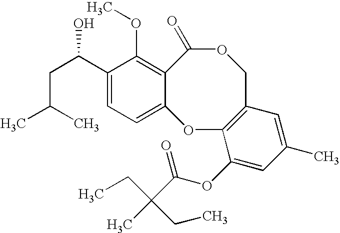 | LC-MS (Method 5): Rₜ = 5.29 min. MS (ESIpos): m/z = 485 (M + H)⁺ |
| 32 | 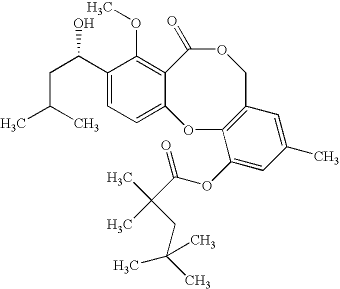 | ¹H-NMR (300 MHz, CDCl₃): δ = 0.97 (t, 6 H), 1.05 (s, 9 H), 1.41 (s, 6 H), 1.65-1.82 (m, 2 H), 1.84 (s, 2 H), 1.91 (d, 1 H), 2.28 (s, 3 H), 3.97 (s, 3 H), 4.77 (dd, 1 H), 5.04-5.12 (m, 3 H), 6.72 (d, 1 H), 6.89 (d, 1 H), 6.97 (d, 1 H), 7.58 (d, 1 H) ppm. MS (ESIpos): m/z = 535 (M + Na)⁺ |
| 33 | 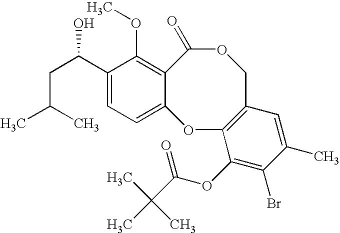 | ¹H-NMR (200 MHz, CDCl₃): δ = 0.98 (dd, 6 H), 1.45 (s, 9 H), 1.42-2.00 (m, 4 H), 2.37 (s, 3 H), 3.98 (s, 3 H), 4.98-5.15 (m, 3 H), 6.85 (br. s, 1 H), 6.93 (d, 1 H), 7.60 (d, 1 H) ppm. MS (DCI): m/z = 552 (M + NH₄)⁺ |
| 34 | 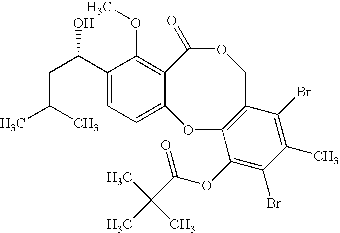 | ¹H-NMR (200 MHz, CDCl₃): δ = 0.98 (dd, 6 H), 1.43 (s, 9 H), 1.45 (s, 9 H), 1.40-1.89 (m, 3 H), 1.96 (d, 1 H), 2.41 (s, 3 H), 3.99 (s, 3 H), 5.19 (m, 1 H), 5.43 (br. s, 2 H), 6.89 (d, 1 H), 7.61 (d, 1 H). MS (DCI): m/z = 630 (M + NH₄)⁺HPLC (Method 1): Rₜ = 5.96 min. |
| 35 | 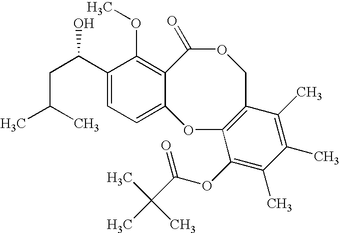 | ¹H-NMR (300 MHz, CDCl₃): δ = 0.98 (dd, 6 H), 1.41 (s, 9 H), 1.46-1.87 (m, 4H), 2.12 (s, 3 H), 2.14 (s, 3 H), 2.18 (s, 3 H), 3.99 (s, 3 H), 5.07 (m, 1 H), 5.25 (m, 2 H), 6.90 (d, 1 H), 7.55 (d, 1 H) ppm. MS (ESIpos): m/z = 485 (M + H)⁺HPLC (Method 1): Rₜ = 5.47 min. |
| 36 | 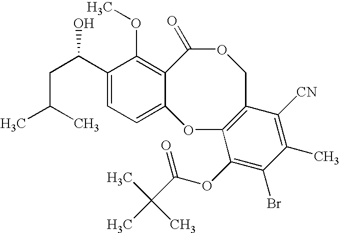 | ¹H-NMR (300 MHz, CDCl₃): δ = 0.99 (dd, 6 H), 1.40-1.90 (m, 4 H), 1.45 (s, 9H), 2.66 (s, 3 H), 3.99 (s, 3 H), 5.10 (m, 1 H), 5.39 (m, 2 H), 6.89 (d, 1 H), 7.64 (d, 1 H) ppm. MS (DCI): m/z = 577/579 (M + NH₄)⁺HPLC (Method 1): Rₜ = 5.57 min. |
| 37 | 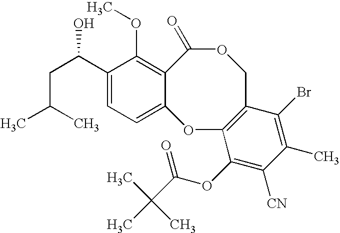 | ¹H-NMR (300 MHz, CDCl₃): δ = 0.98 (dd, 6 H), 1.45 (s, 9H), 1.59-1.98 (m, 4 H), 2.62 (s, 3 H), 3.99 (s, 3 H), 5.09 (m, 1 H), 5.45 (m, 2 H), 6.89 (d, 1 H), 7.64 (d, 1 H) ppm. MS (DCI): m/z = 577/579 (M + NH₄)⁺HPLC (Method 1): Rₜ = 5.53 min. |

### EXAMPLE A-38

### 8-Allyl-3-[(1S)-1-hydroxy-3-methylbutyl]-11-(isopentyloxy)-4-methoxy-9-methyl-5H,7H-dibenzo[b,g][1,5]dioxocin-5-one

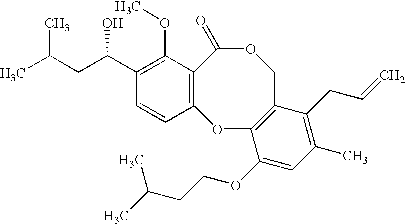

50 mg (95.89 μmol) of the compound from Example A-IX are dissolved in 1 ml of dimethylformamide, and 6 mg (4.79 μmol) of tetrakis(triphenylphosphine)-palladium(0) are added. For 5 minutes, argon is passed through the reaction mixture. 149 μl (479.44 μmol) of allyltributyltin are then added dropwise. The reaction vessel is closed and the mixture is stirred at 90° C. overnight. For work-up, water is added to the reaction mixture after cooling, and the mixture is extracted twice with ethyl acetate. The organic phase is dried over sodium sulphate and concentrated under reduced pressure. The crude product is purified on a short silica gel column (mobile phase: cyclohexane/ethyl acetate 20:1→2:1). This gives 43 mg (94% of theory) of product.

¹H-NMR (300 MHz, CDCl₃): δ=0.94-1.01 (m, 12H), 1.25-2.04 (m, 7H), 2.23 (s, 3H), 3.23-3.24 (m, 2H), 3.97 (s, 3H), 4.08 (t, 2H), 4.72 (dd, 1H), 5.01 (dd, 1H), 5.07 (m, 1H), 5.17 (s, 2H), 5.75-5.88 (m, 1H), 6.81 (s, 1H), 6.92 (d, 1H), 7.55 (d, 1H) ppm.

MS (ESIpos): m/z=483 (M+H)⁺

The examples listed in the table below are prepared analogously to the procedures described above, from the corresponding starting materials:

| Example | | |
|---|---|---|
| A- | Structure | Analytical data |
| 39 | 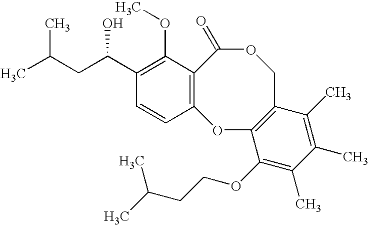 | ¹H-NMR (200 MHz, CDCl₃): δ = 0.93-1.00 (m, 12 H), 1.42-2.2 (m, 7 H), 2.09 (s, 3 H), 2.14 (s, 3 H), 2.25 (s, 3 H), 3.99 (s, 3 H), 4.03 (t, 2 H), 5.07 (m, 1 H), 5.26 (s, 2 H), 6.98 (d, 1 H), 7.54 (d, 1 H) ppm. |
| 40 | 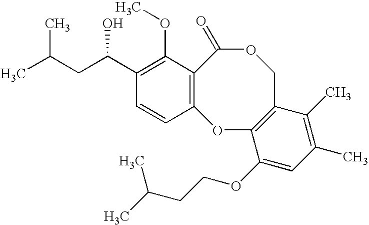 | ¹H-NMR (200 MHz, CDCl₃): δ = 0.94-1.01 (m, 12 H), 1.26 (t, 2 H), 1.42-1.52 (m, 1 H), 1.65-1.95 (m, 4 H), 2.05 (s, 3 H), 2.23 (s, 3 H), 3.98 (s, 3 H), 4.04-4.14 (m, 2 H), 5.08 (quintet, 1 H), 5.26 (s, 2 H), 6.80 (s, 1 H), 6.92 (d, 1 H), 7.55 (d, 1 H) ppm. MS (ESIpos): m/z = 479 (M + Na)⁺ |

### EXAMPLE A-41

### 3-[(1S)-1-Hydroxy-3-methylbutyl]-11-(isopentyloxy)-4-methoxy-9-methyl-8-vinyl-5H,7H-dibenzo[b,g][1,5]dioxocin-5-one

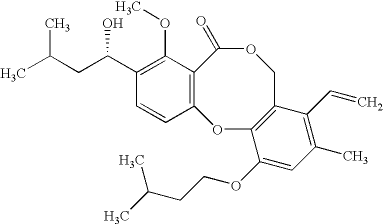

50 mg (95.89 μmol) of the compound from Example A-IX are dissolved in 1 ml of dimethylformamide, and 15.5 mg (22.05 μmol) of bis(triphenylphosphine)-palladium(II) chloride and 15 μl of triethylamine are added. For 5 minutes, argon is passed through the reaction mixture. 140 μl (479.44 μmol) of tributylvinyltin are then added dropwise. The reaction vessel is closed and stirred at 80° C. overnight. For work-up, water is added to the reaction mixture after cooling, and the mixture is extracted twice with ethyl acetate. The organic phase is dried over sodium sulphate and concentrated under reduced pressure. The crude product is purified by preparative thick-layer chromatography (mobile phase: cyclohexane/ethyl acetate 2:1). This gives 20 mg (44% of theory) of product.

LC-MS (Method 3): Rₜ=3.78 min.

MS (ESIpos): m/z=469 (M+H)⁺

### EXAMPLE A-42

### 9-[(1S)-1-Hydroxy-3-methylbutyl]-8-methoxy-3-methyl-7-oxo-5H,7H-dibenzo[b,g]-[1,5]-dioxocin-1-yl 2-propanesulphonate

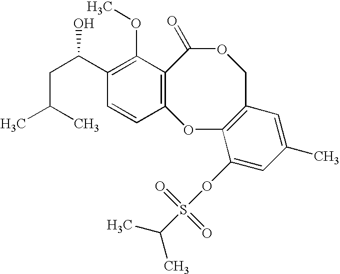

Under argon, 100 mg (0.27 mmol) of penicillide are dissolved in 1 ml of tetrahydrofuran, and 11.3 mg (0.28 mmol) of sodium hydride are added at 0° C. After 5 minutes, 40.2 mg (0.28 mmol) of 2-propanesulphonyl chloride and 110 μl (0.81 mmol) of triethylamine are added to the reaction solution, and the mixture is stirred at room temperature for 2 hours. For work-up, the reaction mixture is diluted with 25 ml of dichloromethane and washed twice with water. The organic phase is dried over magnesium sulphate and concentrated under reduced pressure. The residue is purified by preparative HPLC. This gives 70 mg (54% of theory) of product.

¹H-NMR (300 MHz, CDCl₃): δ=0.98 (t, 6H), 1.45-1.80 (m, 3H), 1.59 (d, 6H), 1.92 (br. s, 1H), 2.30 (s, 3H), 3.62 (quintet, 1H), 3.98 (s, 3H), 5.06-5.16 (m, 3H), 6.79 (s, 1H), 7.20 (d, 1H), 7.27 (s, 1H), 7.61 (d, 1H) ppm.

MS (ESIpos): m/z=501 (M+Na)⁺

HPLC (Method 1): Rₜ=4.78 min.

The examples listed in the table below are prepared analogously to the procedures described above, from the corresponding starting materials:

| Example | | |
|---|---|---|
| A- | Structure | Analytical data |
| 43 | 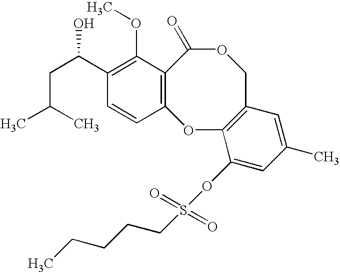 | ¹H-NMR (400 MHz, CDCl₃): δ = 0.86 (t, 3 H), 0.98 (dd, 6 H), 1.31 (q, 2 H), 1.42 (quintet, 2 H), 1.44-1.84 (m, 3 H); 1.93 (d, 1 H), 2.00 (quintet, 2 H), 2.31 (s, 3 H), 3.34-3.38 (m, 2 H), 3.98 (s, 3 H), 5.05-5.13 (m, 3 H), 6.81 (s, 1 H), 7.15 (d, 1 H), 7.27 (s, 1 H), 7.61 (d, 1 H) ppm. MS (ESIpos): m/z = 507 |
| #(M + H)⁺HPLC (Method 1): Rₜ = 4.89 min. | | |
| 44 | 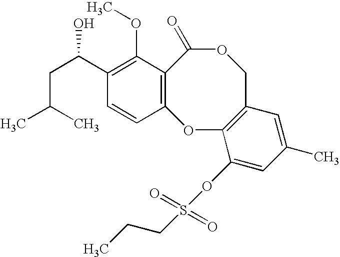 | ¹H-NMR (300 MHz, CDCl₃): δ = 0.98 (t, 6 H), 1.09 (t, 3 H), 1.45-1.84 (m, 3 H), 1.93 (d, 1 H), 2.05 (sextet, 2 H), 2.30 (s, 3 H), 3.32-3.38 (m, 2 H), 3.98 (s, 3 H), 5.06-5.15 (m, 3 H), 6.80 (s, 1 H), 7.15 (d, 1 H), 7.27 (s, 1 H), 7.61 (d, 1 H) ppm. MS (ESIpos): m/z = 501 (M + Na)⁺ |
| #HPLC (Method 1): Rₜ = 4.81 min. | | |
| 45 | 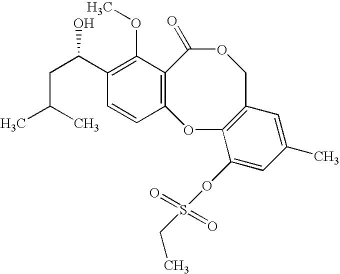 | ¹H-NMR (300 MHz, CDCl₃): δ = 0.98 (t, 6 H), 1.45-1.84 (m, 3 H), 1.55 (t, 3 H), 1.93 (d, 1 H), 2.31 (s, 3 H), 3.41 (q, 2 H), 3.98 (s, 3 H), 5.05-5.14 (m, 3 H), 6.80 (s, 1 H), 7.15 (d, 1 H), 7.27 (s, 1 H), 7.61 (d, 1 H) ppm. MS (ESIpos): m/z = 487 (M + Na)⁺HPLC (Method 1): Rₜ = 4.71 min. |
| 46 | 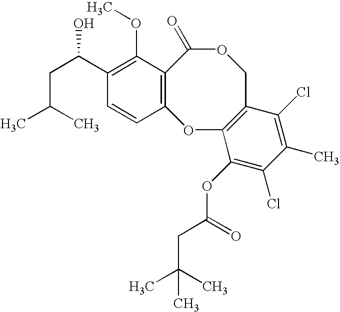 | ¹H-NMR (200 MHz, CDCl₃): δ = 0.99 (dd, 6 H), 1.16 (s, 9 H), 1.41-1.90 (m, 3 H), 1.95 (d, 1 H), 2.46 (s, 3 H), 2.61 (s, 2 H), 3.98 (s, 3 H), 5.04-5.16 (m, 1 H), 5.33-5.51 (m, 2 H), 6.92 (d, 1 H), 7.61 (d, 1 H) ppm. MS (ESIpos): m/z = 561 (M + Na)⁺HPLC (Method 2): Rₜ = 6.03 min. |
| 47 | 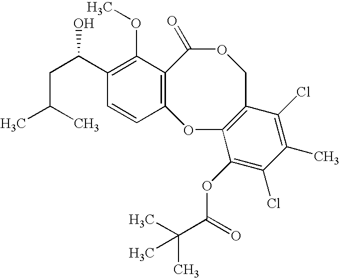 | ¹H-NMR (200 MHz, CDCl₃): δ = 0.99 (dd, 6 H), 1.43 (s, 9 H), 1.41-1.90 (m, 3 H), 1.94 (d, 1 H), 2.47 (s, 3 H), 3.98 (s, 3 H), 5.04-5.16 (m, 1 H), 5.32-5.54 (m, 2 H), 6.87 (d, 1 H), 7.60 (d, 1 H) ppm. MS (ESIpos): m/z = 547 (M + Na)⁺HPLC (Method 2): Rₜ = 5.87 min. |

### EXAMPLE A-48

### 8-Methoxy-3-methyl-9-[(1Z)-3-methyl-1-butenyl]-7-oxo-5H,7H-dibenzo[b,g][1,5]-dioxocin-1-yl 2-propanesulphonate

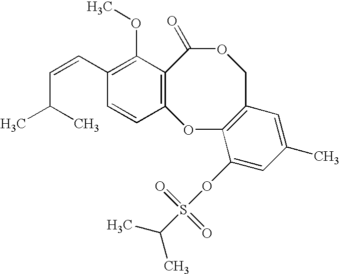

The compound is formed as a by-product in the preparation of Example A-42. 22 mg (17% of theory) is obtained.

¹H-NMR (300 MHz, CDCl₃): δ=1.10 (d, 6H), 1.59 (d, 6H), 2.30 (s, 3H), 2.49 (quintet, 1H), 3.61 (quintet, 1H), 3.91 (s, 3H), 5.05-5.11 (m, 2H), 6.21 (dd, 1H), 6.56 (d, 1H), 6.78 (s, 1H), 7.16 (d, 1H), 7.27 (s, 1H), 7.58 (d, 1H) ppm.

MS (ESIpos): m/z=483 (M+Na)⁺

HPLC (Method 1): Rₜ=5.31 min.

### EXAMPLE A-49

### 8-Methoxy-3-methyl-9-[(1Z)-3-methyl-1-butenyl]-7-oxo-5H,7H-dibenzo[b,g][1,5]-dioxocin-1-yl 1-propanesulphonate

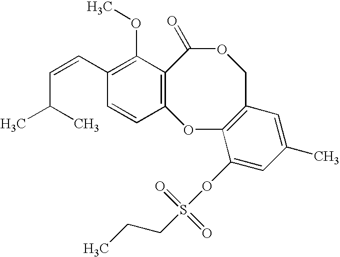

The compound is formed as a by-product in the preparation of Example A-44. 100 mg (0.27 mmol) of penicillide give 59 mg (48% of theory) of the title compound.

¹H-NMR (300 MHz, CDCl₃): δ=1.06-1.11 (m, 9H), 2.05 (sextet, 2H), 2.30 (s, 3H), 2.51 (sextet, 1H), 3.35 (t, 2H), 3.92 (s, 3H), 5.05-5.14 (m, 2H), 6.21 (dd, 1H), 6.55 (d, 1H), 6.79 (s, 1H), 7.10 (d, 1H), 7.27 (s, 1H), 7.58 (d, 1H) ppm.

MS (ESIpos): m/z=483 (M+Na)⁺

HPLC (Method 1): Rₜ=5.34 min.

### EXAMPLE A-50

### 3-(1-Hydroxy-2-methyl-3-butenyl)-11-(isopentyloxy)-4-methoxy-9-methyl-5H,7H-dibenzo[b,g][1,5]dioxocin-5-one

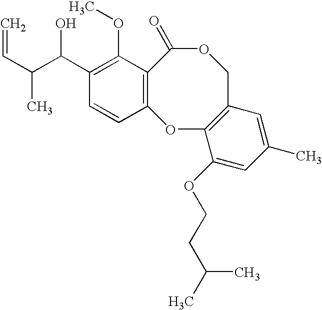

50 mg (130 μmol) of the compound from Example A-XIII are dissolved in 2 ml of tetrahydrofuran and, at −78° C., 520 μl of a 0.5 M solution of 1-methylprop-2-enyl-magnesium chloride in tetrahydrofuran are added dropwise. The mixture is stirred at room temperature-overnight. 1 ml of saturated ammonium chloride solution is added to the reaction solution, and the mixture is filtered through a 1.8 g Extrelut/silica gel cartridge. The mixture is eluted with dichloromethane and the filtrate is concentrated under reduced pressure. The residue is purified by preparative HPLC. This gives 39 mg (68% of theory) of product.

LC-MS (Method 4): Rₜ=3.04 min.

MS (ESIpos): m/z=441 (M+H)⁺

The examples listed in the table ‘below are prepared ’ analogously to the procedures described above, from the corresponding starting materials:

| Example | | |
|---|---|---|
| A- | Structure | Analytical data |
| 51 | 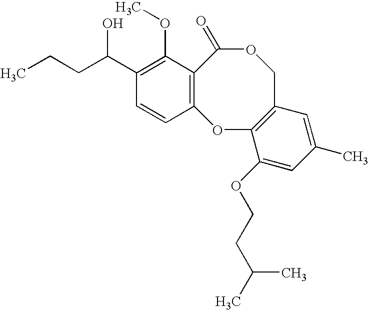 | LC-MS (Method 4): Rₜ = 3.05 min. MS (ESIpos): m/z = 429 (M + H)⁺ |
| 52 | 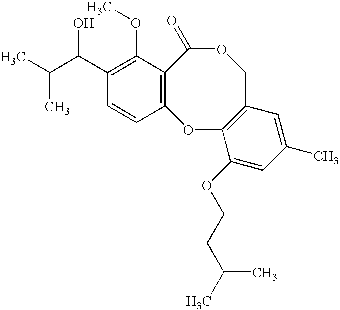 | LC-MS (Method 4): Rₜ = 3.12 min. MS (ESIpos): m/z = 429 (M + H)⁺ |
| 53 | 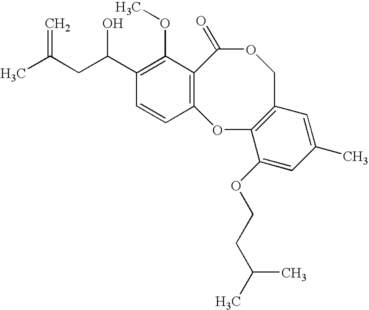 | R_{f} = 0.22 (cyclohexane/ethyl acetate 2:1) MS (ESIpos): m/z = 463 (M + Na)⁺ |

### EXAMPLE A-54

### 3-(1-Hydroxy-4-pentenyl)-11-(isopentyloxy)-4-methoxy-9-methyl-5H,7H-dibenzo[b,g][1,5]dioxocin-5-one

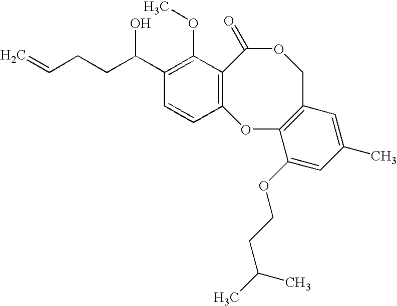

To prepare the Grignard reagent, 243 mg (10 mmol) of magnesium turnings are initially charged, dried by heating under reduced pressure and, after cooling under argon, covered with 2 ml of dry diethyl ether. A few drops of bromomethyl-cyclopropane are then added, and the mixture is warmed until the reaction starts. The remaining bromomethylcyclopropane [in total 970 μl (10 mmol)], dissolved in 3 ml of diethyl ether, is added dropwise, and the mixture is then heated in an oil bath under reflux for another 30 minutes until most of the magnesium has dissolved. After cooling, 160 μl (about 2 eq.) of the Grignard solution are added to a solution, cooled to −78° C., of 60 mg (0.16 mmol) of the compound from Example A-XIII in 1.6 ml of tetrahydrofuran. After 3 hours at −78° C., the reaction solution is hydrolysed using saturated ammonium chloride solution. The mixture is diluted with water and extracted with diethyl ether. The combined organic phases are washed with saturated sodium chloride solution, dried over magnesium sulphate and concentrated under reduced pressure. The residue is purified by preparative HPLC. 44 mg (62% of theory) of the title compound are isolated.

¹H-NMR (300 MHz, CDCl₃): δ=1.00 (d, 6H), 1.35-2.23 (m, 8H), 2.27 (s, 3H), 3.96 (s, 3H), 4.09 (t, 2H), 4.98-5.09 (m, 5H), 5.78-5.91 (m, 1H), 6.41 (br. s, 1H), 6.79 (br. s, 1H), 6.94 (d, 1H), 7.55 (d, 1H) ppm.

MS (ESIpos): m/z=423 (M+Na)⁺

HPLC (Method 2): Rₜ=5.55 min.

### EXAMPLE A-55

### 3-(1-Hydroxy-3,3-dimethylbutyl)-11-(isopentyloxy)-4-methoxy-9-methyl-5H,7H-dibenzo[b,g][1,5]dioxocin-5-one

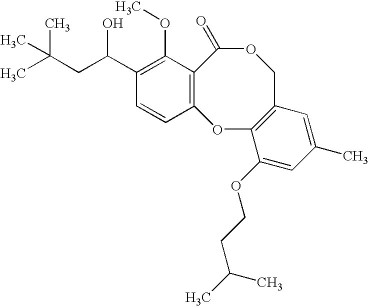

Under argon, over a period of 5 minutes, 170 μl of a 1.7 M solution of tert-butyllithium in n-pentane are added dropwise to a solution, cooled to −78° C., of 22 mg of 1-bromo-2,2-dimethylpropane (0.14 mmol) in 0.5 ml of dry tetrahydrofuran. The mixture is stirred at this temperature for 30 minutes. This solution is then added to a solution, cooled to −78° C., of 50 mg (0.13 mmol) of the compound from Example A-XIII in 0.5 ml of tetrahydrofuran. The mixture is stirred for another 18 hours, warming to room temperature. The reaction solution is then hydrolysed using 0.5 ml of saturated ammonium chloride solution and filtered through a 1.8 g Extrelut/silica gel cartridge. The filtrate is diluted with water and extracted with diethyl ether. The resulting solution is concentrated under reduced pressure. The residue is purified by preparative HPLC. 10 mg (16% of theory) of the title compound are isolated.

¹H-NMR (200 MHz, CDCl₃): δ=0.97-1.04 (m, 15H), 1.58-2.00 (m, 6H), 2.27 (s, 3H), 3.97 (s, 3H), 4.09 (t, 2H), 5.08 (s, 2H), 5.15 (dd, 1H), 6.41 (br. s, 1H), 6.79 (br. s, 1H), 6.93 (d, 1H), 7.58 (d, 1H) ppm.

MS (ESIpos): m/z=479 (M+Na)⁺

### EXAMPLE A-56

### 3-(1-Hydroxy-3-methylbutyl)-4-methoxy-9-methyl-11-[3-methyl-3-(1H-pyrrol-1-yl)-1-butynyl]-5H,7H-dibenzo[b,g][1,5]dioxocin-5-one

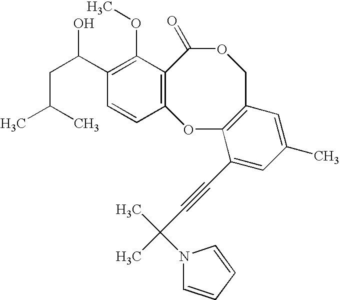

88.5 mg (0.18 mmol) of the compound from Example A-XVIII are dissolved in 2 ml of warm methanol. With ice-cooling, 10.3 mg (0.27 mmol) of sodium borohydride are added. The reaction mixture is stirred for 70 minutes. During this time, the reaction mixture is warmed to room temperature. The mixture is then concentrated under reduced pressure. The residue is taken up in dichloromethane and 0.4 ml of water and filtered through a 500 mg Extrelut/silica gel cartridge. The residue is eluted using dichloromethane and the filtrate is concentrated under reduced pressure. The residue is purified on a 3 g silica gel cartridge (mobile phase: cyclohexane/ethyl acetate 100:0→40:60). This gives 66 mg (74% of theory) of product.

¹H-NMR (200 MHz, CDCl₃): δ=0.98 (dd, 6H), 1.40-1.96 (m, 4H), 1.90 (s, 6H), 2.26 (s, 3H), 3.99 (s, 3H), 5.09 (br. s, 3H), 6.18 (t, 2H), 6.83 (br. s, 1H), 6.99 (d, 1H), 7.06 (t, 2H), 7.28 (br. s, 1H), 7.56 (d, 1H) ppm.

MS (ESIpos): m/z=510 (M+Na)⁺

HPLC (Method 8): Rₜ=5.25 min.

The examples listed in the table below are prepared analogously to the procedures described above, from the corresponding starting materials:

| Example | | |
|---|---|---|
| A- | Structure | Analytical data |
| 57 | 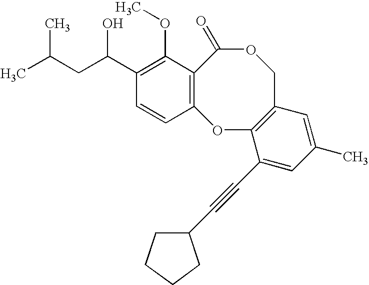 | ¹H-NMR (200 MHz, CDCl₃): δ = 0.98 (dd, 6 H), 1.42-2.06 (m, 12 H), 2.24 (s, 3 H), 2.90 (quintet, 1 H), 3.97 (s, 3 H), 5.07 (br. s, 3 H), 6.74 (br. s, 1 H), 7.06 (d, 1 H), 7.23 (br. s, 1 H), 7.58 (d, 1 H) ppm. |
| 58 | 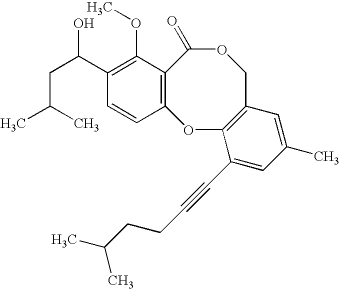 | ¹H-NMR (300 MHz, CDCl₃): δ = 0.92-0.99 (m, 12 H), 1.42-1.85 (m, 6 H), 1.95 (br. s, 1 H), 2.24 (s, 3 H), 2.48 (t, 2 H), 3.97 (s, 3 H), 5.06 (br. s, 3 H), 6.74 (br. s, 1 H), 7.05 (d, 1 H), 7.23 (br. s, 1 H), 7.57 (d, 1 H) ppm. MS (ESIpos): m/z = 473 (M + H)⁺HPLC (Method 1): Rₜ = 5.75 min. |
| 59 | 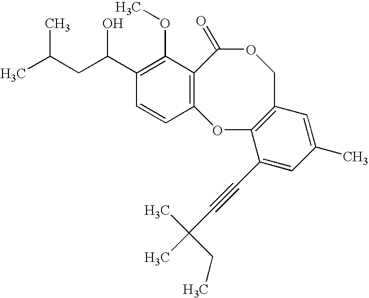 | ¹H-NMR (300 MHz, CDCl₃): δ = 0.98 (m, 6 H), 1.06 (t, 3 H), 1.30 (s, 6 H), 1.48-1.88 (m, 5 H), 1.93 (d, 1 H), 2.24 (s, 3 H), 3.98 (s, 3 H), 5.06 (br. s, 3 H), 6.73 (br. s, 1 H), 7.05 (d, 1 H), 7.23 (br. s, 1 H), 7.57(d, 1 H) ppm. MS (ESIpos): m/z = 473 (M + Na)⁺HPLC (Method 1): Rₜ = 5.73 min. |
| 60 | 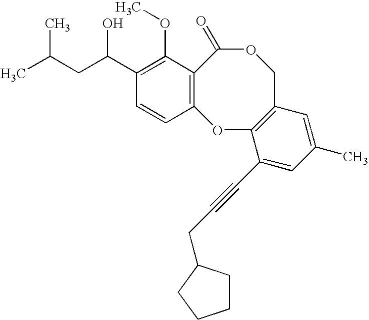 | ¹H-NMR (200 MHz, CDCl₃): δ = 0.97 (t, 6 H), 1.32-1.95 12 H), 2.17 (quintet, 1 H), 2.24 (s, 3 H), 2.48 (d, 2 H), 3.97 (s, 3 H), 5.06 (br. s, 3 H), 6.74 (br. s, 1 H), 7.06 (d, 1 H), 7.23 (br. s, 1 H), 7.57 (d, 1 H) ppm. MS (ESIpos): m/z = 485 (M + Na)⁺HPLC (Method 1): Rₜ = 5.84 min. |
| 61 | 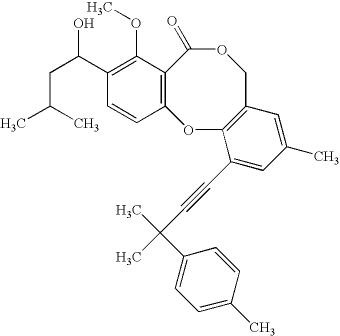 | ¹H-NMR (300 MHz, CDCl₃): δ = 0.97 (t, 6 H), 1.42-1.85 (m, 3 H), 1.69 (s, 6 H), 1.93 (d, 1 H), 2.25 (s, 3 H), 2.32 (s, 3 H), 3.98 (s, 3 H), 5.08 (br. s, 3 H), 6.77 (br. s, 1 H), 7.03 (d, 1 H), 7.13 (d, 2 H), 7.29 (br. s, 1 H), 7.53 (dd, 3 H) ppm. MS (ESIpos): m/z = 435 (M + Na)⁺ |
| #HPLC (Method 1): Rₜ = 5.90 min. | | |
| 62 | 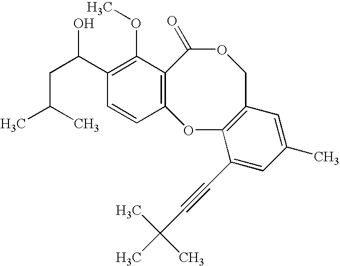 | ¹H-NMR (200 MHz, CDCl₃): δ = 0.98 (dd, 6 H), 1.35 (s, 9 H), 1.40-1.90 (m, 3 H), 1.95 (d, 1 H), 2.24 (s, 3 H), 3.98 (s, 3 H), 5.03-5.16 (m, 3 H), 6.74 (br. s, 1 H), 7.07 (d, 1 H), 7.22 (br. s 1 H), 7.58 (d, 1 H) ppm. MS (ESIpos): m/z = 459 (M + Na)⁺HPLC (Method 1): Rₜ = 5.59 min. |
| 63 | 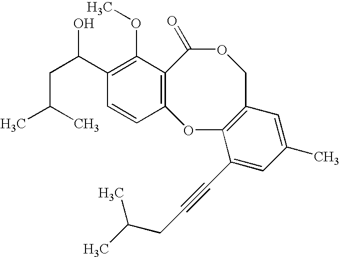 | ¹H-NMR (200 MHz, CDCl₃): δ = 0.98 (dd, 6 H), 1.06 (d, 6 H), 1.45-1.91 (m, 4 H), 1.96 (d, 1 H), 2.24 (s, 3 H), 2.38 (d, 2 H), 3.98 (s, 3 H), 5.03-5.15 (m, 3 H), 6.75 (br. s, 1 H), 7.07 (d, 1 H), 7.23 (br. s, 1 H), 7.57 (d, 1 H) ppm. MS (DCI): m/z = 454 (M + NH₄)⁺HPLC (Method 1): Rₜ = 5.60 min. |
| 64 | 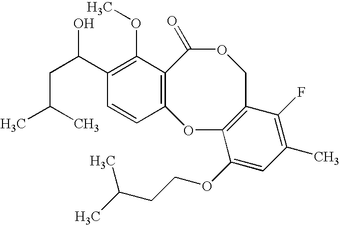 | ¹H-NMR (400 MHz, CDCl₃): δ = 0.89-1.00 (m, 12 H), 1.51 (m, 1 H), 1.56-1.92 (m, 6H), 2.13 (br. s, 3 H), 3.90 (s, 3 H), 3.99 (t, 2 H), 5.01 (m, 1 H), 5.11 (dd, 2 H), 6.71 (d, 1 H), 6.86 (d, 1 H), 7.50 (d, 1 H) ppm. MS (ESIpos): m/z = 483 (M + Na)⁺HPLC (Method 2): Rₜ = 6.23 min. |
| 65 | 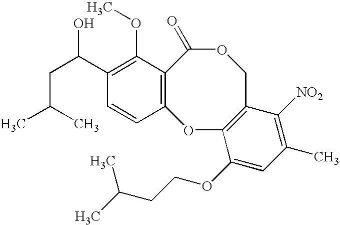 | ¹H-NMR (300 MHz, CDCl₃): δ = 0.96-1.02 (m, 12 H), 1.46-1.96 (m, 6 H), 1.93 (d, 1 H), 2.33 (s, 3 H), 3.97 (s, 3 H), 4.15 (t, 2 H), 5.10 (br. s, 3 H), 6.82 (br. s, 1 H), 6.88 (d, 1 H), 7.61 (d, 1 H) ppm. MS (ESIpos): m/z = 510 (M + Na)⁺ |
| 66 | 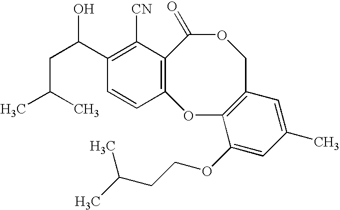 | ¹H-NMR (300 MHz, CDCl₃): δ = 0.99-1.07 (m, 12 H), 1.59-1.70 (m, 3 H), 1.78 (q, 2 H), 1.92 (sep., 1 H), 2.04 (sep., 1 H), 2.28 (s, 3 H), 4.10 (t, 2 H), 5.07 (s, 2 H), 5.49 (dd, 1 H), 6.44 (br. s, 1 H), 6.82 (br. s, 1 H), 7.51 (s, 2 H) ppm. MS (DCI): m/z = 456 (M + NH₄)⁺ |
| #HPLC (Method 2): Rₜ = 5.54 min. | | |

### EXAMPLE A-67

### 3-(1-Hydroxy-3-methylbutyl)-1-(isopentyloxy)-4-methoxy-9-methyl-8-nitro-5H,7H-dibenzo[b,g][1,5]dioxocin-5-one

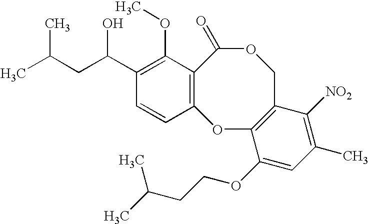

The preparation is carried out analogously to Example A-56 from 170 mg (350 μmol) of the compound from Example A-XXX. Subsequent chromatographic separation of the enantiomers on a chiral phase [column: stationary silica gel phase with the covalently attached selector poly(N-methacryloyl-L-leucine tert-butyl ester), 20 mm×250 mm; mobile phase: isohexane/ethyl acetate 80:20; flow rate: 25 ml/min; room temperature: detection: 254 nm] gives 40 mg (24% of theory) of a pure enantiomer whose configuration was not determined.

HPLC (Method 1): Rₜ=5.57 min.

MS (ESIpos): m/z=510 (M+Na)⁺

Rₜ=11.69 min. [column: Chiracel OD 10 μM; mobile phase: hexane/isopropanol 91:9; flow rate: 1 ml/min; room temperature; detection: 254 nm].

### EXAMPLE A-68

### 11-(1-Heptynyl)-3-(1-hydroxy-3-methylbutyl)-4-methoxy-9-methyl-5H,7H-dibenzo[b,g][1,5]dioxocin-5-on

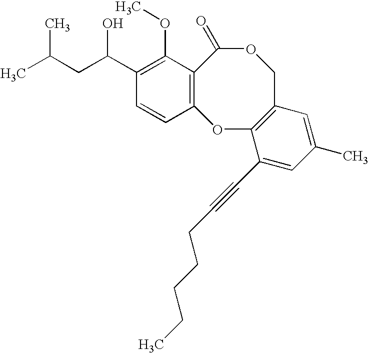

The preparation is carried out analogously to Example A-56 from 60 mg (134 μmol) of the compound from Example A-XXIV.

Racemate:

¹H-NMR (200 MHz, CDCl₃): δ=0.89 (t, 3H), 0.98 (dd, 6H), 1.25-1.85 (m, 9H), 1.95 (d, 1H), 2.25 (s, 3H), 2.48 (t, 2H), 3.97 (s, 3H), 5.07 (br. s, 3H), 6.76 (br. s, 1H), 7.06 (d, 1H), 7.23 (br. s, 1H), 7.57 (d, 1H) ppm.

MS (ESIpos): m/z=473 (M+Na)⁺.

Subsequent chromatographic separation of the enantiomers on a chiral phase [column: Chiracel OC 10 μM, 20 mm×250 mm; mobile phase: isohexane/isopropanol 90:10; flow rate: 20 ml/min; room temperature; detection: 254 nm] gives 15 mg (25% of theory) of a pure enantiomer whose configuration was not determined.

Rₜ=7.85 min. [column: Chiracel OD 10 μM; mobile phase: hexane/isopropanol 91:9; flow rate: 1 ml/min; room temperature; detection: 254 nm].

### EXAMPLE A-69 AND EXAMPLE A-70

### 11-(2-Cyclopentylethen-1-yl)-3-(1-hydroxy-3-methylbutyl)-4-methoxy-9-methyl-5H,7H-dibenzo[b,g][1,5]dioxocin-5-one (Example A-69) and 11-(2-Cyclopentylethan-1-yl)-3-(1-hydroxy-3-methylbutyl)-4-methoxy-9-methyl-5H,7H-dibenzo[b,g][1,5]dioxocin-5-one (Example A-70)

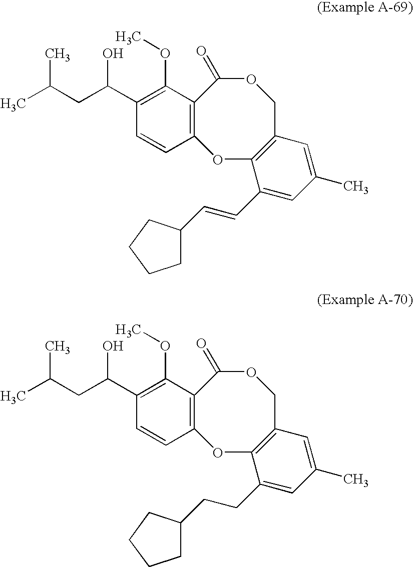

The preparation is carried out analogously to Example A-56 from 87 mg (0.19 mmol) of the compound from Example A-XXVII. The crude product is purified by preparative HPLC. This gives 12 mg (14% of theory) of the compound of Example A-69 and 25 mg (29% of theory) of the compound of Example A-70.

### EXAMPLE A-69

¹H-NMR (300 MHz, CDCl₃): δ=0.97 (t, 6H), 1.40-1.95 (m, 12H), 2.26 (s, 3H), 2.68 (sextet, 1H), 3.98 (s, 3H), 5.03-5.13 (m, 3H), 6.32 (dd, 1H), 6.68 (br. s, 1H), 6.82-6.93 (m, 2H), 7.32 (br. s, 1H), 7.55 (d, 1H) ppm.

MS (DCI): m/z=468 (M+NH₄)⁺

### EXAMPLE A-70

¹H-NMR (300 MHz, CDCl₃): δ=0.97 (t, 6H), 1.10-1.92 (m, 15H), 2.25 (s, 3H), 2.79-2.85 (m, 2H), 3.98 (s, 3H), 5.05-5.10 (m, 3H), 6.67 (br. s, 1H), 6.87 (d, 1H), 7.04 (br. s, 1H), 7.56 (d, 1H) ppm.

MS (ESIpos): m/z=475 (M+Na)⁺

### EXAMPLE A-71

### 4-Ethoxy-3-(1-hydroxy-3-methylbutyl)-11-(isopentyloxy)-9-methyl-5H,7H-dibenzo[b,g][1,5]dioxocin-5-one

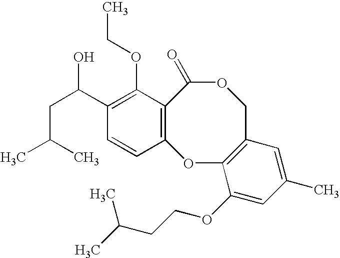

The preparation is carried out analogously to Example A-56 from 100 mg (220 μmol) of the compound from Example A-XXX. The crude product is purified by preparative HPLC.

Racemate:

¹H-NMR (300 MHz, CDCl₃): δ=0.95-1.01 (m, 12H), 1.42 (t, 3H), 1.47-1.97 (m, 7H), 2.27 (s, 3H), 4.06-4.21 (m, 4H), 5.05-5.12 (m, 3H), 6.40 (s, 1H), 6.79 (s, 1H), 6.93 (d, 1H), 7.56 (d, 1H) ppm.

MS (ESIpos): m/z=479 (M+Na)⁺

HPLC (Method 1): Rₜ=5.52 min.

Subsequent chromatographic separation of the enantiomers on a chiral phase [column: stationary silica gel phase with the covalently attached selector poly(N-methacryloyl-L-leucine tert-butyl ester), 20 mm×250 mm; mobile phase: isohexane/ethyl acetate 85:15; flow rate: 10 ml/min; room temperature; detection: 280 nm] gives 22 mg (22% of theory) of a pure enantiomer whose configuration was not determined.

Rₜ=4.48 min. [column: stationary silica gel phase with the covalently attached selector poly(N-methacryloyl-L-leucine tert-butyl ester); mobile phase: hexane/isopropanol 80:20; flow rate: 1 ml/min; room temperature; detection: 254 nm].

### EXAMPLE A-72

### 11-Allyl-3-[(1S)-1-hydroxy-3-methylbutyl]-4-methoxy-9-methyl-5H,7H-dibenzo[b,g][1,5]dioxocin-5-one

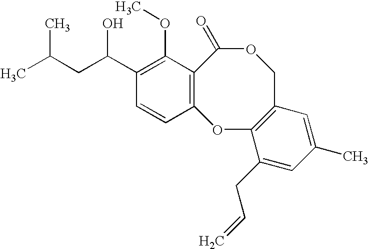

100 mg (187.8 μmol) of the compound from Example A-XVI are dissolved in 4 ml of dimethylformamide, and 50 mg (43.2 μmol) of tetrakis(triphenylphosphine)-palladium(0) are added. For 5 minutes, argon is passed through the reaction mixture, 291 μl (939 μmol) of allyltributyltin are then added and the reaction vessel is closed and heated at 90° C. overnight. For work-up, water is added to the reaction mixture after cooling, and the mixture is extracted twice with ethyl acetate. The organic phase is dried over sodium sulphate and concentrated under reduced pressure. The residue is purified using a 6 g silica gel cartridge (mobile phase: cyclohexane/ethyl acetate 20:1→5:1). This gives 66 mg (89% of theory) of the title compound.

¹H-NMR (200 MHz, CDCl₃): δ=0.98 (dd, 6H), 1.43-1.83 (m, 3H), 1.95 (d, 1H), 2.26 (s, 3H), 3.61 (d, 2H), 3.98 (s, 3H), 5.06-5.17 (m, 5H), 5.94-6.15 (m, 1H), 6.72 (br. s, 1H), 6.90 (d, 1H), 7.04 (br. s, 1H), 7.55 (d, 1H) ppm.

MS (ESIpos): m/z=419 (M+Na)⁺

### EXAMPLE A-73

### 11-(Cyclopentylethynyl)-3-[(1S)-1-hydroxy-3-methylbutyl]-4-methoxy-9-methyl-5H,7H-dibenzo[b,g][1,5]dioxocin-5-one

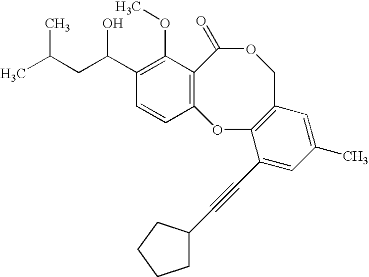

90 mg (189 μmol) of the compound from Example A-XXXIII are suspended in 5 ml of methanol, 68 μl of 26% strength aqueous ammonia solution are added and the mixture is stirred at room temperature for one hour. For work-up, the reaction mixture is concentrated under reduced pressure. The residue is purified using a 6 g silica gel cartridge (mobile phase: cyclohexane/ethyl acetate 20:1→5:1). This gives 69 mg (81% of theory) of product.

¹H-NMR (200 MHz, CDCl₃): δ=0.98 (dd, 6H), 1.46-2.08 (m, 11H), 1.96 (d, 1H), 2.24 (s, 3H), 2.90 (quintet, 1H), 3.97 (s, 3H), 5.07 (br. s, 3H), 6.75 (br. s, 1H), 7.06 (d, 1H), 7.24 (br. s, 1H), 7.58 (d, 1H) ppm.

MS (ESIpos): m/z=471 (M+Na)⁺

The examples listed in the table below are prepared analogously to the procedures described above, from the corresponding starting materials:

| Example | | |
|---|---|---|
| A- | Structure | Analytical data |
| 74 | 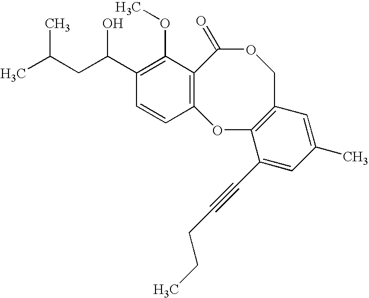 | ¹H-NMR (300 MHz, CDCl₃): δ = 0.97 (t, 6 H), 1.44-1.82 (m, 3 H), 1.93 (d, 1 H), 2.26 (s, 3 H), 3.47 (s, 3 H), 3.97 (s, 3 H), 4.38 (s, 2 H), 5.07 (br. s, 3 H), 6.81 (br. s, 1 H), 7.06 (d, 1 H), 7.29 (br. s, 1 H), 7.58 (d, 1 H) ppm. MS (ESIpos): m/z = 447 (M + Na)⁺ |
| 75 | 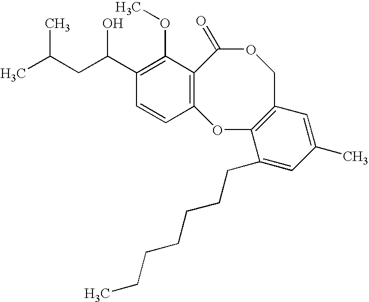 | ¹H-NMR (200 MHz, CDCl₃): δ = 0.88 (t, 3 H), 0.98 (dd, 6 H), 1.28-1.88 (m, 13 H), 1.97 (d, 1 H), 2.25 (s, 3 H), 2.81 (t, 2 H), 3.98 (s, 3 H), 5.06 (br. s, 3 H), 6.67 (s, 1 H), 6.87 (d, 1 H), 7.03 (s, 1 H), 7.56 (d, 1 H) ppm. MS (ESIpos): m/z = 477 (M + Na)⁺ |
| 76 | 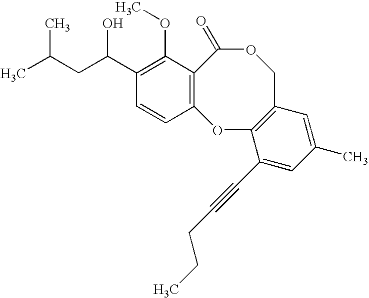 | ¹H-NMR (300 MHz, CDCl₃): δ = 0.97 (t, 6 H), 1.06 (t, 3 H), 1.44-1.82 (m, 3 H), 1.66 (q, 2 H), 1.93 (d, 1 H), 2.24 (s, 3 H), 2.46 (t, 2 H), 3.97 (s, 3 H), 5.06 (br. s, 3 H), 6.74 (br. s, 1 H), 7.06 (d, 1 H), 7.25 (br. s, 1 H), 7.57 (d, 1 H) ppm. MS (ESIpos): m/z = 445 (M + Na)⁺ |

### EXAMPLE A-77

### 8-Methoxy-3-methyl-9-(3-methylbutanoyl)-7-oxo-5H,7H-dibenzo[b,g][1,5]dioxocin-1-yl methanesulphonate

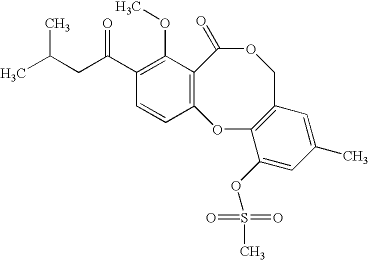

200 mg (0.41 mmol) of the compound from Example A-XLI are taken up in 5 ml of oxygen-free toluene, and the mixture is heated to the boil. At boiling point, a solution of 10 mg (40 mmol) of 2,2′-azobis-2-methylpropanenitrile and 170 μl (0.62 mmol) of tri-n-butyltin hydride in 5 ml of oxygen-free toluene is then added dropwise over a period of 3 hours. After 3 hours, the reaction mixture is cooled and concentrated under reduced pressure. The residue is purified chromatographically on silica gel (mobile phase: cyclohexane/ethyl acetate gradient). The resulting colourless oil is triturated with ether and crystallized. This gives 95 mg (51% of theory) of product.

R_{f}=0.39 (cyclohexane/ethyl acetate 2:1)

¹H-NMR (200 MHz, CDCl₃): δ=0.96 (d, 6H), 2.18 (sep., 1H), 2.37 (s, 3H), 2.90 (d, 2H), 3.64 (s, 3H), 3.96 (s, 3H), 5.40 (br. s, 2H), 7.19 (s, 1H), 7.24 (d, 1H), 7.56 (s, 1H), 7.98 (d, 1H) ppm.

MS (ESIpos): m/z=449 (M+H)⁺

HPLC (Method 2): Rₜ=4.95 min.

### EXAMPLE A-78

### 9-[(1S)-1-Hydroxy-3-methylbutyl]-1-(isopentyloxy)-8-methoxy-3-methyl-7-oxo-5H,7H-dibenzo[b,g][1,5]dioxocin-4-carbonitrile

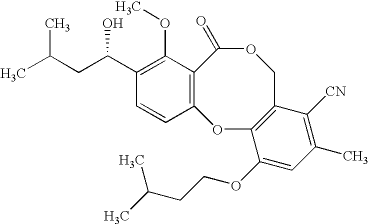

Under argon, 50 mg (0.10 mmol) of the compound from Example A-IX, 16.9 mg (0.14 mmol) of zinc cyanide and 4.4 mg (3.8 μmol) of tetrakis-(triphenylphosphine)palladium(0) are dissolved in 2 ml of dimethylformamide and stirred at 120° C. for 8 hours. After cooling, 10 ml of diethyl ether are added to the reaction mixture, and the mixture is washed in each case once with saturated ammonium chloride solution and water. The organic phase is dried over magnesium sulphate and filtered through silica gel. The product is eluted with ethyl acetate and the filtrate is concentrated under reduced pressure. The residue is purified by preparative thick-layer chromatography (mobile phase: cyclohexane/ethyl acetate 2:1). This gives 36 mg (80% of theory) of product.

¹H-NMR (200 MHz, CDCl₃): δ=0.95-1.02 (m, 12H), 1.26-1.95 (m, 7H), 2.5 (s, 3H), 3.98 (s, 3H), 4.14 (t, 2H), 5.07-5.14 (m, 1H), 5.4 (s, 2H), 6.84-6.88 (m, 2H), 7.60 (d, 1H).

### EXAMPLE A-79

### 3-(1-Hydroxy-3-methylbutyl)-11-(isopentyloxy)-4-methoxy-9-methyl-5H,7H-dibenzo[b,g][1,5]dioxocin-5-one

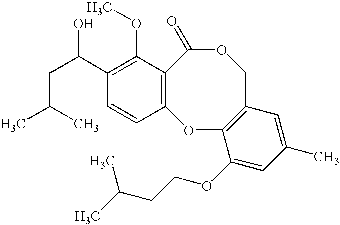

Under argon, 250 mg (0.57 mmol) of the compound from Example A-XXIX are initially charged in 10 ml of tetrahydrofuran, and 340 μl (0.68 mmol) of a 2 M solution of isopropylmagnesium chloride in tetrahydrofuran are added with stirring.

After 16 hours, water and ethyl acetate are added to the reaction solution, and the mixture is poured into a 1 N solution of hydrochloric acid. The phases are separated and the aqueous phase is extracted once with ethyl acetate. The organic phase is dried over sodium sulphate and concentrated under reduced pressure. The residue is purified by preparative HPLC. 78 mg (31% of theory) of the title compound are isolated.

¹H-NMR (300 MHz, CDCl₃): δ=0.95-1.01 (m, 12H), 1.43-1.97 (m, 7H), 2.27 (s, 3H), 3.97 (s, 3H), 4.09 (t, 2H), 5.05-5.12 (m, 3H), 6.41 (s, 1H), 6.80 (s, 1H), 6.94 (d, 1H), 7.57 (d, 1H) ppm.

MS (ESIpos): m/z=465 (M+Na)⁺

HPLC (Method 1): Rₜ=5.42 min.

### EXAMPLE A-80

### [(1S)-1-Hydroxy-3-methylbutyl]-11-(isopentyloxy)-4-methoxy-7,9-dimethyl-5H,7H-dibenzo[b,g][1,5]dioxocin-5-one

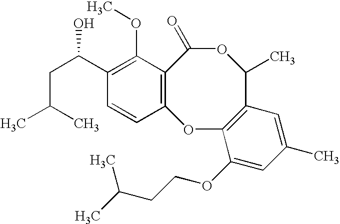

150 mg (0.26 mmol) of the compound from Example A-XLVIII are weighed into a Schlenk flask which has been dried thoroughly by heating and is flushed with argon, and the compound is dried under reduced pressure to remove traces of water. 0.40 ml (0.40 mmol) of a 1 M solution of tetrabutylammonium fluoride in THF is then added, and the mixture is stirred at room temperature for another 30 min. The mixture is filtered through silica gel, the product is eluted with ethyl acetate and the filtrate is concentrated. The product is obtained as a mixture of the epimers (108 mg, 90% of theory).

R_{f}=0.36 (cyclohexane/ethyl acetate 2:1)

¹H-NMR (300 MHz, CDCl₃): δ=1.0 (m, 12H), 1.50 (m, 1H), 1.62-1.97 (m, 9H), 2.31 (s, 3H), 3.96 (s, 3H), 4.05 (m, 2H), 5.10 (m, 1H), 5.51 (m, 1H), 6.56 (br. s, 1H), 6.79 (br. s, 1H), 6.98 (d, 1H), 7.54 (d, 1H) ppm.

MS (ESIpos): m/z=479 (M+Na)

HPLC (Method 1): Rₜ=5.30/5.41 min.

The epimeric products can be separated by preparative HPLC (see general method). 98 mg (0.21 mmol) of the mixture of epimers gives 34 mg (0.07 mmol, 35% of theory) of the first isomer (Example A-81) and 34 mg (0.07 mmol, 35% of theory) of the second isomer (Example A-82).

### EXAMPLE A-81

Rₜ=9.28 min.

¹H-NMR (300 MHz, CDCl₃): δ=1.0 (m, 12H), 1.50 (m, 1H), 1.62-1.97 (m, 9H), 2.31 (s, 3H), 3.96 (s, 3H), 4.05 (t, 2H), 5.10 (dd, 1H), 5.51 (q, 1H), 6.56 (br. s, 1H), 6.79 (br. s, 1H), 6.98 (d, 1H), 7.54 (d, 1H) ppm.

### EXAMPLE A-82

Rₜ=10.27 min.

¹H-NMR (300 MHz, CDCl₃): δ=1.0 (m, 12H), 1.50 (m, 1H), 1.62-1.97 (m, 9H), 2.31 (s, 3H), 3.96 (s, 3H), 4.05 (t, 2H), 5.02 (dd, 1H), 5.51 (q, 1H), 6.56 (br. s, 1H), 6.80 (br. s, 1H), 6.98 (d, 1H), 7.54 (d, 1H) ppm.

Part B:

Starting Materials:

### EXAMPLE B-I

### 8,10-Dibromo-11-hydroxy-3-[(1S)-1-hydroxy-3-methylbutyl]-4-methoxy-9-methyl-5H,7H-dibenzo[b,g][1,5]dioxocin-5-one

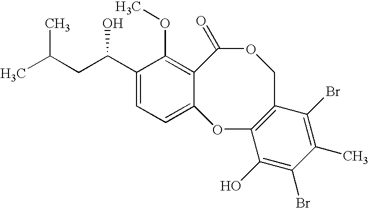

1 g of (2.69 mmol) of penicillide [T. Sassa et al., Agr. Biol. Chem. 37, 1221 (1973), Tetrahedron Lett., 2333 (1973), Tetrahedron Lett., 3941 (1974); Compound (Ib) in EP-A-411 268] is dissolved in 15 ml of ethanol. 436 mg (2.69 mmol) of iron trichloride are dissolved in 5 ml of water and added dropwise to the reaction solution. 277 μl (5.37 mmol) of bromine are then added, and the mixture is stirred at room temperature overnight. The reaction mixture is diluted with dichloromethane and washed once with 10% strength potassium iodide solution, once with water, once with 10% bisulphite solution and once with water. The organic phase is dried over sodium sulphate and concentrated under reduced pressure. This gives 1.33 g (93% of theory) of product.

¹H-NMR (300 MHz, CDCl₃): δ=0.98 (t, 6H), 1.18-1.31 (m, 1H), 1.43-1.52 (m, 1H), 1.63-1.72 (m, 1H), 1.75-1.85 (m, 1H), 2.58 (s, 3H), 3.99 (s, 3H), 5.09 (dd, 1H), 5.43 (q, 2H), 6.52 (br. s, 1H), 6.87 (d, 1H), 7.60 (d, 1H) ppm.

MS (DCI): m/z=548 (M+NH₄)⁺

HPLC (Method 1): Rₜ=5.21 min.

### EXAMPLE B-11 AND EXAMPLE B-III

### 8-Bromo-11-hydroxy-3-[(1S)-1-hydroxy-3-methylbutyl]-4-methoxy-9-methyl-5H,7H-dibenzo[b,g][1,5]dioxocin-5-one (Example B-II) and 10-Bromo-11-hydroxy-3-[(1S)-1-hydroxy-3-methylbutyl]-4-methoxy-9-methyl-5H,7H-dibenzo[b,g][1,5]dioxocin-5-one (Example B-III)

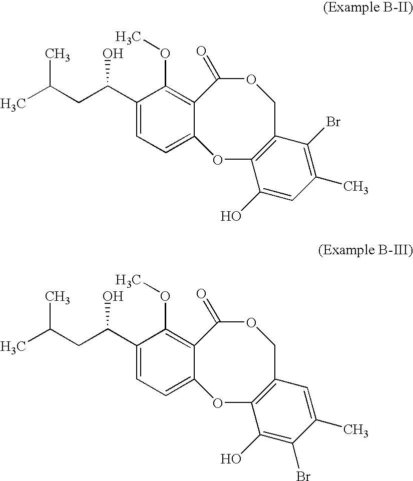

At 0° C., 1 g (2.69 mmol) of penicillide is dissolved in 15 ml of ethanol, and 436 mg (2.69 mmol) of iron trichloride, dissolved in 5 ml of water, are added. 131 μl (2.55 mmol) of bromine, dissolved in 2 ml of ethanol, are then added dropwise over a period of 30 minutes, and the mixture is stirred at room temperature for 10 hours. The reaction solution is diluted with dichloromethane and washed once with 10% strength potassium iodide solution, once with water, once with 10% bisulphite solution and once with water. The organic phase is dried over sodium sulphate and concentrated under reduced pressure. The residue is purified by preparative HPLC. This gives 431 mg (36% of theory) of the compound of Example B-II and 52 mg (4% of theory) of the compound of Example B-III.

### EXAMPLE B-II

¹H-NMR (300 MHz, CDCl₃): δ=0.98 (t, 6H), 1.43-1.52 (m, 1H), 1.63-1.72 (m, 1H), 1.75-1.85 (m, 1H), 1.95 (d, 1H), 2.34 (s, 3H), 3.99 (s, 3H), 5.09 (quintet, 1H), 5.44 (q, 2H), 6.02 (s, 1H), 6.84 (d, 1H), 6.98 (s, 1H), 7.60 (d, 1H) ppm.

MS (DCI): m/z=468/470 (M+NH₄)⁺

HPLC (Method 1): Rₜ=4.87 min

### EXAMPLE B-III

¹H-NMR (300 MHz, CDCl₃): δ=0.98 (t, 6H), 1.43-1.52 (m, 1H), 1.63-1.86 (m, 2H), 1.94 (d, 1H), 2.33 (s, 3H), 3.98 (s, 3H), 5.01-5.13 (m, 3H), 6.30 (s, 1H), 6.51 (s, 1H), 6.93 (d, 1H), 7.60 (d, 1H) ppm.

MS (DCI): m/z=468/470 (M+NH₄)⁺

HPLC (Method 1): Rₜ=4.78 min.

### EXAMPLE B-IV

### 8,10-Dimethyl-11-hydroxy-3-[(1S)-1-hydroxy-3-methylbutyl]-4-methoxy-9-methyl-5H,7H-dibenzo[b,g][1,5]dioxocin-5-one

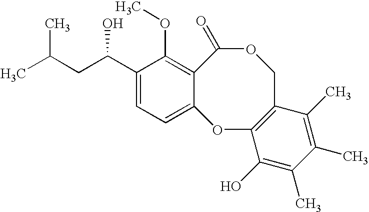

500 mg (0.94 mmol) of the compound from Example B-I are dissolved in 20 ml of dimethylformamide, and 3.92 ml (28.3 mmol) of tetramethyltin and 251 mg (0.22 mmol) of tetrakis(triphenylphosphine)palladium(0) are added under argon. The reaction vessel is closed and heated at 120° C. for 1 hour whilst being irradiated with microwaves (power 200 watt) in a microwave oven (MLS Ethos 1600). The reaction mixture is then cooled to room temperature, 20 ml of water are added and the reaction mixture is extracted a total of four times with in each case 10 ml of ethyl acetate. The combined organic phases are filtered through a 2 g Extrelut/silica gel cartridge (1:1) and the solvent is then removed under reduced pressure. The residue is purified chromatographically on silica gel (mobile phase: cyclohexane/ethyl acetate 100:0→10:90). This gives 339 mg (90% of theory) of product.

¹H-NMR (200 MHz, CDCl₃): δ=0.98 (dd, 6H), 1.40-1.85 (m, 3H), 2.00 (d, 1H), 2.08 (s, 3H), 2.15 (s, 3H), 2.26 (s, 3H), 4.00 (s, 3H), 4.91 (dd, 1H), 5.09 (quintet, 1H), 5.19-5.38 (m, 2H), 6.09 (s, 1H), 6.86 (d, 1H), 7.57 (d, 1H) ppm.

MS (DCI): m/z=418 (M+NH₄)⁺

HPLC (Method 1): Rₜ=4.87 min.

### EXAMPLE B-V AND EXAMPLE B-VI

### 8-Cyano-10-bromo-11-hydroxy-3-[(1S)-1-hydroxy-3-methylbutyl]-4-methoxy-9-methyl-5H,7H-dibenzo[b,g][1,5]dioxocin-5-one (Example B-V) and 8-Bromo-10-cyano-11-hydroxy-3-[(1S)-1-hydroxy-3-methylbutyl]-4-methoxy-9-methyl-5H,7H-dibenzo[b,g][1,5]dioxocin-5-one (Example B-VI)

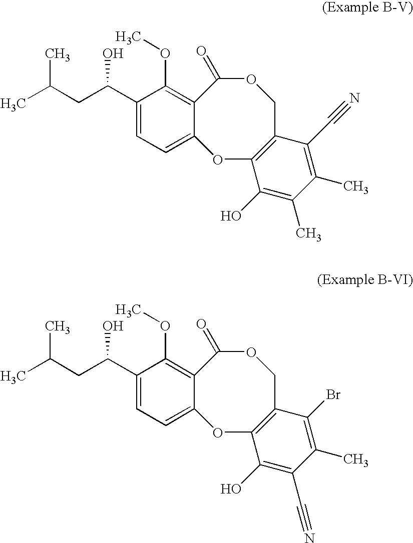

500 mg (0.94 mmol) of the compound from Example B-I are dissolved in 20 ml of dimethylformamide, and 554 mg (4.72 mmol) of zinc cyanide and 109 mg (0.09 mmol) of tetrakis(triphenylphosphine)palladium(0) are added under argon. The reaction vessel is closed and heated twice, in each case for 1 hour, with an interruption of 30 minutes, at 160° C. whilst being irradiated with microwaves (power 200 watt) in a microwave oven (MLS Ethos 1600). The reaction mixture is then cooled to room temperature, 20 ml of diethyl ether are added and the reaction mixture is washed in each case once with in each case 10 ml of saturated ammonium chloride solution and with water. The organic phase is filtered through a 2 g Extrelut/silica gel cartridge (1:1), and the cartridge is eluted with 10 ml of diethyl ether. The solvent is then removed under reduced pressure. The residue is purified chromatographically on silica gel (mobile phase: cyclohexane/ethyl acetate 100:0→10:90). This gives 104 mg (23% of theory) of the compound of Example B-V and 56 mg (12% of theory) of the compound of Example B-VI.

### EXAMPLE B-V

¹H-NMR (400 MHz, CDCl₃): δ=0.98 (t, 6H), 1.43-1.52 (m, 1H), 1.55-1.74 (m, 2H), 2.48 (br. s, 1H), 2.60 (s, 3H), 3.98 (s, 3H), 5.05 (m, 1H), 5.48 (q, 2H), 6.81 (d, 1H), 7.52 (d, 1H), 7.95 (br. s, 1H) ppm.

MS (DCI): m/z=493/495 (M+NH₄)⁺

HPLC (Method 2): Rₜ=4.82 min

### EXAMPLE B-VI

¹H-NMR (300 MHz, CDCl₃): δ=0.99 (t, 6H), 1.40-1.74 (m, 3H), 2.15 (br. s, 1H), 2.58 (s, 3H), 3.98 (s, 3H), 5.01-5.15 (m, 1H), 5.44 (q, 2H), 6.82 (d, 1H), 7.30 (br. s, 1H), 7.60 (d, 1H) ppm.

MS (DCI): m/z=493/495 (M+NH₄)⁺

HPLC (Method 1): Rₜ=4.65 min.

### EXAMPLE B-VII

### 11-Hydroxy-3-[(1S)-1-hydroxy-3-methylbutyl]-4-methoxy-9-methyl-8-vinyl-5H,7H-dibenzo[b,g][1,5]dioxocin-5-one

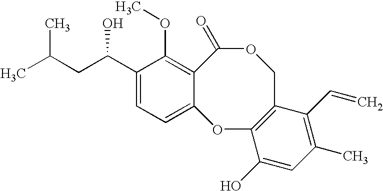

Under argon, 417 mg (0.924 mmol) of the compound from Example B-II are dissolved in 16 ml of toluene, and 36 mg (0.031 mmol) of tetrakis(triphenylphosphine)palladium(0) and 0.54 ml (1.85 mmol) of tributylvinyltin are added. The reaction vessel is closed immediately and the mixture is stirred at 100° C. overnight. After cooling, the reaction mixture is concentrated under reduced pressure. The residue is purified chromatographically on silica gel (mobile phase: cyclohexane/ethyl acetate 100:0→40:60). This gives 180 mg (49% of theory) of product.

¹H-NMR (300 MHz, CDCl₃): δ=0.98 (t, 6H), 1.43-1.85 (m, 3H), 1.97 (d, 1H), 2.18 (s, 3H), 3.99 (s, 3H), 4.91 (dd, 1H), 5.09 (quintet, 1H), 5.30 (br. s, 2H), 5.52 (dd, 1H), 5.97 (s, 1H), 6.56 (dd, 1H), 6.87 (d, 1H), 6.88 (s, 1H), 7.60 (d, 1H) ppm.

MS (ESIpos): m/z=421 (M+Na)⁺

HPLC (Method 1): Rₜ=4.76 min.

### EXAMPLE B-VIII

### 8-Allyl-11-hydroxy-3-[(1S)-1-hydroxy-3-methylbutyl]-4-methoxy-9-methyl-5H,7H-dibenzo[b,g][1,5]dioxocin-5-one

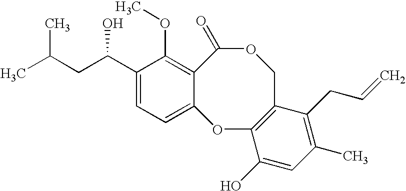

The preparation is carried out analogously to Example B-VII from 310 mg (0.687 mmol) of the compound from Example B-II. This gives 155 mg (55% of theory) of product.

¹H-NMR (300 MHz, CDCl₃): δ=0.97 (t, 6H), 1.43-1.85 (m, 3H), 1.97 (d, 1H), 2.20 (s, 3H), 3.22-3.24 (m, 2H), 3.98 (s, 3H), 4.75 (dd, 1H), 5.00 (dd, 1H), 5.09 (quintet, 1H), 5.17 (br. s, 2H), 5.77-5.92 (m, 1H), 6.04 (s, 1H), 6.87 (d, 1H), 6.88 (s, 1H), 7.58 (d, 1H) ppm.

MS (ESIpos): m/z=435 (M+Na)⁺

HPLC (Method 1): Rₜ=4.81 min.

### EXAMPLE B-IX

### 11-(Isopentyloxy)-4-methoxy-9-methyl-3-[(1E)-3-methyl-1-butenyl]-5H,7H-dibenzo[b,g][1,5]dioxocin-5-one

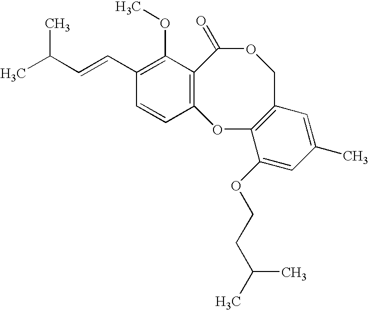

100 mg (0.23 mmol) of the compound from Example B-XLI are initially charged in 1.5 ml of toluene, 20 mg of molecular sieve (4 Å) and a catalytic amount of p-toluenesulphonic acid are added and the mixture is heated at 100° C. for 2 hours. The reaction mixture is cooled, three times its volume of diethyl ether is added and the mixture is stirred at room temperature for 2 hours. The mixture is then washed with saturated sodium bicarbonate solution, dried over sodium sulphate and concentrated under reduced pressure. The residue is purified chromatographically on 7 g of silica gel (mobile phase: ethyl acetate/cyclohexane 1:7). This gives 62 mg (65% of theory) of a white solid.

¹H-NMR (300 MHz, CDCl₃): δ=1.00 (d, 6H), 1.10 (d, 6H), 1.77 (q, 2H), 1.92 (sep., 1H), 2.26 (s, 3H), 2.49 (sextet, 1H), 3.91 (s, 3H), 4.09 (t, 2H), 5.04 (s, 2H), 6.13-6.21 (m, 1H), 6.40 (s, 1H), 6.55 (d, 1H), 6.78 (s, 1H), 6.88 (d, 1H), 7.54 (d, 1H) ppm.

HPLC (Method 1): Rₜ=5.88 min.

### EXAMPLE B-X

### 11-(Isopentyloxy)-4-methoxy-9-methyl-5-oxo-5H,7H-dibenzo[b,g][1,5]dioxocin-3-carbaldehyde

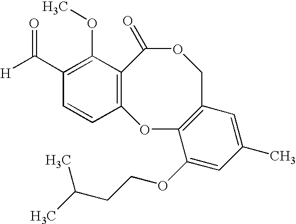

1.11 g (2.62 mmol) of the compound from Example B-IX are initially charged in 52 ml of dioxane, and 3.3 ml of osmium tetroxide (2.5% by weight strength solution in tert-butanol) are added. After 5 minutes, a solution of 2.8 g (13.07 mmol) of sodium periodate in 26 ml of water is added. A colourless suspension is formed. After 90 minutes, the mixture is filtered, the filtercake is washed with dichloromethane and the filtrate is partitioned between dichloromethane and water. The phases are separated and the aqueous phase is extracted two more times with dichloromethane. The combined organic phases are washed with saturated sodium chloride solution, dried over magnesium sulphate and concentrated under reduced pressure. The dark oily residue is purified chromatographically on silica gel (mobile phase: cyclohexane/ethyl acetate 100:0→4:1). This gives 671 mg (67% of theory) of a greenish-grey solid.

¹H-NMR (200 MHz, CDCl₃): δ=1.00 (d, 6H), 1.78 (q, 2H), 1.91 (sep., 1H), 2.28 (s, 3H), 4.07-4.12 (m, 5H), 5.11 (s, 2H), 6.43 (s, 1H), 6.81 (s, 1H), 7.03 (d, 1H), 8.00 (d, 1H), 10.35 (s, 1H) ppm.

MS (DCI): m/z=402 (M+NH₄)⁺

HPLC (Method 2): Rₜ=5.23 min.

### EXAMPLE B-XI

### 3-(1-Hydroxy-4-pentenyl)-11-(isopentyloxy)-4-methoxy-9-methyl-5H,7H-dibenzo[b,g][1,5]dioxocin-5-one

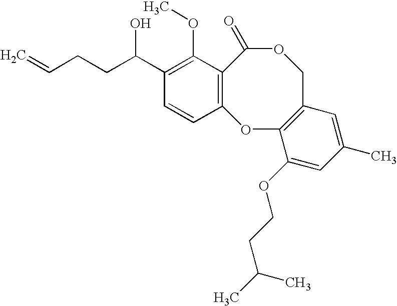

To prepare the Grignard reagent, 243 mg (10 mmol) of magnesium turnings are initially charged, dried by heating under reduced pressure and, after cooling under argon, covered with 2 ml of dry diethyl ether. A few drops of bromomethyl-cyclopropane are then added, and the mixture is warmed until the reaction starts. The remaining bromomethylcyclopropane [in total 970 μl (10 mmol)], dissolved in 3 ml of diethyl ether, is added dropwise, and the mixture is then heated in an oil bath under reflux for another 30 minutes until most of the magnesium has dissolved. After cooling, 160 μl (about 2 eq.) of the Grignard solution are added to a solution, cooled to −78° C., of 60 mg (0.16 mmol) of the compound from Example B-X in 1.6 ml of tetrahydrofuran. After 3 hours at −78° C., the reaction solution is hydrolysed using saturated ammonium chloride solution. The mixture is diluted with water and extracted with diethyl ether. The combined organic phases are washed with saturated sodium chloride solution, dried over magnesium sulphate and concentrated under reduced pressure. The residue is purified by preparative HPLC. 44 mg (62% of theory) of the title compound are isolated.

¹H-NMR (300 MHz, CDCl₃): δ=1.00 (d, 6H), 1.35-2.23 (m, 8H), 2.27 (s, 3H), 3.96 (s, 3H), 4.09 (t, 2H), 4.98-5.09 (m, 5H), 5.78-5.91 (m, 1H), 6.41 (br. s, 1H), 6.79 (br. s, 1H), 6.94 (d, 1H), 7.55 (d, 1H) ppm.

MS (ESIpos): m/Z=423 (M+Na)⁺

HPLC (Method 2): Rₜ=5.55 min.

### EXAMPLE B-XII

### 11-Hydroxy-3-[(1S)-1-hydroxy-3-methylbutyl]-4-methoxy-9-methyl-8-nitro-5H,7H-dibenzo[b,g][1,5]dioxocin-5-one

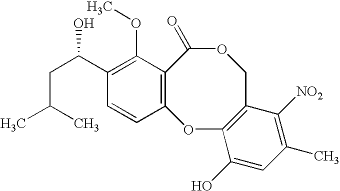

Under argon, 300 mg (0.806 mmol) of penicillide are dissolved in 30 ml of dichloromethane and cooled to −78° C. 118 mg (0.886 mmol) of nitronium-tetrafluoroborate are added, and the temperature is slowly increased to −20° C., with HPLC control. Three new products are formed. Water is added to the reaction mixture, and the mixture is diluted with ethyl acetate. The organic phase is washed three times with saturated sodium bicarbonate solution and once with water. The organic phase is then dried over magnesium sulphate and concentrated under reduced pressure. The residue is purified by preparative HPLC. This gives 16 mg (5% of theory) of a product mixture having a regioisomer ratio of 4.3:1.

¹H-NMR (200 MHz, CDCl₃): δ=0.99 (dd, 6H), 1.40-2.02 (m, 4H), 2.31 (s, 3H), 3.98 (s, 3H), 5.14 (m, 3H), 6.53 (s, 1H), 6.85 (d, 1H), 6.96 (s, 1H), 7.64 (d, 1H) ppm.

MS (DCI): m/z=435 (M+NH₄)⁺

### EXAMPLE B-XIII

### 1-(11-Hydroxy-4-methoxy-9-methyl-5-oxo-5H,7H-dibenzo[b,g][1,5]dioxocin-3-yl)-3-methylbutyl formate

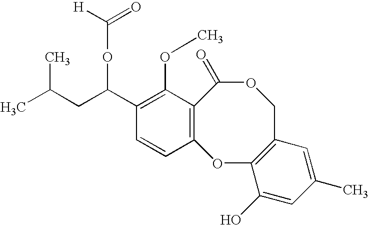

Under argon, 6 g (16.11 mmol) of penicillide are dissolved in 24 ml (644 mmol) of formic acid and heated at 40° C. for one hour. After cooling, the reaction solution is concentrated under reduced pressure. The residue is purified chromatographically on silica gel (mobile phase: cyclohexane/ethyl acetate 100:1→5:1). The product fractions are concentrated under reduced pressure and the residue is triturated with pentane. The precipitate is filtered off with suction and dried under high vacuum.

This gives 5.6 g (86% of theory) of product.

R_{f}=0.40 (cyclohexane/ethyl acetate 2:1)

¹H-NMR (200 MHz, CDCl₃): δ=0.94-0.97 (m, 6H), 1.42-1.87 (m, 3H), 2.24 (s, 3H), 4.03 (s, 3H), 4.98-5.17 (m, 2H), 6.06 (s, 1H), 6.26 (dd, 1H), 6.38 (br. s, 1H), 6.86 (br. s, 1H), 6.88 (d, 1H), 7.47 (d, 1H), 8.07 (s, 1H) ppm.

HPLC (Method 1): Rₜ=4.86 min.

MS (ESIpos): m/z=423 (M+Na)⁺

### EXAMPLE B-XIV

### Methyl 2,3-dihydroxybenzoate

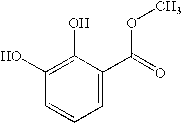

2 g (12.98 mmol) of 2,3-dihydroxybenzoic acid are suspended in 40 ml of methanol, a catalytic amount of p-toluenesulphonic acid is added and the mixture is heated to the boil. After a total of 36 h under reflux and two further additions of p-toluenesulphonic acid, the reaction solution is cooled and 80 ml of water are added. The precipitate is filtered off with suction and washed with a mixture of methanol and water in a ratio of 1:2. Drying under high vacuum gives 1 g (46% of theory) of product.

¹H-NMR (300 MHz, CDCl₃): δ=3.95 (s, 3H), 5.64 (s, 1H), 6.79 (t, 1H), 7.10 (dd, 1H), 7.36 (dd, 1H), 10.87 (s, 1H) ppm.

MS (DCI): m/z=186 (M+NH₄)⁺

HPLC (Method 2): Rₜ=3.65 min

### EXAMPLE B-XV

### Methyl 3-(benzyloxy)-2-hydroxybenzoate

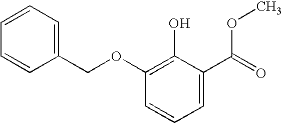

Under argon, 8.4 g (49.96 mmol) of the compound from Example B-XIV are initially charged in 100 ml of dimethylformamide, and 2.6 g (109.9 mmol) of 60% sodium hydride are added a little at a time (exothermic reaction). 6.54 ml (54.95 mmol) of benzyl bromide are then added dropwise at 15-20° C. After 30 minutes, the reaction mixture is poured onto a mixture of ice and dilute hydrochloric acid and extracted with diethyl ether. The organic phase is dried over sodium sulphate and concentrated under reduced pressure. The residue is recrystallized from methanol. This gives 4.1 g (30% of theory) of product.

¹H-NMR (300 MHz, CDCl₃): δ=3.95 (s, 3H), 5.17 (s, 2H), 6.74 (t, 1H), 7.05 (dd, 1H), 7.27-7.39 (m, 3H), 7.42-7.46 (m, 3H), 10.99 (s, 1H) ppm.

MS (ESIpos): m/z=281 (M+Na)⁺

HPLC (Method 1): Rₜ=4.84 min.

### EXAMPLE B-XVI

### 2-(Benzyloxy)-6-(hydroxymethyl)phenol

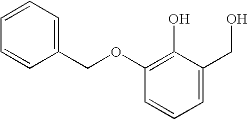

Under argon, 3.9 g (103 mmol) of sodium borohydride are suspended in 25 ml of tetrahydrofuran. A solution of 6.6 g (26 mmol) of the compound from Example B-XV in 45 ml of tetrahydrofuran is added dropwise. At an internal temperature of 50° C., 16 ml of methanol are added dropwise. After 2 hours, 40 ml of water are added to the mixture. The mixture is then adjusted to pH 1 using dilute hydrochloric acid, diluted with water and extracted with dichloromethane. The organic phase is concentrated under reduced pressure. The residue is purified chromatographically on silica gel (mobile phase: dichloromethane). This gives 4 g (65% of theory) of product.

¹H-NMR (200 MHz, CDCl₃): δ=2.26 (br. t, 1H), 4.74 (br. d, 2H), 5.11 (s, 2H), 6.02 (s, 1H), 6.77-6.92 (m, 3H), 7.35-7.43 (m, 5H) ppm.

MS (DCI): m/z=248 (M+NH₄)⁺

HPLC (Method 1): Rₜ=4.05 min.

### EXAMPLE B-XVII

### 2-(Benzyloxy)-6-[(tetrahydro-2H-pyran-2-yloxy)methyl]phenol

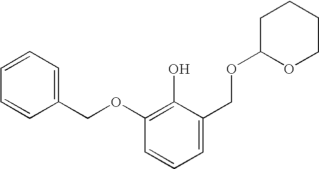

Under argon, 2.90 g (12.59 mmol) of the compound from Example B-XVI are initially charged in 40 ml of dichloromethane, and a catalytic amount of p-toluene-sulphonic acid is added. At −10° C., 1.21 ml (13.22 mmol) of 3,4-dihydro-2H-pyran are added dropwise over a period of 15 minutes. After 30 minutes, the reaction mixture is poured into saturated sodium bicarbonate solution. The organic phase is washed twice with water, dried over magnesium sulphate and concentrated under reduced pressure. The residue is purified chromatographically on silica gel (mobile phase: cyclohexane/ethyl acetate 4:1). This gives 3.83 g (97% of theory) of product.

¹H-NMR (400 MHz, CDCl₃): δ=1.52-1.88 (m, 6H), 3.54-3.57 (m, 1H), 3.93-3.99 (m, 1H), 4.63 (d, 1H), 4.75 (br. t, 1H), 4.86 (d, 1H), 5.11 (s, 2H), 6.35 (s, 1H), 6.77-6.81 (m, 1H), 6.88 (dd, 1H), 6.94 (dd, 1H), 7.32-7.44 (m, 5H) ppm.

MS (ESIpos): m/z=337 (M+Na)⁺

HPLC (Method 1): Rₜ=4.87 min.

### EXAMPLE B-XVIII

### Methyl 6-{2-(benzyloxy)-6-[(tetrahydro-2H-pyran-2-yloxy)methyl]phenoxy}-3-formyl-2-methoxybenzoate

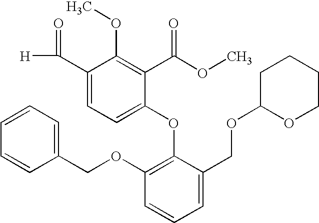

Under argon, 6.51 g (23.86 mmol) of methyl 6-bromo-3-formyl-2-methoxybenzoate [DE-A-4 039 860, Example 5] and 15 g (47.71 mmol) of the compound from Example B-XVII are dissolved in 380 ml of acetonitrile, 3.79 g (59.64 mmol) of copper, 4.74 g (59.64 mmol) of copper(II) oxide and 8.74 g (71.57 mmol) of 4-dimethylaminopyridine are added and the mixture is heated at 80° C. overnight. For work-up, the mixture is cooled and filtered with suction through kieselguhr, and the product is eluted with dichloromethane. The filtrate is concentrated under reduced pressure. The residue is purified chromatographically on silica gel (mobile phase: cyclohexane/ethyl acetate 4:1). This gives 8 g (61% of theory) of product.

¹H-NMR (300 MHz, CDCl₃): δ=1.41-1.67 (m, 6H), 3.45-3.49 (m, 1H), 3.75-3.79 (m, 1H), 3.83 (s, 3H), 3.93 (s, 3H), 4.47 (d, 1H), 4.66 (br. t, 1H), 4.72 (d, 1H), 5.01 (s, 2H), 6.42 (dd, 1H), 6.99 (dd, 1H), 7.11-7.26 (m, 7H), 7.71 (d, 1H), 10.21 (s, 1H) ppm.

MS (DCI): m/z=524 (M+NH₄)⁺

### EXAMPLE B-XIX

### Methyl 6-{2-(benzyloxy)-6-[(tetrahydro-2H-pyran-2-yloxy)methyl]phenoxy}-3-(1-hydroxy-3-methylbutyl)-2-methoxybenzoate

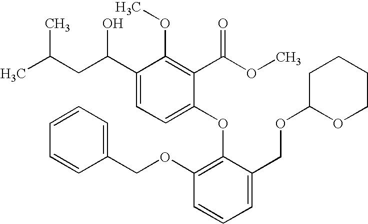

Under argon, 6 g (11.85 mmol) of the compound from Example B-XVIII are dissolved in 75 ml of tetrahydrofuran and cooled to −78° C. At this temperature, 11 ml (17.77 mmol) of a 15% strength solution of isobutyllithium in heptane are slowly added dropwise. The temperature should not exceed 65° C. After the addition, stirring at −78° C. is continued for 5 minutes, and the reaction solution is then stirred at room temperature for one hour. A 10% strength solution of ammonium chloride is added to the reaction mixture and the mixture is diluted with water and extracted with ethyl acetate. The organic phase is washed once with water, dried over magnesium sulphate and concentrated under reduced pressure. The residue is purified chromatographically on silica gel (mobile phase: cyclohexane/ethyl acetate 100:0→100:25). This gives 4.2 g (63% of theory) of product.

R_{f}=0.22 (dichloromethane/methanol 100:1)

¹H-NMR (200 MHz, CDCl₃): δ=0.95 (dd, 6H), 1.42-1.87 (m, 10H), 3.42-3.52 (m, 1H), 3.75-3.85 (m, 1H), 3.87 (s, 3H), 3.91 (s, 3H), 4.52 (dd, 1H), 4.65-4.79 (m, 2H), 4.96-5.10 (m, 3H), 6.34 (d, 1H), 6.93-6.98 (m, 1H), 7.14-7.25 (m, 8H) ppm.

### EXAMPLE B-XX

### Methyl-6-[2-(benzyloxy)-6-(hydroxymethyl)phenoxy]-3-(1-hydroxy-3-methylbutyl)-2-methoxybenzoate

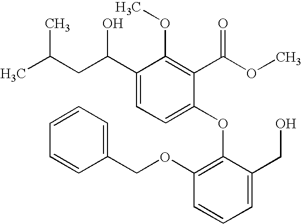

300 mg (0.531 mmol) of the compound from Example B-XIX are dissolved in a mixture of 3 ml of glacial acetic acid, 1 ml of tetrahydrofuran and 0.5 ml of water, and the mixture is heated at 50° C. for 3 hours. After cooling, the reaction solution is concentrated under reduced pressure. The residue is taken up twice in toluene and in each case once more concentrated under reduced pressure. The residue is then purified chromatographically on silica gel (mobile phase: cyclohexane/ethyl acetate 4:1→2:1). This gives 157 mg (62% of theory) of product.

R_{f}=0.33 (dichloromethane/methanol 100:5)

¹H-NMR (200 MHz, CDCl₃): δ=0.93-0.98 (m, 6H), 1.40-1.80 (m, 3H), 1.82 (d, 1H), 2.77 (t, 1H), 3.88 (s, 3H), 3.94 (s, 3H), 4.60 (d, 2H), 4.98-5.10 (m, 3H), 6.36 (d, 1H), 6.97-7.05 (m, 2H), 7.12-7.30 (m, 7H) ppm.

MS (ESIpos): m/z=503 (M+Na)⁺

### EXAMPLE B-XXI

### 11-(Benzyloxy)-3-(1-hydroxy-3-methylbutyl)-4-methoxy-5H,7H-dibenzo[b,g]-[1,5]dioxocin-5-one

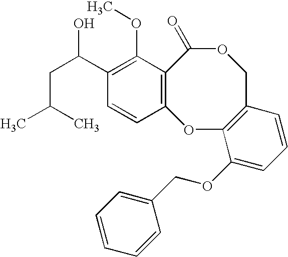

Under argon, 2 g (4.16 mmol) of the compound from Example B-XX are dissolved in 15 ml of methanol, 2.33 g (41.62 mmol) of potassium hydroxide are added and the mixture is stirred under reflux for 7 hours. After cooling, the reaction mixture is concentrated under reduced pressure. The residue is taken up in water and extracted three times with in each case 100 ml of dichloromethane. The aqueous phase is adjusted to pH 1 using 6 N hydrochloric acid and extracted three times with in each case 100 ml of dichloromethane. The organic phases are dried over sodium sulphate and concentrated under reduced pressure. The residue is dissolved in 12 ml of acetonitrile, and 4.35 ml (31.21 mmol) of triethylamine are added. Using a syringe pump, this solution is, over a period of 10 hours and under argon, metered into a solution, of a temperature of 80° C., of 4 g (15.82 mmol) of 2-chloro-1-methyl-pyridinium iodide in 238 ml of acetonitrile. After the addition, the mixture is stirred at 80° C. for 8 hours. After cooling, the reaction solution is concentrated under reduced pressure. The residue is taken up in dichloromethane and washed three times with water. The organic phase is dried over sodium sulphate and concentrated under reduced pressure. The residue is purified chromatographically on silica gel (mobile phase: cyclohexane/ethyl acetate 5:1→2:1). This gives 1.58 g (85% of theory) of product.

R_{f}=0.23 (cyclohexane/ethyl acetate 2:1)

¹H-NMR (300 MHz, CDCl₃): δ=0.97 (t, 6H), 1.42-1.85 (m, 4H), 3.98 (s, 3H), 5.06-5.11 (m, 1H), 5.14 (s, 2H), 5.22 (s, 2H), 6.66 (dd, 1H), 6.95-7.05 (m, 3H), 7.33-7.42 (m, 3H), 7.49 (dd, 2H), 7.56 (d, 1H) ppm.

MS (ESIpos)=471 (M+Na)⁺

HPLC (Method 1): Rₜ=4.92 min.

### EXAMPLE B-XXII

### 11-Hydroxy-3-(1-hydroxy-3-methylbutyl)-4-methoxy-5H,7H-dibenzo[b,g]-[1,5]dioxocin-5-one

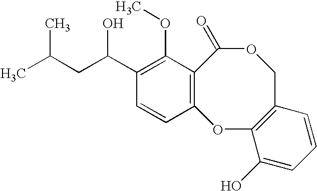

200 mg (0.446 mmol) of the compound from Example B-XXI are dissolved in 25 ml of ethanol, and 200 mg of 10% palladium-on-carbon are added. With stirring, the suspension is evacuated, vented with argon and heated to 75° C. At this temperature, 2.5 ml (26.76 mmol) of 1,4-cyclohexadiene are added a little at a time (0.5 ml every 30 minutes). After the last addition, the mixture is stirred at 75° C. for another hour. After cooling, the reaction mixture is filtered through kieselguhr and eluted with ethanol. The filtrate is concentrated under reduced pressure. The residue is purified by preparative HPLC. This gives 148 mg (93% of theory) of product.

R_{f}=0.15 (cyclohexane/ethyl acetate 2:1)

¹H-NMR (300 MHz, CDCl₃): δ=0.98 (t, 6H), 1.42-1.85 (m, 3H), 1.99 (d, 1H), 3.98 (s, 3H), 5.07-5.12 (m, 3H), 6.16 (s, 1H), 6.58 (dd, 1H), 6.90 (d, 1H), 6.97 (t, 1H), 7.03-7.06 (m, 1H), 7.60 (d, 1H) ppm.

MS (ESIpos)=341 [(M+H)-H₂O]⁺

HPLC (Method 1): Rₜ=4.36 min.

### EXAMPLE B-XXIII

### 1-(11-Hydroxy-4-methoxy-9-methyl-5-oxo-5H,7H-dibenzo[b,g][1,5]dioxocin-3-yl)-3-methylbutyl formate

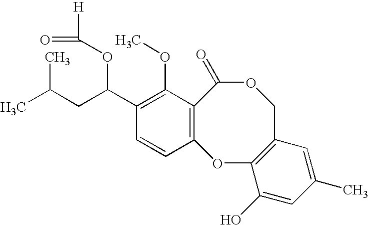

15 g (40.3 mmol) of penicillide, together with 80 ml of formic acid, are heated at 40° C. After one hour, the reaction solution is cooled and concentrated under reduced pressure. The residue is purified chromatographically on silica gel (mobile phase: cyclohexane/ethyl acetate 10:1→5:1). This gives 14.6 g (91% of theory) of product.

¹H-NMR (200 MHz, CDCl₃): δ=0.96 (d, 6H), 1.42-1.90 (m, 3H), 2.24 (s, 3H), 4.03 (s, 3H), 4.98-5.18 (m, 2H), 6.00 (s, 1H), 6.26 (dd, 1H), 6.38 (br. s, 1H), 6.86-6.90 (m, 2H), 7.48 (d, 1H), 8.07 (s, 1H) ppm.

MS (ESIpos)=423 (M+Na)⁺

### EXAMPLE B-XXIV

### 9-[1-(Formyloxy)-3-methylbutyl]-8-methoxy-3-methyl-7-oxo-5H,7H-dibenzo[b,g]-[1,5]dioxocin-1-yl trifluoromethanesulphonate

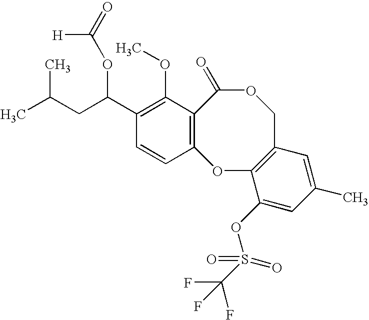

14 g (34.96 mmol) of the compound from Example B-XXII are dissolved in 160 ml of dichloromethane and, at 0° C., 20 ml (245 mmol) of pyridine are added. This is followed by the addition of 24 ml (140 mmol) of trifluoromethanesulphonic anhydride. After 3 hours at room temperature, the reaction solution is poured into ice-water and extracted twice with dichloromethane. The organic phase is washed once with saturated ammonium chloride solution, dried over sodium sulphate and concentrated under reduced pressure. The residue is purified chromatographically on silica gel (mobile phase: cyclohexane/ethyl acetate 20:1→5:1). This gives 18 g (97% of theory) of product.

¹H-NMR (300 MHz, CDCl₃): δ=0.96 (dd, 6H), 1.49-1.89 (m, 3H), 2.33 (s, 3H), 4.04 (s, 3H), 5.11 (q, 2H), 6.27 (dd, 1H), 6.90 (br. d, 1H), 7.10 (d, 1H), 7.14 (br. d, 1H), 7.52 (d, 1H), 8.06 (s, 1H) ppm.

MS (ESIpos): m/z=555 (M+Na)⁺

### EXAMPLE B-XXV

### 9-[1-Hydroxy-3-methylbutyl]-8-methoxy-3-methyl-7-oxo-5H,7H-dibenzo[b,g][1,5]-dioxocin-1-yl trifluoromethanesulphonate

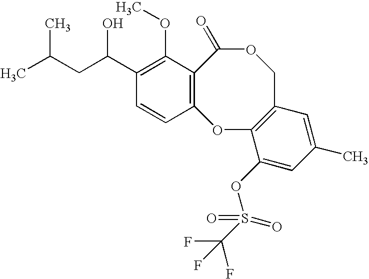

3.7 g (6.95 mmol) of the compound from Example B-XXIV are dissolved in 40 ml of methanol, and 2.5 ml (34.8 mmol) of a 26% strength solution of ammonia are added. After one hour at room temperature, the reaction solution is concentrated under reduced pressure and the residue is dried under high vacuum. This gives 3.5 g (99% of theory) of product.

¹H-NMR (300 MHz, CDCl₃): δ=0.98 (t, 6H), 1.44-1.85 (m, 3H), 1.95 (br. s, 1H), 2.33 (s, 3H), 3.98 (s, 3H), 5.10 (br. s, 3H), 6.90 (br. d, 1H), 7.10 (d, 1H), 7.15 (br. d, 1H), 7.63 (d, 1H) ppm.

MS (ESIpos): m/z=527 (M+Na)⁺

### EXAMPLE B-XXVI

### 8-Methoxy-3-methyl-9-(3-methylbutanoyl)-7-oxo-5H,7H-dibenzo[b,g][1,5]dioxocin-1-yl trifluoromethanesulphonate

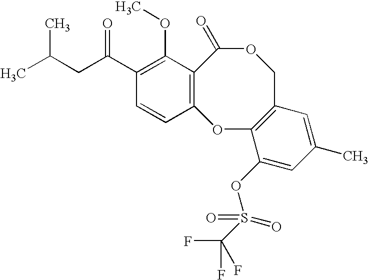

3.63 g (7.2 mmol) of the compound from Example B-XXV are dissolved in 100 ml of dichloromethane, and 1.5 g (14.4 mmol) of basic alumina and 3.1 g (14.4 mmol) of pyridinium chlorochromate are added. The reaction mixture is stirred at room temperature for one hour. For work-up, the mixture is filtered through a short silica gel column and eluted with 1500 ml of dichloromethane. The filtrate is concentrated under reduced pressure. This gives 3.3 g (91% of theory) of product.

¹H-NMR (200 MHz, CDCl₃): δ=0.97 (d, 6H), 2.22 (sep., 1H), 2.35 (s, 3H), 2.84 (d, 2H), 3.96 (s, 3H), 5.16 (br. s, 2H), 6.92 (br. s, 1H), 7.12-7.17 (m, 2H), 7.72 (d, 1H) ppm.

MS (DCI): m/z=520 (M+NH₄)⁺

### EXAMPLE B-XXVII

### 4-Methoxy-9-methyl-3-(3-methylbutanoyl)-11-vinyl-5H,7H-dibenzo[b,g][1,5]-dioxocin-5-one

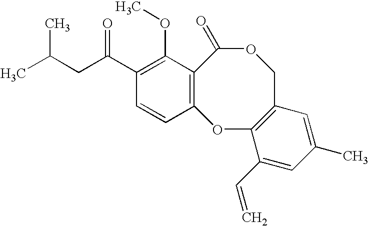

3.1 g (6.2 mmol) of the compound from Example B-XXVI are dissolved in 75 ml of dimethylformamide, and 143 mg (0.124 mmol) of tetrakis(triphenylphosphine)-palladium(0) are added. For 5 minutes, argon is passed through the reaction solution. 9.3 ml (30.95 mmol) of tributylvinyltin are then added and the flask is closed immediately and heated at 90° C. overnight. After cooling, water is added to the reaction mixture, and the mixture is extracted twice with ethyl acetate. The organic phase is dried over sodium sulphate and concentrated under reduced pressure. The residue is purified chromatographically on silica gel (mobile phase: cyclohexane/ethyl acetate 20:1→5:1). This gives 1.89 g (80% of theory) of product.

¹H-NMR (200 MHz, CDCl₃): δ=0.95 (d, 6H), 2.21 (sep., 1H), 2.30 (s, 3H), 2.84 (d, 2H), 3.97 (s, 3H), 5.11 (br. s, 2H), 5.44 (d, 1H), 5.86 (d, 1H), 6.78 (br. s, 1H), 6.90 (d, 1H), 7.18-7.33 (m, 1H), 7.38 (br. s, 1H), 7.67 (d, 1H) ppm.

MS (ESIpos): m/z=381 (M+H)⁺

HPLC (Method 1): Rₜ=5.51 min

### EXAMPLE B-XXVIII

### 11-(1,2-Dihydroxyethyl)-4-methoxy-9-methyl-3-(3-methylbutanoyl)-5H,7H-dibenzo-[b,g][1,5]dioxocin-5-one

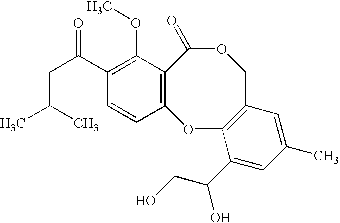

1.89 g (4.97 mmol) of the compound from Example B-XXVII are dissolved in a mixture of 30 ml of ethyl acetate and 30 ml of acetonitrile. At 0° C., a solution of 78 mg (348 μmol) of ruthenium(III) chloride hydrate and 1.59 g (7.45 mmol) of sodium periodate in 10 ml of water is added dropwise. After the addition, the reaction has ended. 61 ml of a saturated solution of sodium bisulphite are added to the reaction mixture, and the mixture is reacted twice with ethyl acetate. The organic phase is dried over sodium sulphate and concentrated under reduced pressure. The residue is purified chromatographically on silica gel (mobile phase: cyclohexane/ethyl acetate 10:1→2:1→ethyl acetate). This gives 1.35 g (66% of theory) of product.

¹H-NMR (300 MHz, CDCl₃): δ=0.96 (d, 6H), 2.00 (br. s, 1H), 2.21 (sep., 1H), 2.30 (s, 3H), 2.84 (d, 3H), 3.72 (br. s, 1H), 3.92-3.98 (m, 1H), 3.96 (s, 3H), 5.09 (br. dd, 2H), 5.39-5.41 (m, 1H), 6.82 (d, 1H), 7.01 (d, 1H), 7.38 (br. s, 1H), 7.68 (d, 1H) ppm.

MS (DCI): m/z=432 (M+NH₄)⁺

### EXAMPLE B-XXIX

### 8-Methoxy-3-methyl-9-(3-methylbutanoyl)-7-oxo-5H,7H-dibenzo[b,g][1,5]dioxocin-1-carboxylic acid

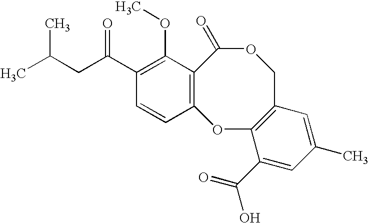

200 mg (0.483 mmol) of the compound from Example B-XXVIII are dissolved in 2.5 ml of carbon tetrachloride and 2.5 ml of acetonitrile, and a solution of 2.2 mg (9.65 μmol) of ruthenium(III) chloride hydrate and 619 mg (2.9 mmol) of sodium periodate in 5 ml of water is added. At room temperature, the reaction mixture is stirred vigorously overnight. For work-up, water (pH 1-2) is added to the mixture and the mixture is extracted twice with ethyl acetate. The organic phase is dried over sodium sulphate and concentrated under reduced pressure. The residue is purified by preparative HPLC. This gives 170 mg (88% of theory) of product.

¹H-NMR (300 MHz, CDCl₃): δ=0.96 (d, 6H), 2.22 (sep., 1H), 2.35 (s, 3H), 2.83 (d, 3H), 3.97 (s, 3H), 5.15 (br. s, 2H), 7.10 (br. s, 1H), 7.23 (d, 1H), 7.70 (d, 1H), 7.85 (br. s, 1H) ppm.

MS (DCI): m/z=416 (M+NH₄)⁺

### EXAMPLE B-XXX

### 2-Methyl-2-propenyl 8-methoxy-3-methyl-9-(3-methylbutanoyl)-7-oxo-5H,7H-dibenzo[b,g][1,5]dioxocin-1-carboxylate

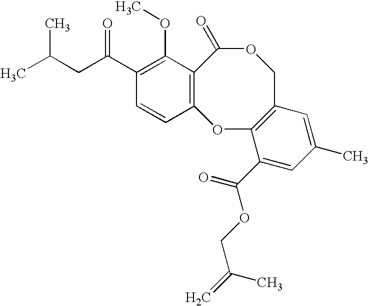

50 mg (126 μmol) of the compound from Example B-XXIX are dissolved in 3 ml of dichloromethane and, at −5° C., 91 mg (1.25 mmol) of 2-methyl-2-propen-1-ol, 19 mg (75.3 μmol) of scandium(III) triflate and 77 mg (627 μmol) of 4-dimethylaminopyridine are added. After 30 minutes at −5° C., a solution of 48 mg (250 μmol) of N′-(3-dimethylaminopropyl)-N-ethylcarbodiimide hydrochloride in 1 ml of dichloromethane is added. After 30 minutes at −5° C., the reaction solution is stirred at room temperature overnight. The solution is filtered through a 10 g silica gel cartridge and eluted first twice with 20 ml of dichloromethane and then once with 10 ml of ethyl acetate. The appropriate product fractions are concentrated under reduced pressure. The residue is dried thoroughly under high vacuum. This gives 50 mg (88% of theory) of product.

¹H-NMR (300 MHz, CDCl₃): δ=0.96 (d, 6H), 1.82 (s, 3H), 2.22 (sep., 1H), 2.33 (s, 3H), 2.84 (d, 2H), 3.96 (s, 3H), 4.79 (s, 2H), 4.97-5.12 (m, 4H), 7.03 (br. d, 1H), 7.34 (d, 1H), 7.61 (br. s, 1H), 7.69 (d, 1H) ppm.

MS (ESIpos): m/z=453 (M+H)⁺

The examples listed in the table below are prepared analogously to the procedures described above, from the corresponding starting materials:

| Example | | |
|---|---|---|
| B- | Structure | Analytical data |
| XXXI | 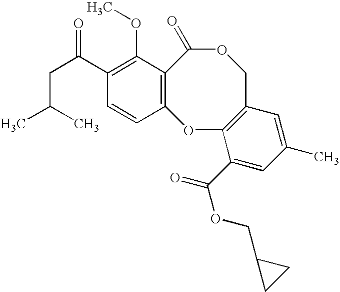 | ¹H-NMR (300 MHz, CDCl₃): δ = 0.50-0.56 (m, 2 H), 0.59-0.65 (m, 2 H), 0.96 (d, 6 H), 1.23-1.32 (m, 1 H), 2.22 (sep., 1 H), 2.33 (s, 3 H), 2.84 (d, 2 H), 3.97 (s, 3 H), 4.20 (d, 2 H), 5.13 (br. s, 2 H), 7.02 (br. d, 1 H), 7.42 (d, 1 H), 7.60 (br. d, 1 H), 7.69 (d, 1 H) ppm. MS (ESIpos): m/z = 453 (M + H)⁺ |
| XXXII | 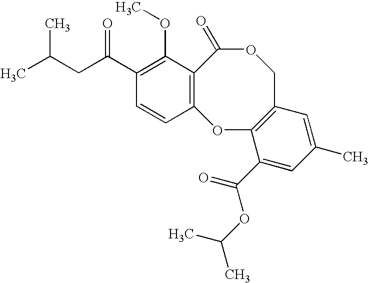 | ¹H-NMR (300 MHz, CDCl₃): δ = 0.96 (d, 6 H), 1.39 (d, 6 H), 2.22 (sep., 1 H), 2.32 (s, 3 H), 2.84 (d, 2 H), 3.97 (s, 3 H), 5.11 (br. s, 2 H), 5.31 (quintet, 1 H), 7.00 (br. d, 1 H), 7.37 (d, 1 H), 7.54 (br. d, 1 H), 7.69 (d, 1 H) ppm. MS (ESIpos): m/z = 441 (M + H)⁺ |
| XXXIII | 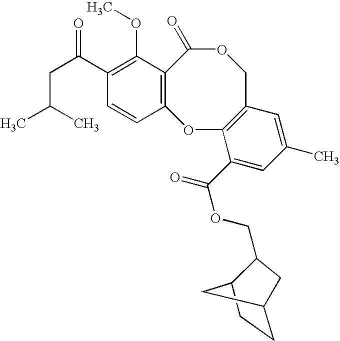 | ¹H-NMR (300 MHz, CDCl₃): δ = 0.96 (d, 6 H), 1.10-2.24 (m, 12 H), 232 (s, 3 H), 2.84 (d, 2 H), 3.97 (s, 3 H), 4.22-4.43 (m, 2 H), 5.11 (br. s, 2 H), 7.01 (br. d, 1 H), 7.34 (d, 1 H), 7.57 (br. d, 1 H), 7.69 (d, 1 H) ppm. MS (ESIpos): m/z = 507 (M + H)⁺ |
| XXXIV | 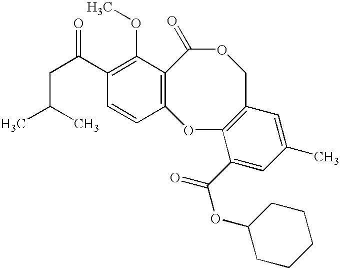 | ¹H-NMR (200 MHz, CDCl₃): δ = 0.96 (d, 6 H), 1.20-2.10 (m, 10 H), 2.22 (sep., 1 H), 2.33 (s, 3 H), 2.85 (d, 2 H), 3.97 (s, 3 H), 4.98-5.14 (m, 3 H), 7.00 (br. d, 1 H), 7.37 (d, 1 H), 7.55 (br. d, 1 H), 7.70 (d, 1 H) ppm. MS (ESIpos): m/z = 481 (M + H)⁺ |
| XXXV | 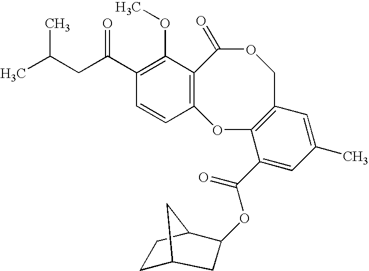 | LC-MS (Method 3): Rₜ = 5.59 min. MS (ESIpos): m/z = 493 (M + H)⁺ |
| XXXVI | 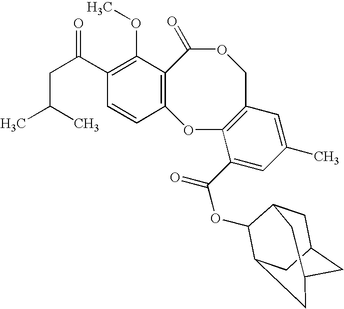 | LC-MS (Method 3): Rₜ = 6.09 min. MS (ESIpos): m/z = 533 (M + H)⁺ |

### EXAMPLE B-XXXVII

tert-Butyl 8-methoxy-3-methyl-9-(3-methylbutanoyl)-7-oxo-5H,7H-dibenzo[b,g]-[1,5]dioxocin-1-carbamate

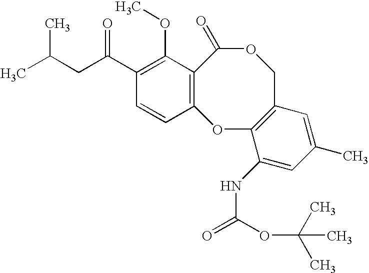

A solution of 926 mg (2.32 mmol) of the compound from Example B-XXIX and 2.22 ml (23.24 mmol) tert-butanol, together with 712 μl (5.11 mmol) of triethylamine, is initially charged in 2 ml of dry dioxane, and 678 mg (2.79 mmol) of diphenylphosphoryl azide are then added at room temperature. The mixture is stirred at room temperature for 1 hour and then heated under reflux for 5 hours. After cooling, the solvent is removed under reduced pressure and the residue is taken up in 5 ml of ethyl acetate. The solution is washed once with 2 ml of saturated sodium chloride solution, dried over sodium sulphate and filtered. After removal of the solvent, the residue is dried under high vacuum and purified chromatographically on silica gel (mobile phase: cyclohexane/ethyl acetate 10:1→2:1→ethyl acetate). This gives 548 mg (49% of theory) of product.

¹H-NMR (300 MHz, CDCl₃): δ=0.98 (d, 6H), 1.57 (s, 9H), 2.10-2.38 (m, 4H), 2.84 (d, 2H), 3.97 (s, 3H), 5.1 (br. s, 2H), 6.51 (br. d, 1H), 6.91 (d, 1H), 7.03 (br. d, 1H), 7.35 (br. s, 1H), 7.70 (d, 1H), 8.07 (br. d, 1H) ppm.

MS (DCI): m/z=487 (M+NH₄)⁺

HPLC (Method 2): Rₜ=5.49 min.

### EXAMPLE B-XXXVIII

### tert-Butyl 8-methoxy-3-methyl-9-(3-methylbutanoyl)-7-oxo-5H,7H-dibenzo[b,g]-[1,5]dioxocin-1-methylcarbamate

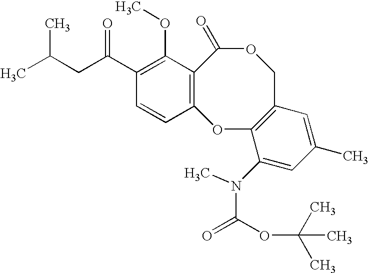

3 ml of dimethylformamide are added to a solution of 253 mg (0.54 mmol) of the compound from Example B-XXXVII in 2 ml of tetrahydrofuran, and the mixture is cooled to 0° C. A little at a time, 23.7 mg (0.59 mmol) of sodium hydride (60% in mineral oil) are introduced into this solution. After 15 minutes, 67 μl of iodomethane are added. The reaction mixture is stirred for 16 h, during which time the mixture is warmed to room temperature. The solvent is then removed under reduced pressure and the residue is taken up in 5 ml of dichloromethane. The solution is washed once with 2 ml of saturated ammonium chloride solution, dried over magnesium sulphate and filtered. After removal of the solvent, the residue is dried under high vacuum and purified chromatographically on silica gel (mobile phase: cyclohexane/ethyl acetate 4:1). This gives 183 mg (70% of theory) of product.

¹H-NMR (300 MHz, CDCl₃): δ=0.97 (d, 6H), 1.48 (br. s, 9H), 2.21 (sept, 1H), 2.29 (s, 3H), 2.82 (d, 2H), 3.23 (br. s, 3H), 3.97 (s, 3H), 5.10 (br. s, 2H), 6.79 (br. s, 1H), 6.87-7.23 (br. m, 2H), 7.66 (d, 1H) ppm.

MS (DCI): m/z=501 (M+NH₄)⁺

HPLC (Method 2): Rₜ=5.36 min.

### EXAMPLE B-XXXIX

### 4-Methoxy-9-methyl-3-(3-methylbutanoyl)-11-methyl amino-5H,7H-dibenzo[b,g]-[1,5]dioxocin-5-one

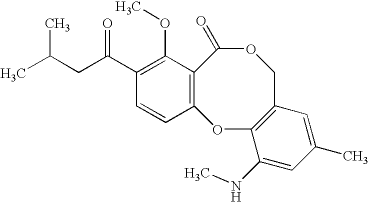

Under argon, 0.75 ml of trifluoroacetic acid is added dropwise to a solution, cooled with ice, of 148 mg (0.31 mmol) of the compound from Example B-XXXVIII in 2.25 ml of dry dichloromethane. The reaction mixture is stirred in an ice bath for 60 minutes. The solvent is then removed under reduced pressure, the residue is taken up in 5 ml of dichloromethane and 0.5 ml of water are added. The mixture is stirred for another 5 minutes, filtered through an Extrelut/silica gel cartridge (eluent dichloromethane, then ethyl acetate) and evaporated to dryness under reduced pressure. The crude product is purified by chromatography on silica gel (Chromabond cartridge, 10 g of silica gel, mobile phase: cyclohexane/ethyl acetate 100:0→40:60). This gives 94 mg (68% of theory) of product.

¹H-NMR (300 MHz, CDCl₃): δ=0.97 (d, 6H), 2.21 (sept, 1H) 2.26 (s, 3H), 2.83 (d, 2H), 2.93 (br. d, 3H), 3.97 (s, 3H), 4.61 (br. s, 1H), 5.09 (br. s, 2H), 6.15 (br. s, 1H), 6.51 (br. s, 1H), 6.94 (d, 1H), 7.66 (d, 1H) ppm.

MS (ESIpos): m/z=384 (M+H)⁺

HPLC (Method 2): Rₜ=4.80 min.

### EXAMPLE B-XL

### N-[8-Methoxy-3-methyl-9-(3-methylbutanoyl)-7-oxo-5H,7H-dibenzo[b,g][1,5]-dioxocin-1-yl]-N,2-dimethylbicyclo[2.2.1]heptane-2-carboxamide

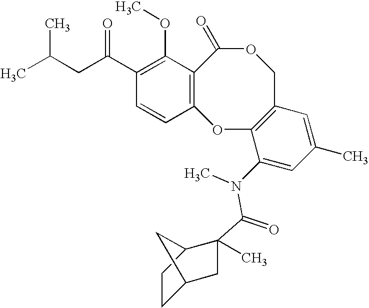

Under argon, 22 μl (0.124 mmol) of N,N-diisopropylethylamine are added dropwise to a solution, cooled with ice, of 30 mg (0.078-mmol) of the compound from Example B-XXXIX in 2.25 ml of dry dichloromethane. 20 mg (0.117 mmol) of 2-methylbicyclo[2.2.1]heptane-2-carbonyl chloride are then added. The mixture is stirred at this temperature for 30 minutes and then at room temperature for 260 minutes. For work-up, the mixture is hydrolysed with 0.5 ml of saturated ammonium chloride solution and filtered through an Extrelut/silica gel cartridge. The filtrate is evaporated to dryness under reduced pressure and purified by preparative HPLC. This gives 27 mg (66% of theory) of product.

MS (ESIpos): m/z=520 (M+H)⁺, 542 (M+Na)⁺

### EXAMPLE B-XLI

### 3-[(1S)-1-Hydroxy-3-methylbutyl]-11-(isopentyloxy)-4-methoxy-9-methyl-5H,7H-dibenzo[b,g][1,5]dioxocin-5-one

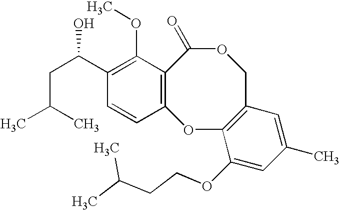

Under argon, 54 g (145 mmol) of penicillide are dissolved in 200 ml of tetrahydrofuran, and 6.09 g (152 mmol) of 60% sodium hydride are added a little at a time at 0° C. After 5 minutes, 5.35 g (14.5 mmol) of tetra-n-butylammonium iodide and 34.7 ml (290 mmol) of 3-methylbutyl bromide are added to the reaction solution, and the mixture is heated at 60° C. overnight. For work-up, the reaction mixture is cooled, water is added and the mixture is extracted with ethyl acetate. The organic phase is washed once with water, dried over sodium sulphate and concentrated under reduced pressure. The residue is triturated with pentane, filtered off with suction and dried at 40° C. under high vacuum. This gives 50 g (76% of theory) of product.

¹H-NMR (300 MHz, CDCl₃): δ=0.95-1.01 (m, 12H), 1.44-1.52 (m, 1H), 1.64-1.80 (m, 4H), 1.90 (quintet, 1H), 1.97 (d, 1H), 2.27 (s, 3H), 3.96 (s, 3H), 4.11 (t, 2H), 5.04-5.10 (m, 3H), 6.41 (s, 1H), 6.79 (s, 1H), 6.94 (d, 1H), 7.55 (d, 1H) ppm.

MS (DCI): m/z=460 (l+NH₄)⁺

### EXAMPLE B-XLII

### 8-Bromo-3-[(1S)-1-hydroxy-3-methylbutyl]-11-(isopentyloxy)-4-methoxy-9-methyl-5H,7H-dibenzo[b,g][1,5]dioxocin-5-one

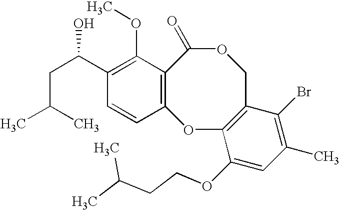

940 mg (2.1 mmol) of the compound from Example B-II are reacted analogously to Example B-XLI. This gives 711 mg (65% of theory) of product.

¹H-NMR (400 MHz, CDCl₃): δ=0.96-1.00 (m, 12H), 1.42-1.52 (m, 2H), 1.65-1.95 (m, 5H), 2.36 (s, 3H), 3.98 (s, 3H), 4.08 (t, 2H), 5.08 (quintet, 1H), 5.44 (q, 2H), 6.88 (d, 1H), 6.89 (s, 1H), 7.57 (d, 1H) ppm.

MS (DCI): m/z=540 (M+NH₄)⁺

### EXAMPLE B-XLIII

### 3-((1S)-1-{[tert-Butyl(dimethyl)silyl]oxy}-3-methylbutyl)-11-(isopentyloxy)-4-methoxy-9-methyl-5H,7H-dibenzo[b,g][1,5]dioxocin-5-one

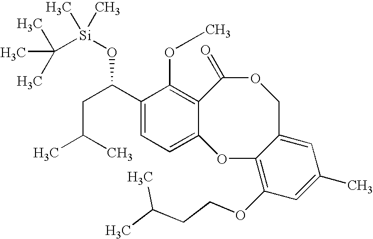

Under argon, 2 g (4.5 mmol) of the compound from Example B-XLI are dissolved in 15 ml of dimethylformamide, 923 mg (13.6 mmol) of imidazole and 1.02 g (6.78 mmol) of tert.-butyldimethylsilyl chloride are added and the mixture is stirred at 60° C. overnight. For work-up, the reaction mixture is cooled, a saturated solution of ammonium chloride is added and the mixture is extracted twice with diethyl ether. The organic phase is washed once with saturated sodium chloride solution, dried over sodium sulphate and concentrated under reduced pressure. The residue is purified chromatographically on silica gel (mobile phase: cyclohexane/ethyl acetate 20:1→10:1). This gives 2.12 g (84% of theory) of product.

¹H-NMR (200 MHz, CDCl₃): δ=−0.24 (s, 3H), 0.00 (s, 3H), 0.75-0.96 (m, 21H), 1.10-1.95 (m, 6H), 2.22 (s, 3H), 3.89 (s, 3H), 4.03 (t, 2H), 4.98-5.10 (m, 3H), 6.37 (br. s, 1H), 6.74 (br. s, 1H), 6.88 (d, 1H), 7.54 (d, 1H) ppm.

MS (ESIpos): m/z=579 (M+Na)⁺

### EXAMPLE B-XLIV

### 3-((1S)-1-{[tert-Butyl(dimethyl)silyl]oxy}-3-methylbutyl)-8-bromo-11-(isopentyloxy)-4-methoxy-9-methyl-5H,7H-dibenzo[b,g][1,5]dioxocin-5-one

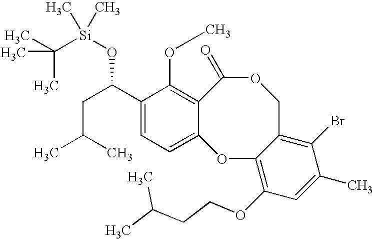

1370 mg (2.6 mmol) of the compound from Example B-XLII are reacted analogously to Example B-XLIII. This gives 1600 mg (96% of theory) of product.

¹H-NMR (200 MHz, CDCl₃): δ=−0.20 (s, 3H), 0.03 (s, 3H), 0.75-1.02 (m, 21H), 1.18-1.95 (m, 6H), 2.36 (s, 3H), 3.94 (s, 3H), 4.07 (t, 2H), 5.08 (q, 1H), 5.30-5.47 (m, 2H), 6.84 (d, 1H), 6.88 (br. s, 1H), 7.60 (d, 1H) ppm.

MS (ESIpos): m/z=657/659 (M+Na)⁺

HPLC (Method 8): Rₜ=11.05 min.

### EXAMPLE B-XLV

### 3-((1S)-1-{[tert-Butyl(dimethyl)silyl]oxy}-3-methylbutyl)-8-vinyl-11-(isopentyloxy)-4-methoxy-9-methyl-5H,7H-dibenzo[b,g][1,5]dioxocin-5-one

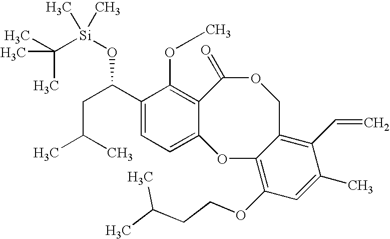

Under argon, 503 mg (0.787 mmol) of the compound from Example B-XLIV and 15.5 mg (0.022 mmol) of bis(triphenylphosphine)palladium(II) chloride are dissolved in 5 ml of dimethylformamide, and 1.15 ml (3.93 mmol) of tributylvinyltin are added. The reaction mixture is then stirred under argon and at 80° C. for 16 hours. Another 15.5 mg (0.022 mmol) of bis(triphenylphosphine)palladium(II) chloride are added to the reaction mixture, and stirring at 80° C. is continued for another 16 hours. After cooling, the reaction mixture is concentrated under reduced pressure. The residue is purified chromatographically on silica gel (mobile phase: cyclohexane/ethyl acetate 98:2→90:10). This gives 118 mg (26% of theory) of product.

¹H-NMR (300 MHz, CDCl₃): δ=−0.19 (s, 3H), 0.05 (s, 3H), 0.85 (s, 9H), 0.91 (d, 3H), 0.96 (d, 3H), 1.00 (d, 6H), 1.22-1.36 (m, 2H), 1.63 (m, 1H), 1.73-1.97 (m, 3H), 2.22 (s, 3H), 3.95 (s, 3H), 4.10 (t, 2H), 4.89 (dd, 1H), 5.11 (dd, 1H), 5.25 (dd, 2H), 5.51 (dd, 1H), 6.57 (dd, 1H), 6.82 (s, 1H), 6.90 (d, 1H), 7.60 (d, 1H) ppm.

MS (ESIpos): m/z=605 (M+Na)⁺

HPLC (Method 8): Rₜ=10.57 min.

### EXAMPLE B-XLVI

### 3-((1S)-1-{[tert-Butyl(dimethyl)silyl]oxy}-3-methylbutyl)-8-formyl-11-(isopentyl-oxy)-4-methoxy-9-methyl-5H,7H-dibenzo[b,g][1,5]dioxocin-5-one

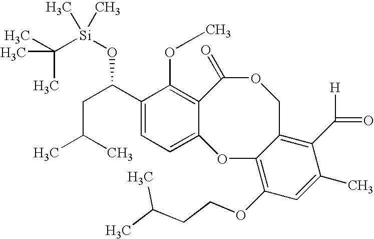

125 mg (0.214 mmol) of the compound from Example B-XLV are reacted analogously to Example B-X. This gives 82 mg (66% of theory) of product.

¹H-NMR (200 MHz, CDCl₃): δ=−0.17 (s, 3H), 0.06 (s, 3H), 0.86 (s, 9H), 0.91 (d, 3H), 0.96 (d, 3H), 1.02 (d, 6H), 1.19-1.36 (m, 2H), 1.06 (m, 6H), 2.62 (s, 3H), 3.96 (s, 3H), 4.17 (t, 2H), 5.11 (dd, 1H), 5.58 (br. s, 2H), 6.79 (d, 1H), 6.79 (s, 1H), 7.59 (d, 1H), 10.34 (s, 1H) ppm.

MS (ESIpos): m/z=607 (M+Na)⁺

HPLC (Method 8): Rₜ=8.75 min.

### EXAMPLE B-XLVII

### 9-(Bromomethyl)-3-((1S)-1-{[tert-butyl(dimethyl)silyl]oxy}-3-methylbutyl)-11-(isopentyloxy)-4-methoxy-5H,7H-dibenzo[b,g][1,5]dioxocin-5-one

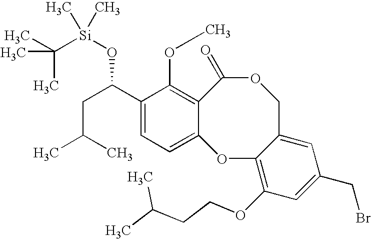

224 mg (1.26 mmol) of N-bromosuccinimide and 15 mg (0.090 mmol) of 2,2′-azobis-2-methylpropionitrile are added to a solution, heated at reflux, of 500 mg (0.898 mmol) of the compound from Example B-XLIII in 5 ml of carbon tetrachloride. After addition of a further two equivalents of 2,2′-azobis-2-methylpropionitrile and 6 hours under reflux, the reaction mixture is cooled and filtered. The filtrate is concentrated under reduced pressure and the residue is purified chromatographically on silica gel (mobile phase: cyclohexane/ethyl acetate 20:1→5:1). This gives 74 mg (13% of theory) of product.

¹H-NMR (300 MHz, CDCl₃): δ=−0.19 (s, 3H), 0.04 (s, 3H), 0.84-1.00 (m, 21H), 1.18-1.95 (m, 6H), 3.92 (s, 3H), 4.09 (t, 2H), 4.39 (s, 2H), 5.00-5.11 (m, 3H), 6.64 (d, 1H), 6.89 (d, 1H), 6.99 (d, 1H), 7.59 (d, 1H) ppm.

### EXAMPLE B-XLVIII

### 9-((1S)-1-{[tert-Butyl(dimethyl)silyl]oxy}-3-methylbutyl)-1-(isopentyloxy)-8-methoxy-7-oxo-5H,7H-dibenzo[b,g][1,5]dioxocin-3-carbaldehyde

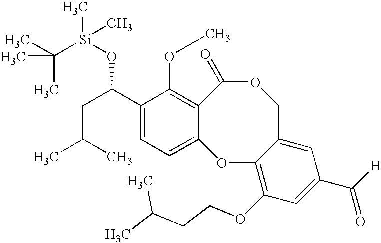

25 mg (214 μmol) of N-methylmorpholino N-oxide, together with 100 mg of powdered molecular sieve (4 Å), are suspended in 5 ml of acetonitrile, and 68 mg (107 μmol) of the compound from Example B-XLVII, dissolved in 1 ml of acetonitrile, are added. After 4 hours at, room temperature, the reaction mixture is filtered through 2 g of silica gel and eluted with dichloromethane. The filtrate is concentrated under reduced pressure. This gives 52 mg (85% of theory) of product.

¹H-NMR (300 MHz, CDCl₃): δ=−0.18 (s, 3H), 0.05 (s, 3H), 0.85-1.02 (m, 21H), 1.20-1.95 (m, 6H), 3.96 (s, 3H), 4.18 (t, 2H), 5.04-5.18 (m, 3H), 6.91 (d, 1H), 7.17 (d, 1H), 7.50 (d, 1H), 7.65 (d, 1H), 9.86 (s, 1H) ppm.

### EXAMPLE B-IL

### 3-((1S)-1-{[tert-Butyl(dimethyl)silyl]oxy}-3-methylbutyl)-9-(difluoromethyl)-11-isopentyloxy)-4-methoxy-5H,7H-dibenzo[b,g][1,5]dioxocin-5-one

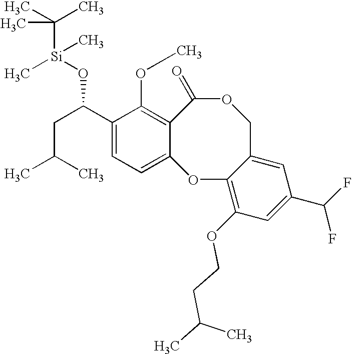

Under argon and at 0° C., 27 μl (210 μmol) of diethylaminosulphur trifluoride are added to 20 mg (35 μmol) of the compound from Example B-XLVIII in 5 ml of 1,2-dichloroethane, and the mixture is stirred under reflux overnight. For work-up, the reaction solution is cooled to room temperature and washed with 5 ml of saturated sodium bicarbonate solution. The aqueous phase is extracted once with dichloromethane. The combined organic phases are dried over sodium sulphate and the solvent is removed under reduced pressure. The residue is purified by preparative thick-layer chromatography (mobile phase: cyclohexane/ethyl acetate 4:1). This gives 16 mg (76% of theory) of product.

¹H-NMR (300 MHz, CDCl₃): δ=−0.21 (s, 3H), 0.04 (s, 3H), 0.84 (s, 9H), 0.85-1.04 (m, 12H), 1.20-2.00 (m, 6H), 3.93 (s, 3H), 4.12 (t, 2H), 5.01-5.11 (m, 3H), 6.55 (t, 1H), 6.76 (s, 1H), 6.89 (d, 1H), 7.11 (s, 1H), 7.61 (d, 1H) ppm.

MS (ESIpos): m/z=615 (M+Na)⁺

### EXAMPLE B-L

### 3-((1S)-1-{[tert-Butyl(dimethyl)silyl]oxy}-3-methylbutyl)-8-(difluoromethyl)-11-(isopentyloxy)-4-methoxy-9-methyl-5H,7H-dibenzo[b,g][1,5]dioxocin-5-one

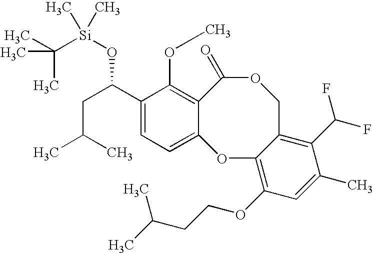

60 mg (0.103 mmol) of the compound from Example B-XLVI are reacted analogously to Example B-IL. This gives 41 mg (66% of theory) of product.

¹H-NMR (200 MHz, CDCl₃): δ=−0.17 (s, 3H), 0.06 (s, 3H), 0.86 (s, 9H), 0.91 (d, 3H), 0.96 (d, 3H), 1.02 (d, 6H), 1.19-1.36 (m, 2H), 1.06 (m, 6H), 2.62 (s, 3H), 3.96 (s, 3H), 4.17 (t, 2H), 5.11 (dd, 1H), 5.58 (br. s, 2H), 6.79 (d, 1H), 6.79 (s, 1H), 7.59 (d, 1H), 10.34 (s, 1H) ppm.

MS (DCI): m/z=624 (M+NH₄)⁺

HPLC (Method 8): Rₜ=8.97 min.

### EXAMPLE B-LI

### 8,10-Dichloro-11-hydroxy-3-[(1S)-1-hydroxy-3-methylbutyl]-4-methoxy-9-methyl-5H,7H-dibenzo[b,g][1,5]dioxocin-5-one

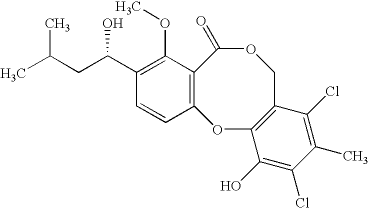

1.5 g (4.03 mmol) of penicillide are initially charged in 30 ml of ethanol/water (1:1), 1.18 g (8.86 mmol) of N-chlorosuccinimide and 1.83 g (7.81 mmol) of iron(III) chloride hexahydrate are added and the mixture is stirred at room temperature over the weekend. For work-up, the reaction mixture is diluted with ethyl acetate and washed with water. The organic phase is dried over sodium sulphate and concentrated under reduced pressure. The residue is purified chromatographically on silica gel (mobile phase: cyclohexane/ethyl acetate 10:1→5:1). This gives 1.23 g (69% of theory) of product.

¹H-NMR (300 MHz, CDCl₃): δ=0.98 (t, 6H), 1.44-1.86 (m, 3H), 2.00 (br. s, 1H), 2.44 (s, 3H), 3.99 (s, 3H), 5.05-5.12 (m, 1H), 5.41 (q, 2H), 6.40 (s, 1H), 6.87 (d, 1H), 7.59 (d, 1H) ppm.

MS (ESIpos): m/z=464 (M+Na)⁺

HPLC (Method 1): Rₜ=5.07 min.

### EXAMPLE B-LII

### 8-Chloro-11-hydroxy-3-[(1S)-1-hydroxy-3-methylbutyl]-4-methoxy-9-methyl-5H,7H-dibenzo[b,g][1,5]dioxocin-5-one

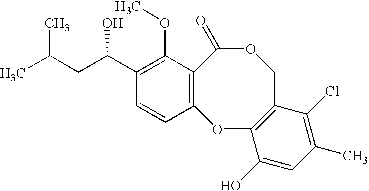

2.25 g (6.04 mmol) of penicillide are initially charged in 45 ml of ethanol/water (1:1), 847 mg (6.34 mmol) of N-chlorosuccinimide and 1.58 g (5.86 mmol) of iron(III) chloride hexahydrate are added and the mixture is stirred at room temperaure over the weekend. For work-up, the reaction mixture is diluted with 100 ml of ethyl acetate and washed with water. The organic phase is washed once with saturated sodium chloride solution, dried over sodium sulphate and concentrated under reduced pressure. The residue is purified chromatographically on silica gel (mobile phase: cyclohexane/ethyl acetate 5:1→3:1). This gives 2.21 g (75% of theory) of product.

¹H-NMR (300 MHz, CDCl₃): δ=0.98 (t, 6H), 1.42-1.86 (m, 3H), 2.04 (br. s, 1H), 2.29 (s, 3H), 3.99 (s, 3H), 5.09 (dd, 1H), 5.35-5.48 (m, 2H), 6.05 (br. s, 1H), 6.84 (d, 1H), 6.94 (br. s, 1H), 7.59 (d, 1H) ppm.

MS (ESIpos): m/z=429 (M+Na)⁺

HPLC (Method 2): Rₜ=4.86 min.

### EXAMPLE B-LIII

### 10-Bromo-8-chloro-11-hydroxy-3-[(1S)-1-hydroxy-3-methylbutyl]-4-methoxy-9-methyl-5H,7H-dibenzo[b,g][1,5]dioxocin-5-one

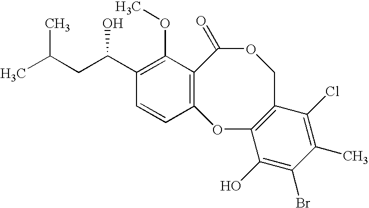

320 mg (0.47 ml, 4.42 mmol) of tert-butylamine are dissolved in 20 ml of toluene, and this solution is cooled to −30° C. Over a period of 5 minutes, a solution of 1.41 g (0.46 ml, 8.85 mmol) of bromine in 25 ml of dichloromethane is slowly added dropwise. The mixture is then cooled to −78° C., and a solution of the compound from Example B-LII in 25 ml of dichloromethane is added. With vigorous stirring, the mixture is warmed to room temperature and allowed to stand for 4-5 hours. The mixture is washed with 1 M hydrochloric acid and then with water. The organic phase is dried over sodium sulphate, filtered and concentrated. The residue is separated chromatographically (silica gel, mobile phase cyclohexane/ethyl acetate 5:1→3:1) and then purified further by preparative HPLC. This gives 791 mg (purity 88%, 39% of theory) of product.

R_{f}=0.35 (cyclohexane/ethyl acetate 2:1)

¹H-NMR (300 MHz, CDCl₃): δ=0.99 (m, 6H), 1.43 (m, 1H), 1.62 (m, 2H), 1.80 (m, 1H), 1.95 (m, 1H), 2.52 (s, 3H), 3.99 (s, 3H), 5.09 (dd, 1H), 5.40 (m, 2H), 6.38 (s, 1H), 6.87 (d, 1H), 7.62 (d, 1H) ppm.

MS (DCI): m/z=503 (M+NH₄)⁺

HPLC (Method 1): Rₜ=4.97 min.

### WORKING EXAMPLES

### EXAMPLE B-1

### 8,10-Dibromo-3-[(1S)-1-hydroxy-3-methylbutyl]-4-methoxy-9-methyl-11-(4,4,4-trifluorobutoxy)-5H,7H-dibenzo[b,g][1,5]dioxocin-5-one

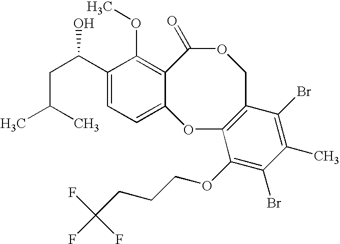

Under argon, 1.06 g (2 mmol) of the compound from Example B-I are dissolved in 14 ml of dimethylformamide, and 780 mg (2.4 mmol) of caesium carbonate and 480 mg (2.4 mmol) of 1-bromo-4,4,4-trifluorobutane are added. The reaction vessel is closed immediately, and the mixture is stirred at 60° C. After one hour, the mixture is cooled and slowly stirred into ice-cold 0.15 N hydrochloric acid; this results in the precipitation of the product. After 20 hours, the precipitate is filtered off with suction and washed once with water. The solid is taken up in dichloromethane and the solution is dried over sodium sulphate and concentrated under reduced pressure. The residue is purified chromatographically on a silica gel column (mobile phase:

cyclohexane/ethyl acetate 20:1→6:1). This gives 906 mg (71% of theory) of product.

¹H-NMR (300 MHz, CDCl₃): δ=0.98 (t, 6H), 1.52-1.81 (m, 3H), 1.92 (d, 1H), 2.07-2.14 (m, 2H), 2.39-2.52 (m, 2H), 2.6 (s, 3H), 3.99 (s, 3H), 4.18 (t, 2H), 5.05-5.13 (m, 1H), 5.42 (d, 2H), 6.92 (d, 1H), 7.60 (d, 1H) ppm.

MS (DCI): m/z=658 (M+NH₄)⁺

HPLC (Method 1): Rₜ=6.15 min.

The examples listed in the table below are prepared analogously to the procedures described above, from the corresponding starting materials:

| Example | | |
|---|---|---|
| B- | Structure | Analytical data |
| 2 | 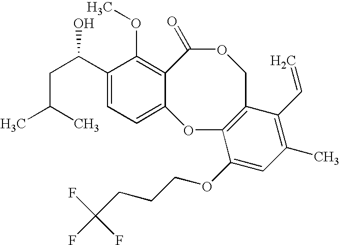 | ¹H-NMR (400 MHz, CDCl₃): δ = 0.91 (t, 6 H), 1.39-1.76 (m, 4 H), 2.00-2.09 (m, 2 H), 2.14 (t, 3 H), 2.27-2.39 (m, 2 H), 3.91 (s, 3 H), 4.06 (t, 2 H), 4.87 (dd, 1 H), 4.99-5.05 (m, 1 H), 5.23 (s, 2 H), 5.47 (dd, 1 H), 6.49 (dd, 1 H), 6.73 (s, 1 H), 6.83 (d, 1 H), 7.51 (d, 1 H) ppm. MS (ESIpos): m/z = 509 |
| #(M + H)⁺HPLC (Method 1): Rₜ = 5.44 min. | | |
| 3 | 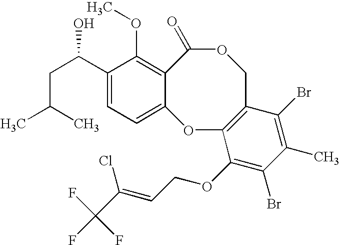 | MS (EIpos): m/z = 672/674 [M]⁺HPLC (Method 1): Rₜ = 6.25 min. |

### EXAMPLE B-4

### 3-[(1S)-1-Hydroxy-3-methylbutyl]-4-methoxy-9-methyl-11-(4,4,4-trifluorobutoxy)-5H,7H-dibenzo[b,g][1,5]dioxocin-5-one

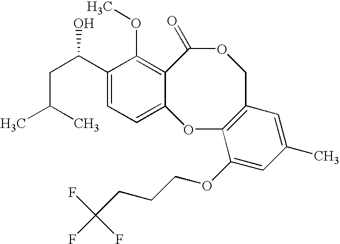

Under argon, 100 mg (0.27 mmol) of penicillide are dissolved in 1 ml of tetrahydrofuran, the mixture is cooled to 0° C. and 11 mg (0.28 mmol) of sodium hydride are added. After 5 minutes, 77 mg (0.40 mmol) of 1-bromo-4,4,4-trifluorobutane and a catalytic amount of tetrabutylammonium iodide are added, and the mixture is heated at 60° C. overnight. After cooling, 1.5 ml of water are added to the reaction mixture and the mixture is diluted with dichloromethane and filtered through an Extrelut NT 3 cartridge. The cartridge is eluted three times with in each case 5 ml of dichloromethane and the filtrate is concentrated under reduced pressure.

The residue is purified by preparative HPLC. This gives 93 mg (60% of theory) of product.

R_{f}=0.39 (cyclohexane/ethyl acetate 2:1)

¹H-NMR (300 MHz, CDCl₃): δ=0.97 (t, 6H), 1.43-1.81 (m, 4H), 2.08-2.18 (m, 2H), 2.27 (s, 3H), 2.32-2.48 (m, 2H), 3.97 (s, 3H), 4.12 (t, 2H), 5.05-5.12 (m, 3H), 6.46 (s, 1H), 6.78 (s, 1H), 6.91 (d, 1H), 7.57 (d, 1H) ppm.

MS (DCI): m/z=505 (M+Na)⁺

HPLC (Method 1): Rₜ=4.99 min.

### EXAMPLE B-5

### 3-[(1S)-1-Hydroxy-3-methylbutyl]-4-methoxy-9-methyl-11-(bicyclo[2.2.1]hept-5-en-2-ylmethoxy)-5H,7H-dibenzo[b,g][1,5]dioxocin-5-one

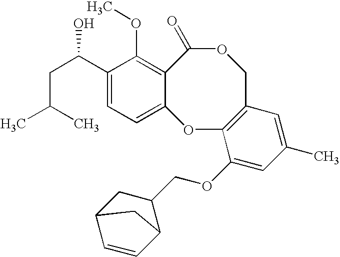

Under argon, 100 mg (0.27 mmol) of penicillide are dissolved in 1 ml of dimethylformamide, the mixture is cooled to 0° C. and 11 mg (0.28 mmol) of sodium hydride are added. After 15 minutes, 77 mg (0.40 mmol) of 5-chloromethyl-bicyclo[2.2.1]hept-2-ene, a catalytic amount of tetrabutylammonium iodide and 2 drops of 18-crown-6 are added. The mixture is then heated at 100° C. for 36 hours. A further 57 mg (0.4 mmol) of 5-chloromethyl-bicyclo[2.2.1]hept-2-ene are added, and the mixture is stirred at 100° C. for another 16 h. After cooling, 5 ml of water are added and the reaction mixture is extracted twice with in each case 10 ml of ethyl acetate. The combined organic phases are washed with 5 ml of water, dried over sodium sulphate and filtered, and the solvent is removed under reduced pressure. The residue is purified by preparative HPLC. This gives 28 mg (21% of theory) of product.

R_{f}=0.16 (cyclohexane/ethyl acetate 2:1)

¹H-NMR (400 MHz, CDCl₃): δ=0.99 (dd, 6H), 1.30-1.56 (m, 4H), 1.66-1.87 (m, 2H), 1.91-2.07 (m, 2H), 2.26+2.27 (2s, 3H), 2.64-2.72 (m, 1H), 2.89 (br. s, 1H), 3.02 (d, 1H), 3.60-3.83 (m, 1H), 3.93-4.16 (m, 4H), 5.05-5.16 (m, 4H), 6.00-6.23 (m, 2H), 6.42+6.43 (2s, 1H), 6.73+6.82 (2s, 1H), 6.98+7.01 (2d, 1H), 7.58+7.60 (2d, 1H) ppm.

MS (ESIpos): m/z=501 (M+Na)⁺

HPLC (Method 1): Rₜ=5.73 min.

### EXAMPLE B-6

### 9-[(1S)-1-Hydroxy-3-methylbutyl]-8-methoxy-3-methyl-7-oxo-5H,7H-dibenzo[b,g]-[1,5]-dioxocin-1-yl cyclopropanecarboxylate

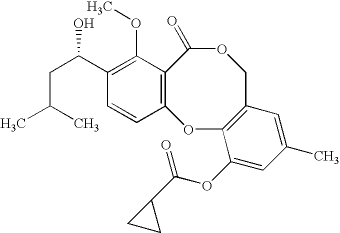

Under argon, 150 mg (0.40 mmol) of penicillide are dissolved in 2 ml of tetrahydrofuran and, at 0° C., 24 mg (0.60 mmol) of sodium hydride (60%) are added. After 5 minutes, 50 mg (0.48 mmol) of cyclopropanecarbonyl chloride are added and the mixture is stirred at room temperature. After one hour, the reaction mixture is diluted with dichloromethane and 1.5 ml of water are added. The mixture is filtered through an Extrelut cartridge and eluted with dichloromethane, and the filtrate is concentrated under reduced pressure. The residue is purified chromatographically on silica gel (mobile phase: cyclohexane/ethyl acetate 10:1→3:1). This gives 141 mg (79% of theory) of product.

¹H-NMR (300 MHz, CDCl₃): δ=0.98 (m, 6H), 1.02-1.08 (m, 2H), 1.22-1.27 (m, 2H), 1.65-2.06 (m, 5H), 2.30 (s, 3H), 3.97 (s, 3H), 5.05-5.12 (m, 3H), 6.71 (br. s, 1H), 6.92 (d, 1H), 6.92 (br. s, 1H), 7.58 (d, 1H) ppm.

MS (ESIpos): m/z=463 (M+Na)⁺

HPLC (Method 1): Rₜ=5.01 min.

The examples listed in the table below are prepared analogously to the procedures described above, from the corresponding starting materials:

| Example | | |
|---|---|---|
| B- | Structure | Analytical data |
| 7 | 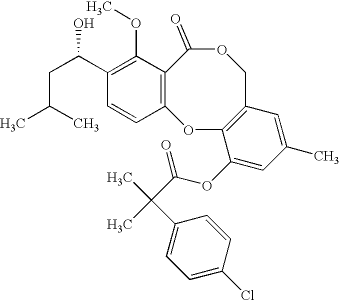 | ¹H-NMR(300 MHz, CDCl₃): δ = 0.98(t, 6H), 1.42-1.82(m, 3H), 1.74(s, 6H), 1.91(d, 1H), 2.27(s, 3H), 3.97(s, 3H), 5.02-5.15(m, 3H), 6.53(d, 1H), 6.71 (br. s, 1H), 6.80(br. s, 1H), 7.25 (d, 2H), 7.42(d, 2H), 7.49(d, 1H) ppm. MS(ESIpos): m/z = 575(M + Na)⁺HPLC(Method 1): Rₜ = 5.55 min. |
| 8 | 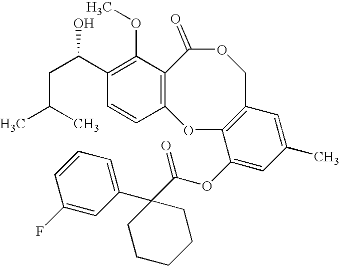 | ¹H-NMR(300 MHz, CDCl₃): δ = 0.98(t, 6H), 1.42-1.92(m, 12H), 2.23(s, 3H), 2.61-2.72(m, 2H), 3.97(s, 3H), 5.03-5.12(m, 3H), 6.65-6.70(m, 3H), 6.87-6.94 (m, 1H), 7.25-7.35(m, 3H), 7.49 (d, 1H) ppm. MS(ESIpos): m/z = 599(M + Na)⁺HPLC(Method 1): Rₜ = 5.72 min. |
| 9 | 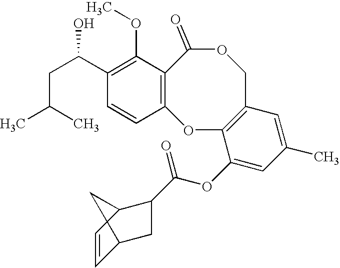 | ¹H-NMR(300 MHz, CDCl₃): δ = 0.98(m, 6H), 1.35-2.09(m, 8H), 2.27(s, 3H), 2.97(s, 1H), 3.26-3.32(m, 1H), 3.42(s, 1H), 3.97(s, 3H), 5.02-5.12(m, 3H), 6.05-6.25(m, 2H), 6.71(br. s, 1H), 6.93(br. s, 1H), 7.00(d, 1H), 7.55 (d, 1H) ppm. MS(ESIpos): m/z = 515(M + Na)⁺HPLC(Method 1): Rₜ = 5.29 min. |
| 10 | 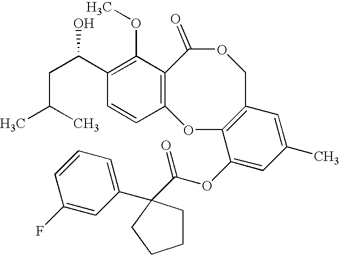 | ¹H-NMR(300 MHz, CDCl₃): δ = 0.98(m, 6H), 1.42-2.07(m, 10H), 2.22(s, 3H), 2.78-2.90(m, 2H), 3.96(s, 3H), 5.02-5.12(m, 3H), 6.64(d, 1H), 6.69(d, 2H), 6.89-6.94(m, 1H), 7.19-7.29(m, 3H), 7.50(d, 1H) ppm. MS(ESIpos): m/z =585(M + Na)⁺HPLC(Method 1): Rₜ = 5.58 min. |
| 11 | 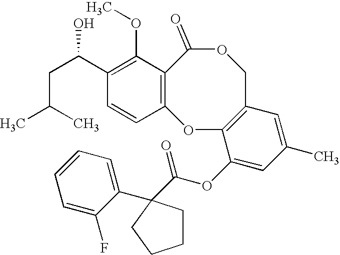 | ¹H-NMR(300 MHz, CDCl₃): δ = 0.97(t, 6H), 1.44-1.93(m, 7H), 2.12-2.23(m, 2H), 2.24(s, 3H), 2.67-2.79(m, 2H), 3.96(s, 3H), 5.02-5.12(m, 3H), 6.64(d, 1H), 6.69(d, 1H), 6.81(d, 1H), 6.85(d, 1H), 7.02-7.18(m, 2H), 7.23-7.32(m, 1H), 7.43(dt, 1H), 7.53 (d, 1H) ppm. MS(ESIpos): m/z = 585(M + Na)⁺HPLC(Method 1): Rₜ = 5.52 min. |
| 12 | 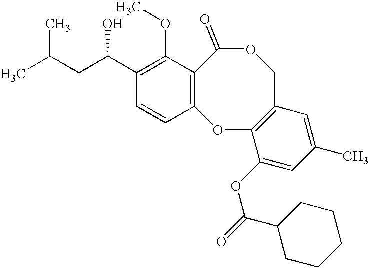 | ¹H-NMR(300 MHz, CDCl₃): δ = 0.97(t, 6H), 1.25-2.12(m, 14H), 2.27(s, 3H), 2.60-2.70(m, 1H), 3.97(s, 3H), 5.06-5.10(m, 3H), 6.71(br. s, 1H), 6.93(br. s, 1H), 7.00(d, 1H), 7.55(d, 1H) ppm. MS(ESIpos): m/z =505(M + Na)⁺HPLC(Method 1): Rₜ = 5.40 min. |
| 13 | 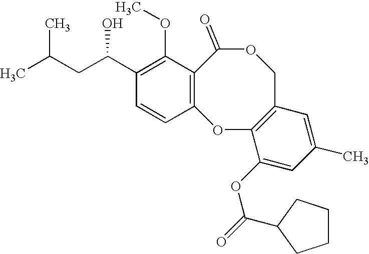 | ¹H-NMR(300 MHz, CDCl₃): δ = 0.97(t, 6H), 1.44-1.91(m, 8H), 2.04(q, 4H), 2.28(s, 3H), 3.07(m, 1H), 3.97(s, 3H), 5.06-5.10(m, 3H), 6.71(br. s, 1H), 6.93 (br. s, 1H), 7.00(d, 1H), 7.55(d, 1H) ppm. MS(ESIpos): m/z = 491(M + Na)⁺HPLC(Method 1): Rₜ = 5.21 min. |
| 14 | 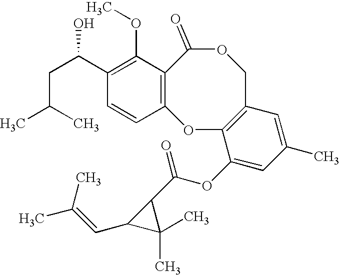 | ¹H-NMR(200 MHz, DMSO-d₆): δ = 0.90(m, 6H), 1.08-2.18(m, 18H), 2.25(s, 3H), 3.82(s, 3H), 4.89(quintet, 1H), 5.10-5.22(m, 3H), 6.80-7.12(m, 3H), 7.60(m, 1H) ppm. MS(ESIpos): m/z = 545(M + Na)⁺HPLC(Method 2): Rₜ = 5.58 min. |
| 15 | 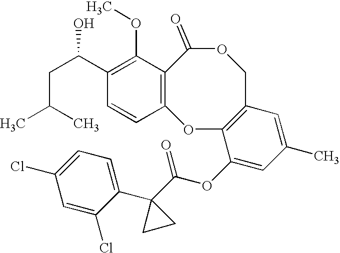 | ¹H-NMR(200 MHz, CDCl₃): δ = 0.98(m, 6H), 1.25-2.00(m, 8H), 2.25(s, 3H), 3.97(s, 3H), 5.02-5.15(m, 3H), 6.71(s, 1H), 6.74(d, 1H), 6.92(s, 1H), 7.33(s, 1H), 7.38-7.42(m, 1H), 7.45(d, 1H), 7.53(d, 1H) ppm. MS(ESIpos): m/z =608(M + Na)⁺HPLC(Method 2): Rₜ = 5.81 min. |
| 16 | 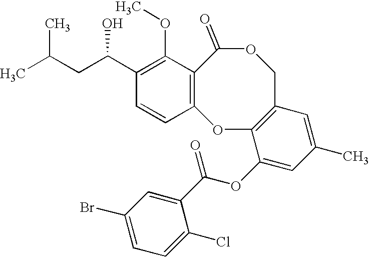 | ¹H-NMR(300 MHz, CDCl₃): δ = 0.97(m, 6H), 1.42-1.95(m, 4H), 2.32(s, 3H), 3.97(s, 3H), 5.02-5.15(m, 3H), 6.82(d, 1H), 7.03(d, 1H), 7.09(d, 1H), 7.41(d, 1H), 7.58(d, 1H), 7.62(dd, 1H), 8.24(d, 1H) ppm. MS(DCI): m/z = 608(M + NH₄)⁺HPLC(Method 1): Rₜ = 5.49 min. |
| 17 | 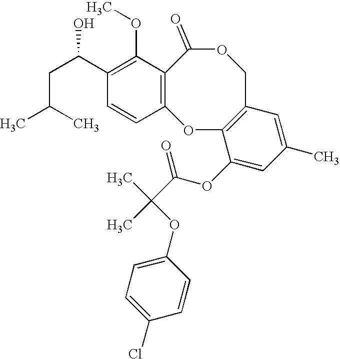 | ¹H-NMR(300 MHz, CDCl₃): δ = 0.98(t, 6H), 1.42-1.91(m, 4H), 1.77(s, 6H), 2.28(s, 3H), 3.98(s, 3H), 5.05-5.12(m, 3H), 6.75-6.78(m, 2H), 6.83(s, 1H), 7.05(dd, 4H), 7.50(d, 1H) ppm. MS(ESIpos): m/z = 591(M + Na)⁺HPLC(Method 2): Rₜ = 5.21 min. |
| 18 | 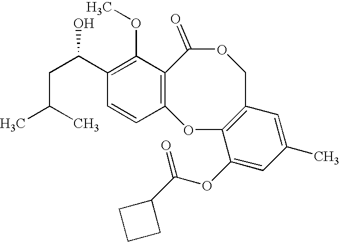 | ¹H-NMR(300 MHz, CDCl₃): δ = 0.97(t, 6H), 1.42-1.50(m, 1H), 1.65-2. 14(m, 5H), 2.28(s, 3H), 2.29-2.56(m, 4H), 3.46 (quintet, 1H), 3.97(s, 3H), 5.05-5.12(m, 3H), 6.71(hr. s, 1H), 6.93 (br. s, 1H), 7.00(d, 1H), 7.55(d, 1H) ppm. MS(ESIpos): m/z = 477(M + Na)⁺HPLC(Method 2): Rₜ = 5.63 min. |
| 19 | 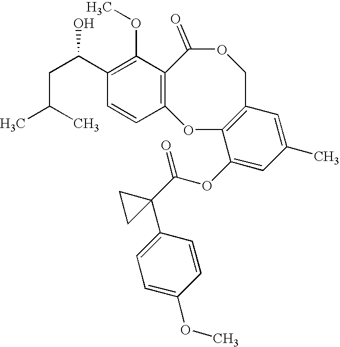 | ¹H-NMR(200 MHz, CDCl₃): δ = 0.98(q, 6H), 1.25-1.84(m, 4H), 2.24(s, 3H), 3.52-3.69(m, 4H), 3.80(s, 3H), 3.98(s, 3H), 5.05-5.12(m, 3H), 6.69(d, 1H), 6.77(d, 1H), 6.87(d, 2H), 6.90(d, 1H), 7.39(d, 2H), 7.53(d, 1H) ppm. MS(ESIpos): m/z = 569(M + Na)⁺HPLC(Method 2): Rₜ = 5.36 min. |
| 20 | 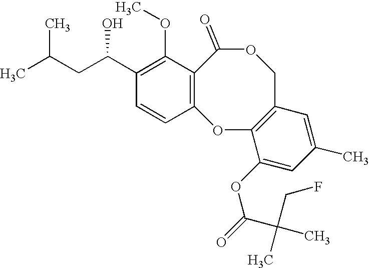 | ¹H-NMR(200 Mhz, CDCl₃): δ = 0.97(m, 6H), 1.27-1.76(m, 4H), 1.42(s, 6H), 2.28(s, 3H), 3.97(s, 3H), 4.69(s, 1H), 4.45(s, 1H), 5.05-5.12(m, 3H), 6.71(br. s, 1H), 6.93(br. s, 1H), 7.00(d, 1H), 7.55(d, 1H) ppm. MS(ESIpos): m/z = 497(M + Na)⁺HPLC(Method 2): Rₜ = 5.12 min. |
| 21 | 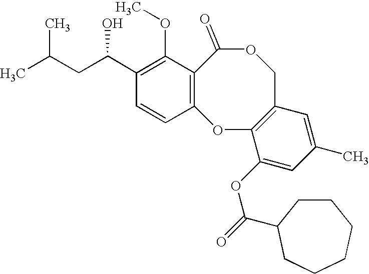 | ¹H-NMR(300 MHz, CDCl₃): δ = 0.97(m, 6H), 1.43-1.95(m, 13H), 2.06(d, 1H), 1.98-2.20(m, 2H), 2.27(s, 3H), 2.82(sep., 1H), 3.97(s, 3H), 5.02-5.12(m, 3H), 6.71(br. s, 1H), 6.93(br. s, 1H), 7.00(d, 1H), 7.55(d, 1H) ppm. MS(ESIpos): m/z = 519(M + Na)⁺HPLC(Method 2): Rₜ = 5.70 min. |
| 22 | 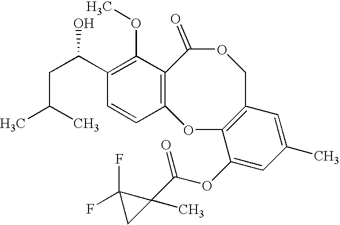 | ¹H-NMR(200 MHz, CDCl₃): δ = 0.97(t, 6H), 1.42(s, 3H), 1.45-1.80(m, 6H), 2.29(s, 3H), 3.97(s, 3H), 5.02-5.12(m, 3H), 6.71(br. s, 1H), 6.93(br. s, 1H), 7.00(d, 1H), 7.55(d, 1H) ppm. MS(ESIpos): m/z = 513(M + Na)⁺HPLC(Method 2): Rₜ = 5.16 min. |
| 23 | 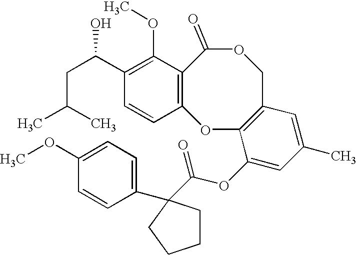 | ¹H-NMR(200 MHz, CDCl₃): δ = 0.98(m, 6H), 1.10-2.08(m, 12H), 2.22(s, 3H), 3.75(s, 3H), 3.96(s, 3H), 5.00-5.12(m, 3H), 6.53(d, 1H), 6.69-6.73(m, 2H), 6.83(d, 2H), 7.40-7.48(m, 3H) ppm. MS(ESIpos): m/z = 597(M + Na)⁺HPLC(Method 1): Rₜ = 5.69 min. |
| 24 | 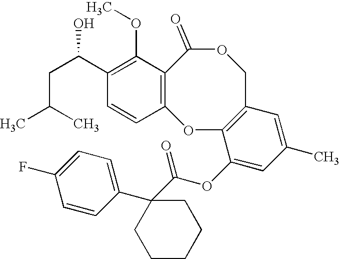 | ¹H-NMR(200 MHz, CDCl₃): δ = 0.98(m, 6H), 1.15-1.98(m, 12H), 2.23(s, 3H), 2.60-2.75(m, 2H), 3.97(s, 3H), 5.00-5.12(m, 3H), 6.56(d, 1H), 6.69(d, 2H), 6.99(t, 2H), 7.44-7.55(m, 3H) ppm. MS(ESIpos): m/z = 599(M + Na)⁺HPLC(Method 1): Rₜ = 5.71 min. |
| 25 | 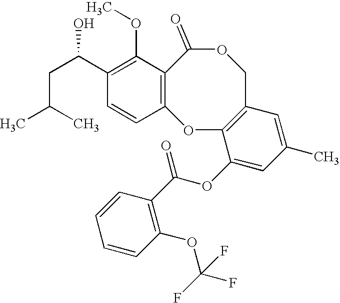 | ¹H-NMR(300 MHz, CDCl₃): δ = 0.96(m, 6H), 1.43-1.78(m, 3H), 1.90(d, 1H), 2.32(s, 3H), 3.96(s, 3H), 5.02-5.12(m, 3H), 6.80(br. s, 1H), 7.00(d, 1H), 7.09 (br. s, 1H), 7.55(d, 1H), 7.41-7.47 (m, 2H), 7.66(dd, 1H), 8.21(dd, 1H) ppm. MS(ESIpos): m/z = 583(M + Na)⁺HPLC(Method 1): Rₜ = 5.18 min. |
| 26 | 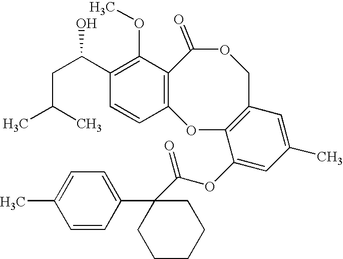 | ¹H-NMR(300 MHz, CDCl₃): δ = 0.98(m, 6H), 1.25-1.91(m, 12H), 2.22(s, 3H), 2.27(s, 3H), 2.62-2.72(m, 2H), 3.96(s, 3H), 5.02-5.12(m, 3H), 6.57(d, 1H), 6.69(d, 2H), 7.12(d, 2H), 7.41-7.46(m, 3H) ppm. MS(ESIpos): m/z = 595(M + Na)⁺HPLC(Method 2): Rₜ = 5.68 min. |
| 27 | 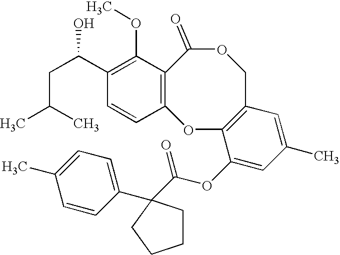 | ¹H-NMR(300 MHz, CDCl₃): δ = 0.98(m, 6H), 1.43-2.07(m, 10H), 2.21(s, 3H), 2.29(s, 3H), 2.78-2.89(m, 2H), 3.96(s, 3H), 5.02-5.12(m, 3H), 6.56(d, 1H), 6.69(d, 2H), 7.11(d, 2H), 7.39(d, 2H), 7.46(d, 1H) ppm. MS(ESIpos): m/z = 581(M + H)⁺HPLC(Method 2): Rₜ = 5.51 min. |
| 28 | 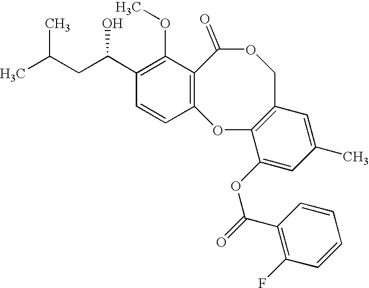 | ¹H-NMR(200 MHz, CDCl₃): δ = 0.96(q, 6H), 1.39-1.90(m, 4H), 2.32(s, 3H), 3.95(s, 3H), 5.04-5.12(m, 3H), 6.81(s, 1H), 7.05(d, 1H), 7.09(s, 1H), 7.18-7.32(m, 2H), 7.55-7.70(m, 2H), 8.11-8.19(m,1H)ppm. LC-MS(Method 3): Rₜ = 4.74 min. MS(ESIpos): m/z = 517(M + Na)⁺ |
| 29 | 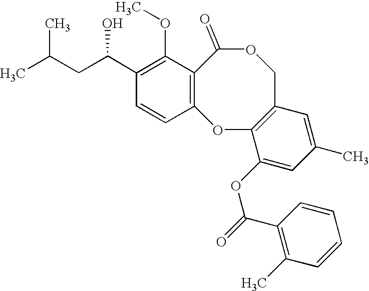 | ¹H-NMR(200 MHz, CDCl₃): δ = 0.96(q, 6H), 1.39-1.83(m, 3H), 1.95(d, 1H), 2.32(s, 3H), 2.71(s, 3H), 3.96(s, 3H), 5.03-5.12(m, 3H), 6.80(s, 1H), 6.99(d, 1H), 7.07(s, 1H), 7.28-7.37(m, 2H), 7.44-7.51(m, 1H), 7.56(d, 1H), 8.19-8.26(m, 1H) ppm. LC-MS(Method 3): Rₜ = 4.99 min. MS(ESIpos): m/z =513(M + Na)⁺ |
| 30 | 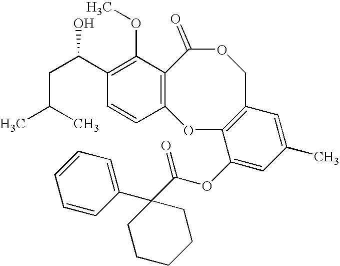 | ¹H-NMR(200 MHz, CDCl₃): δ = 0.97(q, 6H), 1.25-1.90(m, 12H), 2.22(s, 3H), 2.63-2.74(m, 2H), 3.96(s, 3H), 5.03-5.12(m, 3H), 6.61-6.68(m, 3H), 7.17-7.36 (m, 3H), 7.47(d, 1H), 7.5 1-7.60 (m, 2H) ppm. LC-MS(Method 3): Rₜ = 5.43 min. MS(ESIpos): m/z = 581(M + Na)⁺MS(ESIpos): m/z = 559(M + H)⁺ |
| 31 | 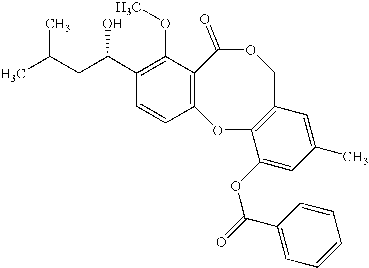 | ¹H-NMR(200 MHz, CDCl₃): δ = 0.96(q, 6H), 1.25-1.82(m, 3H), 1.95(d, 1H), 2.32(s, 3H), 3.94(s, 3H), 5.03-5.12(m, 3H), 6.80(s, 1H), 6.99(d, 1H), 7.08(s, 1H), 7.49-7.69(m, 4H), 8.23-8.28 (m, 2H) ppm. LC-MS(Method 3): Rₜ = 4.81 min. MS(ESIpos): m/z = 499(M + Na)⁺ |
| 32 | 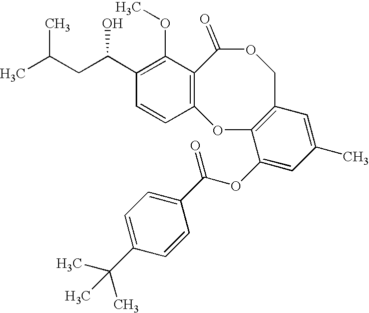 | ¹H-NMR(200 MHz, CDCl₃): δ = 0.96(q, 6H), 1.36(s, 9H), 1.25-1.82(m, 3H), 1.95(d, 1H), 2.31(s, 3H), 3.94(s, 3H), 5.03-5.12(m, 3H), 6.79(s, 1H), 7.00(d, 1H), 7.06(s, 1H), 7.5 1-7.56(m, 4H), 8.18(d, 1H) ppm. LC-MS(Method 3): Rₜ = 5.46 min MS(ESIpos): m/z = 515(M − OH)⁺ |
| 33 | 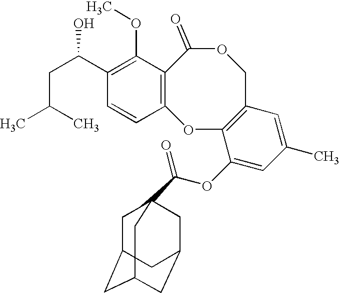 | ¹H-NMR(200 MHz, CDCl₃): δ = 0.98(q, 6H), 1.40-1.75(m, 2H), 1.76(s, 5H), 2.09(s, 9H), 2.27(s, 3H), 3.97(s, 3H), 5.00-5.15(m, 3H), 6.73(s, 1H), 6.90(s, 1H), 6.95(d, 1H), 7.59(d, 1H) ppm. LC-MS(Method 3): Rₜ = 5.59 min. MS(ESIpos): m/z = 517(M-OH)⁺ |
| 34 | 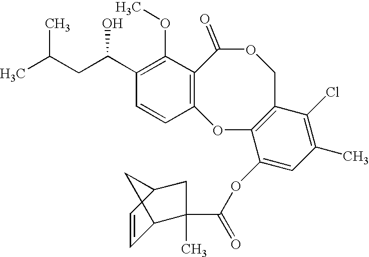 | ¹H-NMR(400 MHz, CDCl₃): δ = 0.98(m, 6H), 1.42-1.80(m, 10H), 2.31(2s, 3H), 2.91-3.21(m, 2H), 3.99(s, 3H), 5.08(m, 1H), 5.39(d, 1H), 5.43(d, 1H), 6.10-6.21(m, 1H), 6.38(m, 1H), 6.89 (m, 1H), 7.01(br. s, 1H), 7.59(m, 1H) ppm. MS(DCI): m/z = 558(M + NH₄)⁺HPLC(Method 1): Rₜ = 5.6, 5.7 min. |
| 35 | 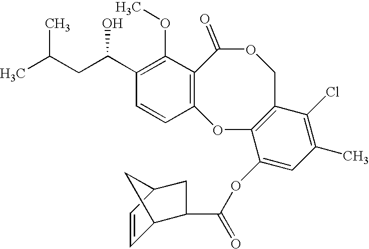 | ¹H-NMR(200 MHz, CDCl₃): δ = 0.98(m, 6H), 1.42-1.80(m, 8H), 2.31(2s, 3H), 2.91-3.21(m, 2H), 4.00(s, 3H), 5.08(m, 1H), 5.39(d, 1H), 5.43(d, 1H), 6.02-6.25(m, 2H), 6.89(d, 1H), 7.01 (br. S, 1H), 7.59(d, 1H) ppm. MS(ESIpos): m/z = 549(M + Na)⁺HPLC(Method 1): Rₜ = 5.4 min. |
| 36 | 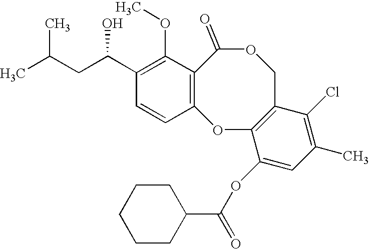 | ¹H-NMR(200 MHz, CDCl₃): δ = 0.98(m, 6H), 1.21-2.28(m, 14H), 2.31(s, 3H), 2.64(m, 1H), 3.99(s, 3H), 5.08(m, 1H), 5.39(d, 1H), 5.43(d, 1H), 6.89(d, 1H), 7.01(br. s, 1H), 7.59(m, 1H) ppm. MS(DCI): m/z = 534(M + NH₄)⁺HPLC(Method 1): Rₜ = 5.6 min. |
| 37 | 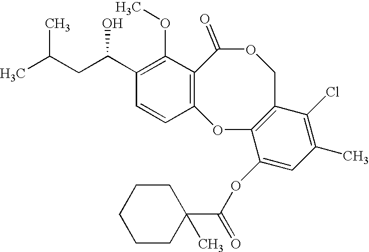 | ¹H-NMR(200 MHz, CDCl₃): δ = 0.98(m, 6H), 1.21-2.28(m, 17H), 2.31(s, 3H), 3.99(s, 3H), 5.08(m, 1H), 5.39(d, 1H), 5.43 (d, 1H), 6.92(d, 1H), 6.99(br. s, 1H), 7.59(m, 1H)ppm. MS(DCI): m/z = 548(M + NH₄)⁺HPLC(Method 1): Rₜ = 5.7 min. |
| 38 | 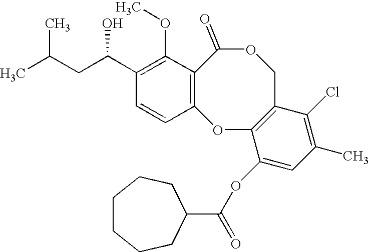 | ¹H-NMR(200 MHz, CDCl₃): δ = 0.98(m, 6H), 1.21-2.28(m, 16H), 2.31(s, 3H), 2.80(m, 1H), 3.99(s, 3H), 5.08(m, 1H), 5.39(d, 1H), 5.41(d, 1H), 6.89(d, 1H), 7.02 (br. s, 1H), 7.59(m, 1H) ppm. MS(DCI): m/z = 548(M + NH₄)⁺HPLC(Method 1): Rₜ = 5.7 min. |
| 39 | 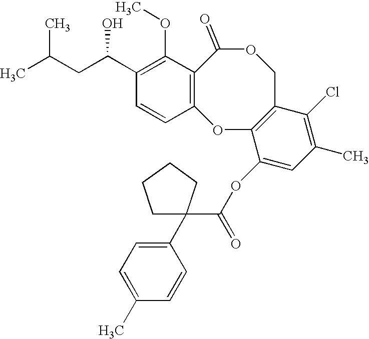 | ¹H-NMR(200 MHz, CDCl₃): δ = 0.98(m, 6H), 1.41-2.10(m, 10H), 2.28(s, 3H), 2.31(s, 3H), 2.82(m, 2H), 3.99(s, 3H), 5.08 (m, 1H), 5.39(d, 1H), 5.43(d, 1H), 6.50(d, 1H), 6.80(br. s, 1H), 7.10(d, 2H), 7.38(d, 2H), 7.48 (m, 1H)ppm. MS(ESIpos): m/z = 615(M + Na)⁺HPLC(Method 1): Rₜ = 6.0 min. |
| 40 | 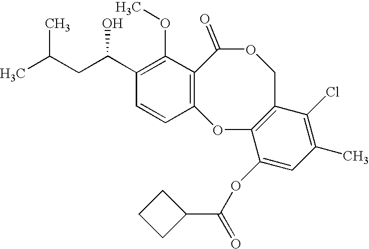 | ¹H-NMR(200 MHz, CDCl₃): δ = 0.98(m, 6H), 1.21-2.28(m, 10H), 2.31(s, 3H), 3.49(m, 1H), 3.99(s, 3H), 5.08(m, 1H), 5.39(d, 1H), 5.43(d, 1H), 6.92(d, 1H), 7.01 (br. s, 1H), 7.59(m, 1H) ppm. MS(DCI: m/z = 506(M + NH₄)⁺HPLC(Method 1): Rₜ = 5.3 min |
| 41 | 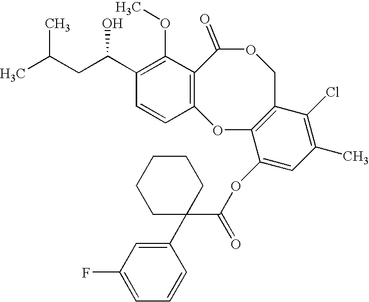 | ¹H-NMR(300 MHz, CDCl₃): δ = 0.98(m, 6H), 1.20-1.89(m, 12H), 2.29(s, 3H), 2.62(m, 2H), 3.97(s, 3H), 5.08(m, 1H), 5.39(d, 1H), 5.43(d, 1H), 6.58(d, 1H), 6.79.(s, 1H), 6.90(m, 1H), 7.18-7.31(m, 3H), 7.59(m, 1H) ppm. MS(ESIpos): m/z = 633(M + Na)⁺HPLC(Method 1): Rₜ = 6.2 min |
| 42 | 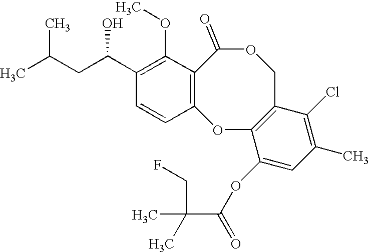 | ¹H-NMR(300 MHz, CDCl₃): δ = 0.98(m, 6H), 1.29-1.90(m, 10H), 2.31(s, 3H), 3.99(s, 3H), 4.53(d, 2H), 5.08(m, 1H), 5.39 (d, 1H), 5.43(d, 1H), 6.89(d, 1H), 7.02(br. s, 1H), 7.59(d, 1H) ppm. MS(ESIpos): m/z = 531(M + Na)⁺ |
| 43 | 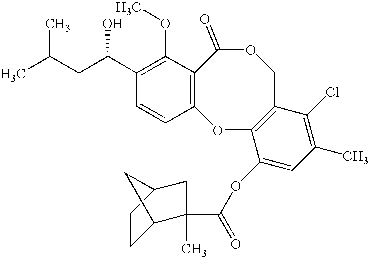 | ¹H-NMR(200 MHz, CDCl₃): δ = 0.98(m, 6H), 1.19-1.89(m, 17H), 1.95(d, 1H), 2.31(s, 3H), 2.30-2.75(m, 2H), 3.99(s, 3H), 5.08(m, 1H), 5.39(d, 1H), 5.43 (d, 1H), 6.89(m, 1H), 6.99(br. s, 1H), 7.59(d, 1H) ppm. MS(ESIpos): m/z = 565(M + Na)⁺HPLC(Method 1): Rₜ = 5.9 min |
| 44 | 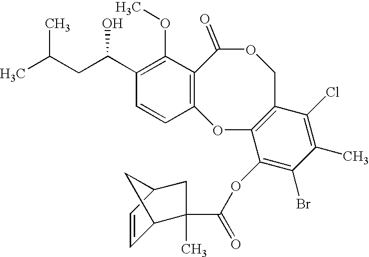 | ¹H-NMR(200 MHz, CDCl₃): δ = 0.98(m, 6H), 1.39-2.66(m, 11H), 3.00(m, 1H), 3.32-3.52(m, 1H), 3.99(s, 3H), 5.08(m, 1H), 5.39(m, 1H), 6.08-6.21(m, 1H), 6.87(m, lH), 7.59(m, 1H) ppm. MS(DCI): m/z = 622(M + NH₄)⁺HPLC(Method 1): Rₜ = 6.0, 6.1 min. |
| 45 | 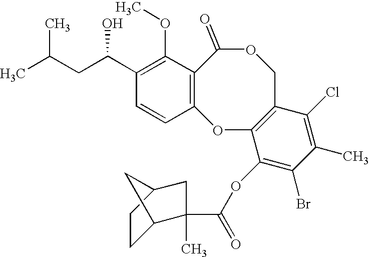 | ¹H-NMR(200 MHz, CDCl₃): δ = 0.98(m, 6H), 1.20-1.90(m, 12H), 2.30-2.83(m, 2H), 2.52(s, 3H), 3.99(s, 3H), 5.08(m, 1H), 5.40(m, 2H), 6.89(d, 1H), 7.59 (d, 1H) ppm. MS(DCI): m/z = 638(M + NH₄)⁺HPLC(Method 1): Rₜ = 6.52, 6.60 min. |
| 46 | 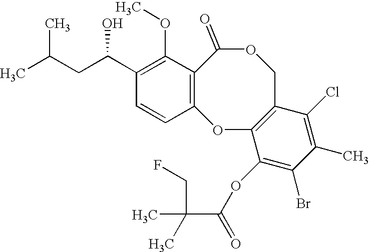 | ¹H-NMR(300 MHz, CDCl₃): δ = 0.98(m, 6H), 1.42(s, 3H), 1.49(s, 3H), 1.63-1.88(m, 3H), 1.90(d, 1H), 2.53(s, 3H), 3.99(s, 3H), 4.60(d, 2H), 5.08(m, 1H), 5.39(m, 2H), 6.88(d, 1H), 7.59 (d, 1H) ppm. MS(DCI): m/z = 604(M + NH₄)⁺HPLC(Method 1): Rₜ = 5.3 min. |
| 47 | 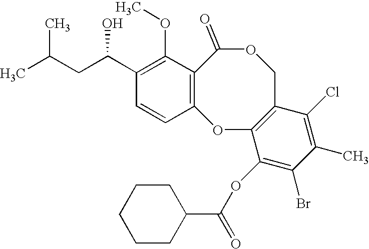 | ¹H-NMR(300 MHz, CDCl₃): δ = 0.98(m, 6H), 1.40-1.88(m, 13H), 1.90(d, 1H), 2.15(m, 1H), 2.52(s, 3H), 3.98(s, 3H), 5.08(m, 1H), 5.40(m, 2H), 6.90(d, 1H), 7.59(d, 1H) ppm. MS(DCI): m/z =612(M + NH₄)⁺HPLC(Method 1): Rₜ = 6.3 min. |
| 48 | 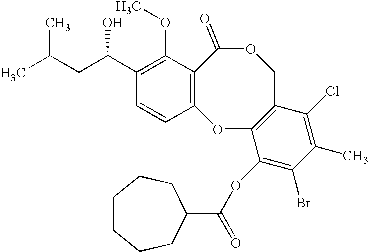 | ¹H-NMR(300 MHz, CDCl₃): δ = 0.98(m, 6H), 1.47-2.00(m, 16H), 2.52(s, 3H), 2.89(m, 1H), 3.99(s, 3H), 5.08(m, 1H), 5.40 (m, 2H), 6.88(d, 1H), 7.59(d, 1H) MS(ESIpos): m/z =631(M + Na)⁺HPLC(Method 1): Rₜ = 6.4 min. |
| 49 | 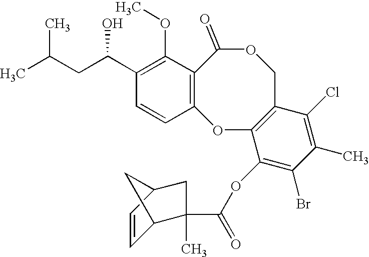 | ¹H-NMR(300 MHz, CDCl₃): δ = 0.98(m, 6H), 1.18-1.79(m, 11H), 2.52(s, 3H), 2.62-2.80(m, 2H), 3.99(s, 3H), 5.08(m, 1H), 5.40(m, 2H), 6.16(m, 1H), 6.28 (m, 1H), 6.90(d, 1H), 7.59(d, 1H) ppm. MS(DCI): m/z = 637(M + NH₄)⁺HPLC(Method 1): Rₜ = 5.8 min. |
| 50 | 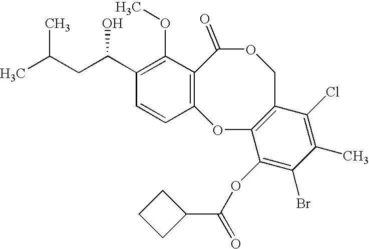 | ¹H-NMR(300 MHz, CDCl₃): δ = 0.98(m, 6H), 1.42(m, 1H), 1.61-2.49(m, 9H), 2.52(s, 3H), 3.99(s, 3H), 5.08(m, 1H), 5.39 (m, 2H), 6.90(d, 1H), 7.59(d, 1H) ppm. MS(DCI): m/z = 584(M + NH₄)⁺HPLC(Method 1): Rₜ = 5.4 min. |

### EXAMPLE B-51

### 9-[(1S)-1-Hydroxy-3-methylbutyl]-8-methoxy-3-methyl-7-oxo-5H,7H-dibenzo[b,g]-[1,5]-dioxocin-1-yl 1-methylcyclohexanecarboxylate

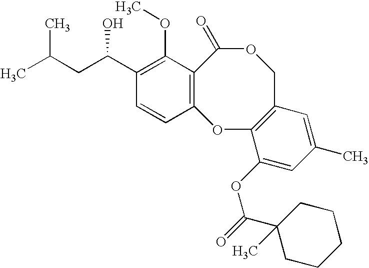

Under argon, 100 mg (0.27 mmol) of penicillide are dissolved in 1 ml of tetrahydrofuran, 60 μl (0.40 mmol) of 1,8-diazabicyclo[5.4.0]undec-7-ene and 65 mg (0.40 mmol) of 1-methylcyclohexanecarbonyl chloride are added and the mixture is stirred at room temperaure. After 2 hours, 1 ml of water and 5 drops of 1 N hydrochloric acid are added and the reaction mixture is diluted with 5 ml of ethyl acetate and filtered through an Extrelut cartridge. The cartridge is eluted with 40 ml of ethyl acetate and the filtrate is concentrated under reduced pressure. The residue is purified by preparative HPLC. This gives 97 mg (73% of theory) of product.

R_{f}=0.51 (cyclohexane/ethyl acetate 2:1)

¹H-NMR (300 MHz, CDCl₃): δ=0.97 (t, 6H), 1.25-1.84 (m, 14H), 1.90-1.96 (m, 1H), 2.20-2.28 (m, 2H), 2.28 (s, 3H), 3.97 (s, 3H), 5.04-5.10 (m, 3H), 6.72 (s, 1H), 6.89 (s, 1H), 6.98 (d, 1H), 7.57 (d, 1H) ppm.

MS (ESIpos): m/z=519 (M+H)⁺.

The examples listed in the table below are prepared analogously to the procedures described above, from the corresponding staring materials:

| Example | | |
|---|---|---|
| B- | Structure | Analytical data |
| 52 | 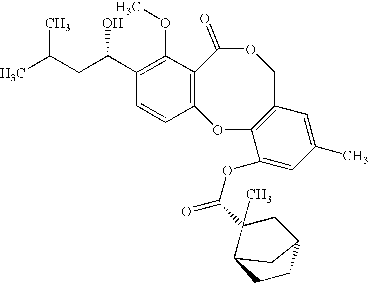 | ¹H-NMR(300 MHz, CDCl₃): δ = 0.97(t, 6H), 1.14-1.60(m, 12H), 1.64-1.96(m, 4H), 2.27(s, 4H),(s, 3H), 5.04-5.10(m, 3H), 6.72(s, 1H), 6.89(s, 1H), 6.94-6.99(m, 1H), 7.57(d, 1H) ppm. MS(ESIpos): m/z = 530(M + H)⁺ |
| 53 | 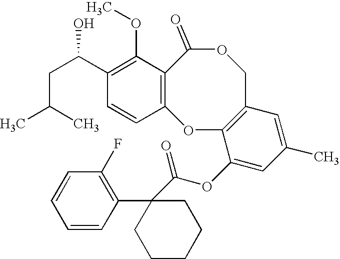 | ¹H-NMR(300 MHz, CDCl₃): δ = 0.97(t, 6H), 1.38-2.07(m, 12H), 2.25(s, 3H), 2.54(d, 2H), 3.97(s, 3H), 5.04-5.10(m, 3H), 6.68(s, 1H), 6.82-6.85(m, 2H), 7.00-7.07(m, 1H), 7.14-7.25(m, 2H), 7.50-7.54(m, 2H) ppm. HPLC(Method 2): Rₜ = 5.64 min. |
| 54 | 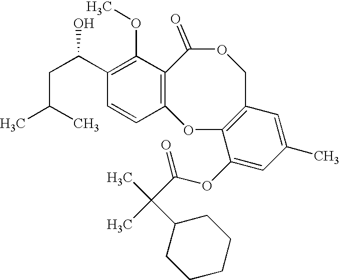 | ¹H-NMR(300 MHz, CDCl₃): δ = 0.97(t, 6H), 1.07-1.25(m, 5H) 1.28(s, 6H), 1.44-1.93(m, 10H), 2.28(s, 3H), 3.97(s, 3H), 5.04-5.12(m, 3H), 6.72(s; 1H), 6.86(s, 1H), 6.96(d, 1H), 7.57(d, 1H) ppm. MS(ESIpos): m/z = 546(M + Na)⁺HPLC(Method 2): Rₜ = 6.00 min. |
| 55 | 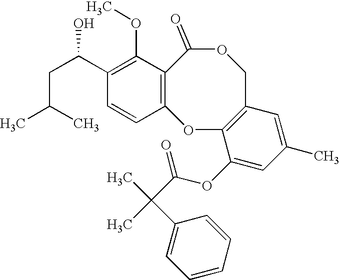 | ¹H-NMR(300 MHz, CDCl₃): δ = 0.97(t, 6H), 1.44-1.95(m, 4H), 1.76(s, 6H), 2.24(s, 3H), 3.97(s, 3H), 5.04-5.12(m, 3H), 6.66-6.69(m, 2H), 6.80(s, 1H), 7.21-7.35(m, 3H), 7.47-7.50(m, 3H) ppm. MS(DCI): m/z = 536(M + NH₄)⁺HPLC(Method 2): Rₜ = 5.45 min. |
| 56 | 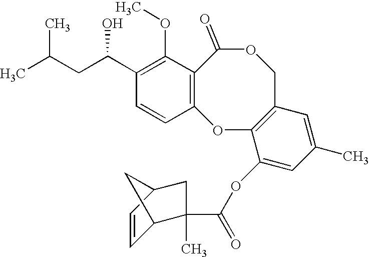 | ¹H-NMR(300 MHz, CDCl₃): δ = 0.97(t, 6H), 1.33 + 1.67(2s, 3H), 1.45-1.88(m, 6H), 1.92(br. s, 1H), 2.26 + 2.28(2s, 3H), 2.62 + 2.10(2dd, 1H), 2.90 + 3.0(2s, 1H), 3.28 + 3.49(2s, 1H), 3.98(s, 3H), 5.03-5.13(m, 3H), 6.11-6.22 (m, 1H), 6.24-6.33(m, 1H), 6.70-6.75(m, 1H), 6.82-6.97(m, 2H), 7.54-7.60(m, 1H) ppm. MS(ESIpos): m/z = 529(M + Na)⁺HPLC(Method 2): |
| # Rₜ = 5.54, 5.61 min. | | |
| 57 | 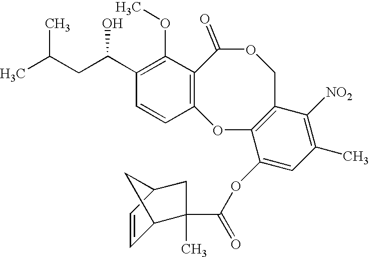 | ¹H-NMR(300 MHz, CDCl₃): δ = 0.92(t, 6H), 1.20-1.85(m, 9H), 1.90(br. s, 1H), 2.25(dd, 3H), 2.52 + 2.01(2dd, 1H),2.72-2.87(m, 1H), 2.85-3.19(m, 1H), 3.89 + 3.91(2s, 3H), 4.93-5.08(m, 3H), 6.02-6.26(m, 2H), 6.80-7.02 (m, 2H), 7.52-7.60(m, 1H) ppm. MS(DCI): m/z = 574(M + Na)⁺ |
| 58 | 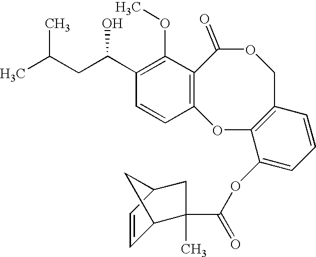 | Rf(cyclohexane/ethyl acetate 2:1) =0.25 HPLC(Method 1): Rₜ = 5.41, 5.47 min. |
| 59 | 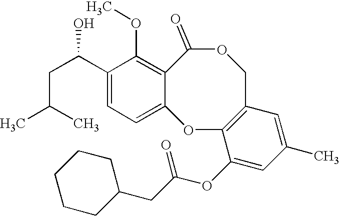 | LC-MS(Method 3): Rₜ = 5.36 min. MS(ESIpos): m/z = 497(M + H)⁺ |
| 60 | 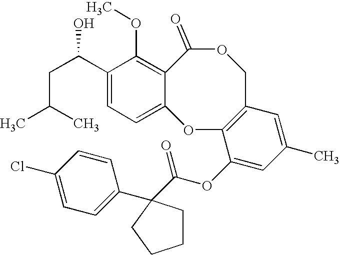 | LC-MS(Method 3): Rₜ = 5.60 min. MS(ESIpos): m/z = 579(M + H)⁺ |
| 61 | 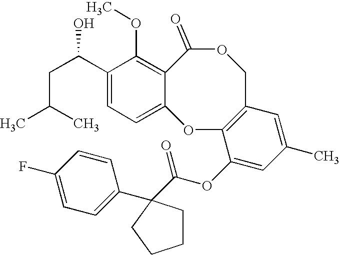 | ¹H-NMR(300 MHz, CDCl₃): δ = 0.98(m, 6H), 1.44-2.06(m, 10H), 2.22(s, 3H), 2.80-2.92(m, 2H), 3.96(s, 3H), 5.00-5.12(m, 3H), 6.55(d, 1H), 6.69(s, 2H), 6.99(t, 2H), 7.45-7.49(m, 3H) ppm. MS(ESIpos): m/z = 585(M + Na)⁺HPLC(Method 1): Rₜ = 5.44 min. |
| 62 | 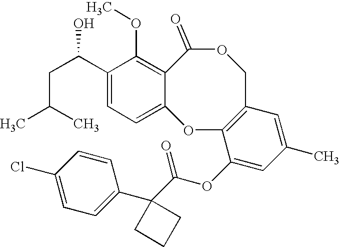 | LC-MS(Method 3): Rₜ = 5.45 min. MS(ESIpos): m/z = 565(M + H)⁺ |
| 63 | 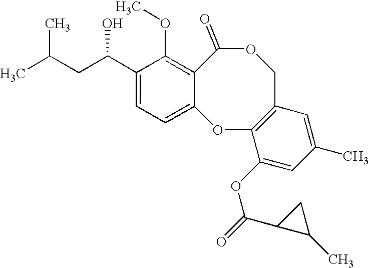 | LC-MS(Method 3): Rₜ = 4.83 min. MS(ESIpos): m/z = 455(M + H)⁺ |
| 64 | 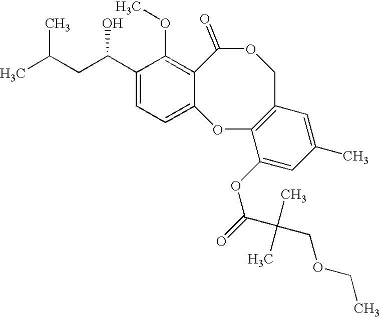 | LC-MS(Method 3): Rₜ = 5.04 min. MS(ESIpos): m/z = 501(M + H)⁺ |
| 65 | 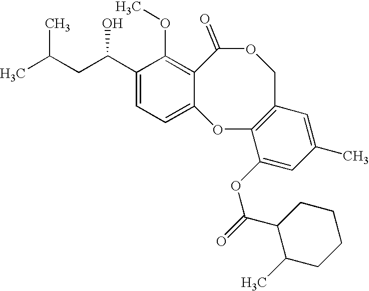 | LC-MS(Method 3): Rₜ = 5.40 min. MS(ESIpos): m/z = 497(M + H)⁺ |
| 66 | 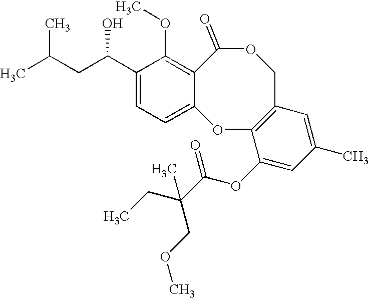 | LC-MS(Method 3): Rₜ = 5.00 min. MS(ESIpos): m/z = 501(M + H)⁺ |
| 67 | 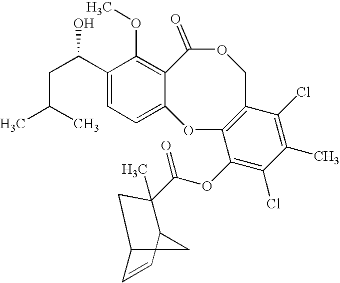 | MS(DCI): m/z = 593(M + NH₄)⁺HPLC(Method 1): Rₜ = 6.16, 6.31 min. |
| 68 | 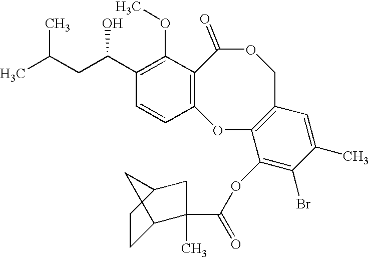 | ¹H-NMR(300 MHz, CDCl₃): δ = 0.98(dd, 6H), 1.10-1.89(m, 13H), 2.02(br. s, 1H), 2.20-2.85 (m, 3H), 2.37(s, 3H), 3.98(s, 3H), 5.00-5.11(m, 3H), 6.84(br. s, 1H), 6.95(d, 1H), 7.59(d, 1H) ppm. MS(DCI): m/z = 606(M + NH₄)⁺ |
| 69 | 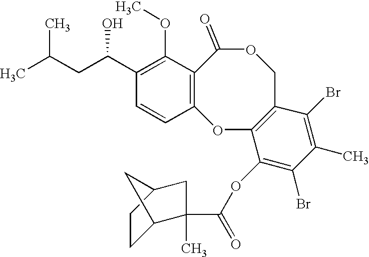 | ¹H-NMR(200 MHz, CDCl₃): δ = 0.98(dd, 6H), 1.10-1.98(m, 14H), 2.20-2.85(m, 3H), 2.41(s, 3H), 3.99(s, 3H), 5.03-5.15(m, 1H), 5.33-5.48(m, 2H), 6.91(d, 1H), 7.61(d, 1H) ppm. HPLC(Method 2): Rₜ = 6.48, 6.57 min. |
| 70 | 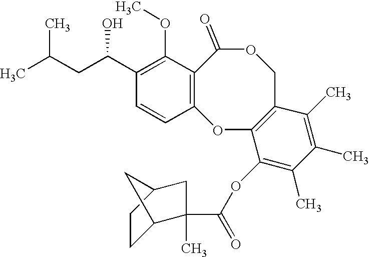 | ¹H-NMR(300 MHz, CDCl₃): δ = 0.98(dd, 6H), 1.05-1.88(m, 14H), 2.01-2.80(m, 12H), 3.99(s, 3H),5.07(m, 1H),5.15-5.31(m, 2H), 6.95 + 6.92(d, 1H), 754(d, 1H) ppm. MS(DCI): m/z =554(M + NH₄)⁺HPLC(Method 1): Rₜ = 5.95 min. |
| 71 | 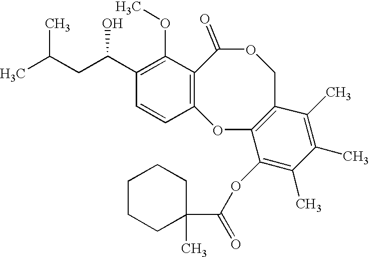 | ¹H-NMR(300 MHz, CDCl₃): δ = 0.98(dd, 6H), 1.20-1.88(m, 12H), 2.01-2. 80(m, 14H), 3.99(s, 3H), 5.04(m, 1H), 5.30(m, 2H), 6.94(d, 1H), 7.55(d, 1H) ppm. MS(DCI): m/z = 542(M + NH₄)⁺HPLC(Method 1): Rₜ = 5.88 min. |
| 72 | 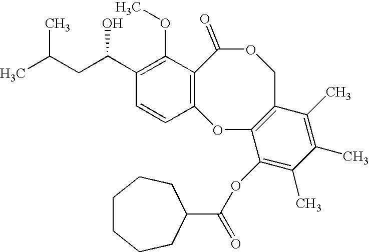 | ¹H-NMR(300 MHz, CDCl₃): δ = 0.98(dd, 6H), 1.21-2.28(m, 25H), 2.84(m, 1H), 3.99(s, 3H), 5.07(m, 1H), 5.22(m, 2H), 6.92 (d, 1H), 7.54(d, 1H) ppm. MS(DCI): m/z = 542(M + NH₄)⁺HPLC(Method 1): Rₜ = 5.90 min. |
| 73 | 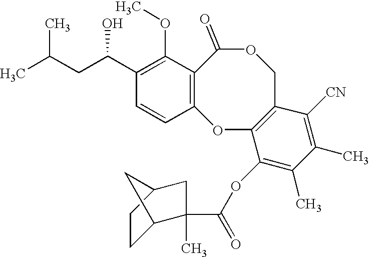 | ¹H-NMR(300 MHz, CDCl₃): δ = 0.98(dd, 6H), 1.10-1.97(m, 14H), 2.26-2.82(m, 3H), 2.65(s, 3H), 3.98(s, 3H), 5.10(m, 1H), 5.37(m, 2H), 6.90 + 6.95(d, 1H), 7.63(d, 1H)ppm. MS(DCI): m/z = 629/631 (M + NH₄)⁺HPLC(Method 1): Rₜ = 5.98, 6.05 min. |
| 74 | 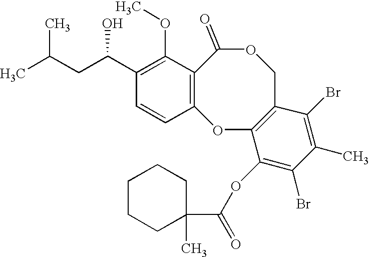 | ¹H-NMR(300 MHz, CDCl₃): δ = 0.98(dd, 6H), 1.10-1.97(m, 14H), 2.26-2.82(m, 3H), 2.65(s, 3H), 3.98(s, 3H), 5.10(m, 1H), 5.37(m, 2H), 6.90 + 6.95(d, 1H), 7.63(d, 1H) ppm. MS(DCI): m/z = 629/631 (M + NH₄)⁺HPLC(Method 1): Rₜ = 5.98, 6.05 min. |
| 75 | 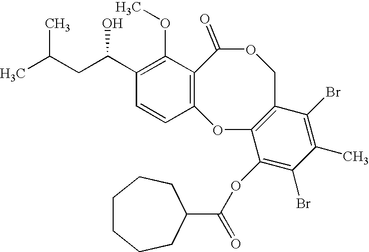 | ¹H-NMR(300 MHz, CDCl₃): δ = 0.99(dd, 6H), 1.20-2.04(m, 14H), 2.13-2.27(m, 2H), 2.62(s, 3H), 2.88(m, 1H), 3.99(s, 3H), 5.08(m, 1H), 5.43(m, 2H), 6.90 (d, 1H), 7.60(d, 1H) ppm. MS(DCI): m/z = 672(M + NH₄)⁺HPLC(Method 1): Rₜ = 6.57 min. |
| 76 | 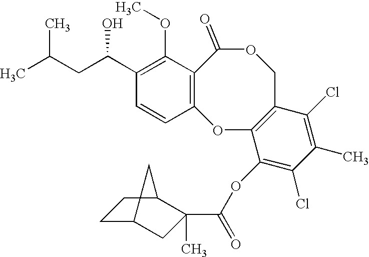 | ¹H-NMR(300 MHz, CDCl₃): δ = 0.99(dd, 6H), 1.12-1.88(m, 13H), 1.92(br. s, 1H), 2.15-2.34 (m, 2H), 2.48(s, 3H), 2.50-2.61/2.76-2.81(m, 1H), 3.98(s, 3H), 5.05-5.13(m, 1H), 5.30-5.51 (m, 2H), 6.91(dd, 1H), 7.61(d, 1H) ppm. MS(ESIpos): m/z = 577(M)⁺HPLC(Method 2): Rₜ = 6.52, 6.64 min. |
| 77 | 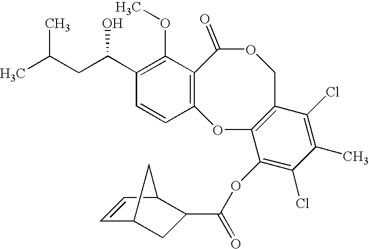 | ¹H-NMR(200 MHz, CDCl₃): δ = 0.99(dd, 6H), 1.35-1.90(m, 6H), 1.95(d, 1H), 2.01-2. 16(m, 1H), 2.46(s, 3H), 2.98(br. s, 1H), 3.31-3.42(m, 1H), 3.48(br. s, 1H), 3.98(s, 3H), 5.05-5.16(m, 1H), 5.33-5.50(m, 2H), 6.03-6.12 (m, 1H), 6.18-6.26(m, 1H), 6.85 (d, 1H), 7.59(d, 1H) ppm. MS(ESIpos): m/z = 583(M + Na)⁺HPLC(Method 2): Rₜ = 5.96 min. |
| 78 | 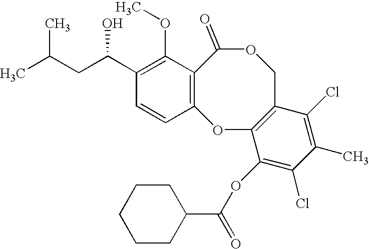 | ¹H-NMR(200 MHz, CDCl₃): δ = 0.99(dd, 6H), 1.25-1.90(m, 11H), 1.93(d, 1H), 2.08-2.22(m, 2H), 2.47(s, 3H), 2.53-2.82(m, 1H), 3.98(s, 3H), 5.03-5.15(m, 1H), 5.30-5.51(m, 2H), 6.89(d, 1H), 7.60(d, 1H) ppm. MS(ESIpos): m/z = 573(M + Na)⁺HPLC(Method 2): Rₜ = 6.20 min. |
| 79 | 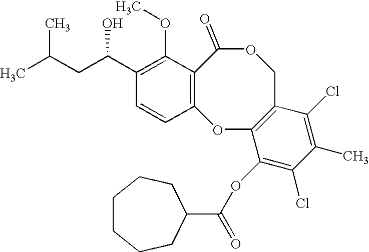 | ¹H-NMR(200 MHz, CDCl₃): δ = 0.99(dd, 6H), 1.36-2.07(m, 12H), 2.10-2.28(m, 2H), 2.47(s, 3H), 2.43-2.61(m, 2H), 2.80-2.96 (m, 1H), 3.98(s, 3H), 5.10(dd, 1H), 5.29-5.5 1(m, 2H), 6.89(d, 1H), 7.61(d, 1H) ppm. MS(ESIpos): m/z = 587(M + Na)⁺HPLC(Method 2): Rₜ = 6.68 min. |
| 80 | 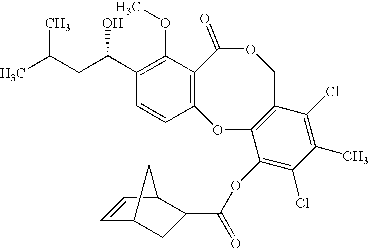 | ¹H-NMR(200 MHz, CDCl₃): δ = 0.99(dd, 6H), 1.39-1.88(m, 5H), 1.93(d, 1H), 2. 10-2.25(m, 1H), 2.48(s, 3H), 2.59-2.70(m, 1H), 3.01(br. s, 3.37(br. s, 1H), 3.73(s, 1H), 3.98(s, 3H), 5.03-5.18(m, 1H), 5.35-5.48(br. s, 2H), 6.13-6.25(m, 2H), 6.89(d, 1H), 7.60(d, 1H) ppm. MS(ESIpos): m/z = 583(M + Na)⁺HPLC(Method 2): Rₜ = 6.08 min. |
| 81 | 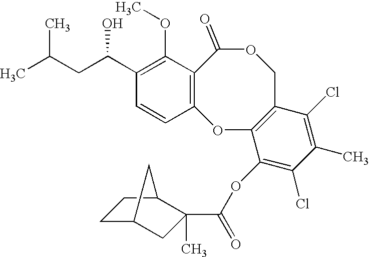 | ¹H-NMR(400 MHz, CDCl₃): δ = 0.99(dd, 6H), 1.17-1.88(m, 13H), 1.92(br. s, 1H), 2.21(d, 1H), 2.41(s, 1H), 2.44(s, 1H), 2.48 (s, 3H), 3.98(s, 3H), 5.09 (dd, 1H), 5.30-5.51(m, 2H), 6.92 (d, 1H), 7.61(d, 1H) ppm. |
| 82 | 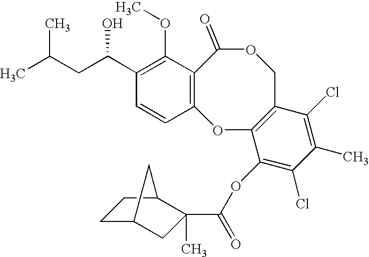 | ¹H-NMR(400 MHz, CDCl₃): δ = 0.99(dd, 6H), 1.08(dd, 1H), 1.16-1.35(m, 3H), 1.42-1.88(m, 10H), 2.30(s, 1H), 2.48(s, 3H), 2.50-2.60(m, 1H), 2.80(s, 1H), 3.98(s, 3H), 5.10(dd, 1H), 5.30-5.51(m, 2H), 6.90(d, 1H), 7.61 (d, 1H) ppm. |
| 83 | 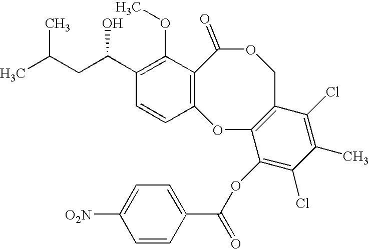 | ¹H-NMR(300 MHz, CDCl₃): δ = 0.98(dd, 6H), 1.40-1.86(m, 3H), 1.91(br. s, 1H), 2.53(s, 3H), 3.96(s, 3H), 5.04-5.13(m, 1H), 5.38-5.53(m, 2H), 6.90(d, 1H), 7.59(d, 1H), 8.35-8.49(m, 4H) ppm. MS(ESIpos): m/z = 607 (M + NH₄)⁺HPLC(Method 2): Rₜ = 5.73 min. |
| 84 | 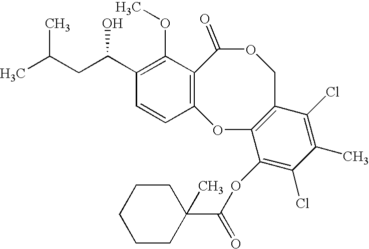 | ¹H-NMR(300 MHz, CDCl₃): δ = 0.99(dd, 6H), 1.25-1.89(m, 14H), 1.92(d, 1H), 2.21-2.32(m, 2H), 2.48(s, 3H), 3.98(s, 3H), 5.04-5.13(m, 1H), 5.29-5.51(br. s, 2H), 6.92(d, 1H), 7.60(d, 1H) ppm. MS(ESIpos): m/z = 582 (M + NH₄)⁺HPLC(Method 2): Rₜ = 6.48 min. |
| 85 | 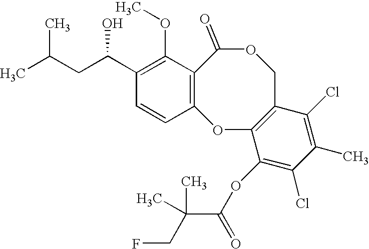 | ¹H-NMR(300 MHz, CDCl₃): δ = 0.99(dd, 6H), 1.45-1.88(m, 3H), 1.47(s, 6H), 1.90(d, 1H), 2.48(s, 3H), 3.98(s, 3H), 4.51(s, 1H), 4.67(s, 1H), 5.04-5.13(m, 1H), 5.30-5.49(m, 2H), 6.86(d, 1H), 7.60(d, 1H) ppm. MS(ESIpos): m/z = 560 (M + NH₄)⁺HPLC(Method 2): Rₜ = 5.63 min. |
| 86 | 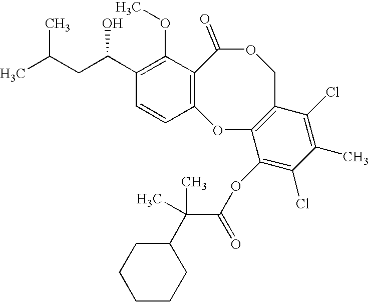 | ¹H-NMR(300 MHz, CDCl₃): δ = 0.99(dd, 6H), 1.05-1.36(m, 11H), 1.43-1.92(m, 10H), 2.48(s, 3H), 3.98(s, 3H), 5.05-5.12(m, 1H), 5.30-5.48(m, 2H), 6.91(d, 1H), 7.59(d, 1H)ppm. MS(ESIpos): m/z = 610 (M + NH₄)⁺HPLC(Method 2): Rₜ = 7.18 min. |
| 87 | 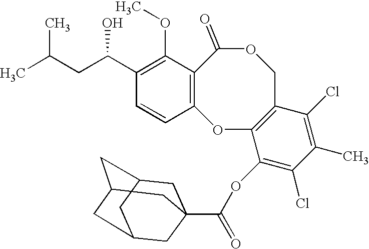 | ¹H-NMR(300 MHz, CDCl₃): δ = 0.99(dd,6H), 1.43-1.88(m, 10H), 1.91(br. s, 1H), 2.08-2.18 (m, 9H), 2.47(s, 3H), 3.98(s, 3H), 5.10(dd, 1H), 5.29-5.49(m, 2H), 6.88(d, 1H), 7.60(d, 1H) ppm. MS(ESIpos): m/z = 620 (M + NH₄)⁺HPLC(Method 2): Rₜ = 7.23 min. |
| 88 | 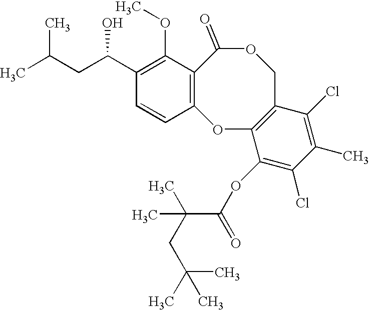 | ¹H-NMR(300 MHz, CDCl₃): δ = 0.99(dd, 6H), 1.02(s, 9H), 1.46-1.88(m, 5H), 1.48(s, 6H), 1.92(br. s, 1H), 2.48(s, 3H), 3.98 (s, 3H), 5.06-5.12(m, 1H), 5.30-5.47(m, 2H), 6.93(d, 1H), 7.60 (d, 1H) ppm. MS(ESIpos): m/z = 598 (M + NH₄)⁺HPLC(Method 2): Rₜ = 6.82 min. |
| 89 | 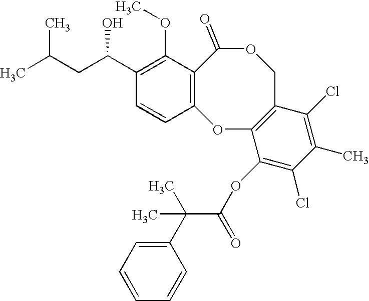 | ¹H-NMR(300 MHz, CDCl₃): δ = 0.99(dd, 6H), 1.42-1.88(m, 9H), 1.92(br. s, 1H), 2.43(s, 3H), 3.98(s, 3H), 5.04-5.12(m, 1H), 5.37(dd, 2H), 6.55(d, 1H), 7.18-7.32(m, 3H), 7.46-7.53(m, 3H) ppm. MS(ESIpos): m/z = 604 (M + NH₄)⁺HPLC(Method 2): Rₜ = 6.02 min. |
| 90 | 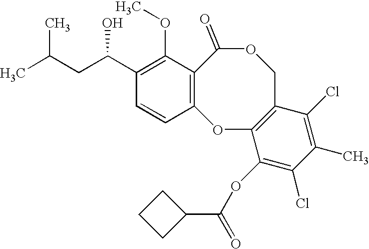 | ¹H-NMR(300 MHz, CDCl₃): δ = 0.99(dd, 6H), 1.42-1.87(m, 3H), 1.91(br. s, 1H), 2.01-2.16 (m, 2H), 2.32-2.46(m, 2H), 2.48 (s, 3H), 2.49-2.62(m, 2H), 3.54 (quintet, 1H), 3.98(s, 3H), 5.05-5.12(m, 1H), 5.39(dd, 2H), 6.90 (d, 1H), 7.60(d, 1H) ppm. MS(ESIpos): m/z = 540 (M + NH₄)⁺HPLC(Method 2): Rₜ = 5.79 min. |
| 91 | 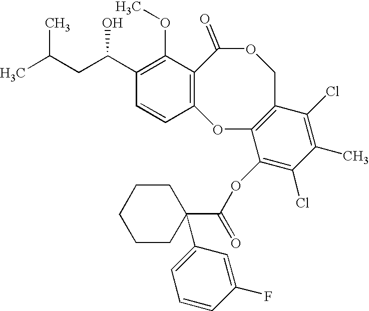 | ¹H-NMR(300 MHz, CDCl₃): δ = 0.99(dd, 6H), 1.28-2.01(m, 12H), 2.40(s, 3H), 2.66-2.75(m, 2H), 3.98(s, 3H), 5.04-5.11(m, 1H), 5.33(dd, 2H), 6.50(d, 1H), 6.79-6.89(m, 1H), 7.18-7.37(m, 3H), 7.49(d, 1H) ppm. MS(ESIpos): m/z = 662 (M + NH₄)⁺HPLC(Method 2): Rₜ = 6.63 min. |

### EXAMPLE B-92 AND EXAMPLE B-93

### 2,4-Dichloro-9-[(1S)-1-hydroxy-3-methylbutyl]-8-methoxy-3-methyl-7-oxo-5H,7H-dibenzo[b,g][1,5]dioxocin-1-yl 2-methylbicyclo[2.2.1]hept-5-ene-2-carboxylate (diastereomers I and II)

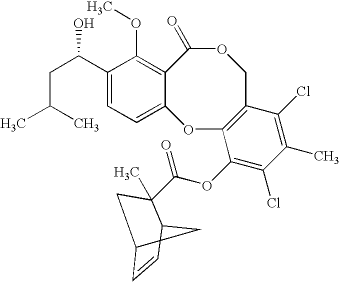

The preparation is carried out analogously to Example B-51 using 73 mg (165 μmol) of the compound from Example B-LI. The resulting mixture of diastereomers is, after pre-purification on an Extrelut/silica gel cartridge, separated chromatographically [column: Kromasil 100 C18, 5 μM, 20 mm×250 mm; mobile phase: water/acetonitrile 20:80; flow rate: 25 ml/min; temperature: 40° C.; detection: 210 nm]. This gives 32 mg (33% of theory) of the pure diastereomer I (Example B-92) and 33 mg (34% of theory) of the pure diastereomer II (Example B-93) whose configuration was not determined.

Diastereomer I:

¹H-NMR (400 MHz, CDCl₃): δ=0.92-1.03 (m, 6H), 1.45-1.90 (m, 10H), 2.07-2.32 (m, 1H), 2.47 (s, 3H), 2.55-3.04 (m, 2H), 3.99 (s, 3H), 5.06-5.58 (m, 3H), 6.10-6.39 (m, 2H), 6.83-6.91 (m, 1H), 7.57-7.67 (m, 1H) ppm.

Rₜ=8.63 min. [column: Kromasil 100 C18, 5 μM, 4 mm×250 mm; mobile phase: water/acetonitrile 20:80; flow rate: 1 ml/min; temperature: 40° C.; detection: 210 nm].

Diastereomer II:

¹H-NMR (400 MHz, CDCl₃): δ=0.96-1.00 (m, 6H), 1.37 (s, 3H), 1.47-1.88 (m, 7H), 2.47 (s, 3H), 2.62-2.70 (m, 1H), 2.90 (br. s, 1H), 3.33 (br. s, 1H), 3.98 (s, 3H), 5.08 (dd, 1H), 5.22-5.58 (m, 2H), 6.14-6.17 (m, 1H), 6.28-6.31 (m, 1H), 6.90 (d, 1H), 7.60 (d, 1H) ppm.

Rₜ=9.78 min. [column: Kromasil 100 C18, 5 μM, 4 mm×250 mm; mobile phase: water/acetonitrile 20:80; flow rate: 1 ml/min; temperature: 40° C.; detection: 210 nm].

### EXAMPLE B-94 AND EXAMPLE B-95

### 9-[(1S)-1-Hydroxy-3-methylbutyl]-8-methoxy-3-methyl-7-oxo-5H,7H-dibenzo[b,g]-[1,5]-dioxocin-1-yl 2-methylbicyclo[2.2.1]hept-5-ene-2-carboxylate (Diastereomers I and II)

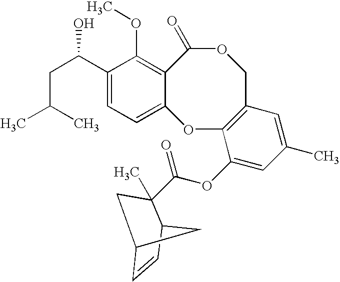

The preparation is carried out analogously to Example B-51 using 120 mg (320 mol) of penicillide. The residue obtained after work-up is pre-purified by preparative HPLC. The resulting mixture of diastereomers (=Example B-56) is separated chromatographically [column: Kromasil, 20 mm×250 mm; mobile phase: water/acetonitrile 30:70; flow rate: 25 ml/min; temperature: 40° C.; detection: 210 nm]. This gives 5 mg (3% of theory) of the pure diastereomer I (Example B-94) and 13 mg (8% of theory) of the pure diastereomer II (Example B-95) whose configuration was not determined.

Diastereomer I:

Rₜ=12.63 min. [column: Kromasil; mobile phase: water/acetonitrile 35:65; flow rate: 1 ml/min; temperature: 40° C.; detection: 210 nm].

Diastereomer II:

Rₜ=11.32 min. [column: Kromasil; mobile phase: water/acetonitrile 35:65; flow rate: 1 ml/min; temperature: 40° C.; detection: 210 nm].

### EXAMPLE B-96 AND EXAMPLE B-97

### 9-[(1S)-1-Hydroxy-3-methylbutyl]-8-methoxy-3-methyl-7-oxo-5H,7H-dibenzo[b,g]-[1,5]-dioxocin-1-yl tetrahydro-3-furancarboxylate (Diastereomers I and II)

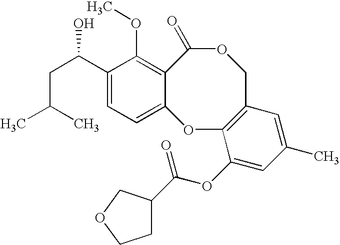

The preparation is carried out analogously to Example B-51 using 100 mg (270 μmol) of penicillide. The resulting mixture of diastereomers is, after pre-purification, separated chromatographically by preparative HPLC [column: Daicel Chiralpak AD, 20-mm×250 mm; mobile phase: isohexane/ethanol 85:15; flow rate: 15 ml/min; temperature: 30° C.; detection: 220 nm]. This gives 26 mg (20% of theory) of the pure diastereomer I (Example B-96) and 28 mg (22% of theory) of the pure diastereomer II (Example B-97) whose configuration was not determined.

Mixture of Diastereomers:

¹H-NMR (300 MHz, CDCl₃): δ=0.97 (t, 6H), 1.44-1.54 (m, 1H), 1.64-1.84 (m, 2H), 1.95 (d, 1H), 2.22-2.47 (m, 2H), 2.26 (s, 3H), 3.38-3.45 (m, 1H), 3.83-3.99 (m, 2H), 3.97 (s, 3H), 4.08-4.21 (m, 2H), 5.04-5.10 (m, 3H), 6.76 (s, 1H), 6.92 (d, 1H), 6.94 (s, 1H), 7.58 (d, 1H) ppm.

MS (DCI): m/z=488 (M+NH₄)⁺.

Diastereomer I:

Rₜ=14.41 min. [column: Daicel Chiralpak; mobile phase: isohexane/ethanol 85:15; flow rate: 1 ml/min; temperature: 40° C.; detection: 220 nm].

Diastereomer II:

Rₜ=16.79 min. [column: Daicel Chiralpak; mobile phase: isohexane/ethanol 85:15; flow rate: 1 ml/min; temperature: 40° C.; detection: 220 nm].

### EXAMPLE B-98

### 9-[(1S)-1-Hydroxy-3-methylbutyl]-8-methoxy-3-methyl-7-oxo-5H,7H-dibenzo[b,g]-[1,5]-dioxocin-1-yl 2,2,3,3-tetramethylcyclopropanecarboxylate

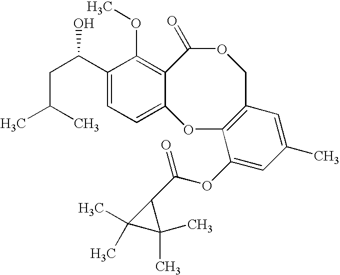

Under argon, 172 mg (1.21 mmol) of 2,2,3,3-tetramethylcyclopropanecarboxylic acid are dissolved in 2 ml of dichloromethane and a catalytic amount of dimethylformamide. 0.14 ml (1.61 mmol) of oxalyl chloride is then (if appropriate with cooling) added dropwise, and the mixture is stirred for one hour. The reaction mixture is concentrated under reduced pressure. In a second flask, 150 mg (0.40 mmol) of penicillide are dissolved under argon in 2 ml of tetrahydrofuran, and 19 mg (0.48 mmol) of sodium hydride are added a little at a time at 0° C. After 5 minutes, the reaction mixture is added to the acid chloride prepared, and the mixture is stirred at room temperature for 1.5 hours. The mixture is diluted with dichloromethane, 1 ml of water is added and the mixture is filtered through an Extrelut cartridge. The cartridge is eluted with dichloromethane and the filtrate is concentrated under reduced pressure. The residue is purified chromatographically on silica gel (mobile phase: cyclohexane/ethyl acetate 9:1→5:1). This gives 108 mg (54% of theory) of product.

¹H-NMR (200 MHz, CDCl₃): δ=0.96 (d, 3H), 0.98 (d, 3H), 1.20-2.00 (m, 5H), 1.27 (s, 61), 1.33 (s, 61), 2.27 (s, 3H), 3.97 (s, 3H), 5.02-5.15 (m, 3H), 6.71 (br. s, 1H), 6.93 (br. s, 1H), 7.00 (d, 1H), 7.55 (d, 1H) ppm.

MS (ESIpos): m/z=519 (M+Na)⁺

HPLC (Method 2): Rₜ=5.58 min.

The examples listed in the table below are prepared analogously to the procedures described above, from the corresponding starting materials:

| Example | | |
|---|---|---|
| B- | Structure | Analytical data |
| 99 | 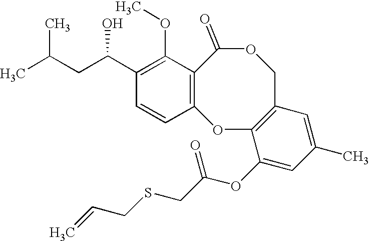 | ¹H-NMR(300 MHz, CDCl₃): δ = 0.97(t, 6H), 1.49-1.98(m, 4H), 2.29(s, 3H), 3.34(d, 2H), 3.46(s, 2H), 3.97(s, 3H), 5.03-5.12(m, 3H), 5.16(s, 1H), 5.21(d, 1H), 5.76(quintet, 1H), 6.71(br. s, 1H), 6.93(br. s, 1H), 7.00(d, 1H), 7.55 (d, 1H) ppm. MS(ESIpos): m/z =509(M + Na)⁺HPLC(Method 2): Rₜ = 5.02 min. |
| 100 | 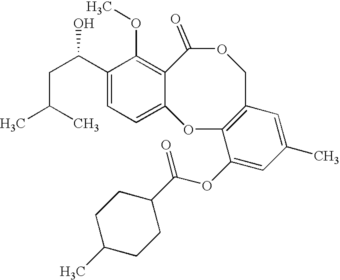 | ¹H-NMR(200 MHz, CDCl₃): δ = 0.98(m, 6H), 1.25-1.95(m, 11H), 1.42(s, 3H), 2.07-2.24(m, 2H), 2.28(s, 3H), 2.80-2.94(m, 1H), 3.97(s, 3H), 5.02-5.15(m, 3H), 6.71(br. s, 1H), 6.93(br. s, 1H), 7.00(d, 1H), 7.55(d, 1H) ppm. MS(ESIpos): m/z = 519(M + Na)⁺HPLC(Method 2): Rₜ = 5.64 min. |
| 101 | 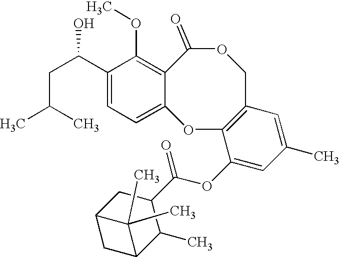 | ¹H-NMR(200 MHz, CDCl₃): δ = 0.98(m, 6H), 1.05(s, 3H), 1.21-1.24(m, 8H), 1.45-2.10(m, 5H), 2.29(s, 3H), 2.30-2.42(m, 3H), 2.50-2.65(m, 1H), 2.95-3.08 (m, 1H), 3.97(s, 3H), 5.00-5.12 (m,3H), 6.71(br. s, 1H), 6.93(br. s, 1H), 7.00(d, 1H), 7.55(d, 1H) ppm. MS(ESIpos): m/z = 559(M + Na)⁺HPLC(Method 1): Rₜ = 6.03 min. |
| 102 | 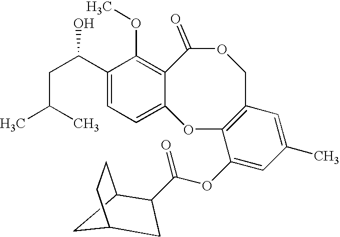 | ¹H-NMR(200 MHz, CDCl₃): δ = 0.98(m, 6H), 1.25-1.85(m, 9H), 2.28(s, 3H), 2.29-2.40(m, 2H), 2.52-2.62(m, 1H), 2.70-2.90 (m, 2H), 3.03-3.18(m, 1H), 3.97 (s, 3H), 5.02-5.14(m, 3H), 6.73(s, 1H), 6.89-7.03(m, 2H), 7.57(d, 1H) ppm. MS(ESIpos): m/z = 517(M + Na)⁺HPLC(Method 2): Rₜ = 5.52 min. |
| 103 | 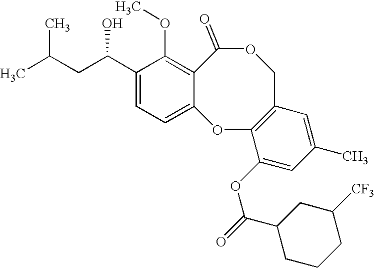 | ¹H-NMR(200 MHz, CDCl₃): δ = 0.98(q, 6H), 1.25-2.75(m, 14H), 2.29(s, 3H), 3.97(s, 3H), 5.02-5.14(m, 3H), 6.72-6.78(m, 1H), 6.87-6.97(m, 2H), 7.59(d, 1H) ppm. MS(ESIpos): m/z = 573(M + Na)⁺HPLC(Method 2): Rₜ = 5.46 min. |
| 104 | 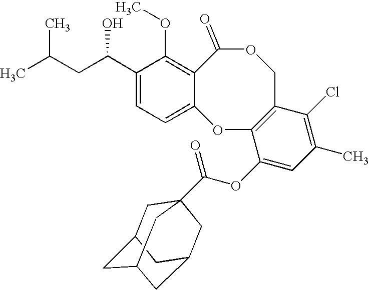 | ¹H-NMR(300 MHz, CDCl₃): δ = 0.98(m, 6H), 1.47-2.10(m, 17H), 2.33(s, 3H), 3.97(s, 3H), 4.53(d, 2H), 5.08(m, 1H), 5.39 (d, 1H), 5.42(d, 1H), 6.89(d, 1H), 7.00(br. s, 1H), 7.59(d, 1H) ppm. MS(ESIpos): m/z = 591(M + Na)⁺ |
| 105 | 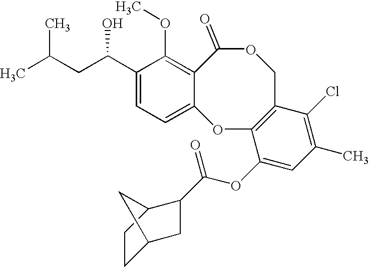 | ¹H-NMR(300 MHz, CDCl₃): δ = 0.98(m, 6H), 1.21-1.90(m, 12H), 2.32(s, 3H), 2.61-2.73(m, 2H), 3.10(m, 1H), 3.99(s, 3H), (d, 1H), 6.92(m, 1H), 7.01(br. s, 5.08(m, 1H), 5.39(d, 1H), 5.43 1H), 7.59(d, 1H) ppm. MS(DCI): m/z = 546(M + NH₄)⁺HPLC(Method 1): Rₜ = 5.8 min. |

### EXAMPLE B-106

### 9-[(1S)-1-Hydroxy-3-methylbutyl]-8-methoxy-3-methyl-7-oxo-5H,7H-dibenzo[b,g]-[1,5]-dioxocin-1-yl 4,4,5,5,5-pentafluoro-1-pentanesulphonate

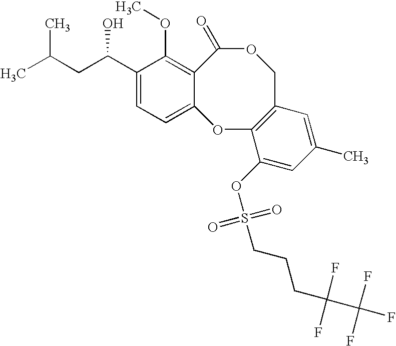

Under argon, 100 mg (0.27 mmol) of penicillide are dissolved in 1 ml of tetrahydrofuran and, at 0° C., 11 mg (0.28 mmol) of sodium hydride are added a little at a time. After 5 minutes, 73 mg (0.28 mmol) of 4,4,5,5,5-pentafluoro-1-pentanesulphonyl chloride and 0.11 ml (0.81 mmol) of triethylamine are added, and the mixture is stirred at room temperature for 2 hours. The reaction mixture is diluted with 25 ml of dichloromethane and washed twice with water. The orgainc phase is dried over magnesium sulphate and concentrated under reduced pressure. The residue is purified by preparative HPLC. This gives 120 mg (75% of theory) of product.

¹H-NMR (200 MHz, CDCl₃): δ=0.98 (dd, 6H), 1.48-1.77 (m, 3H), 1.95 (d, 1H), 2.12-2.40 (m, 4H), 2.32 (s, 3H), 3.46 (t, 2H), 3.98 (s, 3H), 5.06-5.17 (m, 3H), 6.85 (s, 1H), 7.10 (d, 1H), 7.26 (s, 1H), 7.63 (d, 1H) ppm.

MS (ESIpos): m/z=619 (M+Na)⁺

HPLC (Method 1): Rₜ=5.16 min.

### EXAMPLE B-107

### 9-[(1S)-1-Hydroxy-3-methylbutyl]-8-methoxy-3-methyl-4-chloro-7-oxo-5H,7H-dibenzo[b,g][1,5]-dioxocin-1-yl 4,4,5,5,5-pentafluoro-1-pentanesulphonate

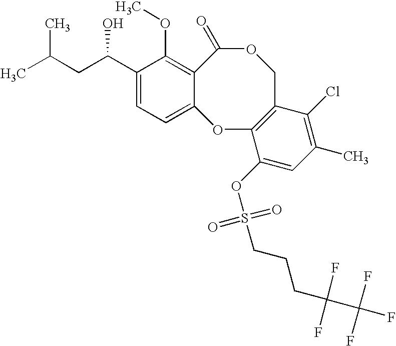

60 mg (0.147 mmol) of the compound from Example B-LII are dissolved in 1 ml of dichloromethane. Triethylamine (41 μl, 30 mg, 0.295 mmol) is added at 0° C., followed by slow dropwise addition of a solution of 4,4,5,5,5-pentafluoro-1-pentanesulphonyl chloride (46 mg, 0.177 mmol) in 1 ml of dichloromethane. The mixture is continued to be stirred at room temperature. Once the reaction has gone to completion (reaction monitored by TLC) the mixture is washed first with 1 M hydrochloric acid and then with saturated sodium bicarbonate solution. The organic phase is dried over magnesium sulphate and concentrated under reduced pressure.

The residue is purified chromatographically on silica gel (mobile phase: cyclohexane/ethyl acetate 8:1→5:1). This gives 53 mg (57% of theory) of product.

¹H-NMR (200 MHz, CDCl₃): δ=0.98 (m, 6H), 1.41-2.37 (m, 8H), 2.39 (s, 3H), 3.48 (t, 2H), 3.99 (s, 3H), 5.08 (m, 1H), 5.42 (br. s, 2H), 7.06 (d, 1H), 7.33 (br. s, 1H), 7.63 (d, 1H).

MS (DCI): m/z=648 (M+NH₄)⁺

HPLC (Method 1): Rₜ=5.4 min.

### EXAMPLE B-108

### 9-[(1S)-1-Hydroxy-3-methylbutyl]-8-methoxy-3-methyl-7-oxo-5H,7H-dibenzo[b,g]-[1,5]-dioxocin-1-yl cyclopropanesulphonate

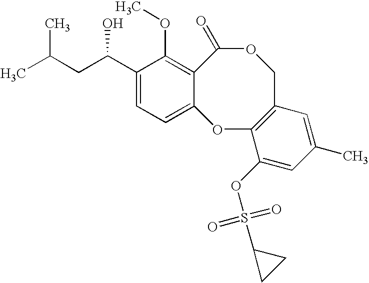

The preparation is carried out analogously to Example B-106 using 100 mg (0.27 mmol) of penicillide. This gives 89 mg (69% of theory) of product.

¹H-NMR (300 MHz, CDCl₃): δ=0.98 (t, 6H), 1.08-1.15 (m, 2H), 1.23-1.29 (m, 2H), 1.45-1.99 (m, 4H), 2.30 (s, 3H), 2.71-2.80 (m, 1H), 3.97 (s, 3H), 5.07-5.10 (m, 3H), 6.80 (s, 1H), 7.14 (d, 1H), 7.26 (s, 1H), 7.61 (d, 1H) ppm.

MS (ESIpos): m/z=499 (M+Na)⁺

HPLC (Method 1): Rₜ=4.76 min.

### EXAMPLE B-109

### 3-(1-Hydroxy-3-methyl-3-phenylbutyl)-11-(isopentyloxy)-4-methoxy-9-methyl-5H,7H-dibenzo[b,g][1,5]dioxocin-5-one

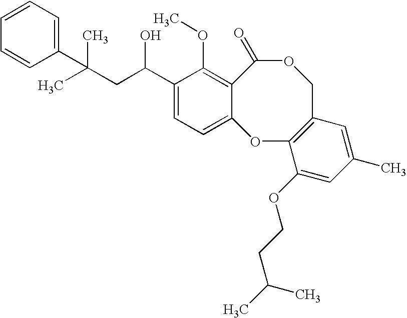

Under argon, 50 mg (130 μmol) of the compound from Example B-X are dissolved in 2 ml of tetrahydrofuran and cooled to −78° C. 520 μl (260 μmol) of a 0.5 M solution of 2-methyl-2-phenylpropylmagnesium chloride in diethyl ether are then added dropwise. The ice-bath is removed and the mixture is stirred at room temperature over night. 1 ml of a saturated solution of ammonium chloride is carefully added, and the reaction solution is filtered through a 1.8 g Extrelut/silica gel cartridge. The cartridge is eluted with dichloromethane. The filtrate is concentrated under reduced pressure. The residue is purified on a 20 g silica gel cartridge (mobile phase: cyclohexane/ethyl acetate 98:2→50:50). This gives 61 mg (90% of theory) of product.

LC-MS (Method 3): Rₜ=5.5 min.

MS (ESIpos): m/z=541 (M+Na)⁺

### EXAMPLE B-110

### 3-[Hydroxy(4-methoxyphenyl)methyl]-11-(isopentyloxy)-4-methoxy-9-methyl-5H,7H-dibenzo[b,g][1,5]dioxocin-5-one

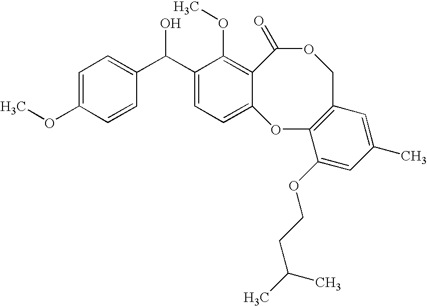

The preparation is carried out analogously to Example B-109 using 50 mg (0.13 mmol) of the compound from Example B-X. This gives 48 mg (75% of theory) of product.

LC-MS (Method 3): Rₜ=5.00 min.

MS (ESIpos): m/z=493 (M+H)⁺

### EXAMPLE B-111

### 3-(3-Cyclopropyl-1-hydroxy-propyl)-11-(isopentyloxy)-4-methoxy-9-methyl-5H,7H-dibenzo[b,g][1,5]dioxocin-5-one

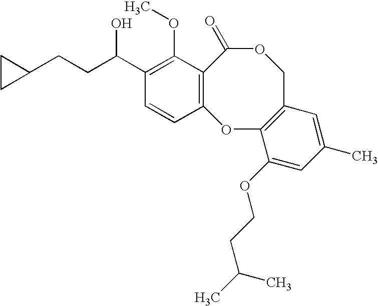

Under argon and at 0° C., 140 μl of a solution of diethylzinc (15% by weight in hexane) and 7.3 μl (90 μmol) of diiodomethane are added to a solution of 20 mg (45 μmol) of the compound from Example B-XI in 0.5 ml of dry dichloromethane. The mixture is stirred at this temperature for 30 minutes and for a further 18 hours at room temperature. At room temperature, a further 140 μl of the diethylzinc solution and 10 μl (123 μmol) of diiodomethane are then added. The mixture is stirred at room temperature for a further 16 hours, and 0.5 ml of a saturated solution of ammonium chloride is then added. The mixture is filtered through a 1.1 g Extrelut/silica gel cartridge. The cartridge is eluted with 10 ml of ethyl acetate and the filtrate is concentrated under reduced pressure. The residue is purified by preparative thick-layer chromatography (mobile phase: toluene/ethyl acetate 9:1). This gives 3 mg (15% of theory) of product.

¹H-NMR (200 MHz, CDCl₃): δ=0.01-0.04 (m, 2H), 0.40-0.44 (m, 2H), 1.00 (d, 6H), 1.60-1.99 (m, 8H), 2.04 (d, 1H), 2.27 (s, 3H), 3.97 (s, 3H), 4.09 (t, 2H), 4.99-5.12 (m, 3H), 6.42 (br. s, 1H), 6.79 (br. s, 1H), 6.95 (d, 1H), 7.55 (d, 1H) ppm.

### EXAMPLE B-112

### 9-[(1S)-1-Hydroxy-3-methylbutyl]-8-methoxy-3-methyl-7-oxo-4-vinyl-5H,7H-dibenzo[b,g][1,5]dioxocin-1-yl 3-azabicyclo[3.2.0]heptane-3-carboxylate

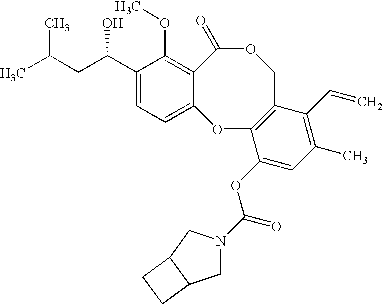

Under argon, 16 mg (100 μmol) of 3-azabicyclo[3.2.0]heptane-3-carbonyl chloride are initially charged, and 27 mg (70 μmol) of the compound from Example B-VII, dissolved in 150 μl of tetrahydrofuran, are added. 20 μl (100 μmol) of 1,8-di-azabicyclo[5.4.0]undec-7-ene, dissolved in 100 μl of tetrahydrofuran, are then added, and the mixture is stirred at room temperature overnight. 0.8 ml of water and 3 drops of 1 N hydrochloric acid are added and the mixture is diluted with 3 ml of ethyl acetate and filtered through a 1.1 g Extrelut/silica gel cartridge. The cartridge is eluted with 12 ml of ethyl acetate and the filtrate is concentrated under reduced pressure. The residue is purified by preparative HPLC. This gives 56 mg (56% of theory) of product.

¹H-NMR (300 MHz, CDCl₃): δ=0.97 (t, 6H), 1.45-1.77 (m, 5H), 1.92-1-98 (m, 1H), 2.2-2.25 (m, 5H), 2.96-3.02 (m, 2H), 3.4-3.58 (m, 2H), 3.74-3.83 (m, 2H), 3.97 (s, 3H), 4.97 (dd, 1H), 5.04-5.11 (m, 1H), 5.29 (s, 2H), 5.57 (dd, 1H), 6.59 (dd, 1H), 7.07 (s, 1H), 7.20 (d, 1H), 7.57 (d, 1H) ppm.

MS (ESIpos): m/z=543 (M+Na)⁺

The examples listed in the table below are prepared analogously to the procedures described above, from the corresponding starting materials:

| Example | | |
|---|---|---|
| B- | Structure | Analytical data |
| 113 | 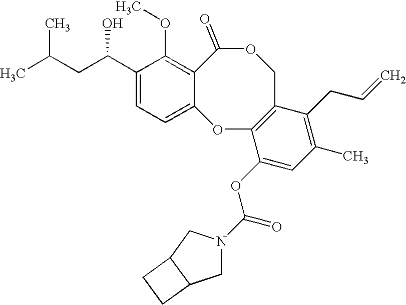 | ¹H-NMR(300 MHz, CDCl₃): δ = 0.97(t, 6H), 1.45-1.76(m, 5H), 1.92-1-98(m, 1H), 2.2-2.24 (m, 5H), 2.88-2.98(m, 2H), 3.27-3.29(m, 2H), 3.4-3.6(m, 2H), 3.80(dd, 2H), 3.97(s, 3H), 4.80 (dd, 1H), 5.01-5.05(m, 2H), 5.17 (s, 2H), 5.77-5.90(m, 1H), 7.08(s, 1H), 7.18(d, 1H), 7.56(d, 1H) ppm. MS(ESIpos): m/z = 557(M + Na)⁺ |
| 114 | 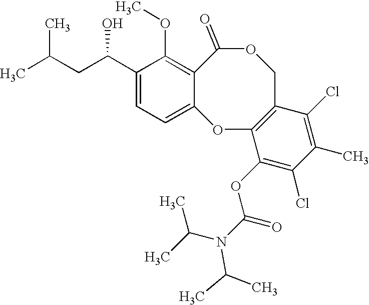 | ¹H-NMR(200 MHz, CDCl₃): δ = 0.99(dd, 6H), 1.21-1.42(m, 12H), 1.43-1.89(m, 3H), 1.94(d, 1H), 2.47(s, 3H), 3.98(s, 3H), 3.99-4.21(m, 2H), 5.03-5.12(m, 1H), 5.13-5.28(m, 1H), 5.55-5.73 (m, 1H), 7.13(d, 1H), 7.58(d, 1H) ppm. MS(ESIpos): m/z = 590(M + Na)⁺ |
| 115 | 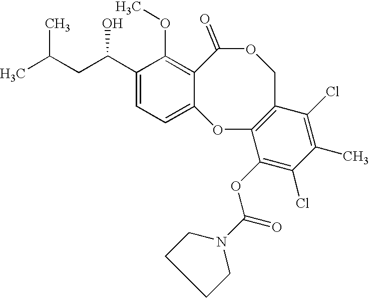 | ¹H-NMR(300 MHz, CDCl₃): δ = 0.99(dd, 6H), 1.42-1.87(m, 7H), 1.95(d, 1H), 2.48(s, 3H) 3.52(t, 2H), 3.69(t, 2H), 3.97(s, 3H), 5.09(dd, 1H), 5.28-5.51(br. s, 2H), 7.19(d, 1H), 7.59(d, 1H) ppm. MS(ESIpos): m/z = 560(M + Na)⁺HPLC(Method 2): Rₜ = 5.38 min. |

### EXAMPLE B-116

### 9-[(1S)-1-Hydroxy-3-methylbutyl]-8-methoxy-3-methyl-7-oxo-5H,7H-dibenzo[b,g]-[1,5]-dioxocin-1-yl 1,3,3-trimethyl-6-azabicyclo[3.2.1]octane-6-carboxylate

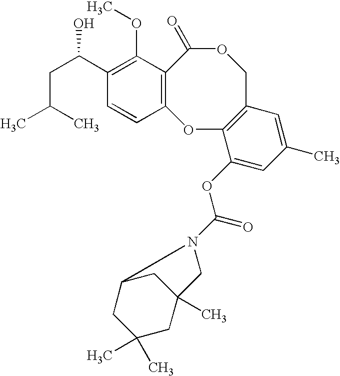

Under argon, 100 mg (0.27 mmol) of penicillide are dissolved in 1 ml of dimethylformamide and, at 0° C., 11 mg (0.28 mmol) of sodium hydride are added. 87 mg (0.40 mmol) of 1,3,3-trimethyl-6-azabicyclo[3.2.1]octane-6-carbonyl chloride and a catalytic amount of tetrabutylammonium iodide are then added, and the mixture is heated at 60° C. overnight. After cooling, 1.5 ml of water are added and the reaction mixture is diluted with dichloromethane and filtered through an NT 3 Extrelut cartridge. The cartridge is eluted three times with in each case 5 ml of dichloromethane and the filtrate is concentrated under reduced pressure. The residue is purified by preparative HPLC. This gives 115 mg (78% of theory) of product.

R_{f}=0.21 (cyclohexane/ethyl acetate 2:1)

¹H-NMR (200 MHz, CDCl₃): δ=0.94-0.99 (m, 9H), 1.05 (d, 3H), 1.10 (d, 3H), 1.25-2.15 (m, 10H), 2.27 (s, 3H), 3.13-3.29 (m, 1H), 3.44-3.62 (m, 1H), 3.97 (s, 3H), 4.28-4.45 (m, 1H), 5.02-5.15 (m, 3H), 6.70 (s, 1H), 6.99 (s, 1H), 7.15-7.23 (m, 1H), 7.50-7.60 (m, 1H) ppm.

The examples listed in the table below are prepared analogously to the procedures described above, from the corresponding starting materials:

| Example | | |
|---|---|---|
| B- | Structure | Analytical data |
| 117 | 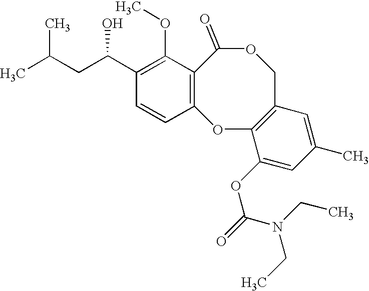 | ¹H-NMR(200 MHz, CDCl₃): δ = 0.97(q, 6H), 1.24(q, 6H), 1.42-1.87(m, 3H), 1.94(d, 1H), 2.27(s; 3H), 3.36-3.57(m, 4H), 3.97(s, 3H), 5.02-5.15(m, 3H), 6.71(br. s, 1H), 6,93(br. s, 1H), 7.00(d, 1H), 7.55(d, 1H) ppm. MS(ESIpos): m/z = 494(M + Na)⁺HPLC(Method 1): Rₜ = 5.06 min. |
| 118 | 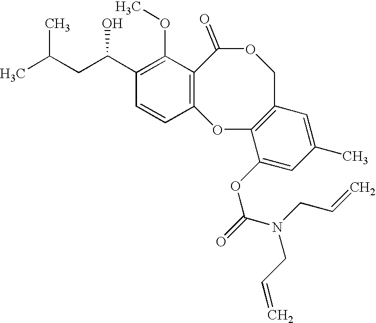 | ¹H-NMR(300 MHz, CDCl₃): δ = 0.98(m, 6H), 1.40-1.95(m, 4H), 2.68(s, 3H), 3.97(s, 3H), 4.00-4.10(m, 4H), 5.01-5.03(m, 3H), 5.13-5.28(m, 4H), 5.20-5.97 (m, 2H), 6.71(br. s, 1H), 6.93(br. s, 1H), 7.00(d, 1H), 7.55(d, 1H) ppm. MS(ESIpos): m/z = 518(M + Na)⁺HPLC(Method 1): Rₜ = 5.1 min. |
| 119 | 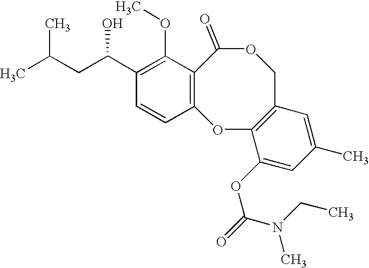 | ¹H-NMR(200 MHz, CDCl₃): δ = 0.98(m, 6H), 1.22(m, 3H), 1.40-1.95(m, 4H), 2.68(s, 3H), 3.00-3.09(m, 3H), 3.37-3.59(m, 2H), 3.97(s, 3H), 5.0 1-5.03(m, 3H), 6.71(br. s, 1H), 6.93(br. s, 1H), 7.00(d, 1H), 7.55(d, 1H) ppm. MS(ESIpos): m/z = 480(M + Na)⁺HPLC(Method 1): Rₜ = 4.8 min. |
| 120 | 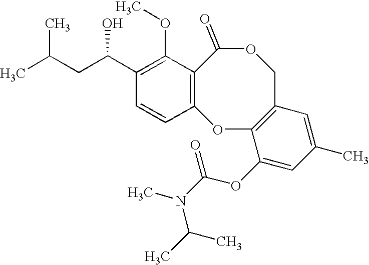 | ¹H-NMR(200 MHz, CDCl₃): δ = 0.98(m, 6H), 1.2(t, 6H), 1.4-1.95(m, 4H), 2.68(s, 3H), 2.90 (m, 3H), 3.97(s, 3H), 5.01-5.03 (m, 3H), 5.13-5.28(m, 4H), 5.20-5.97(m, 2H), 6.71(br. s, 1H), 6.99 (br. s, 1H), 7.20(m, 1H), 7.55(d, 1H) ppm. MS(ESIpos): m/z = 494(M + Na)⁺HPLC(Method 1): Rₜ = 5.0 min. |
| 121 | 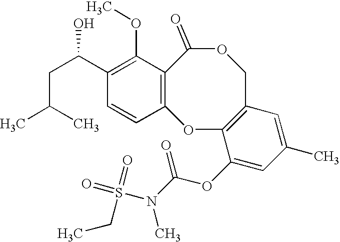 | ¹H-NMR(200 MHz, CDCl₃): δ = 0.97(t, 6H), 1.38(dt, 3H), 1.42-1.87(m, 3H), 1.95(d, 1H), 2.3(s, 3H), 3.46(s, 3H), 3.64(q, 2H), 3.97(s, 3H), 5.02-5.13(m, 3H), 6.71(br. s, 1H), 6.93(br. s, 1H), 7.00(d, 1H), 7.55(d, 1H) ppm. MS(ESIpos): m/z = 543(M + Na)⁺HPLC(Method 1): Rₜ = 4.83 min. |
| 122 | 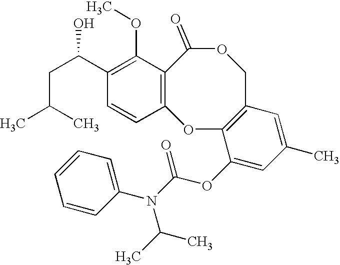 | ¹H-NMR(300 MHz, CDCl₃): δ = 0.98(m, 6H), 1.2(d, 6H), 1.42-1.88(m, 3H), 1.93(d, 1H), 2.23(s, 3H), 3.98(s, 3H), 4.69(m, 1H), 5.04-5.1(m, 3H), 6.65(s, 1H), 6.96(m, 2H), 7.25(m, 2H), 7.3-7.4(m, 3H), 7.52(d, 1H) ppm. MS(ESIpos): m/z = 556(M + Na)⁺HPLC(Method 1): Rₜ = 5.4 min. |
| 123 | 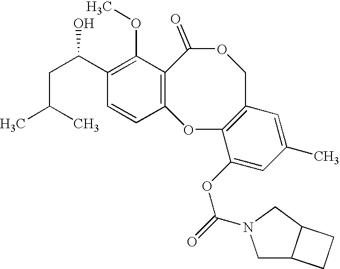 | ¹H-NMR(300 MHz, CDCl₃): δ = 0.97(t, 6H), 1.42-1.83(m, 5H), 1.93(d, 1H), 2.17-2.28(m, 2H), 2.28(s, 3H), 2.98(s, 2H), 3.41-3.9(m, 4H), 3.97(s, 3H), 5.02-5.13(m, 3H), 6.71(br. s, 1H), 7.03(br. s, 1H), 7.20(d, 1H), 7.55(d, 1H) ppm. MS(ESIpos): m/z = 518(M + Na)⁺HPLC(Method 1): Rₜ = 5.1 min. |
| 124 | 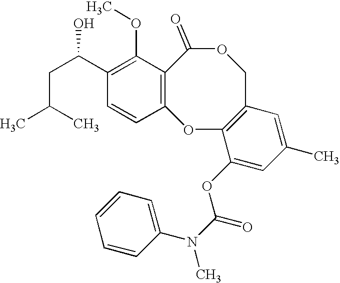 | ¹H-NMR(200 MHz, CDCl₃): δ = 0.98(q, 6H), 1.4-1.87(m, 3H), 1.95(d, 1H), 2.25(s, 3H), 3.45(s, 3H), 3.98(s, 3H), 5.05-5.15(m, 3H), 6.70(s, 1H), 6.96-7.04(m, 2H), 7.29-7.32(m, 1H), 7.37-7.41 (m, 4H), 7.54(d, 1H) ppm. |
| 125 | 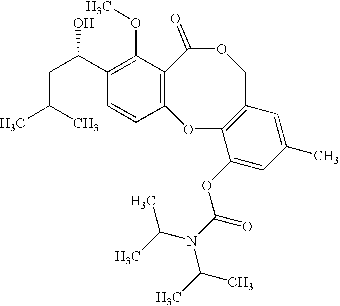 | ¹H-NMR(200 MHz, CDCl₃): δ = 0.97(q, 6H), 1.33(d, 12H), 1.40-1.87 (m, 3H), 1.92(d, 1H), 2.27(s, 3H), 3.97(s, 3H), 4.00-4.20(m, 2H), 5.05-5.15(m, 3H), 6.70(s, 1H), 7.01(s, 1H), 7.17(d, 1H), 7.56(d, 1H) ppm. |
| 126 | 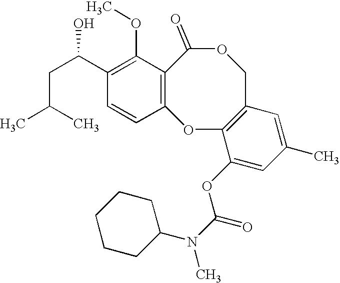 | ¹H-NMR(200 MHz, CDCl₃): δ = 0.97(sq, 6H) 1.30-1.90(m, 13H), 1.93(d, 1H), 2.27(s, 3H), 2.97(d, 3H), 3.97(s, 3H), 4.00-4.20(m, 1H), 5.05-5.15(m, 3H), 6.71(s, 1H), 7.01(s, 1H), 7.14(d, 1H), 7.57(d, 1H) ppm. |
| 127 | 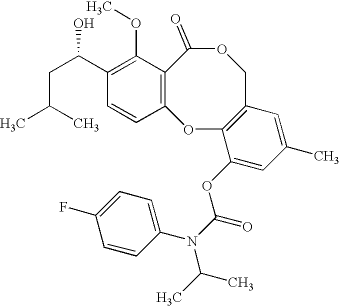 | ¹H-NMR(200 MHz, CDCl₃): δ = 0.98(q, 6H), 1.19(d, 6H), 1.45-1.82(m, 3H), 1.94(d, 1H), 2.24(s, 3H), 3.98(s, 3H), 4.60-4.80(m, 1H), 5.05-5.15(m, 3H), 6.67(s, 1H), 6.90-7.00(m, 2H), 7.07(t, 2H), 7.19-7.25(m, 2H), 7.55(d, 1H) ppm. |
| 128 | 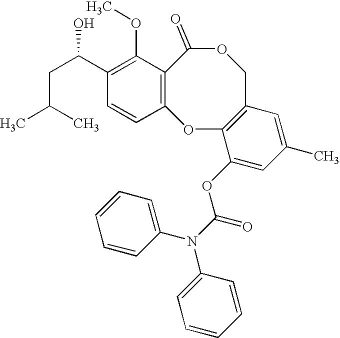 | ¹H-NMR(200 MHz, CDCl₃): δ = 0.98(q, 6H), 1.45-1.85(m, 3H), 1.93(d, 1H), 2.26(s, 3H), 3.99(s, 3H), 5.02-5.15(m, 3H), 6.71(s, 1H), 6.94(d, 1H), 7.05(s, 1H), 7.25-7.39(m, 10H), 7.51(d, 1H) ppm. |
| 129 | 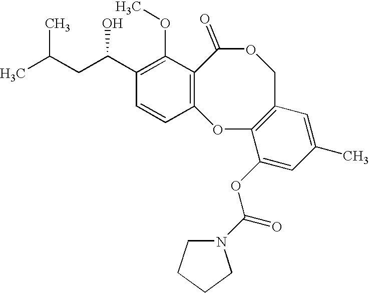 | ¹H-NMR(300 MHz, CDCl₃): δ = 0.97(t, 6H), 1.43-1.52(m, 1H), 1.65-1.82(m, 2H), 1.92-2.00 (m, 5H), 2.27(s, 3H), 3.49(t, 2H), 3.63(t, 2H), 3.96(s, 3H), 5.02-5.15(m, 3H), 6.71(s, 1H), 7.02(s, 1H), 7.19(d, 1H), 7.57(d, 1H) ppm. |
| 130 | 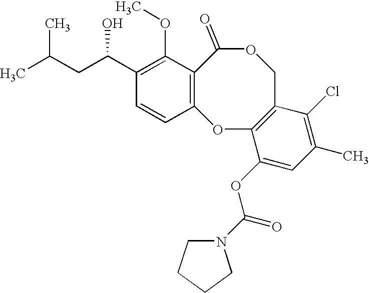 | ¹H-NMR(300 MHz, CDCl₃): δ = 0.98(m, 6H), 1.64(m, 2H), 1.70(m, 1H), 1.95(m, 4H), 2.31 (s, 3H), 3.50(dd, 2H), 3.64(dd, 2H), 3.96(s, 3H), 5.08(m, 1H), 5.39(d, 1H), 5.43(d, 1H), 7.12 (br. s, 1H), 7.14(d, 1H), 7.59(d, 1H) ppm. MS(ESIpos): m/z = 526(M + Na)⁺HIPLC(Method 1): Rₜ = 5.0 min. |
| 131 | 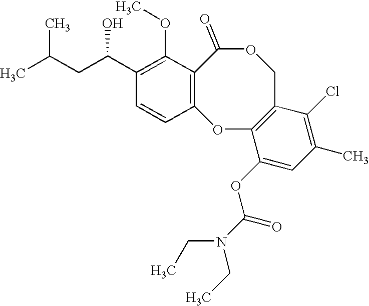 | ¹H-NMR(400 MHz, CDCl₃): δ = 0.98(m, 6H), 1.19-1.31(m, 6H), 1.49(m, 1H), 1.69(m, 1H), 1.80(m, 1H), 1.92(d, 1H), 2.31(s, 3H), 3.3 8-3.52(m, 4H), 3.99(s, 3H), 5.08(m, 1H), 5.39(d, 1H), 5.43(d, 1H), 7.10(d, 1H), 7.10 (br. s, 1H), 7.59(m, 1H) ppm. MS(ESIpos): m/z = 528(M + Na)⁺HPLC(Method 1): Rₜ = 5.2 min. |
| 132 | 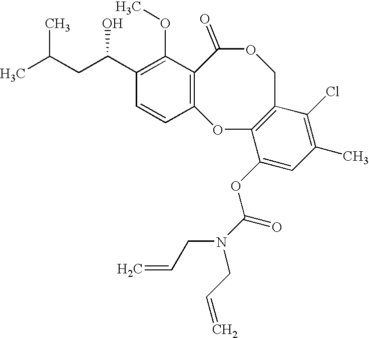 | ¹H-NMR(400 MHz, CDCl₃): δ = 0.98(m, 6H), 1.49(m, 1H), 1.69(m, 1H), 1.80(m, 1H), 1.92 (d, 1H), 2.31(s, 3H), 3.38-3.52 (m, 4H), 3.99(s, 3H), 4.00(m, 4H), 5.08(m, 1H), 5.17-5.29(m, 4H), 5.39(d, 1H), 5.43(d, 1H), 5.72-5.93(m, 2H), 7.10(d, 1H), 7.10(br. s, 1H), 7.59(m, 1H) ppm. LC-MS(Method 6): Rₜ = 5.43 min., m/z = 552(M + Na)⁺ |
| 133 | 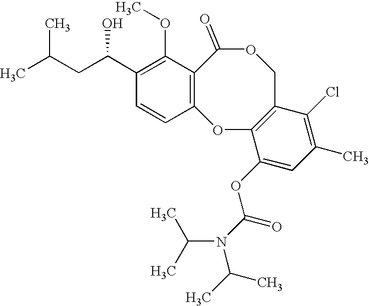 | ¹H-NMR(200 MHz, CDCl₃): δ = 0.98(m, 6H), 1.20-1.93(m, 16H), 2.31(s, 3H), 3.96(s, 3H), 4.02(m, 2H), 5.08(m, 1H), 5.39 (d, 1H), 5.43(d, 1H), 7.10(d, 1H), 7.10(br. s, 1H), 7.59(m, 1H) ppm. LC-MS(Method 4, ESIneg): Rₜ =4.40 min., m/z = 578 (M − H + HCOOH)⁻ |

### EXAMPLE B-134

### 9-[(1S)-1-Hydroxy-3-methylbutyl]-8-methoxy-3-methyl-7-oxo-5H,7H-dibenzo[b,g]-[1,5]-dioxocin-1-yl 4-morpholinecarboxylate

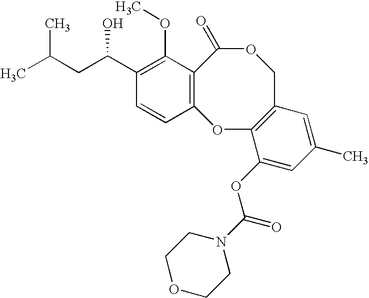

Under argon, 35 μl (0.40 mmol) of morpholine are dissolved in 1.0 ml of dichloromethane. 33 μl (0.40 mmol) of pyridine and 57 mg (0.28 mmol) of 4-nitro-phenyl chloroformate are then added and the mixture is heated at reflux. After one hour, the reaction solution is cooled, diluted with dichloromethane and washed twice with saturated sodium bicarbonate solution and twice with water. The organic phase is dried over sodium sulphate and concentrated under reduced pressure. In a second flask, 100 mg (0.27 mmol) of penicillide are, under argon, dissolved in 1 ml of dimethylformamide and, at 0° C., 11 mg (0.28 mmol) of sodium hydride are added. After 10 minutes, 175 mg (0.54 mmol) of caesium carbonate are added and, after brief stirring, the prepared carbamoyl chloride, dissolved in 0.5 ml of dichloro-methane, is added. The reaction mixture is then heated at 50° C. for 72 hours. After cooling, 1.5 ml of 6 N hydrochloric acid are added and the mixture is diluted with dichloromethane and filtered through an Extrelut cartridge. The cartridge is eluted with dichloromethane and the filtrate is concentrated under reduced pressure. The residue is purified by preparative HPLC. This gives 18 mg (14% of theory) of product.

¹H-NMR (300 MHz, CDCl₃): δ=0.97 (t, 6H), 1.42-1.81 (m, 3H), 1.93 (d, 1H), 2.28 (s, 3H), 3.6-3.72 (m, 8H), 3.97 (s, 3H), 5.04-5.10 (m, 3H), 6.72 (s, 1H), 7.01 (s, 1H), 7.10 (d, 1H), 7.58 (d, 1H) ppm.

MS (ESIpos): m/z=508 (M+Na)⁺

HPLC (Method 1): Rₜ=4.59 min.

### EXAMPLE B-135

### 9-[(1S)-1-Hydroxy-3-methylbutyl]-8-methoxy-3-methyl-7-oxo-5H,7H-dibenzo[b,g]-[1,5]-dioxocin-1-yl 1-piperidinecarboxylate

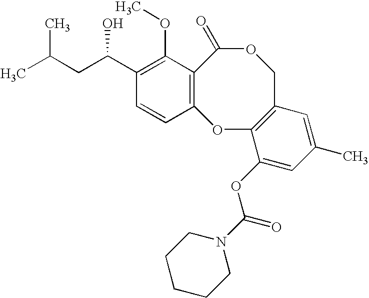

The preparation is carried out analogously to Example B-134 using 100 mg (0.27 mmol) of penicillide. This gives 7 mg (5% of theory) of product.

¹H-NMR (200 MHz, CDCl₃): δ=0.97 (q, 6H), 1.43-1.80 (m, 9H), 1.95 (d, 1H), 2.27 (s, 3H), 3.50-3.70 (m, 4H), 3.97 (s, 3H), 5.05-5.12 (m, 3H), 6.70 (s, 1H), 7.00 (s, 1H), 7.18 (d, 1H), 7.59 (d, 1H) ppm.

MS (ESIpos): m/z=506 (M+Na)⁺

### EXAMPLE B-136

### 9-[(1S)-1-Hydroxy-3-methylbutyl]-8-methoxy-3-methyl-7-oxo-5H,7H-dibenzo[b,g]-[1,5]-dioxocin-1-yl methyl(2,2,2-trifluoroethyl)carbamate

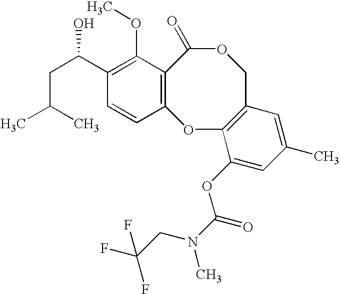

Under argon, 39 μl (0.32 mmol) of trichloromethyl chloroformate are dissolved in 0.3 ml of dichloromethane, and a solution of 105 μl (0.75 mmol) of triethylamine and 85 μl (0.64 mmol) of 2,2,2-trifluoro-N-methylethanamine in 0.2 ml of dichloro-methane is carefully added at 0° C. The ice-bath is removed and the resulting suspension is stirred at room temperature for 5 hours. The suspension is concentrated under reduced pressure and the residue is resuspended in 0.3 ml of tetrahydrofuran. A solution of 80 mg (0.21 mmol) of penicillide and 35 μl (0.24 mmol) of 1,8-diazabicyclo[5.4.0]undec-7-ene in 0.5 ml of tetrahydrofuran is added to this suspension, and the mixture is heated at 60° C. overnight. After cooling, 1.5 ml of water are added and the reaction mixture is diluted with 5 ml of ethyl acetate and filtered through an Extrelut cartridge. The cartridge is eluted with 35 ml of ethyl acetate and the filtrate is concentrated under reduced pressure. The residue is purified by preparative HPLC. This gives 84 mg (76% of theory) of product.

R_{f}=0.24 (cyclohexane/ethyl acetate 2:1)

¹H-NMR (300 MHz, CDCl₃): δ=0.97 (m, 6H), 1.44-1.86 (m, 3H), 1.91 (d, 1H), 2.28 (s, 3H), 3.23 (d, 3H), 3.97 (s, 3H), 4.00-4.16 (m, 2H), 5.00-5.16 (m, 3H), 6.75 (d, 1H), 7.00-7.08 (m, 2H), 7.55-7.59 (m, 1H) ppm.

MS (ESIpos): m/z=534 (M+Na)⁺

The examples listed in the table below are prepared analogously to the procedures described above, from the corresponding starting materials:

| Example | | |
|---|---|---|
| B- | Structure | Analytical data |
| 137 | 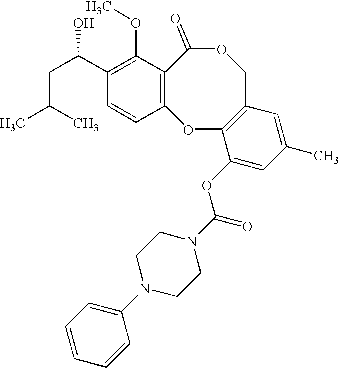 | ¹H-NMR(300 MHz, CDCl₃): δ = 0.97(t, 6H), 1.44-1.82(m, 3H), 1.90(d, 1H), 2.28(s 3H) 3.23(t, 4H), 3.77-3.90(m, 4H), 3.97(s, 3H), 5.05-5.12(m, 3H), 6.73(s, 1H), 6.87-6.96(m, 3H), 7.03(s, 1H), 7.12(d, 1H), 7.25-7.35 (m, 2H), 7.57(d, 1H) ppm. MS(ESIpos): m/z = 561(M + H)⁺ |
| 138 | 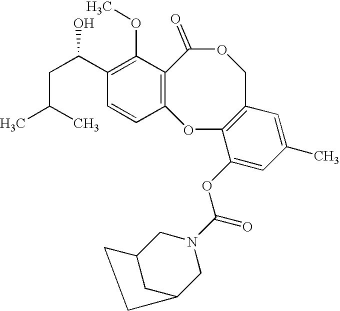 | ¹H-NMR(300 MHz, CDCl₃): δ = 0.97(t, 6H), 1.44-1.82(m, 9H), 1.92(t, 1H), 2.22-2.3(m, 5H), 3.00(d, 1H), 3.16(d, 1H), 3.97-4.12(m, 5H), 5.05-5.16(m, 3H), 6.70(s, 1H), 6.99(s, 1H), 7.16(d, 1H), 7.57(d, 1H) ppm. MS(ESIpos): m/z = 532(M + Na)⁺ |
| 139 | 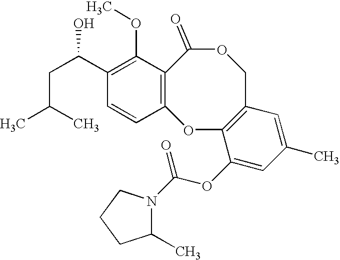 | ¹H-NMR(200 MHz, CDCl₃): δ = 0.97(t, 6H), 1.30(t, 3H), 1.40-2.18(m, 8H), 2.27(s, 3H), 3.48-3.70(m, 2H), 3.96(s, 3H), 4.00-4.30(m, 1H), 5.00-5.16(m, 3H), 6.70(s, 1H), 7.02(s, 1H), 7.13-7.25(m, 1H), 7.57(d, 1H) ppm. MS(ESIpos): m/z = 506(M + Na)⁺ |
| 140 | 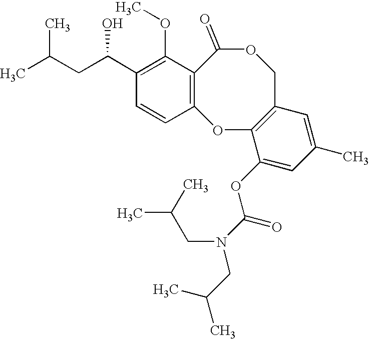 | ¹H-NMR(200 MHz, CDCl₃): δ = 0.89-0.99(m, 12H), 1.25-1.90 (m, 11H), 1.96(d, 1H), 2.27(s, 3H), 3.38(q, 4H), 3.97(s, 3H), 5.00-5.16(m, 3H), 6.70(s, 1H), 6.99(s, 1H), 7.15(d, 1H), 7.56(d, 1H) ppm. MS(ESIpos): m/z = 550(M + Na)⁺ |
| 141 | 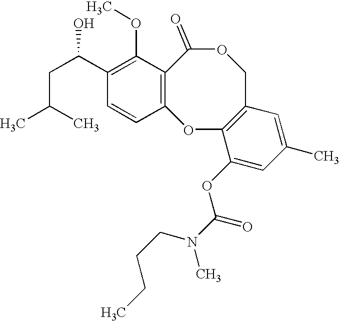 | ¹H-NMR(200 MHz, CDCl₃): δ = 0.89-0.99(m, 9H), 1.25-1.70 (m, 7H), 2.07(br. s, 1H), 2.27(s, 3H), 3.07(d, 3H), 3.42(quintet, 2H), 3.96(s, 3H), 5.00-5.16(m, 3H), 6.70(s, 1H), 7.00(s, 1H), 7.10-7.17(m, 1H), 7.56(d, 1H) ppm. MS(ESIpos): m/z = 508(M + Na)⁺ |
| 142 | 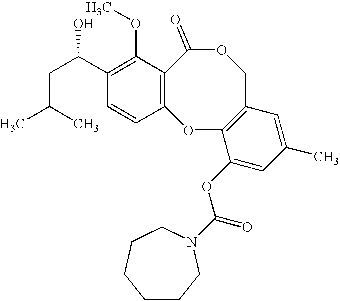 | ¹H-NMR(200 MHz, CDCl₃): δ = 0.97(q, 6H), 1.45-1.88(m, 11H), 2.02(d, 1H), 2.27(s, 3H), 3.45-3.68(m, 4H), 3.97(s, 3H), 5.00-5.16(m, 3H), 6.70(s, 1H), 7.00(s, 1H), 7.18(d, 1H), 7.57(d, 1H) ppm. MS(ESIpos): m/z = 520(M + Na)⁺ |
| 143 | 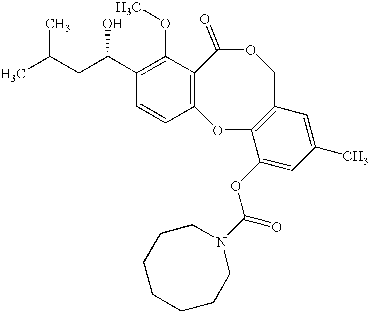 | ¹H-NMR(200 MHz, CDCl₃): δ = 0.85(q, 6H), 1.30-1.78(m, 13H), 1.91(d, 1H), 2.15(s, 3H), 3.35-3.50(m, 4H), 3.84(s, 3H), 4.88-5.03(m, 3H), 6.58(s, 1H), 6.87 (s, 1H), 7.06(d, 1H), 7.44(d, 1H) ppm. MS(ESIpos): m/z = 534(M + Na)⁺ |
| 144 | 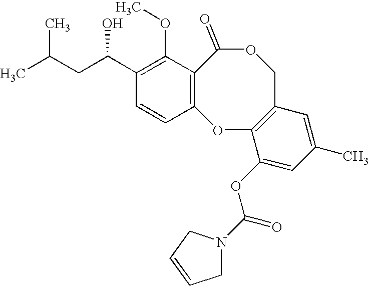 | ¹H-NMR(300 MHz, CDCl₃): δ = 0.97(t, 6H), 1.42-1.88(m, 4H), 2.28(s, 3H), 3.96(s, 3H), 4.29-4.35(m, 2H), 4.41-4.48(m, 2H), 5.05-5.12(m, 3H), 5.81-5.89 (m, 2H), 6.72(s, 1H), 7.04(s, 1H), 7.18(d, 1H), 7.57(d, 1H) ppm. |
| 145 | 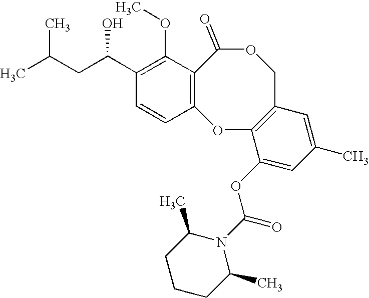 | ¹H-NMR(200 MHz, CDCl₃): δ = 0.97(t, 6H), 1.33(d, 6H), 1.45-1.84(m, 9H), 1.93(d, 1H), 2.27(s, 3H), 3.97(s, 3H), 4.46-4.58(m, 2H), 5.02-5.12(m, 3H), 6.70(s, 1H), 7.01(s, 1H), 7.19(d, 1H), 7.57(d, 1H) ppm. MS(DCI): m/z = 529(M + NH₄)⁺HPLC(Method 1): Rₜ = 5.28 min. |
| 146 | 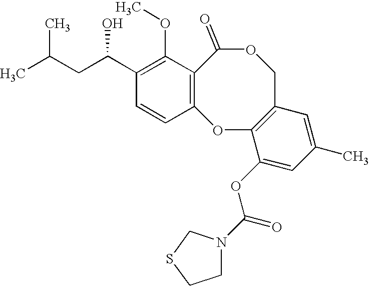 | ¹H-NMR(300 MHz, CDCl₃): δ = 0.97(t, 6H), 1.45-1.84(m, 3H), 1.92(s, 1H), 2.28(s, 3H), 3.03-3.15(m, 2H), 3.85-3.95(m, 2H), 3.97(s, 3H), 4.60-4.75(m, 2H), 5.02-5.12(m, 3H), 6.73(s, 1H), 7.03(s, 1H), 7.11(d, 1H), 7.58(d, 1H) ppm. |
| 147 | 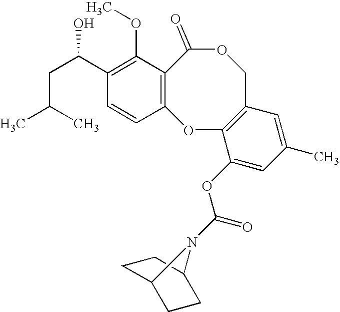 | ¹H-NMR(300 MHz, CDCl₃): δ = 0.97(t, 6H), 1.47-1.91(m, 12H), 2.27(s, 3H), 3.97(s, 3H), 4.47(s, 2H), 5.05-5.10(m, 3H), 6.70(s, 1H), 6.99(s, 1H), 7.14(d, 1H), 7.57(d, 1H) ppm. |
| 148 | 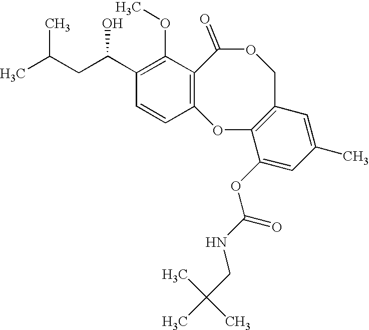 | ¹H-NMR(300 MHz, CDCl₃): δ = 0.90-1.03(m, 15H), 1.43-1.90 (m, 3H), 1.95(d, 1H), 2.29(s, 3H), 2.72-2-82(br. s, 1H), 3.97(s, 3H), 3.99(s, 2H), 5.02-5.15(m, 3H), 6.75(s, 1H), 7.02-7.08(m, 2H), 7.57(d, 1H) ppm. MS(DCI): m/z = 504(M + NH₄)⁺HPLC(Method 1): Rₜ = 5.28 min. |
| 149 | 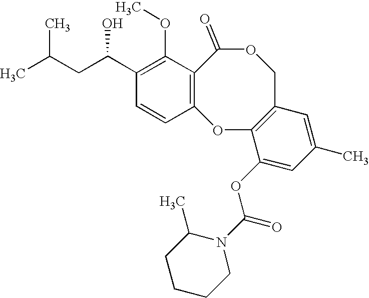 | ¹H-NMR(300 MHz, CDCl₃): δ = 0.98(t, 6H), 1.42-2.25(m, 9H), 2.27(s, 3H), 3.00-3.15(m, 1H), 3.98(s, 3H), 4.12-4.21(m, 1H), 4.58-4.7(m, 1H), 5.05-5.12 (m, 3H), 6.70(s, 1H), 7.00(s, 1H), 7.15(d, 1H), 7.57(d, 1H) ppm. LC-MS(Method 3): Rₜ = 4.94 min. MS(ESIpos): m/z = 520(M + Na)⁺ |
| 150 | 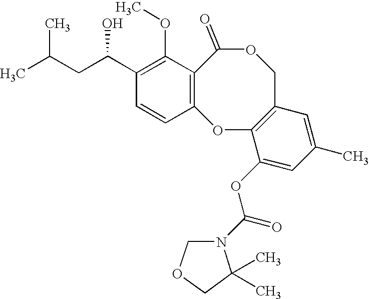 | ¹H-NMR(400 MHz, CDCl₃): δ = 0.97(t, 6H), 1.45-1.55(m, 7H), 1.66-1.79(m,2H), 1.93(d, 1H), 2.28(s, 3H), 3.85(d, 2H), 3.97(s, 3H), 5.06-5.09(m, 4H), 5.16(s, 1H), 6.72(s, 1H), 7.03(s, 1H), 7.12(dd, H), 7.57(d, 1H) ppm. MS(ESIpos): m/z = 522(M + Na)⁺HPLC(Method 1): Rₜ = 6.32 min. |
| 151 | 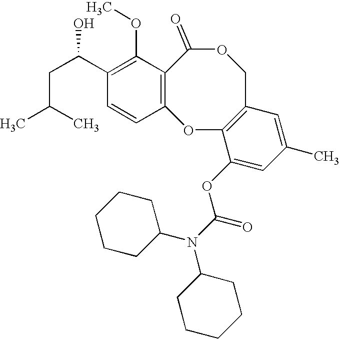 | ¹H-NMR(300 MHz, CDCl₃): δ = 0.97(t, 6H), 1.06-1.91(m, 24H), 2.27(s, 3H), 3.50-3.70(m, 2H), 3.97(s, 3H), 5.06-5.09(m, 3H), 6.69(s, 1H), 7.01(s, 1H), 7.13(d, 1H), 7.56(d, 1H) ppm. MS(ESIpos): m/z = 602(M + Na)⁺ |
| 152 | 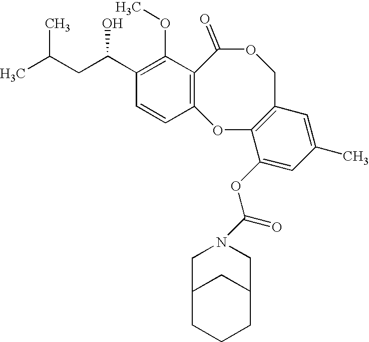 | ¹H-NMR(400 MHz, CDCl₃): δ = 0.97(t, 6H), 1.42-2.06(m, 14H), 2.27(s, 3H), 3.18(d, 1H), 3.35(d, 1H), 3.97(s, 3H), 4.22(d, 1H), 4.32(d, 1H), 5.00-5.15(m, 3H), 6.70(s, 1H), 7.00(s, 1H), 7.16(t, 1H), 7.56(dd, 1H) ppm. MS(ESIpos): m/z =546(M + Na)⁺ |
| 153 | 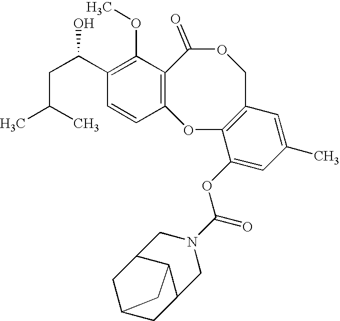 | ¹H-NMR(300 MHz, CDCl₃): δ = 0.97(t, 6H), 1.00-1.14(m, 2H), 1.37(s, 2H), 1.5 1-2.15(m, 10H), 2.27(s, 3H), 2.90(d, 1H), 3.06(d, 1H), 3.97(s, 3H), 4.24(t, 2H), 5.00-5.15(m, 3H), 6.70(s, 1H), 6.99(s, 1H), 7.18(d, 1H), 7.56(a, 1H) ppm. MS(ESIpos): m/z = 558(M + Na)⁺ |
| 154 | 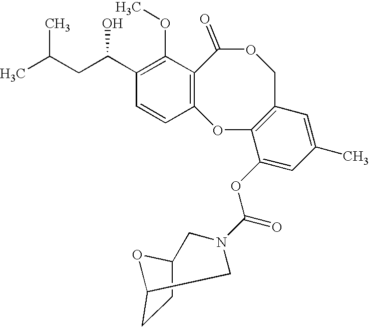 | ¹H-NMR(200 MHz, CDCl₃): δ = 0.95(t, 6H), 1.40-2.05(m, 8H), 2.25(s, 3H), 3.18-3.45(m, 2H), 3.83-3.96(m, 5H), 4.36(br. s, 2H), 5.00-5.18(m, 3H), 6.71(s, 7.56(d, 1H) ppm. MS(ESIpos): m/z = 529 (M + NH₄)⁺HPLC(Method 1): Rₜ = 4.68 min. |
| 155 | 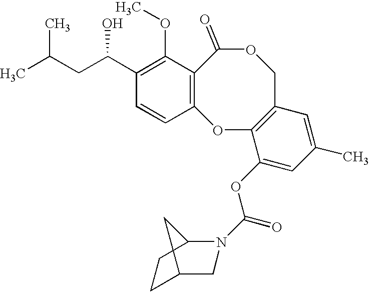 | ¹H-NMR(300 MHz, CDCl₃): δ = 0.98(dd, 6H), 1.42-1.93(m, 10H),2.28(s, 3H), 2.58-2.66/ 3.17-3.22(m, 1H), 3.30+4.48(d, 1H), 3.39-3.58(m, 2H), 3.96(s, 3H), 5.05-5.12(m, 3H), 6.70(br. s, 1H), 7.02(br. s, 1H), 7.11-7.23 (m, 1H), 7.53-7.62(m, 1H) ppm. MS(ESIpos): m/z = 518(M + Na)⁺HPLC(Method 1): Rₜ = 4.94 min. |
| 156 | 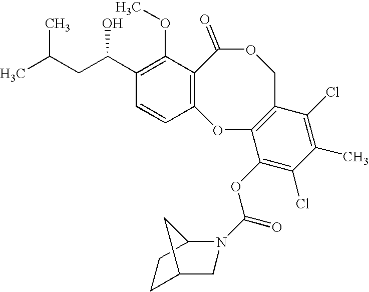 | ¹H-NMR(200 MHz, CDCl₃): δ = 0.99(dd, 6H), 1.16-1.90(m, 9H), 1.95(d, 1H), 2.46(s, 3H), 3.18-3.64(m, 4H), 3.98(s, 3H), 5.02-5.13(m, 1H), 5.20-5.70(br. s, 2H), 7.12.(d, 1H), 7.59(d, 1H) ppm. MS(ESIpos): m/z = 586(M + Na)⁺HPLC(Method 2): Rₜ = 5.91 min. |
| 157 | 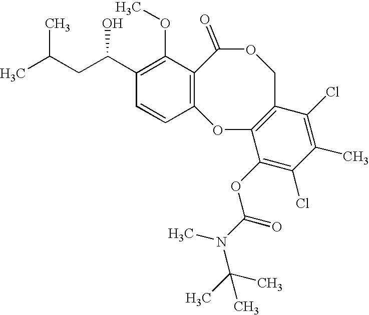 | ¹H-NMR(200 MHz, CDCl₃): δ = 0.99(dd, 6H), 1.50(s, 9H), 1.42-1.88(m, 3H), 1.94(d, 1H), 2.47(s, 3H), 3.18(s, 3H), 3.97(s, 3H), 5.03-5.12(m, 1H), 5.13-5.28 (m, 1H), 5.53-5.73(m, 1H), 7.13 (d, 1H), 7.59(d, 1H) ppm. MS(ESIpos): m/z = 576(M + Na)⁺ |
| 158 | 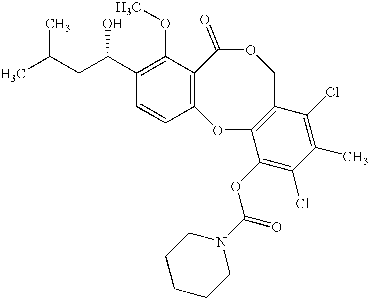 | ¹H-NMR(300 MHz, CDCl₃): δ = 0.99(dd, 6H), 1.42-1.89(m, 9H), 1.93(d, 1H), 2.48(s, 3H), 3.5 1-3.75(m, 4H), 3.98(s, 3H), 5.04-5.15(m, 1H), 5.17-5.63(br. s, 2H), 7.12(d, 1H), 7.60(d, 1H) ppm. MS(ESIpos): m/z = 574(M + Na)⁺HPLC(Method 2): Rₜ = 5.59 min. |
| 159 | 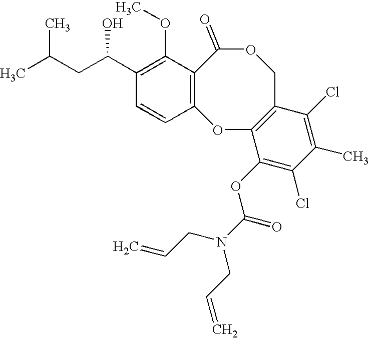 | ¹H-NMR(300 MHz, CDCl₃): δ = 0.99(dd, 6H), 1.42-1.89(m, 3H), 1.93(4,1H), 2.47(s, 3H), 3.96-4.16(m, 4H), 3.98(s, 3H), 5.05-5.12(m, 1H), 5.20-5.71(m, 6H), 5.73-6.01(m, 2H), 7.10(d, 1H), 7.59(d, 1H) ppm. MS(ESIpos): m/z =586(M + Na)⁺HPLC(Method 2): Rₜ = 5.66 min. |
| 160 | 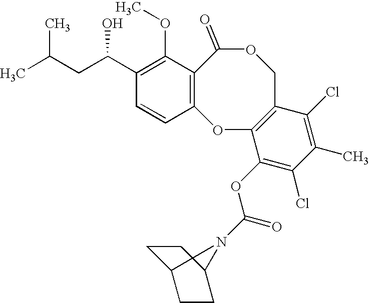 | ¹H-NMR(300 MHz, CDCl₃): δ = 0.99(dd, 6H), 1.42-1.87(m, 7H), 1.89-2.05(m, 5H), 2.47(s, 3H), 3.97(s, 3H), 4.42-4.61(m, 2H), 5.05-5.12(m, 1H), 5.23-5.48 (br. s, 2H), 7.10(d, 1H), 7.60(d, 1H) ppm. MS(ESIpos): m/z = 586(M + Na)⁺HPLC(Method 2): Rₜ = 5.62 min. |
| 161 | 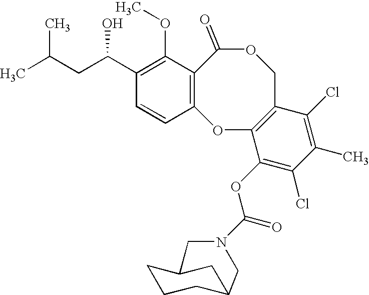 | ¹H-NMR(300 MHz, CDCl₃): δ = 0.99(dd, 6H), 1.42-2.0 1(m, 14H), 2.47(s, 3H), 3.21(d, 1H), 3.40(d, 1H), 3.98(s, 3H), 4.22(d, 1H), 4.38(d, 1H), 5.04-5.12(m, 1H), 5.18-5.61(br. s, 2H), 7.17 (dd, 1H), 7.59(d, 1H) ppm. MS(ESIpos): m/z = 609 (M + NH₄)⁺HPLC(Method 1): Rₜ = 6.01 min. |
| 162 | 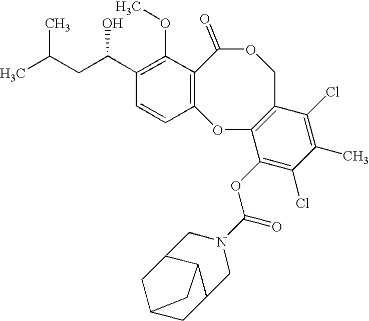 | ¹H-NMR(200 MHz, CDCl₃): δ = 0.99(dd, 6H), 1.31-2.21(m, 14H), 2.48(s, 3H), 2.92(d, 1H), 3.11(d, 1H), 3.98(s, 3H), 4.20(d, 1H), 4.30(d, 1H), 5.02-5.13(m, 1H), 5.23(br. s, 1H), 5.55(br. s, 1H), 7.13(d, 1H), 7.59(d, 1H) ppm. MS(ESIpos): m/z = 621 (M + NH₄)⁺HPLC(Method 1): Rₜ = 6.14 min. |
| 163 | 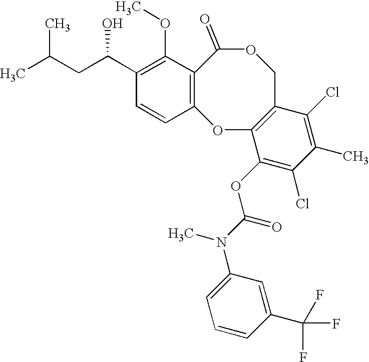 | ¹H-NMR(200 MHz, CDCl₃): δ = 0.99(dd, 6H), 1.40-2.05(m, 4H), 2.47(s, 3H), 3.52(s, 3H), 3.99(s, 3H), 5.09(dd, 1H), 5.39 (br. s, 2H), 6.92(br. s, 1H), 7.49-7.72(m, 5H) ppm. MS(ESIpos): m/z = 659 (M + NH₄)⁺HPLC(Method 2): Rₜ = 5.86 min. |
| 164 | 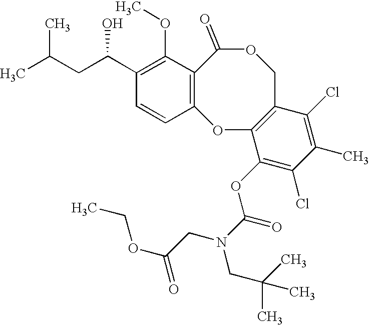 | ¹H-NMR(200 MHz, CDCl₃): δ = 0.92-1.08(m, 15H), 1.24(t, 3H), 1.41-1.88(m, 3H), 1.92(br. 1H), 2.46(s, 3H), 3.40(br. s, 2H), 3.98(s, 3H), 4.08-4.30(m, 4H), 5.03-5.13(m, 1H), 5.21(br. s, 1H), 5.58(br. s, 1H), 7.10(dd, 1H), 7.59(d, 1H) ppm. MS(ESIpos): m/z = 657 (M + NH₄)⁺HPLC(Method 2): Rₜ = 5.96 min. |
| 165 | 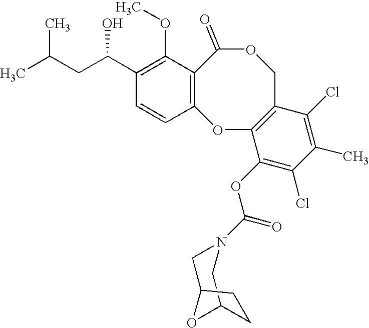 | ¹H-NMR(300 MHz, CDCl₃): δ = 0.99(dd, 6H), 1.42-2.10(m, 8H), 2.47(s, 3H), 3.30(d, 1H), 3.48(d, 1H), 3.88(d, 1H), 3.98(s, 3H), 3.99(d, 1H), 4.40(br. s, 2H), 5.04-5.13(m, 1H), 5.30(br. s, 1H), 5.48(br. s, 1H), 7.09(d, 1H), 7.61(d, 1H) ppm. MS(ESIpos): m/z = 597 (M + NH₄)⁺HPLC(Method 2): Rₜ = 5.26 min. |
| 166 | 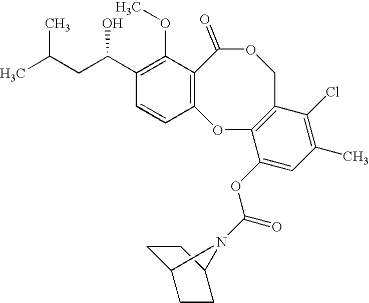 | ¹H-NMR(200 MHz, CDCl₃): δ = 0.98(m, 6H),1.41-1.97(m, 10H), 2.31(s, 3H), 3.99(s, 3H), 4.45(m, 2H), 5.08(m, 1H), 5.39 (d, 1H), 5.43(d, 1H), 7.10(d, 1H), 7.10(br. s, 1H), 7.59(m, 1H) ppm. MS(ESIpos): m/z = 552(M + Na)⁺ |

### EXAMPLE B-167

### 9-[1-(Formyloxy)-3-methylbutyl]-8-methoxy-3-methyl-7-oxo-5H,7H-dibenzo[b,g]-[1,5]dioxocin-1-yl 1-piperidinecarboxylate

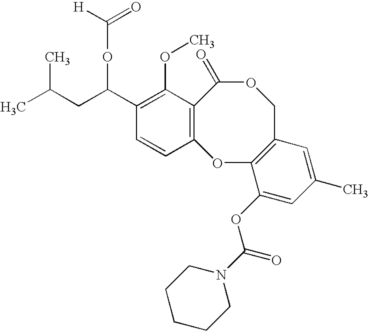

Under argon, 60 mg (0.15 mmol) of the compound from Example B-XIII are dissolved in 1 ml of dichloromethane and 13 μl (0.16 mmol) of pyridine. 36 mg (0.23 mmol) of 1,1′-carbonyldiimidazole are then added, and the mixture is stirred at room temperature for 2 hours. 30 μl (0.30 mmol) of piperidine are then added, and the mixture is stirred at room temperature for 1.5 hours. 1 ml of water and 1 ml of 1 N hydrochloric acid are added and the reaction solution is diluted with 5 ml of ethyl acetate and filtered through an Extrelut cartridge. The cartridge is eluted with 35 ml of ethyl acetate and the filtrate is concentrated under reduced pressure. The residue is purified by preparative HPLC. This gives 23 mg (30% of theory) of product.

R_{f}=0.23 (cyclohexane/ethyl acetate 2:1)

¹H-NMR (300 MHz, CDCl₃): δ=0.95 (d, 6H), 1.52-1.63 (m, 8H), 1.78-1.87 (m, 1H), 2.27 (s, 3H), 3.50-3.72 (m, 4H), 4.03 (s, 3H), 4.99-5.12 (m, 2H), 6.26 (dd, 1H), 6.70 (s, 1H), 6.99 (s, 1H), 7.16 (d, 1H), 7.47 (d, 1H), 8.06 (s, 1H) ppm.

LC-MS (Method 3): Rₜ=5.20 min.

MS (ESIpos): m/z=534 (M+Na)⁺

### EXAMPLE B-168

### Bicyclo[2.2.1]hept-2-ylmethyl(1-hydroxy-3-methylbutyl)-8-methoxy-3-methyl-7-oxo-5H,7H-dibenzo[b,g][1,5]dioxocin-1-carboxylate

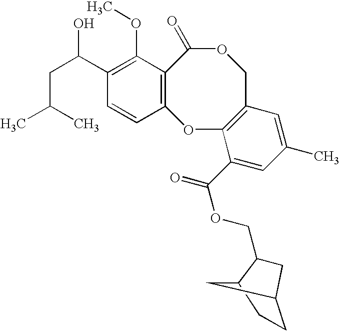

30 mg (59 μmol) of the compound from Example B-XXXIII are dissolved in 1.5 ml of a mixture of methanol and tetrahydrofuran (2:1), and 3.5 mg (88 μmol) of sodium borohydride are added. After one hour at room temperature, 1 ml of water is added and the reaction solution is filtered through a 1.1 g Extrelut/silica gel cartridge. The cartridge is eluted with dichloromethane and the filtrate is concentrated under reduced pressure. The residue is purified by preparative thick-layer chromatography (mobile phase: cyclohexane/ethyl acetate 2:1). This gives 14 mg (47% of theory) of product.

¹H-NMR (200 MHz, CDCl₃): δ=0.98 (dd, 6H), 1.05-2.42 (m, 18H), 3.99 (s, 3H), 4.03-4.46 (m, 2H), 4.96-5.18 (m, 3H), 7.00 (br. d, 1H), 7.30 (d, 1H), 7.53-7.63 (m, 2H) ppm.

MS (ESIpos): m/z=509 (M+H)⁺

HPLC (Method 1): Rₜ=5.66 min.

The examples listed in the table below are prepared analogously to the procedures described above, from the corresponding starting materials:

| Example | | |
|---|---|---|
| B- | Structure | Analytical data |
| 169 | 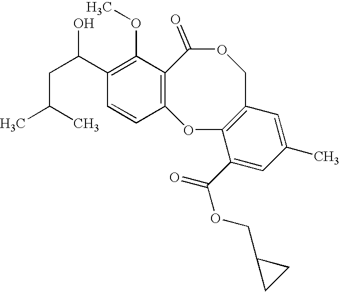 | ¹H-NMR(200 MHz, CDCl₃): δ = 0.33-0.4 1(m, 2H), 0.5 8-0.68 (m, 2H), 0.98(dd, 6H), 1.25-1.86 (m, 4H), 1.97(d, 1H), 2.32(s, 3H), 3.98(s, 3H), 4.20(d, 2H), 5.03-5.12(m, 3H), 7.00(s, 1H), 7.36(d, 1H), 7.57-7.6 1(m, 2H) ppm. HPLC(Method 1): Rₜ = 5.02 min. |
| 170 | 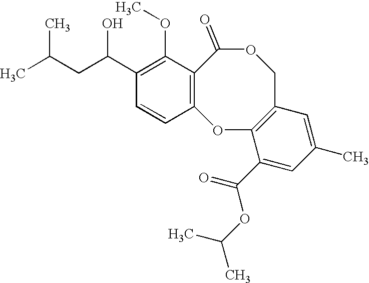 | LC-MS(Method 5): Rₜ = 2.93 min. MS(ESIpos): m/z = 443(M + H)⁺ |
| 171 | 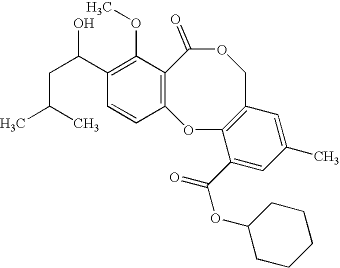 | ¹H-NMR(200 MHz, CDCl₃): δ = 0.98(dd, 6H), 1.25-2. 10(m, 14H), 2.32(s, 3H), 3.99(s, 3H), 4.97-5.16(m, 4H), 6.98(s, 1H), 7.31(d, 1H), 7.53(s, 1H), 7.60(d, 1H) ppm. MS(ESIpos): m/z = 483(M + H)⁺ |
| 172 | 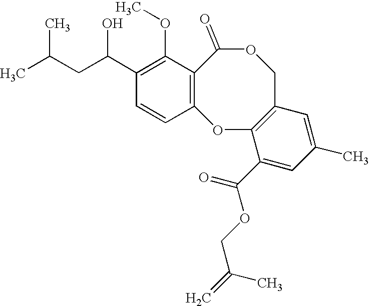 | LC-MS(Method 5): Rₜ = 2.93 min. MS(ESIpos): m/z = 455(M + H)⁺ |
| 173 | 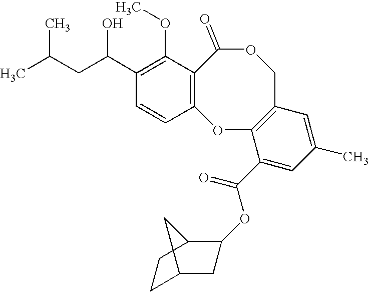 | LC-MS(Method 4): Rₜ = 5.29 min. MS(ESIpos): m/z = 517(M + Na)⁺ |
| 174 | 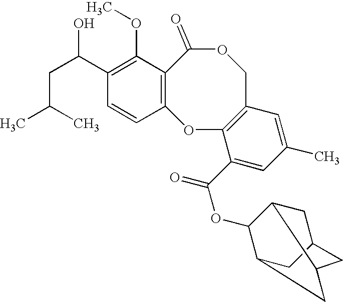 | LC-MS(Method 4): Rₜ = 5.68 min. MS(ESIpos): m/z = 557(M + Na)⁺ |
| 175 | 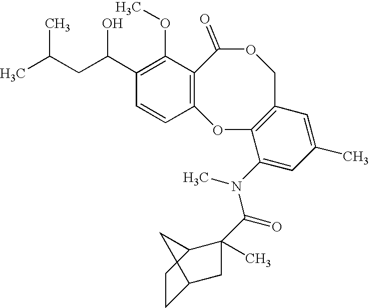 | ¹H-NMR(300 MHz, CDCl₃): δ = 0.98(dd, 6H), 1.14-2.00(m, 14H), 2.20-2.85(m, 3H), 2.27(s, 3H), 3.30 + 3.40(s, 3H), 3.98(s, 3H), 4.80-5.28(m, 3H), 6.79(br. s, 1H), 6.98(br. s, 1H), 6.71(d, 1H), 7.06(d, 1H), 7.55(d, 1H) ppm. MS(ESIpos): m/z = 522(M + H)⁺HPLC(Method 1): Rₜ = 5.21, 5.27 min. |

### EXAMPLE B-176

### 9-(Difluoromethyl)-3-[(1S)-1-hydroxy-3-methylbutyl]-11-(isopentyloxy)-4-methoxy-5H,7H-dibenzo[b,g][1,5]dioxocin-5-one

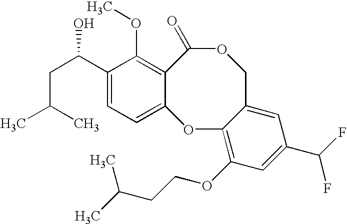

25 mg (0.04 mmol) of the compound from Example B-IL are dissolved in 5 ml of tetrahydrofuran, and 0.5 ml of a 1 M solution of tetra-n-butylammonium fluoride in tetrahydrofuran is added at room temperature. After 18 hours, 1 ml of saturated sodium chloride solution is added and the reaction solution is filtered through a 2 g Extrelut/silica gel cartridge. The cartridge is eluted with 5 ml of ethyl acetate and the filtrate is concentrated under reduced pressure. The residue is purified by preparative thick-layer chromatography (mobile phase: cyclohexane/ethyl acetate 4:1). This gives 10 mg (51% of theory) of product.

¹H-NMR (400 MHz, CDCl₃): δ=0.95-1.02 (m, 12H), 1.25-1.93 (m, 7H), 3.98 (s, 3H), 4.14 (t, 2H), 5.07-5.14 (m, 3H), 6.41-6.70 (m, 1H), 6.77 (s, 1H), 6.93 (d, 1H), 7.13 (s, 1H), 7.59 (d, 1H) ppm.

MS (ESIpos): m/z=501 (M+Na)⁺

### EXAMPLE B-177

### 8-(Difluoromethyl)-3-[(1S)-1-hydroxy-3-methylbutyl]-11-(isopentyloxy)-9-methyl-4-methoxy-5H,7H-dibenzo[b,g][1,5]dioxocin-5-one

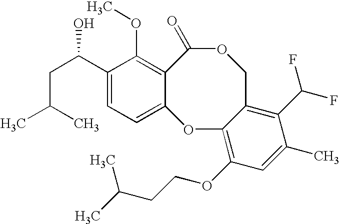

40 mg (0.066 mmol) of the compound from Example B-L are reacted analogously to Example B-176. This gives 32 mg (99% of theory) of product.

¹H-NMR (300 MHz, CDCl₃): δ=0.95-1.01 (m, 12H), 1.45-1.96 (m, 8H), 2.39 (s, 3H), 3.98 (s, 3H), 4.11 (t, 2H), 5.09 (dd, 1H), 5.46 (s, 2H), 6.75 (br. s, 1H), 6.87 (d, 1H), 7.56 (d, 1H) ppm.

MS (DCI): m/z=510 (M+NH₄)⁺

HPLC (Method 1): Rₜ=5.48 min.

Part C:

Starting Materials:

### EXAMPLE C-I

### 3-[(1S)-1-Hydroxy-3-methylbutyl]-11-(benzyloxy)-4-methoxy-9-methyl-5H,7H-dibenzo[b,g][1,5]dioxocin-5-one

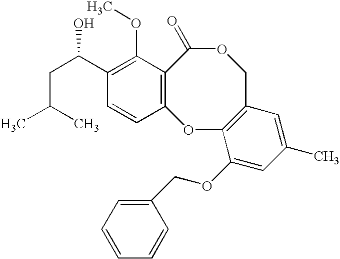

331 mg (0.889 mmol) of penicillide are reacted analogously to Example B-XLI. This gives 331 mg (81% of theory) of product.

¹H-NMR (200 MHz, CDCl₃): δ=0.97 (dd, 6H), 1.39-1.88 (m, 3H), 1.96 (d, 1H), 2.26 (s, 3H), 3.98 (s, 3H), 5.00-5.24 (m, 5H), 6.46 (m, 1H), 6.86 (m, 1H), 6.96 (d, 1H), 7.28-7.59 (m, 6H) ppm

MS (DCI): m/z=480 (M+NH₄)⁺

### EXAMPLE C-II

### 11-(Benzyloxy)-4-methoxy-9-methyl-3-(3-methylbutanoyl)-5H,7H-dibenzo[b,g]-[1,5]dioxocin-5-one

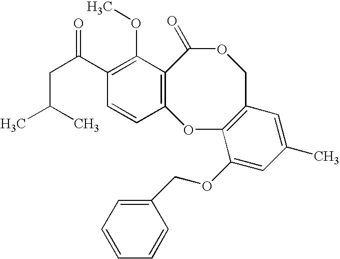

510 mg (1.103 mmol) of the compound from Example C-I are reacted analogously to Example A-XXIX. This gives 366 mg (72% of theory) of product.

¹H-NMR (300 MHz, CDCl₃): δ=0.95 (d, 6H), 2.21 (septet, 1H), 2.26 (s, 3H), 2.83 (d, 2H), 3.96 (s, 3H), 5.12 (s, 2H), 5.19 (s, 2H), 6.47 (m, 1H), 6.87 (m, 1H), 6.98 (d, 1H), 7.29-7.53 (m, 5H), 7.66 (d, 1H) ppm

MS (DCI): m/z=478 (M+NH₄)⁺

### EXAMPLE C-III

### 4-Methoxy-8-fluoro-11-(benzyloxy)-9-methyl-3-(3-methylbutanoyl)-5H,7H-dibenzo-[b,g][1,5]dioxocin-5-one

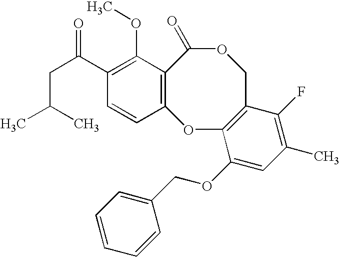

Under argon, 356 mg (0.773 mmol) of the compound from Example C-II are dissolved in 7.5 ml of dry acetonitrile, and 497 mg (0.773 mmol) of 1-fluoro-4-hydroxy-1,4-diazoniabicyclo[2.2.2]octane bistetrafluoroborate (50% on Al₂O₃) are added at room temperature. The mixture is stirred at 60° C. for 18 hours. After cooling to room temperature, the mixture is filtered through Celite and the Celite is washed with 20 ml of acetonitrile. The solvent is removed under reduced pressure and the residue is purified by preparative HPLC. This gives 77 mg (21% of theory) of product.

¹H-NMR (300 MHz, CDCl₃): δ=0.96 (d, 6H), 2.10-2.31 (m, 4H), 2.84 (d, 2H), 3.97 (s, 3H), 5.16 (s, 2H), 5.30 (s, 2H), 6.86 (d, 1H), 6.96 (d, 1H), 7.32-7.54 (m, 5H), 7.67 (d, 1H) ppm

MS (DCI): m/z=479 (M+H)⁺, 496 (M+NH₄)⁺

### EXAMPLE C-IV

### 8-Fluoro-11-hydroxy-4-methoxy-9-methyl-3-(3-methylbutanoyl)-5H,7H-dibenzo-[b,g][1,5]dioxocin-5-one

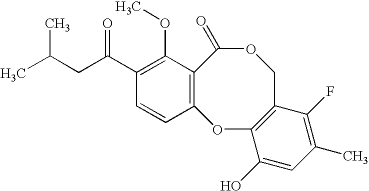

161 mg (0.336 mmol) of the compound from Example C-III are dissolved in 3.4 ml of dichloromethane and, under argon and at 0° C., 218 mg (1.346 mmol) of iron(III) chloride are added. After 1.25 hours of stirring at 0° C., 5 ml of saturated sodium bicarbonate solution are added and the reaction mixture is extracted twice with in each case 10 ml of ethyl acetate. The combined organic phases are washed with in each case 10 ml of water and saturated sodium chloride solution, dried over magnesium sulphate, filtered and concentrated under reduced pressure. The crude product is purified chromatographically (10 g silica gel cartridge; mobile phase: cyclohexane→cyclohexane/ethyl acetate 1:1). This gives 82 mg (63% of theory) of product.

R_{f}=0.23 (toluene/ethyl acetate 9:1)

¹H-NMR (200 MHz, CDCl₃): δ=0.96 (d, 6H), 2.07-2.34 (m, 4H), 2.84 (d, 2H), 3.98 (s, 3H), 5.29 (s, 2H), 5.80 (s, 1H), 6.87 (d, 1H), 6.93 (d, 1H), 7.07 (d, 1H) ppm

MS (DCI): m/z=406 (M+NH₄)⁺

### EXAMPLE C-V

### 8-Fluoro-11-hydroxy-10-iodo-4-methoxy-9-methyl-3-(3-methylbutanoyl)-5H,7H-dibenzo[b,g][1,5]dioxocin-5-one

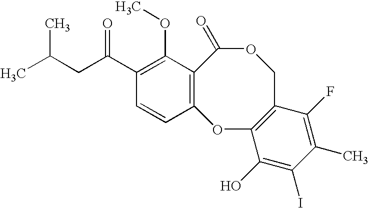

79 mg (0.203 mmol) of the compound from Example C-IV are suspended in 2 ml of ethanol and, under argon and at 0° C., 82 mg (0.305 mmol) of iron(III) chloride hexahydrate, dissolved in 1 ml of water, are added. 50 mg (0.305 mmol) of iodine monochloride, dissolved in about 0.25 ml of ethanol, are then added. After 18 hours of stirring at 22° C., a further 116 mg (0.715 mmol) of iodine monochloride are added and the mixture is stirred at this temperature for another 24 hours. For work-up, 5 ml of 10% strength sodium thiosulphate solution are added and the reaction mixture is extracted twice with in each case 10 ml of ethyl acetate. The combined organic phases are washed with in each case 10 ml of water and saturated sodium chloride solution, dried over magnesium sulphate, filtered and concentrated under reduced pressure. The crude product is once more subjected to the reaction conditions described above. After a further 24 hours, a further 16 mg (0.059 mmol) of iron(III) chloride hexahydrate, dissolved in 1 ml of water, and 20 μl of a 5 M solution of iodine monochloride in ethanol are added. After 24 hours, another 16 mg (0.059 mmol) of iron(III) chloride hexahydrate, dissolved in 1 ml of water, and 20 μl of a 5 M solution of iodine monochloride in ethanol are added. The mixture is stirred at 22° C. for another 24 hours. For work-up, 5 ml of a 10% strength sodium thiosulphate solution are added and the reaction mixture is extracted twice with in each case 10 ml of ethyl acetate. The combined organic phases are washed with in each case 10 ml of water and saturated sodium chloride solution, dried over magnesium sulphate, filtered and concentrated under reduced pressure. The residue is purified by preparative HPLC. This gives 20 mg (19% of theory) of product.

R_{f}=0.33 (toluene/ethyl acetate 9:1)

¹H-NMR (200 MHz, CDCl₃): δ=0.96 (d, 6H), 2.22 (sept, 1H), 2.35 (d, 2H), 2.84 (d, 2H), 3.98 (s, 3H), 5.26 (s, 2H), 6.42 (s, 1H), 6.96 (d, 1H), 7.69 (d, 1H) ppm

MS (DCI): m/z=515 (M+H)⁺, 532 (M+NH₄)⁺

### EXAMPLE C-VI

### 8-Fluoro-9,10-dimethyl-11-hydroxy-4-methoxy-3-(3-methylbutanoyl)-5H,7H-dibenzo[b,g][1,5]dioxocin-5-one

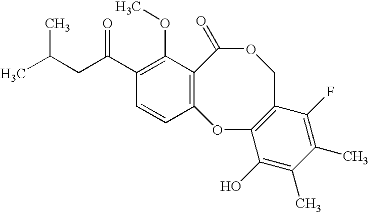

20 mg (0.039 mmol) of the compound from Example C-V are dissolved in 2 ml of dimethylformamide, and 162 μl (1.167 mmol) of tetramethyltin and 9 mg (0.008 mmol) of tetrakis(triphenylphosphine)palladium(0) are added under argon. The reaction vessel is closed and heated at 80° C. for 36 hours. The reaction mixture is then cooled to room temperature, 5 ml of water are added and the mixture is extracted four times in total with in each case 5 ml of ethyl acetate. The combined organic phases are dried over magnesium sulphate, filtered and concentrated. The residue is purified by preparative HPLC. This gives 13 mg (84% of theory) of product.

LC-MS (Method 7): Rₜ=3.62 min.

MS (ESIpos): m/z=403 (M+H)⁺

### EXAMPLE C-VII

### 4-Fluoro-8-methoxy-3-methyl-9-(3-methylbutanoyl)-7-oxo-5H,7H-dibenzo[b,g][1,5]dioxocin-1-yl 2-methylbicyclo[2.2.1]heptane-2-carboxylate

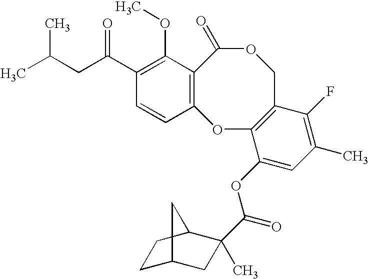

Under argon, 45 mg (0.116 mmol) of the compound from Example C-IV are dissolved in 1 ml of tetrahydrofuran, 26 μl (0.174 mmol) of 1,8-diazabicyclo[5.4.0]undec-7-ene and 30 mg (0.174 mmol) of 2-methyl-bicyclo[2.2.1]heptane-2-carbonyl chloride are added and the mixture is stirred at room temperature. After 18 hours, 1 ml of water and 5 drops of 1 N hydrochloric acid are added and the reaction mixture is diluted with 5 ml of ethyl acetate and filtered through an Extrelut cartridge. The cartridge is eluted with 40 ml of ethyl acetate and the filtrate is concentrated under reduced pressure. The residue is purified by preparative HPLC. This gives 17 mg (27% of theory) of product.

¹H-NMR (400 MHz, CDCl₃): δ=0.96 (d, 6H), 1.14-1.62 (m, 5H), 1.65-1.77 (m, 1H), 2.14-2.74 (m, 8H), 2.83 (d, 2H), 3.96 (s, 3H), 5.27 (m, 2H), 6.92 (m, 1H), 7.01 (m, 1H), 7.67 (d, 1H) ppm

MS (DCI): m/z=525 (M+H)⁺, 542 (M+NH₄)⁺.

The examples listed in the table below are prepared analogously to the procedures described above, from the corresponding starting materials:

| Example | | |
|---|---|---|
| C- | Structure | Analytical data |
| VIII | 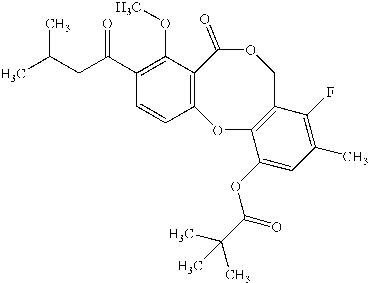 | ¹H-NMR(200 MHz, CDCl₃): δ = 0.96(d, 6H), 1.39(s, 9H), 2.06-2.36(m, 4H), 2.83(d, 2H), 3.96(s, 3H), 5.27(br. s, 2H), 6.89-7.03(m, 2H), 7.69(d, 1H) ppm |
| IX | 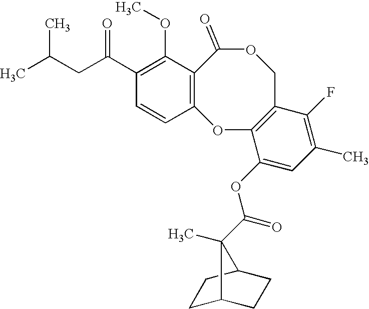 | ¹H-NMR(200 MHz, CDCl₃): δ = 0.96(d, 6H), 1.17-1.66(m, 6H), 1.69-2.07(m, 5H), 2.10-2.45(m, 6H), 2.84(d, 2H), 3.97 (s, 3H), 5.26(br. s, 2H), 6.90(d, 1H), 7.04(d, 1H), 7.69(d, 1H) ppm |
| X | 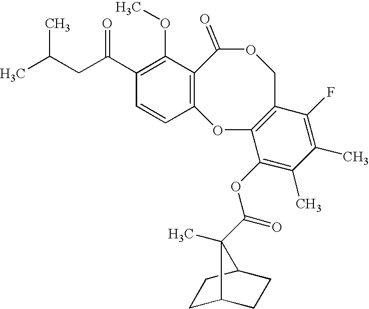 | ¹H-NMR(300 MHz, CDCl₃): δ = 0.96(a, 6H), 1.21-1.38(m, 4H), 1.72-2.03(m, 8H), 2.15(s, 6H), 2.22(sept, 1H), 2.34(m, 2H), 2.83(d, 2H), 3.96(s, 3H), 5.22(br. s, 2H), 7.04(d, 1H), 7.67(d, 1H) ppm MS(DCI): m/z = 539(M + H)⁺, 556(M + NH₄)⁺ |

### EXAMPLE C-XI

### 10-Iodo-11-hydroxy-3-[(1S)-1-hydroxy-3-methylbutyl]-4-methoxy-9-methyl-5H,7H-dibenzo[b,g][1,5]dioxocin-5-one

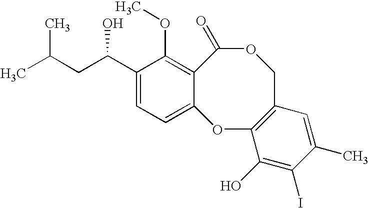

At 0° C., 500 mg (1.34 mmol) of penicillide are dissolved in 10 ml of ethanol/water (1:1) and 347 mg (1.48 mmol) of iron trichloride hexahydrate are added. 240 mg (1.48 mmol) iodine monochloride, dissolved in 1 ml of ethanol, are then added dropwise over a period of 30 minutes and the mixture is stirred at room temperature for 18 hours. The reaction solution is diluted with 10 ml of water and extracted twice with in each case 20 ml of dichloromethane. The combined organic phases are washed once with 10 ml of 10% strength sodium bisulphite solution and once with water. The organic phase is dried over sodium sulphate, filtered through a bed of silica gel and concentrated under reduced pressure. The crude product is purified chromatographically (silica gel; mobile phase: cyclohexane/ethyl acetate 100:0→2:1). This gives 494 mg (74% of theory) of product.

¹H-NMR (300 MHz, CDCl₃): δ=0.98 (t, 6H), 1.48 (m, 1H), 1.62-1.87 (m, 2H), 2.00 (d, 1H), 2.39 (s, 3H), 3.98 (s, 3H), 5.02-5.13 (m, 3H), 6.55 (s, 1H), 6.69 (s, 1H), 6.91 (d, 1H), 7.58 (d, 1H) ppm

MS (DCI): m/z=516 (M+NH₄)⁺

HPLC (Method 1): Rₜ=4.91 min.

### EXAMPLE C-XII

### 9,10-Dimethyl-11-hydroxy-3-[(1S)-1-hydroxy-3-methylbutyl]-4-methoxy-5H,7H-dibenzo[b,g][1,5]dioxocin-5-one

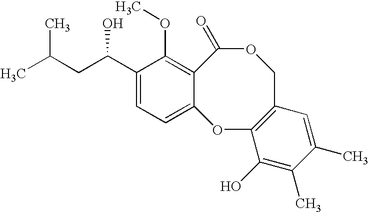

The preparation is carried out analogously to Example B-IV using 200 mg (0.401 mmol) of the compound from Example C-XI. This gives 83 mg (54% of theory) of product.

¹H-NMR (300 MHz, CDCl₃): δ=0.98 (t, 6H), 1.49 (m, 1H), 1.63-1.87 (m, 2H), 1.99 (m, 1H), 2.18 (s, 3H), 2.21 (s, 3H), 3.98 (s, 3H), 5.02-5.13 (m, 3H), 6.19 (br. s, 1H), 6.37 (s, 1H), 6.88 (d, 1H), 7.57 (d, 1H) ppm

MS (DCI): m/z=404 (M+NH₄)⁺

HPLC (Method 1): Rₜ=4.83 min.

### EXAMPLE C-XIII

### 11-Hydroxy-3-[(1S)-1-hydroxy-3-methylbutyl]-4-methoxy-10-ethyl-9-methyl-5H,7H-dibenzo[b,g][1,5]dioxocin-5-one

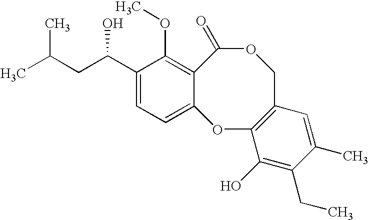

The preparation is carried out analogously to Example B-IV using 100 mg (0.20 mmol) of the compound from Example C-XI and 1.19 ml (6.0 mmol) of tetraethyltin. This gives 32 mg (40% of theory) of product.

¹H-NMR (300 MHz, CDCl₃): δ=0.98 (t, 6H), 1.15 (t, 3H), 1.48 (m, 1H), 1.62-1.87 (m, 2H), 1.97 (m, 1H), 2.22 (s, 3H), 2.70 (q, 2H), 3.98 (s, 3H), 5.01-5.13 (m, 3H), 6.13 (s, 1H), 6.37 (s, 1H), 6.90 (d, 1H), 7.57 (d, 1H) ppm

MS (ESIneg): m/z=399 (M−H)⁻

HPLC (Method 1): Rₜ=4.83 min.

### EXAMPLE C-XIV

### 11-Hydroxy-3-[(1S)-1-hydroxy-3-methylbutyl]-4-methoxy-8,9-dimethyl-5H,7H-dibenzo[b,g][1,5]dioxocin-5-one

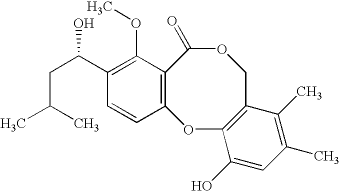

Example C-XIV is prepared analogously to Example B-IV using 200 mg (0.44 mmol) of the compound from Example B-II and 1.84 ml (13.3 mmol) of tetramethyltin. This gives 167 mg (97% of theory) of product.

¹H-NMR (200 MHz, CDCl₃): δ=0.96 (d, 3H), 0.99 (d, 3H), 1.40-1.59 (m, 1H), 1.60-1.89 (m, 2H), 2.01 (br. d, 1H), 2.05 (s, 3H), 2.21 (s, 3H), 4.00 (s, 3H), 5.03-5.14 (m, 1H), 5.18-5.36 (m, 2H), 5.95 (s, 1H), 6.82-6.89 (m, 2H), 7.57 (d, 1H) ppm LC-MS (Method 5): Rₜ=3.79 min.

MS (ESIpos): m/z=450 (M+Na+CH₃CN)⁺.

### EXAMPLE C-XV

### 8-Bromo-10-chloro-11-hydroxy-3-[(1S)-1-hydroxy-3-methylbutyl]-4-methoxy-9-methyl-5H,7H-dibenzo[b,g][1,5]dioxocin-5-one

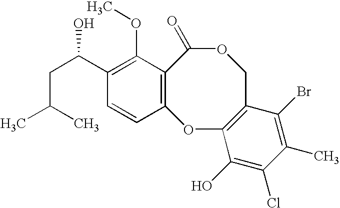

Example C-XV is prepared analogously to Example A-XLI using 150 mg (0.33 mmol) of the compound from Example A-II, 49 mg (0.37 mmol) of N-chloro-succinimide and 87 mg (0.32 mmol) of iron(III) chloride hexahydrate. This gives 65 mg (40% of theory) of product.

¹H-NMR (300 MHz, CDCl₃): δ=0.96 (d, 3H), 0.99 (d, 3H), 1.39-1.90 (m, 3H), 2.09 (br. s, 1H), 2.51 (s, 3H), 3.99 (s, 3H), 5.08 (dd, 1H), 5.33-5.55 (m, 2H), 6.62 (s, 1H), 6.86 (d, 1H), 7.58 (d, 1H) ppm

LC-MS (Method 4): Rₜ=4.45 min.

MS (ESIneg): m/z=483 (M−H)⁻.

### EXAMPLE C-XVI

### 10-Chloro-11-hydroxy-3-[(1S)-1-hydroxy-3-methylbutyl]-4-methoxy-8,9-dimethyl-5H,7H-dibenzo[b,g][1,5]dioxocin-5-one

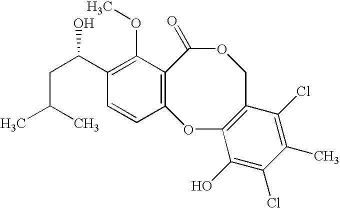

Example C-XVI is prepared analogously to Example B-IV using 140 mg (0.23 mmol) of the compound from Example C-XV and 0.48 ml (3.46 mmol) of tetramethyltin. The yield is 71 mg (73% of theory).

¹H-NMR (300 MHz, CDCl₃): δ=0.97 (d, 3H), 0.99 (d, 3H), 1.43-1.53 (m, 1H), 1.63-1.73 (m, 1H), 1.75-1.88 (m, 1H), 2.02 (br. s, 1H), 2.12 (s, 3H), 2.32 (s, 3H), 3.99 (s, 3H), 5.08 (dd, 1H), 5.19-5.31 (m, 2H), 6.24 (s, 1H), 6.89 (d, 1H), 7.58 (d, 1H) ppm

LC-MS (Method 4): Rₜ=3.58 min.

MS (ESIneg): m/z=419 (M−H)⁻.

### EXAMPLE C-XVII

### 10-Chloro-11-hydroxy-3-[(1S)-1-hydroxy-3-methylbutyl]-4-methoxy-9-methyl-5H,7H-dibenzo[b,g][1,5]dioxocin-5-one

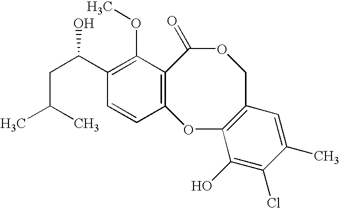

67 mg (0.14 mmol) of the compound from Example C-XV are dissolved in isopropanol (6.7 ml), and 3.9 mg (0.01 mmol) of bis(triphenylphosphine)-palladium(II) chloride and 55.1 mg (0.21 mmol) of potassium phosphate trihydrate are added. The mixture is heated under reflux for 2 h. After cooling, the mixture is filtered through kieselguhr, the kieselguhr is washed with isopropanol and the solvent is removed under reduced pressure. The residue is purified by preparative HPLC. This gives 44 mg (78% of theory) of product.

¹H-NMR (400 MHz, CDCl₃): δ=0.97 (d, 3H), 0.99 (d, 3H), 1.43-1.52 (m, 1H), 1.64-1.72 (m, 1H), 1.74-1.85 (m, 1H), 2.03 (d, 1H), 2.31 (s, 3H), 3.98 (s, 3H), 5.02-5.12 (m, 3H), 6.39 (br. s, 1H), 6.49 (s, 1H), 6.91 (d, 1H), 7.58 (d, 1H) ppm LC-MS (Method 4): Rₜ=3.31 min.

MS (ESIneg): m/z=405 (M−H)⁻.

### EXAMPLE C-XVIII

### 8-Chloro-11-hydroxy-3-[(1S)-1-hydroxy-3-methylbutyl]-4-methoxy-9-methyl-10-vinyl-5H,7H-dibenzo[b,g][1,5]dioxocin-5-one

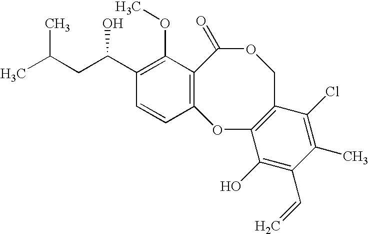

In a Schlenk flask, 400 mg (0.82 mmol) of the compound from Example B-LIII are weighed in under argon and dissolved in 16 ml of dimethylformamide. 218 mg (0.19 mmol) of tetrakis(triphenylphosphine)palladium(0) and 4.81 ml (16.5 mmol) of tributylvinyltin are added. With: stirring, the mixture is heated at 120° C. overnight. Water is then added, the mixture is extracted with ethyl acetate and the catalyst is filtered off. The filtrate is concentrated and the residue is purified by preparative HPLC. This gives 185 mg (51.9% of theory) of product.

¹H-NMR (200 MHz, CDCl₃): δ=0.96 (d, 3H), 0.99 (d, 3H), 1.40-1.90 (m, 3H), 2.00 (br. s, 1H), 2.33 (s, 3H), 3.98 (s, 3H), 5.10 (m, 1H), 5.41 (d, 1H), 5.49 (d, 1H), 5.17-5.23 (m, 2H), 6.32 (s, 1H), 6.72 (dd, 1H), 6.86 (d, 1H), 7.60 (d, 1H) ppm

HPLC (Method 1): Rₜ=5.2 min.

MS (DCI): m/z=450 (M+NH₄)⁺.

### EXAMPLE C-XIX

### 8-Chloro-11-hydroxy-3-[(1S)-1-hydroxy-3-methylbutyl]-4-methoxy-9-methyl-10-ethyl-5H,7H-dibenzo[b,g][1,5]dioxocin-5-one

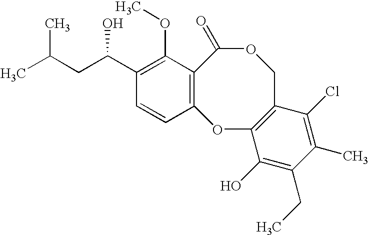

Example C-XIX is prepared analogously to Example B-IV using 60 mg (0.12 mmol) of the compound from Example B-LIII, 33 mg (0.03 mmol) of tetrakis(triphenylphosphine)palladium(0) and 435 mg (1.85 mmol) of tetraethyltin. The yield is 7.7 mg (14% of theory).

¹H-NMR (200 MHz, CDCl₃): δ=0.98 (dd, 6H), 1.13 (t, 3H), 1.38-1.88 (m, 3H), 2.05-2.20 (br. s, 1H), 2.30 (s, 3H), 2.78 (q, 2H), 3.98 (s, 3H), 5.02-5.14 (m, 1H), 5.41 (dd, 2H), 6.34 (s, 1H), 6.82 (d, 1H), 7.57 (d, 1H) ppm.

### EXAMPLE C-XX

### 8-Chloro-11-hydroxy-3-[(1S)-1-hydroxy-3-methylbutyl]-4-methoxy-9-methyl-10-propyl-5H,7H-dibenzo[b,g][1,5]dioxocin-5-one

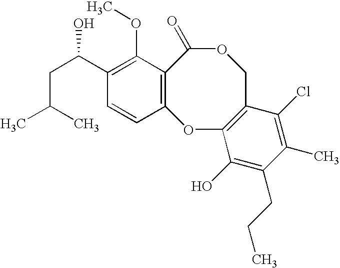

Example C-XX is prepared analogously to Example B-IV using 60 mg (0.124 mmol) of the compound from Example B-LIII, 33 mg (0.028 mmol) of tetrakis(triphenylphosphine)palladium(0) and 539 mg (1.853 mmol) of tetra-n-propyltin. The yield is 18.4 mg (33% of theory).

¹H-NMR (300 MHz, CDCl₃): δ=0.95-1.05 (m, 9H), 1.43-1.88 (m, 5H), 1.92 (d, 1H), 2.30 (s, 3H), 2.70-2.78 (m, 2H), 3.98 (s, 3H), 5.06-5.13 (m, 1H), 5.42 (dd, 2H), 6.08 (s, 1H), 6.82 (d, 1H), 7.60 (d, 1H) ppm.

### EXAMPLE C-XXI

### 8-Chloro-10-cyclopropyl-11-hydroxy-3-[(1S)-1-hydroxy-3-methylbutyl]-4-methoxy-9-methyl-5H,7H-dibenzo[b,g][1,5]dioxocin-5-one

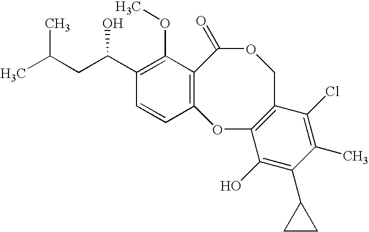

### a) Preparation of 2,4,6-tricyclopropylboroxine

0.79 ml (7 mmol) of trimethyl borate are dissolved in 7 ml of THF and, at −78° C., 14 ml (7 mmol) of a 0.5 M solution of cyclopropyl magnesium bromide in THF are added. The solution is then thawed and stirred at room temperature for 15 min, and 40 ml of 1 M hydrochloric acid are then added. The mixture is extracted with ethyl acetate, the organic extract is concentrated and the residue is directly used further without further purification.

Yield: 537 mg (37% of theory)

GC-MS (Method 9): Rₜ=5.47 min., m/z=204 (M)⁺.

### b) Cyclopropylation

In a Schlenk flask, dried by heating and flushed with argon, 432 mg (0.89 mmol) of the compound from Example B-LIII, 950 mg (4.67 mmol) of 2,4,6-tricyclopropylboroxine, 31 mg (0.03 mmol) of tetrakis(triphenylphosphine)-palladium(0) and 378 mg (1.78 mmol) of potassium phosphate are dissolved in 4.8 ml of toluene and heated at reflux overnight. The mixture is then applied to silica gel and purified chromatographically (Biotage 25S silica gel, mobile phase cyclohexane/ethyl acetate 3+1). This gives 263 mg (56% of theory) of product.

¹H-NMR (200 MHz, CDCl₃): δ=0.51-0.78 (m, 4H), 0.96 (d, 3H), 0.99 (d, 3H), 1.10 (m, 1H), 1.40-2.00 (m, 3H), 2.42 (s, 3H), 4.00 (s, 3H), 5.10 (m, 1H), 5.39 (d, 1H), 5.45 (d, 1H), 6.18 (s, 1H), 6.86 (d, 1H), 7.59 (d, 1H) ppm

HPLC (Method 1): Rₜ=5.3 min.

MS (DCI): m/z=464 (M+NH₄)⁺.

The examples listed in the table below are prepared analogously to the procedures described above, from the corresponding starting materials:

| Example | | |
|---|---|---|
| C- | Structure | Analytical data |
| XXII | 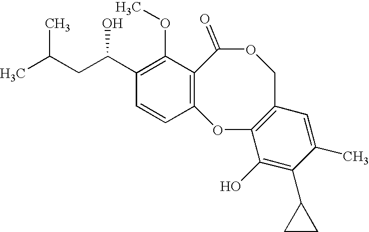 | MS(DCI): m/z = 430 (M + NH₄)⁺ |
| XXIII | 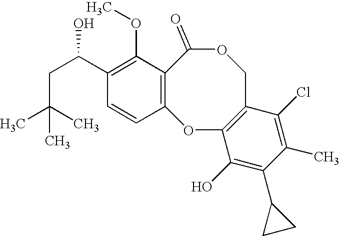 | MS(DCI): m/z = 478 (M + NH₄)⁺LC-MS(Method 5): m/z = 459 [M − H]⁻, Rₜ = 3.77 min. |

### EXAMPLE C-XXIV

### 3-Bromo-2-hydroxy-5-methylbenzoic acid

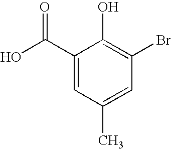

30.0 g (197 mmol) of 2-hydroxy-5-methylbenzoic acid are dissolved in 300 ml of glacial acetic acid. With ice-cooling, 11.17 ml (34.66 g, 217 mmol) of bromine are added. The mixture is then heated at room temperature and stirred for 1 hour. Ice-water is then added and the mixture is allowed to stand in an ice-bath to bring the crystallization to completion. The crystals are filtered off, washed with water until colourless and dried under reduced pressure.

Yield: 40.20 g (purity 83%, 73% of theory)

¹H-NMR (200 MHz, CDCl₃): δ=2.30 (s, 3H), 7.59 (s, 1H), 7.68 (s, 1H), 10.87 (s, 1H) ppm

HPLC (Method 1): Rₜ=4.3 min.

MS (DCI): m/z=248/250 (M+NH₄)⁺.

### EXAMPLE C-XXV

### 2,3-Dihydroxy-5-methylbenzoic acid

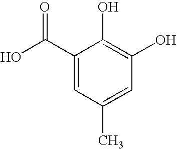

The preparation is carried out analogously to D. D. Weller, E. P. Stirchak, J. Org. Chem. 48, 4873-4879 (1983):

500 ml of water are degassed under reduced pressure and flushed with argon. 56 g (1.4 mol) of sodium hydroxide are then dissolved in this water. 0.57 g (2.31 mmol) of copper(II) sulphate pentahydrate is added, and the mixture is stirred at room temperature for about 20 min. 12.00 g (52 mmol) of the compound from Example C-XXIV are initially charged in a flask which has been evacuated beforehand and then vented with argon, and the aqueous solution of copper sulphate and sodium hydroxide is added. The mixture is heated at reflux overnight. The mixture is then allowed to cool and, with ice-cooling, acidified with concentrated hydrochloric acid. The mixture is extracted with 5 portions of ethyl acetate and the combined organic extracts are boiled with activated carbon for 40 min and then concentrated. The residue contains the product in a purity of 68%.

Yield: 5.63 g (44% of theory)

¹H-NMR (200 MHz, CDCl₃): δ=2.30 (s, 3H), 6.58-6.77 (m, 2H), 7.00 (d, 1H), 7.21 (d, 1H), 10.39 (s, 1H) ppm

HPLC (Method 1): Rₜ=3.4 min.

MS (DCI): m/z=186 (M+NH₄)⁺.

### EXAMPLE C-XXVI

### 3-(Benzyloxy)-2-hydroxy-5-methylbenzoic acid

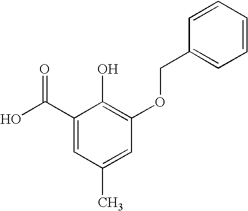

19.00 g (86 mmol) of the compound from Example C-XXV are dissolved in 491 ml of DMF and cooled to 0° C. 11.31 g of sodium hydride (60% suspension in paraffin, 283 mmol) are added, and the mixture is stirred at room temperature. Once the evolution of gas has ceased, after about 30 minutes, 11.21 ml (94.22 mmol) of benzyl bromide are added. The mixture is stirred at 60° C. for 3 h. 100 ml of water are then added and the mixture is, with cooling, acidified to pH 3 using about 70 ml of 6 M hydrochloric acid and then extracted with ethyl acetate. After phase separation, the organic phase is dried over sodium sulphate and concentrated. The crude product is chromatographed on silica gel (mobile phase: dichloromethane/cyclohexane 1:1, then dichloromethane/methanol 100:1).

Yield: 16.14 g (purity 78%, 57% of theory)

¹H-NMR (200 MHz, CDCl₃): δ=2.25 (s, 3H), 5.12 (s, 2H), 6.90 (d, 1H), 7.21-7.50 (m, 6H), 8.00 (s, 1H), 11.37 (s, 1H) ppm

HPLC (Method 1): Rₜ=4.5 min.

MS (DCI): m/z=276 (M+NH₄)⁺.

### EXAMPLE C-XXVII

### 2-(Benzyloxy)-6-(hydroxymethyl)-4-methylphenol

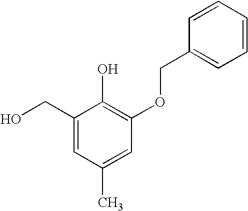

Under argon, 115 g (445.3 mmol) of the compound from Example C-XXVI are initially charged in 4.4 l of tetrahydrofuran. With ice cooling, 1.34 l (1.34 mol) of a 1 M solution of lithium aluminium hydride in tetrahydrofuran are then added dropwise at room temperature. The mixture is stirred at room temperature for 30 min and under reflux for 1 hour. The mixture is cooled and carefully diluted with ethyl acetate (2 l), 1 l of water is added and the mixture is then acidified with 1 N hydrochloric acid (about 4 l). Following addition of 900 g of solid sodium chloride, another 8 l of ethyl acetate are added, the phases are separated and the ethyl acetate phase is dried and concentrated. The residue is purified by flash chromatography on 3 kg of silica gel (mobile phase: cyclohexane/ethyl acetate 2:1→1:1). This gives 72 g (66% of theory) of product.

¹H-NMR (300 MHz, CDCl₃): δ=2.28 (s, 3H), 4.69 (s, 3H), 5.08 (s, 3H), 5.81 (br. s, 1H), 6.70 (s, 1H), 6.72 (s, 1H), 7.31-7.46 (m, 5H) ppm.

### EXAMPLE C-XXVIII

### 2-(Benzyloxy)-4-methyl-6-[(tetrahydro-2H-pyran-2-yloxy)methyl]phenol

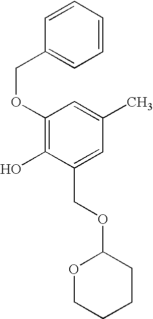

3.20 g (13.10 mmol) of the compound from Example C-XXVII are dissolved in 150 ml of dichloromethane and cooled to 0° C. 70 mg (0.39 mmol) of p-toluenesulphonic acid are added, and 1.43 ml (15.72 mmol) of 3,4-dihydro-2H-pyran are then slowly added dropwise with vigorous stirring. The mixture is stirred at 0° C. until the reaction has gone to completion (monitored by TLC). Saturated sodium bicarbonate solution is then added to the mixture, with vigorous stirring. A little water is added, and the mixture is extracted with dichloromethane. The organic extract is dried over sodium sulphate and concentrated.

Yield: 4.18 g (purity 79%, 77% of theory)

HPLC (Method 1): Rₜ=5.1 min.

### EXAMPLE C-XXIX

### Methyl 6-{2-(benzyloxy)-4-methyl-6-[(tetrahydro-2H-pyran-2-yloxy)methyl]phenoxy}-3-formyl-2-methoxybenzoate

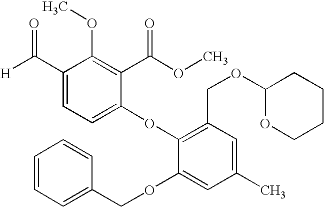

Example C-XXIX is prepared analogously to Example B-XVIII using 10 g (36.6 mmol) of methyl 6-bromo-3-formyl-2-methoxybenzoate and 18 g (54.9 mmol) of the compound from Example C-XXVIII. The yield is 15.1 g (79% of theory).

¹H-NMR (200 MHz, CDCl₃): δ=1.31-1.75 (m, 6H), 2.35 (s, 3H), 3.39-3.87 (m, 2H), 3.95 (s, 3H), 3.99 (s, 3H), 4.36-4.74 (m, 3H), 5.00 (s, 2H), 6.45 (d, 1H), 6.81 (m, 1H), 6.92 (m, 1H), 7.07-7.32 (m, 5H), 7.70 (d, 1H), 10.21 (s, 1H) ppm

HPLC (Method 1): Rₜ=5.35 min.

MS (DCI): m/z=538 (M+NH₄)⁺.

### EXAMPLE C-XXX

### Methyl 6-{2-(benzyloxy)-6-[(tetrahydro-2H-pyran-2-yloxy)methyl]phenoxy}-3-(1-hydroxy-3,3′-dimethylbutyl)-2-methoxybenzoate

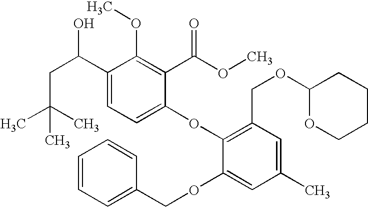

To prepare the Grignard reagent, 752 mg (30.9 mmol) of magnesium turnings are initially charged and dried by heating under reduced pressure, and after cooling under argon, an iodine crystal is added and heated until iodine vapours become visible. Then, after cooling, the mixture is covered with 15 ml of dry diethyl ether. A few drops of neopentyl bromide are then added, and the mixture is heated until the reaction starts. The remaining neopentyl bromide [5.39 ml (30.9 mmol) in total], dissolved in 15 ml of diethyl ether, is added dropwise, and the mixture is then heated in an oil bath under reflux for another 30 minutes until most of the magnesium has dissolved. After cooling, this Grignard solution is added to a solution, cooled to −78° C., of 6.44 g (12.4 mmol) of the compound from Example C-XXIX in 75 ml of tetrahydrofuran. After 3 hours at −78° C., the reaction solution is hydrolysed with saturated ammonium chloride solution. The mixture is diluted with water and extracted with diethyl ether. The combined organic phases are washed with saturated sodium chloride solution, dried over magnesium sulphate, filtered through silica gel and concentrated under reduced pressure. 6.25 g (85% of theory) of the title compound are isolated.

¹H-NMR (200 MHz, CDCl₃): δ=1.00 (s, 9H), 1.35-1.71 (m, 9H), 2.35 (s, 3H), 3.40-3.87 (m, 2H), 3.80 (s, 3H), 3.91 (s, 3H), 4.37-4.77 (m, 3H), 4.99 (s, 2H), 5.09 (m, 1H), 6.34 (d, 1H), 6.79 (m, 1H), 6.93 (m, 1H), 7.10-7.33 (m, 6H), 7.70 (d, 1H) ppm

HPLC (Method 2): Rₜ=5.72 min.

MS (DCI): m/z=610 (M+NH₄)⁺.

### EXAMPLE C-XXXI

### Methyl 6-[2-(benzyloxy)-6-(hydroxymethyl)phenoxy]-3-(1-hydroxy-3,3′-dimethyl-butyl)-2-methoxybenzoate

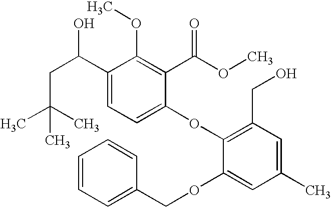

21.8 g (36.7 mmol) of the compound from Example C-XXX are dissolved in 150 ml of methanol, 32 mg (0.18 mmol) of p-toluenesulphonic acid, dissolved in 40 ml of water, are added, and the mixture is heated at 70° C. for 3 hours. After cooling to room temperature, 10 ml of saturated sodium bicarbonate solution are added and the mixture is concentrated under reduced pressure. The crude mixture is taken up in about 50 ml of water and extracted four times with in each case 50 ml of ethyl acetate. The combined organic phases are dried over sodium sulphate, filtered through silica gel and concentrated under reduced pressure. The crude product is purified chromatographically (silica gel, mobile phase: toluene/ethyl acetate 100:0→30:70 over a period of 80 min, flow rate: 80 ml/min). This gives 11.5 g (61% of theory) of product.

¹H-NMR (200 MHz, CDCl₃): δ=1.00 (s, 9H), 1.52-1.77 (m, 3H), 2.35 (s, 3H), 2.68 (t, 1H), 3.89 (s, 3H), 3.94 (s, 3H), 4.55 (d, 2H), 5.01 (s, 2H), 5.10 (m, 1H), 6.47 (d, 1H), 6.80 (m, 1H), 6.85 (m, 1H), 7.12-7.33 (m, 6H), 7.70 (d, 1H) ppm

MS (DCI): m/z=526 (M+NH₄)⁺.

### EXAMPLE C-XXXII

### 6-[2-(Benzyloxy)-6-(hydroxymethyl)phenoxy]-3-(1-hydroxy-3,3′-dimethylbutyl)-2-methoxybenzoic acid

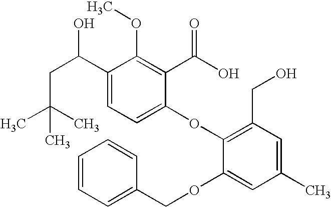

5.5 g (10.8 mmol) of the compound from Example C-XXXI are dissolved in 150 ml of methanol, 12.1 g (216.5 mmol) of potassium hydroxide are added and the mixture is heated under reflux for 9 hours. After cooling to room temperature, the mixture is concentrated under reduced pressure and then taken up in 50 ml of water. The mixture is extracted twice with in each case 50 ml of dichloromethane and the combined organic phases are dried over sodium sulphate, filtered and concentrated under reduced pressure. The crude product is reacted further without further purification. This gives 5.35 g (100% of theory) of product.

HPLC (Method 1): Rₜ=4.76 min.

MS (ESIpos): m/z=495 (M+H)⁺.

### EXAMPLE C-XXXIII

### 11-(Benzyloxy)-3-(1-hydroxy-3,3′-dimethylbutyl)-4-methoxy-5H,7H-dibenzo[b,g]-[1,5]dioxocin-5-one

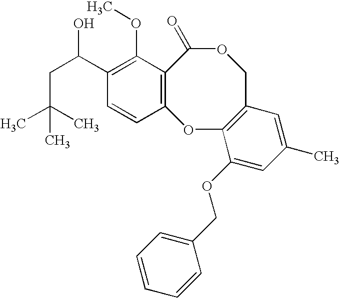

3.11 g (6.29 mmol) of the compound from Example C-XXXII are dissolved in 20 ml of acetonitrile, and 5.47 ml (39.26 mmol) of triethylamine are added. This solution is, using a syringe pump, metered under argon into a solution, heated at 70° C., of 5.02 g (19.63 mmol) of 2-chloro-1-methylpyridinium iodide in 210 ml of acetonitrile, over a period of 8 hours. After the addition, the mixture is stirred at 70° C. for 8 hours. After cooling, the reaction solution is concentrated under reduced pressure. The residue is taken up in dichloromethane and washed three times with water. The organic phase is dried over sodium sulphate and concentrated under reduced pressure. The residue is purified chromatographically on silica gel (mobile phase: cyclohexane/ethyl acetate 100:0→10:90). This gives 2.40 g (80% of theory) of product.

¹H-NMR (200 MHz, CDCl₃): δ=1.01 (s, 9H), 1.49-1.73 (m, 2H), 1.83 (d, 1H), 2.26 (s, 3H), 2.68 (t, 1H), 3.99 (s, 3H), 4.55 (d, 2H), 5.04-5.24 (m, 5H), 6.46 (m, 1H), 6.85 (m, 1H), 6.95 (d, 1H), 7.30-7.54 (m, 5H), 7.57 (d, 1H) ppm

MS (DCI): m/z=494 (M+NH₄)⁺.

### EXAMPLE C-XXXIV

### 11-(Benzyloxy)-4-methoxy-9-methyl-3-(3,3′-dimethylbutanoyl)-5H,7H-dibenzo[b,g]-[1,5]dioxocin-5-one

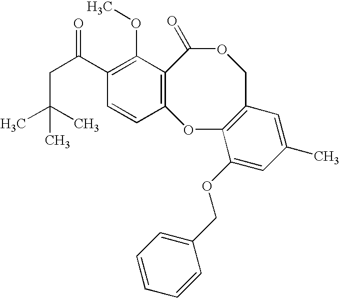

Example C-XXXIV is prepared analogously to Example A-XXIX using 2.05 g (4.30 mmol) of the compound from Example C-XXXII, 1.85 g (8.59 mmol) of pyridinium chlorochromate and 0.88 g (8.59 mmol) of basic alumina. The yield is 1.70 g (83% of theory).

¹H-NMR (300 MHz, CDCl₃): δ=1.00 (s, 9H), 2.27 (s, 3H), 2.90 (s, 2H), 3.96 (s, 3H), 5.10 (s, 2H), 5.19 (s, 2H), 6.47 (m, 1H), 6.88 (m, 1H), 6.98 (d, 1H), 7.30-7.53 (m, 5H), 7.59 (d, 1H) ppm

HPLC (Method 2): Rₜ=5.58 min.

MS (DCI): m/z=475 (M+H)⁺, 492 (M+NH₄)⁺

### EXAMPLE C-XXXV

### 11-Hydroxy-4-methoxy-9-methyl-3-(3,3′-dimethylbutanoyl)-5H,7H-dibenzo[b,g]-[1,5]dioxocin-5-one

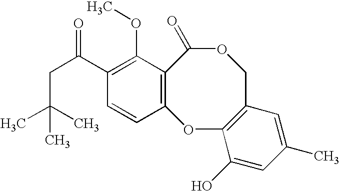

Example C-XXXV is prepared analogously to Example C-IV using 1.42 g (2.99 mmol) of the compound from Example C-XXXIV and 1.45 g (8.96 mmol) of iron trichloride. The yield is 0.72 g (63% of theory).

¹H-NMR (300 MHz, CDCl₃): δ=1.00 (s, 9H), 2.26 (s, 3H), 2.90 (s, 2H), 3.97 (s, 3H), 5.10 (s, 2H), 5.98 (s, 1H), 6.41 (m, 1H), 6.68 (d, 1H), 6.89 (m, 1H), 7.95 (d, 1H) ppm

MS (DCI): m/z=385 (M+H)⁺, 402 (M+NH₄)⁺.

### EXAMPLE C-XXXVI

### 11-Hydroxy-3-[(1S)-hydroxy-3,3′-dimethylbutyl]-4-methoxy-9-methyl-5H,7H-dibenzo[b,g][1,5]dioxocin-5-one

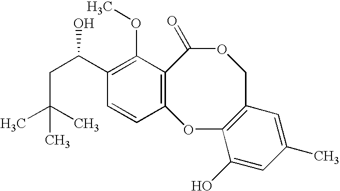

Under argon, 98 mg (0.66 mmol) of (1R,2S)-aminoindan-2-ol are initially charged in 32 ml of tetrahydrofuran, and 5.85 ml (32.88 mmol) of borane/N,N-diethylaniline complex are added. The mixture is stirred at room temperature for 30 minutes and then cooled to 0° C., and 3.16 g (8.2 mmol) of the compound from Example C-XXXV, dissolved in 32 ml of tetrahydrofuran, are added. With stirring, the mixture is, over a period of 16 hours, warmed to room temperature. 15 ml of methanol are then added dropwise with vigorous stirring, the solution is concentrated under reduced pressure and the crude product is chromatographed on silica gel (mobile phase: cyclohexane, then cyclohexane/ethyl acetate 4:1→3:2). This gives 2.642 g (83% of theory) of the product as a mixture of enantiomers. Subsequent chromatographic separation of the enantiomers on a chiral phase [Chiralpak AD, 350×30 mm, 20 μm; mobile phase: isopropanol/methanol+0.2% diethylamine 90:10; flow rate: 100 ml/min; room temperature; detection: 220 nm] gives 1.65 g (52% of theory) of the title compound.

Rₜ=8.17 min. [column: Chiralpak AD, mobile phase: isopropanol/methanol/diethylamine 83:17:0.2; flow rate: 1.0 ml/min; detection: 250 nm]

¹H-NMR (300 MHz, CDCl₃): δ=1.03 (s, 9H), 1.51-1.72 (m, 2H), 1.85 (d, 1H), 2.26 (s, 3H), 3.97 (s, 3H), 4.99 (s, 2H), 5.18 (m, 1H), 6.00 (br. s, 1H), 6.39 (m, 1H), 6.82-6.91 (m, 2H), 7.51 (d, 1H) ppm

MS (DCI): m/z=404 (M+NH₄)⁺.

### EXAMPLE C-XXXVII

### 8-Chloro-9-methyl-11-hydroxy-3-[(1S)-hydroxy-3,3′-dimethylbutyl]-4-methoxy-5H,7H-dibenzo[b,g][1,5]dioxocin-5-one

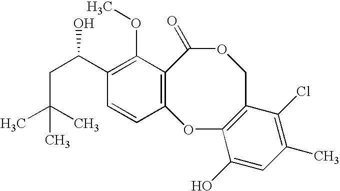

1.52 g (3.94 mmol) of the compound from Example C-XXXVI are dissolved in 15 ml of ethanol and 15 ml of water. 553 mg (4.14 mmol) of N-chlorosuccinimide are added, followed by addition of 1.034 g (3.83 mmol) of iron(III) chloride hexahydrate. The mixture is stirred at room temperature for 2 days and then diluted with twice the amount of water. The aqueous phase is extracted four times with ethyl acetate and the organic phases are combined, dried over sodium sulphate and concentrated. The residue is separated chromatographically on a silica gel column (mobile phase: cyclohexane, then cyclohexane/ethyl acetate 30:70). This gives 1.13 g (68% of theory) of product.

¹H-NMR (400 MHz, CDCl₃): δ=1.03 (s, 9H), 1.56-1.69 (m, 2H), 1.88 (d, 1H), 2.30 (s, 3H), 3.99 (s, 3H), 5.14-5.20 (m, 1H), 5.32-5.51 (m, 2H), 6.08 (s, 1H), 6.82 (d, 1H), 6.94 (s, 1H), 7.61 (d, 1H) ppm

MS (DCI): m/z=438 (M+NH₄)⁺.

### EXAMPLE C-XXXVIII

### 8-Chloro-9-methyl-10-bromo-11-hydroxy-3-[(1S)-hydroxy-3,3′-dimethylbutyl]-4-methoxy-5H,7H-dibenzo[b,g][1,5]dioxocin-5-one

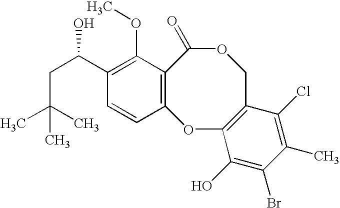

1.13 g (2.69 mmol) of the compound from Example C-XXXVII are dissolved in 35 ml of ethanol, 527 mg (2.96 mmol) of N-bromosuccinimide are added and the mixture is stirred at room temperature for 2 hours. The mixture is diluted with water and the aqueous phase is extracted five times with ethyl acetate. The combined organic phases are dried over magnesium sulphate and concentrated. The residue is chromatographed on a silica gel cartridge (mobile phase: isohexane/ethyl acetate 100:0→10:90). This gives 1.16 g (86% of theory) of product.

¹H-NMR (400 MHz, CDCl₃): δ=1.03 (s, 9H), 1.53-1.68 (m, 2H), 1.92 (d, 1H), 2.51 (s, 3H), 3.99 (s, 3H), 5.13-5.19 (m, 1H), 5.32-5.50 (m, 2H), 6.59 (s, 1H), 6.85 (d, 1H), 7.60 (d, 1H) ppm

MS (ESIpos): m/z=500 (M+H)⁺.

### EXAMPLE C-XXXIX

### 8-Chloro-9,10-dimethyl-11-hydroxy-3-[(1S)-hydroxy-3,3′-dimethylbutyl]-4-methoxy-5H,7H-dibenzo[b,g][1,5]dioxocin-5-one

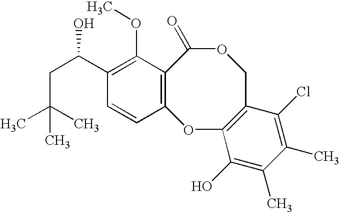

Example C-XXXIX is prepared analogously to Example B-IV using 200 mg (0.40 mmol) of the compound from Example C-XXXVIII, 716 mg (4.00 mmol) of tetramethyltin and 46 mg (0.04 mmol) of tetrakis(triphenylphosphine)palladium(0). The yield is 99 mg (57% of theory).

¹H-NMR (300 MHz, CDCl₃): δ=1.03 (s, 9H), 1.53-1.68 (m, 2H), 1.86 (d, 1H), 2.29 (s, 6H), 3.99 (s, 3H), 5.12-5.21 (m, 1H), 5.32-5.51 (m, 2H), 6.18 (s, 1H), 6.83 (d, 1H), 7.60 (d, 1H) ppm

MS (DCI): m/z=452 (M+NH₄)⁺.

### EXAMPLE C-XL

### 2-(Benzyloxy)-4-bromo-6-[(tetrahydro-2H-pyran-2-yloxy)methyl]phenol

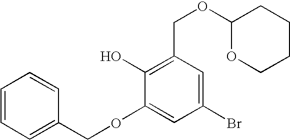

The title compound is prepared from methyl 5-bromo-2,3-dihydroxybenzoate [N. K. Yee, L. J. Nummy, P. P. Roth, Bioorg. Med. Chem. Lett. 6 (19), 2279-2280 (1996)], analogously to Examples B-XV, B-XVI and B-XVII (cf. Scheme 1-15).

¹H-NMR (300 MHz, CDCl₃): δ=1.45-1.95 (m, 6H), 3.52-3.61 (m, 1H), 3.88-3.97 (m, 1H), 4.58 (d, 1H), 4.72 (t, 1H), 4.80 (d, 1H), 5.09 (s, 2H), 6.24 (s, 1H), 6.99 (d, 1H), 7.09 (d, 1H), 7.31-7.45 (m, 5H) ppm

MS (DCI): m/z=410 (M+NH₄)⁺.

### EXAMPLE C-XLI

### Methyl 6-{2-(benzyloxy)-4-bromo-6-[(tetrahydro-2H-pyran-2-yloxy)methyl]phenoxy}-3-formyl-2-methoxybenzoate

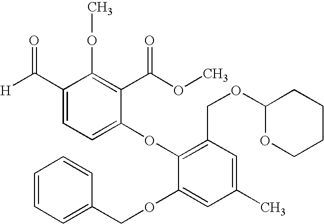

Example C-XLI is prepared analogously to Example B-XVIII using 10.00 g (36.62 mmol) of methyl 6-bromo-3-formyl-2-methoxybenzoate and 14.4 g (36.63 mmol) of the compound from Example C-XL. The yield is 9.8 g (46% of theory).

¹H-NMR (300 MHz, CDCl₃): δ=1.38-1.78 (m, 6H), 3.43-3.52 (m, 1H), 3.72-3.83 (m, 1H), 3.92 (s, 3H), 3.97 (s, 3H), 4.42 (d, 1H), 4.62-4.71 (m, 2H), 4.93 (s, 2H), 6.41 (d, 1H), 7.09-7.16 (m, 3H), 7.20-7.32 (m, 4H), 7.71 (d, 1H) ppm

MS (DCI): m/z=602 (M+NH₄)⁺.

### EXAMPLE C-XLII

### Methyl 6-{2-(benzyloxy)-4-bromo-6-[(tetrahydro-2H-pyran-2-yloxy)methyl]-phenoxy}-3-(1-hydroxy-3-methylbutyl)-2-methoxybenzoate

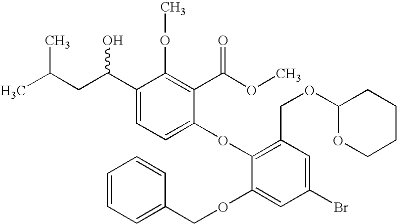

Under argon, 7.5 g (12.81 mmol) of the compound from Example C-XU are dissolved in 300 ml of absolute tetrahydrofuran, 3.66 g (38.4 mmol) of magnesium chloride are added and the mixture is heated under reflux for 20 minutes. After this time, the mixture is cooled and, at room temperature, 3.1 g (19.22 mmol) of isobutylmagnesium chloride (2 M solution in diethyl ether) are added dropwise. After the dropwise addition, the mixture is heated at 60° C. and stirred for 2 hours. The mixture is cooled and 300 ml of 10% strength ammonium chloride solution are added. The mixture is extracted three times with ethyl acetate and the combined organic phases are washed once with saturated sodium chloride solution, dried over sodium sulphate, filtered and concentrated to dryness. The residue is purified chromatographically on a silica gel column (mobile phase: cyclohexane/ethyl acetate 8:1→3:1). This gives 3.6 g (41% of theory) of the product.

¹H-NMR (200 MHz, CDCl₃): δ=0.97 (dd, 6H), 1.38-1.88 (m, 10H), 3.40-3.56 (m, 1H), 3.72-3.95 (m, 7H), 4.48 (dd, 1H), 4.62-4.78 (m, 2H), 4.93-5.08 (m, 3H), 6.32 (d, 1H), 7.06-7.16 (m, 3H), 7.19-7.35 (m, 6H) ppm

MS (DCI): m/z=660 (M+NH₄)⁺.

### EXAMPLE C-XLIII

### Methyl 6-[2-(benzyloxy)-4-bromo-6-(hydroxymethyl)phenoxy]-3-(1-hydroxy-3-methylbutyl)-2-methoxybenzoate

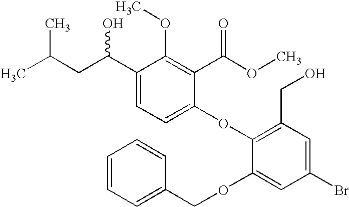

Under argon, 0.5 g (0.78 mmol) of the compound from Example C-XLII is dissolved in 10 ml of ethanol, and 19.5 mg (0.08 mmol) of p-toluenesulphonic acid pyridinium salt are added. The mixture is heated at 55° C. (bath temperature) and stirred at this temperature for 1.5 hours. Triethylamine is added and the mixture is concentrated slightly and directly purified by preparative HPLC. This gives 341 mg (78% of theory) of the product.

¹H-NMR (200 MHz, CDCl₃): δ=0.98 (dd, 6H), 1.38-1.88 (m, 4H), 2.62 (t, 1H), 3.86 (s, 3H), 3.91 (s, 3H), 4.48 (d, 2H), 4.95-5.08 (m, 3H), 6.33 (d, 1H), 7.08-7.18 (m, 3H), 7.19-7.38 (m, 6H) ppm

MS (ESIpos): m/z=581 (M+Na)⁺.

### EXAMPLE C-XLIV

### 11-(Benzyloxy)-9-bromo-3-(1-hydroxy-3-methylbutyl)-4-methoxy-5H,7H-dibenzo-[b,g][1,5]dioxocin-5-one

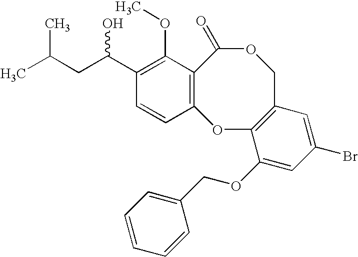

Example C-XLIV is prepared analogously to Example B-XXI from 2.00 g (3.58 mmol) of the compound from Example C-XLIII. The yield is 1.37 g (73% of theory).

¹H-NMR (200 MHz, CDCl₃): δ=0.98 (dd, 6H), 1.38-1.88 (m, 3H), 1.92 (d, 1H), 3.97 (s, 3H), 5.04-5.13 (m, 3H), 5.19 (s, 2H), 6.81 (d, 1H), 6.91 (d, 1H), 7.18 (d, 1H), 7.33-7.53 (m, 5H), 7.58 (d, 1H) ppm

MS (ESIpos): m/z=549 (M+Na)⁺.

### EXAMPLE C-XLV

### 11-(Benzyloxy)-9-bromo-4-methoxy-3-(3-methylbutanoyl)-5H,7H-dibenzo[b,g][1,5]-dioxocin-5-one

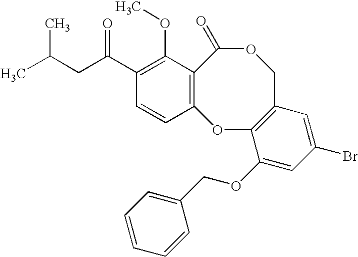

Under argon, 1.93 g (3.66 mmol) of the compound from Example C-XLV are dissolved in 50 ml of dichloromethane. The mixture is cooled to 0° C., and 0.6 g (7.32 mmol) of sodium acetate, 200 mg of 4 Å-molecular sieve, 1.58 g (7.32 mmol) of pyridinium chlorochromate and some silica gel are added. Cooling is removed and the mixture is stirred at room temperature overnight. The mixture is diluted with ethyl acetate and filtered through a layer of silica gel. The silica gel is washed thoroughly with ethyl acetate and the filtrate is concentrated. The residue is purified chromatographically on a silica gel column (mobile phase: cyclohexane/ethyl acetate 5:1). This gives 1.87 g (97% of theory) of the product.

¹H-NMR (400 MHz, CDCl₃): δ=0.97 (dd, 6H), −2.18-2.28 (m, 1H), 2.82 (d, 2H), 3.96 (s, 3H), 5.11 (s, 2H), 5.19 (s, 2H), 6.82 (s, 1H), 6.93 (d, 1H), 7.20 (s, 1H), 7.30-7.53 (m, 5H), 7.58 (d, 1H) ppm

MS (ESIpos): m/z=547 (M+Na)⁺.

### EXAMPLE C-XLVI

### 9-Bromo-11-hydroxy-3-(1-hydroxy-3-methylbutyl)-4-methoxy-5H,7H-dibenzo[b,g]-[1,5]dioxocin-5-one

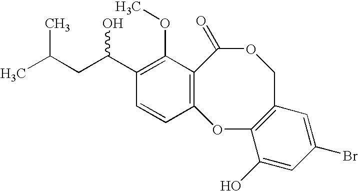 Step a):

150 mg (0.29 mmol) of the compound from Example C-XLV are dissolved in 2 ml of absolute dichloromethane, the mixture is cooled to 0° C., 370 mg (2.28 mmol) of iron(II) chloride are added and the mixture is stirred at this temperature for 1 hour. 1 ml of water is added and the mixture is diluted with ethyl acetate and filtered through an Extrelut cartridge. The cartridge is washed with 35 ml of ethyl acetate and the filtrate is concentrated. The residue is purified chromatographically on a silica gel column (mobile phase: cyclohexane/ethyl acetate 5:1). This gives 111 mg (83% of theory) of the free phenol.

Step b):

Under argon, 105 mg (0.24 mmol) of the phenol are initially charged in 6 ml of tetrahydrofuran, and 0.6 ml of methanol and 18.3 mg (0.48 mmol) of sodium borohydride are added with stirring. The reaction mixture is stirred at room temperature overnight. Another 9.2 mg (0.24 mmol) of sodium borohydride are added, and stirring is continued for 1 hour. 1 ml of water is added and the mixture is filtered through an Extrelut NT3 cartridge and concentrated. This gives 117 mg (95% of theory) of the product.

¹H-NMR (200 MHz, CDCl₃): δ=0.98 (dd, 6H), 1.38-2.02 (m, 4H), 3.97 (s, 3H), 5.03-5.17 (m, 3H), 6.73 (s, 1H), 6.89 (d, 1H), 6.99 (s, 1H), 7.23 (s, 1H), 7.61 (d, 1H) ppm

MS (ESIpos): m/z=459 (M+Na)⁺.

The mixture of enantiomers obtained above can be separated by chromatography on a chiral phase [column: Daicel Chiralpak AD, 20 mm×250 mm; mobile phase: isohexane/ethanol 85:15; flow rate: 15 ml/min; temperature: 25° C.; detection: 220 nm].

The separation of 80 mg of the mixture of enantiomers gives 30 mg (37.5% of theory) of enantiomer 1 and 32 mg (40% of theory) of enantiomer II.

Enantiomer I:

Rₜ=13.18 min. [column: Chiralpak AD, 4.6 mm×250 mm; mobile phase: isohexane/ethanol 85:15; flow rate: 1.0 ml/min; temperature: 40° C.; detection: 220 nm]

Enantiomer II:

Rₜ=18.36 min. [column: Chiralpak AD, 4.6 mm×250 mm; mobile phase: isohexane/ethanol 85:15; flow rate: 1.0 ml/min; temperature: 40° C.; detection: 220 nm].

### EXAMPLE C-XLVII

### 1-(Benzyloxy)-8-methoxy-9-(3-methylbutanoyl)-7-oxo-5H,7H-dibenzo[b,g][1,5]-dioxocin-3-carbonitrile

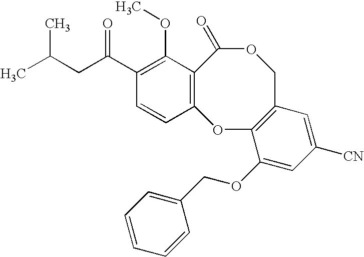

25 mg (0.05 mmol) of the compound from Example C-XLV are dissolved in 0.38 ml of DMF, 5.1 mg (0.06 mmol) of copper(I) cyanide are added and the mixture is stirred at 130° C. for 20 h. Another 5 mg (0.55 mmol) of copper(I) cyanide are added, and stirring is continued for another 4 hours. The mixture is diluted with 5 ml of ethyl acetate and filtered through an Extrelut cartridge. The cartridge is washed with 40 ml of ethyl acetate, the filtrate is concentrated and the residue is purified by preparative TLC (mobile phase: ethyl acetate/cyclohexane 1:2). This gives 11.6 mg (52% of theory) of the product.

¹H-NMR (200 MHz, CDCl₃): δ=0.97 (dd, 6H), 2.15-2.30 (m, 1H), 2.82 (d, 2H), 3.97 (s, 3H), 5.18 (s, 2H), 5.24 (s, 2H), 6.96 (d, 1H), 7.03 (d, 1H), 7.31-7.51 (m, 6H), 7.70 (d, 1H) ppm

MS (ESIpos): m/z=472 (M+H)⁺.

### EXAMPLE C-XLVIII

### 1-Hydroxy-9-(1-hydroxy-3-methylbutyl)-8-methoxy-7-oxo-5H,7H-dibenzo[b,g][1,5]-dioxocin-3-carbonitrile

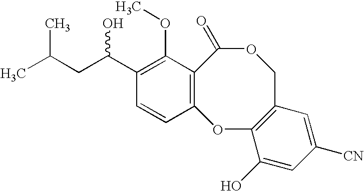

Example C-XLVIII is prepared analogously to Example C-XLVI using 290 mg (0.62 mmol) of the compound from Example C-XLVII and 798 mg (4.92 mmol) of iron trichloride. The yield for the first step is 241 mg (84% of theory). In the second step, 206 mg (0.54 mmol) of the ketone are reacted with 61 mg (1.62 mmol) of sodium borohydride. The yield for the second step is 215 mg (99% of theory).

MS (DCI): m/z=401 (M+NH₄)⁺.

### EXAMPLE C-IL

### 11-(Benzyloxy)-9-chloro-4-methoxy-3-(3-methylbutanoyl)-5H,7H-dibenzo[b,g][1,5]-dioxocin-5-one

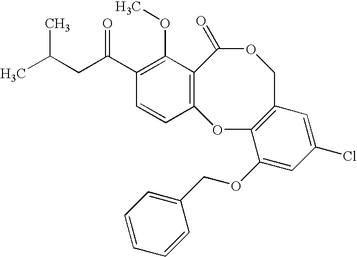

0.5 g (0.95 mmol) of the compound from Example C-XLV is dissolved in 6.5 ml of absolute DMF, 113 mg (1.14 mmol) of copper(I) chloride are added and the mixture is stirred at 130° C. overnight. The mixture is diluted with ethyl acetate and extracted with water. The organic phase is dried over magnesium sulphate and the filtrate is concentrated. The residue is purified chromatographically on a silica gel column (mobile phase: cyclohexane/ethyl acetate 5:1). This gives 428 mg (93% of theory) of the product.

¹H-NMR (200 MHz, CDCl₃): δ=0.97 (dd, 6H), 2.12-2.30 (m, 1H), 2.82 (d, 2H), 3.96 (s, 3H), 5.11 (s, 2H), 5.20 (s, 2H), 6.68 (d, 1H), 6.93 (d, 1H), 7.05 (d, 1H), 7.31-7.52 (m, 5H), 7.68 (d, 1H) ppm

MS (DCI): m/z=481 (M+H)⁺.

### EXAMPLE C-L

### 9-Chloro-11-hydroxy-3-(1-hydroxy-3-methylbutyl)-4-methoxy-5H,7H-dibenzo[b,g]-[1,5]dioxocin-5-one

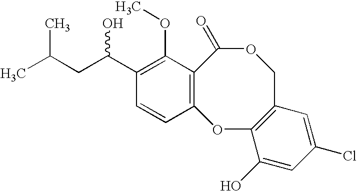

Example C-L is prepared analogously to Example C-XLVI using 410 mg (0.85 mmol) of the compound from Example C-IL and 1.1 g (6.82 mmol) of iron trichloride. The yield for the first step is 281 mg (84% of theory). In the second step, 250 mg (0.64 mmol) of the ketone are reacted with 73 mg (1.92 mmol) of sodium borohydride. The yield for the second step is 264 mg (purity 83%, 87% of theory).

MS (DCI): m/z=410 (M+NH₄)⁺.

The examples listed in the table below are prepared analogously to the procedure of Example A-XLIII, from the corresponding starting materials:

| Example | | |
|---|---|---|
| C- | Structure | Analytical data |
| LI | 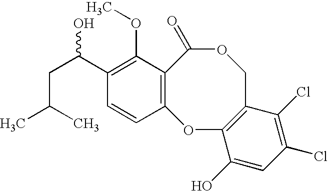 | MS(DCI): m/z = 444 (M + NH₄)⁺ |
| LII | 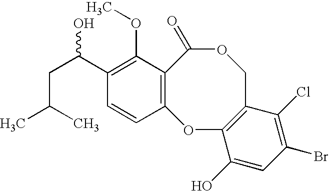 | ¹H-NMR(400 MHz, CDCl₃): δ = 0.99(d, 6H), 1.42-1.88(m, 3H), 1.97 (d, 1H), 3.98(s, 3H), 5.08-5.13(m, 1H), 5.41 (dd, 2H), 6.20(s, 1H), 6.83(d, 1H), 7.38(s, 1H), 7.62(d, 1H) ppm |
| LIII | 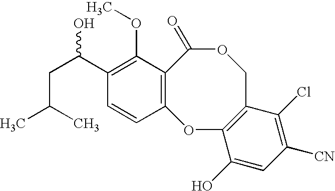 | MS(DCl): m/z = 435 (M + NH₄)⁺ |

The examples listed in the table below are prepared analogously to the procedure of Example A-XLII, from the corresponding starting materials:

| Example | | |
|---|---|---|
| C- | Structure | Analytical data |
| LIV | 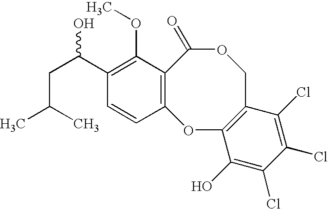 | MS(DCI): m/z = 478 (M + NH₄)⁺ |
| LV | 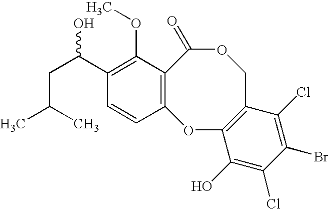 | ¹H-NMR(400 MHz, CDCl₃): δ = 0.99(d, 6H), 1.42-1.88(m, 3H), 1.99 (d, 1H), 3.99(s, 3H), 5.07-5.13(m, 1H), 5.42 (dd, 2H), 6.54(s, 1H), 6.88(d, 1H), 7.62(d, 1H) ppm |
| LVI | 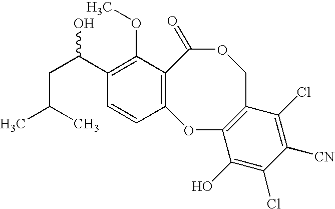 | MS(DCI): m/z = 469 (M + NH₄)⁺ |

### EXAMPLE C-LVII

### 9-Bromo-8-fluoro-11-hydroxy-4-methoxy-3-(3-methylbutanoyl)-5H,7H-dibenzo[b,g][1,5]dioxocin-5-one

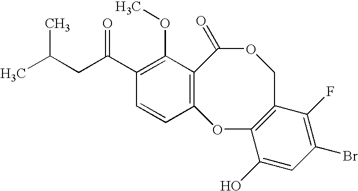

Under argon, 200 mg (0.38 mmol) of the bromide from Example C-XLV are dissolved in 1.8 ml of absolute acetonitrile, and 121 mg (0.48 mmol) of 2,6-dichloro-1-fluoropyridinium tetrafluoroborate are added. The resulting solution is stirred at 60° C. for 14 hours. The mixture is then concentrated and the crude product is purified by preparative HPLC. This gives 24 mg (7% of theory) of the product.

¹H-NMR (300 MHz, CDCl₃): δ 0.97 (dd, 6H), 2.13-2.30 (m, 1H), 2.82 (d, 2H), 3.97 (s, 3H), 5.30 (s, 2H), 6.36 (s, 1H), 6.91 (d, 1H), 7.29 (d, 1H), 7.70 (d, 1H) ppm.

### EXAMPLE C-LVIII

### 9,10-Dibromo-8-fluoro-11-hydroxy-4-methoxy-3-(3-methylbutanoyl)-5H,7H-dibenzo[b,g][1,5]dioxocin-5-one

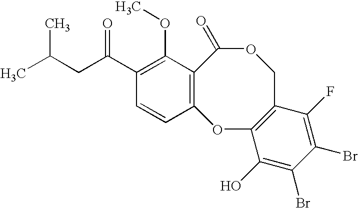

18.5 mg (0.04 mmol) of the phenol from Example C-LVII are dissolved in 0.35 ml of hot ethanol, and 0.2 ml of water, 0.4 mg of 4-toluenesulphonic acid monohydrate and 14.5 mg (0.08 mmol) of N-bromosuccinimide are added. The resulting solution is stirred at room temperature for 16 hours. The same amounts of N-bromosuccinimide and 4-toluenesulphonic acid monohydrate are added again. After a further 4 hours at room temperature, the mixture is diluted with ethyl acetate and washed with water. The aqueous phase is re-extracted once with ethyl acetate and the combined organic phases are washed with saturated sodium chloride solution, dried over sodium sulphate and concentrated. The residue is purified by preparative TLC (mobile phase: ethyl acetate/cyclohexane 1:2). This gives 11.5 mg (purity 79%, 37% of theory) of the product.

¹H-NMR (300 MHz, CDCl₃): δ=0.97 (dd, 6H), 2.16-2.30 (m, 1H), 2.82 (d, 2H), 3.97 (s, 3H), 5.27 (s, 2H), 6.20 (s, 1H), 6.95 (d, 1H), 7.70 (d, 1H) ppm

MS (ESIpos): m/z=533 (M+H)⁺.

### EXAMPLE C-LIX

### 8-Chloro-10-ethyl-11-hydroxy-3-[(1S)-1-hydroxy-3,3-dimethylbutyl]-4-methoxy-9-methyl-5H,7H-dibenzo[b,g][1,5]dioxocin-5-one

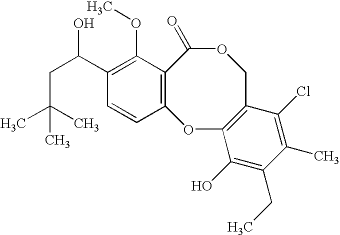

Example C-LIX is prepared analogously to Example B-IV using 130 mg (0.26 mmol) of the compound from Example c-XXXVIII, 917 mg (3.90 mmol) of tetramethyltin and 75 mg (0.065 mmol) of tetrakis(triphenylphosphine)palladium(0) in 1.5 ml of DMF+3.0 ml of toluene. The yield is 26 mg (22% of theory).

¹H-NMR (400 MHz, CDCl₃): δ=1.04 (s, 9H), 1.16 (t, 3H), 1.54-1.68 (m, 2H), 1.73 (d, 1H), 2.30 (s, 3H), 2.78 (q, 2H), 3.98 (s, 3H), 5.16-5.21 (m, 1H), 5.42 (dd, 2H), 6.10 (s, 1H), 6.84 (d, 1H), 7.61 (d, 1H) ppm.

### EXAMPLE C-LX

### 8-Chloro-11-hydroxy-3-(1-hydroxy-3,3-dimethylbutyl)-4-methoxy-10-(methoxy-methyl)-9-methyl-5H,7H-dibenzo[b,g][1,5]dioxocin-5-one

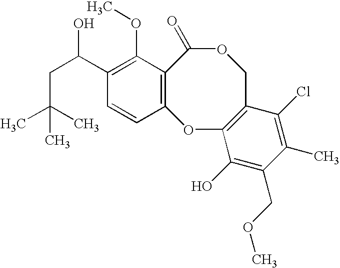

Under argon, 92 mg (0.18 mmol) of the compound from Example C-XXXVIII are dissolved in 2.5 ml of absolute DMF. 370 mg (1.10 mmol) of tributyl(methoxy-methyl)stannane [preparation: J. W. Labadie, D. Tueting, J. K. Stille, J. Org. Chem. 48, 4634-4642 (1983)] and 19.4 mg (0.03 mmol) of bis(triphenyl-phosphine)palladium(II) chloride are added, and the mixture is stirred in a closed vessel at 80° C. for 20 hours. The same amount of catalyst is added again, and stirring at 80° C. is continued for a further 16 hours. After this time, the same amount of catalyst is added again, and stirring at 80° C. is continued for another 16 hours. The mixture is then cooled and filtered through a layer of silica gel, and the silica gel is washed with ethyl acetate. The ethyl acetate phase is washed twice with water and twice with saturated sodium chloride solution, dried over sodium sulphate, filtered and concentrated. To remove remaining stannane, the residue is flash-chromatographed on silica gel (mobile phase: cyclohexane, then cyclohexane/ethyl acetate 2:1). The resulting crude product is then purified again by preparative HPLC. This gives 22 mg (26% of theory) of the product.

¹H-NMR (300 MHz, CDCl₃): δ=1.04 (s, 9H), 1.53-1.71 (m, 2H), 2.03 (br. s, 1H), 2.36 (s, 3H), 3.46 (s, 3H), 3.98 (s, 3H), 4.68 (s, 2H), 5.13-5.23 (m, 1H), 5.46 (dd, 2H), 6.86 (d, 1H), 7.01 (s, 1H), 7.58 (d, 1H) ppm

MS (DCI): m/z=482 (M+NH₄)⁺.

### EXAMPLE C-LXI

### 8-Chloro-11-hydroxy-3-[(1S)-1-hydroxy-3-methylbutyl]-4-methoxy-10-(methoxy-methyl)-9-methyl-5H,7H-dibenzo[b,g][1,5]dioxocin-5-one

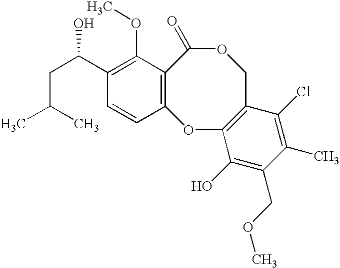

Example C-LXI is prepared analogously to Example C-LX using 60 mg (0.124 mmol) of the compound from Example B-LIII, 124 mg (0.371 mmol) of tributyl(methoxymethyl)stannane and 8.7 mg (0.012 mmol) of bis(triphenyl-phosphine)palladium(II) chloride. The yield is 12 mg (21% of theory).

¹H-NMR (200 MHz, CDCl₃): δ=0.99 (m, 6H), 1.40-1.88 (m, 3H), 2.30 (d, 1H), 2.40 (s, 3H), 3.48 (s, 3H), 3.98 (s, 3H), 4.70 (s, 2H), 5.03-5.15 (m, 1H), 5.46 (dd, 2H), 6.86 (d, 1H), 7.15 (s, 1H), 7.58 (d, 1H) ppm.

### EXAMPLE C-LXII

### 4-Chloro-1-hydroxy-9-[(1S)-1-hydroxy-3-methylbutyl]-8-methoxy-3-methyl-7-oxo-5H,7H-dibenzo[b,g][1,5]dioxocin-2-carbonitrile

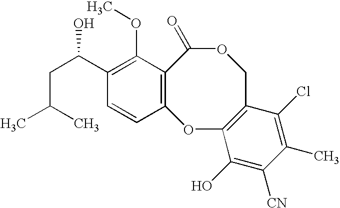

Under argon, 96 mg (0.20 mmol) of the compound from Example B-LIII are dissolved in 1.3 ml of absolute DMF, 26.6 mg (0.30 mmol) copper(I) cyanide are added and the mixture is stirred at 130° C. for 16 hours. The mixture is diluted with ethyl acetate and washed with 0.5 N hydrochloric acid. The organic phase is washed with saturated potassium sodium tartrate solution. The combined organic phases are re-extracted twice with ethyl acetate. The combined organic phases are washed with saturated sodium chloride solution, dried over sodium sulphate and concentrated. The residue is purified by preparative HPLC. This gives 26 mg (30% of theory) of the product.

¹H-NMR (300 MHz, CDCl₃): δ=0.98 (dd, 6H), 1.38-1.88 (m, 3H), 2.20-2.41 (br. s, 1H), 2.52 (s, 3H), 3.98 (s, 3H), 5.05 (dd, 1H), 5.40-5.57 (m, 2H), 6.68 (d, 1H), 7.53 (d, 1H), 7.80 (br. s, 1H) ppm.

### EXAMPLE C-LXIII

### 4-Methoxy-9-methyl-3-((1S)-3-methyl-1-{[2-(trimethylsilyl)ethoxy]methoxy}butyl)-11-{[2-(trimethylsilyl)ethoxy]methoxy}-5H,7H-dibenzo[b,g][1,5]dioxocin-5-one

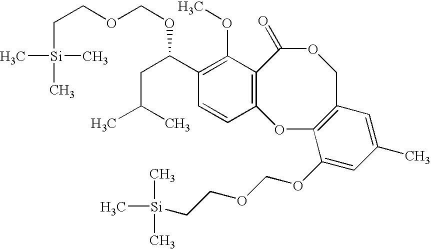

Under argon, 4.0 g (10.74 mmol) of penicillide are dissolved in 100 ml of absolute dichloromethane, and 8.33 g (64.44 mmol) of N,N-diisopropylethylamine are added at room temperature. 5.37 g (32.22 mmol) of (2-chloromethoxyethyl)trimethylsilane are then slowly added dropwise, and the mixture is stirred at room temperature overnight. After this time, another 1.39 g (10.74 mmol) of N,N-diisopropylethyl-amine, 895 mg (5.37 mmol) of (2-chloromethoxyethyl)trimethylsilane and a spatula tip of tetrabutylammonium iodide are added. After 4 hours of stirring, the same volume of water is added to the mixture. The aqueous phase is extracted four times with dichloromethane. The combined organic phases are washed with saturated sodium chloride solution, dried over sodium sulphate and freed from the solvent. The crude product is purified chromatographically on a silica gel column (mobile phase: cyclohexane/ethyl acetate 10:1→8:2). This gives 6.37 g (94% of theory) of the product.

¹H-NMR (300 MHz, CDCl₃): δ=−0.02 (s, 9H), 0.04 (s, 9H), 0.82-1.08 (m, 10H), 1.32-1.88 (m, 3H), 2.28 (s, 3H), 3.42-3.51 (m, 1H); 3.65-3.76 (m, 1H), 3.82-3.91 (m, 2H), 3.99 (s, 3H), 4.52 (d, 1H), 4.63 (d, 1H), 5.03-5.13 (m, 3H), 5.35 (s, 2H), 6.50 (s, 1H), 6.95 (d, 1H), 7.06 (s, 1H), 7.54 (d, 1H) ppm

MS (DCI): m/z=650 (M+NH₄)⁺.

### EXAMPLE C-LXIV

### 6-(2-(Hydroxymethyl)-4-methyl-6-{[2-(trimethylsilyl)ethoxy]methoxy}phenoxy)-2-methoxy-3-((1S)-3-methyl-1-{[2-(trimethylsilyl)ethoxy]methoxy}butyl)benzoic acid

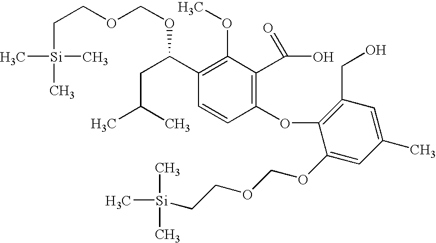

Example C-LXIV is prepared analogously to Example A-XLV using 3.99 g (6.30 mmol) of the compound from Example C-LXIII and 1.62 g (12.61 mmol) of potassium trimethylsilanolate in 25 ml of dichloromethane. The yield is 4.06 g (99% of theory).

¹H-NMR (300 MHz, CDCl₃): δ=−0.03 (s, 9H), 0.01 (s, 9H), 0.84-1.02 (m, 10H), 1.28-1.41 (m, 1H), 1.61-1.88 (m, 2H), 2.35 (s, 3H), 3.42-3.51 (m, 1H), 3.60-3.66 (m, 2H), 3.74-3.82 (m, 1H), 3.94 (s, 3H), 4.45-4.58 (m, 4H), 5.01-5.16 (m, 3H), 6.38 (d, 1H), 6.86 (s, 1H), 7.01 (s, 1H), 7.29 (d, 1H) ppm

MS (DCI): m/z=668 (M+NH₄)⁺.

### EXAMPLE C-LXV

### 6-(2-Formyl-4-methyl-6-{[2-(trimethylsilyl)ethoxy]methoxy}phenoxy)-2-methoxy-3-((1S)-3-methyl-1-{[2-(trimethylsilyl)ethoxy]methoxy}butyl)benzoic acid

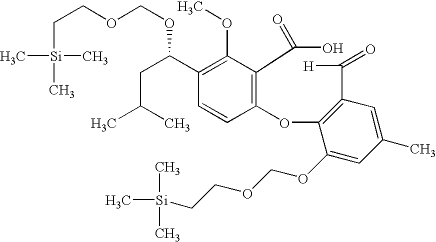

Example C-LXV is prepared analogously to Example A-XLVI using 80 mg (0.12 mmol) of the compound from Example C-LXIV and 94 mg (0.22 mmol) of Dess-Martin periodinane in 0.8 ml of dichloromethane/0.02 ml of pyridine. The yield of crude product is 94 mg (80% purity, 94% of theory).

¹H-NMR (300 MHz, CDCl₃): δ=−0.03 (s, 9H), 0.00 (s, 9H), 0.83-1.02 (m, 10H), 1.26-1.40 (m, 1H), 1.60-1.88 (m, 2H), 2.42 (s, 3H), 3.40-3.51 (m, 1H), 3.60-3.67 (m, 2H), 3.71-3.82 (m, 1H), 3.97 (s, 3H), 4.51 (dd, 2H), 5.08 (dd, 1H), 5.18 (s, 2H), 6.28 (d, 1H), 7.28 (d, 1H), 7.36 (s, 1H), 7.37 (s, 1H), 10.09 (s, 1H) ppm.

### EXAMPLE C-LXVI

### 6-(2-(1-Hydroxyethyl)-4-methyl-6-{[2-(trimethylsilyl)ethoxy]methoxy}phenoxy)-2-methoxy-3-((1S)-3-methyl-1-{[2-(trimethylsilyl)ethoxy]methoxy}butyl)benzoic acid

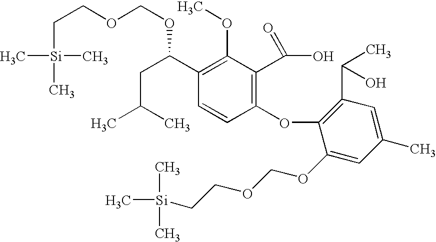

Example C-LXVI is prepared analogously to Example A-XLVII using 4.75 g (7.32 mmol) of the compound from Example C-LXV and 6.83 ml (20.5 mmol) of a 3 M solution of methylmagnesium bromide in diethyl ether in 160 ml of tetrahydrofuran. The yield of crude product is 4.53 g (93% of theory).

¹H-NMR (200 MHz, CDCl₃): δ=0.01 (s, 18H), 0.79-1.08 (m, 10H), 1.29-1.45 (m, 1H), 1.58-1.95 (m, 6H), 2.36 (s, 3H), 3.51-3.62 (m, 2H), 3.68-3.82 (m, 3H), 3.96/3.97 (s, 3H), 4.42-4.60 (m, 2H), 4.94-5.14 (m, 4H), 6.40 (dd, 1H), 6.97 (br. s, 2H), 7.30 (d, 1H) ppm

LC-MS (Method 10): Rₜ=3.34 min.

MS (ESIneg): m/z=663 (M−H)⁻.

### EXAMPLE C-LXVII

### 4-Methoxy-7,9-dimethyl-3-((1S)-3-methyl-1-{[2-(trimethylsilyl)ethoxy]methoxy}-butyl)-11-{[2-(trimethylsilyl)ethoxy]methoxy}-5H,7H-dibenzo[b,g][1,5]dioxocin-5-one

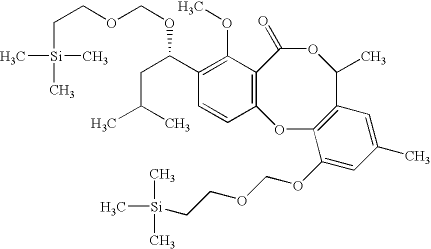

Example C-LXVII is prepared analogously to Example A-XLVIII using 1.44 g (2.16 mmol) of the compound from Example C-LXVI, 1.95 ml (14.05 mmol) of triethylamine and 1.71 g (6.70 mmol) of 2-chloro-1-methylpyridinium iodide in 70 ml of acetonitrile. The yield is 0.779 g (56% of theory).

¹H-NMR (300 MHz, CDCl₃): δ=−0.05 (s, 3H), 0.02 (s, 15H), 0.86-1.00 (m, 10H), 1.25-1.41 (m, 1H), 1.58-1.88 (m, 5H), 2.28 (s, 3H), 3.34-3.51 (m, 1H), 3.58-3.77 (m, 1H), 3.78-3.86 (m, 2H), 3.90/3.97 (s, 3H), 4.43-4.50 (m, 1H), 4.56-4.61 (m, 1H), 5.02-5.10 (m, 1H), 5.26-5.32 (m, 2H), 5.34-5.48 (m, 1H), 6.61 (br. s, 1H), 6.90-6.97 (m, 1H), 7.00-7.04 (m, 1H), 7.44-7.51 (m, 1H) ppm

MS (ESIpos): m/z=664 (M+NH₄)⁺.

### EXAMPLE C-LXVIII

### 11-Hydroxy-3-[(1S)-1-hydroxy-3-methylbutyl]-4-methoxy-7,9-dimethyl-5H,7H-dibenzo[b,g][1,5]dioxocin-5-one

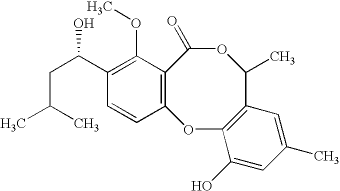

67 mg (0.10 mmol) of the compound from Example C-LXVII and 260 μl (0.26 mmol) of a 1 M solution of tetra-n-butylammonium fluoride are dissolved in tetrahydrofuran (contains about 5% of water), and the solution is then carefully re-concentrated. The residue is taken up in 0.1 ml of 1,3-dimethyltetrahydro-2-(1H)-pyrimidinone, and 60 mg of 4 Å molecular sieve are added. The mixture is stirred at 98° C. for 6 hours. Water is added and the mixture is diluted with ethyl acetate. The organic phase is washed once with 0.5 N hydrochloric acid. The aqueous phases are re-extracted twice with ethyl acetate. The combined organic phases are washed twice with saturated sodium chloride solution, dried over sodium sulphate, filtered and concentrated. The residue is purified by preparative HPLC. This gives 8 mg (21% of theory) of epimer 1 and 8 mg (21% of theory) of epimer 2.

Epimer 1:

¹H-NMR (300 MHz, CDCl₃): δ=0.98 (dd, 6H), 1.40-1.88 (m, 6H), 2.03 (br. s, 1H), 2.24 (s, 3H), 3.99 (s, 3H), 5.06 (dd, 1H), 5.44 (q, 2H), 6.18 (br. s, 1H), 6.50 (s, 1H), 6.80-6.89 (m, 2H), 7.54 (d, 1H) ppm.

Epimer 2:

¹H-NMR (300 MHz, CDCl₃): δ=0.98 (dd, 6H), 1.40-1.88 (m, 6H), 2.22 (br. s, 1H), 2.28 (s, 3H), 3.99 (s, 3H), 5.10 (dd, 1H), 5.49 (q, 2H), 6.05 (br. s, 1H), 6.50 (s, 1H), 6.84-6.89 (m, 2H), 7.57 (d, 1H) ppm.

### EXAMPLE C-LXIX

### 7-Methylbicyclo[2.2.1]heptane-7-carboxylic acid

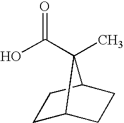

The title compound is prepared by a method known from the literature: R. M. Moriarty, C. C. Chien, T. B. Adams, J. Org. Chem. 44, 2206-2210 (1979).

### EXAMPLE C-LXX

### 7-Methylbicyclo[2.2.1]heptane-7-carbonyl chloride

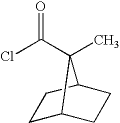

500 mg (3.24 mmol) of 7-methylbicyclo[2.2.1]heptane-7-carboxylic acid are dissolved in 4 ml of dichloromethane, and a drop of DMF is added at 0° C. 0.34 ml (3.9 mmol) of oxalyl chloride are then added, and once the evolution of gas has ceased, the mixture is stirred at room temperature for 30 minutes. The mixture is concentrated under reduced pressure and the crude product is directly reacted further without further purification.

### EMBODIMENTS

The examples listed in the table below are prepared analogously to Example B-6, from the corresponding starting materials:

| Example | | |
|---|---|---|
| C- | Structure | Analytical data |
| 1 | 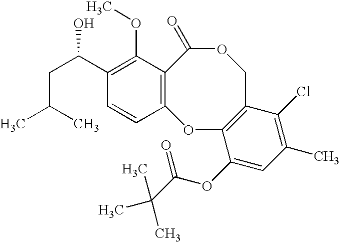 | ¹H-NMR(200 MHz, CDCl₃): δ =0.97(d, 3H), 0.99(d, 3H), 1.39(s, 9H), 1.45-1.53(m, 1H), 1.65-1.73(m, 1H), 1.75-1.86(m, 1H), 1.94(d, 1H), 2.38(s, 3H), 3.99(s, 3H); 5.05-5.12 (m, 1H), 5.43(d, 1H), 5.47(d, 1H), 6.91(d, 1H), 7.04(d, 1H), 7.60(d, 1H); LC-MS(Method 4): Rₜ = 4.74 min.; MS(ESIpos): m/z = 517 (M + H − H₂O)⁺ |
| 2 | 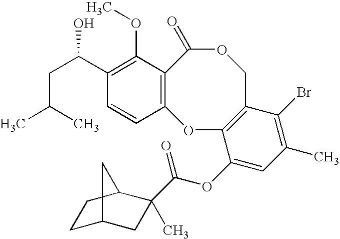 | ¹H-NMR(300 MHz, CDCl₃): δ =0.97(d, 3H), 0.99(d, 3H), 1.05(dd, 1H), 1.16-1.37(m, 3H), 1,45(s, 3H), 1.47-1.57(m, 3H), 1.64-1.75(m, 2H), 1.76-1.86(m, 1H), 1.93(d, 1H), 2.19 (dd, 0.SH), 2.25-2.33(m, 1H), 2.36-2.39(m, 3H), 2.40-2.43(m, 0.5H), 2.46-2.54(m, 0.5H), 2.71(d, 0.5H), 3.99(s, 3H), 5.04-5.12(m, 1H), 5.35-5.50(m, 2H), 6.88-6.95(m, 1H), 7.01 (d, 1H), 7.59(d, 1H); LC-MS(Method 4): |
| # Rₜ = 5.09 min.;nl MS(ESIpos): m/z =569 (M + H − H₂O)⁺ | | |
| 3 | 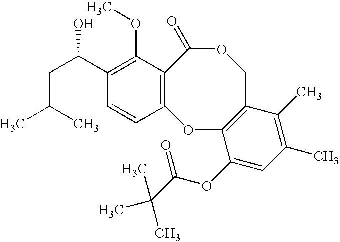 | ¹H-NMR(200 MHz, CDCl₃): δ =0.96(d, 3H), 0.99(d, 3H), 1.39(s, 9H) 1.44-1.52(m, 1H), 1.62-1.88(m, 2H), 1.95(d, 1H), 2.11(s, 3H), 2.24 (s, 3H), 3.99(S. 3H), 5.02-5.13(m, 1H), 5.16-5.33(m, 2H), 6.90(s, 1H), 6.91(d, 1H), 7.57(d, 1H); LC-MS(Method 4): Rₜ = 4.49 min.; MS(ESIpos): m/z = 493(M + Na)⁺ |
| 4 | 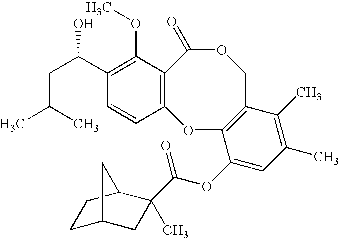 | ¹H-NMR(200 MHz, CDCl₃): δ =0.96(d, 3H), 0.99(d, 3H), 1.13-1.36 (m, 3H), 1.45(s, 3H), 1.47-1.58(m, 2.5H), 1.60-1.87(m, 3H), 1.90-1.99 (m, 1H), 2.11(s, 3H), 2.24(s, 3H), 2.26-2.33(m, 1H), 2.39-2.44(m, 0.5H), 2.45-2.5 8(m, 0.5H), 2.69-2.75 (m, 0.5H), 3.99(s, 3H), 5.01-5.14(m, 1H), 5.15-5.33(m, 2H), 6.88(s, 1H), 6.93(d, 0.5H), 6.95(dd, 0.5H), 7.56 (d, 1H); LC-MS(Method 4): Rₜ = 4.89 min.; |
| # MS(ESIpos): m/z =545(M + Na)⁺ | | |
| 5 | 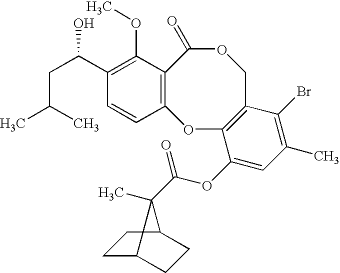 | ¹H-NMR(200 MHz, CDCl₃): δ =0.97(d, 3H), 1.00(d, 3H), 1.28-1.38 (m, 4H), 1.51(s, 3H), 1.40-1.50(m, 1H), 1.61-1.74(m, 1H), 1.78-2.00(m, 6H), 2.28-2.35(m, 2H), 2.38(s, 3H), 3.99(s, 3H), 5.02-5.14(m, 1H), 5.33-5.53(m, 2H), 6.95(d, 1H), 6.99(s, LC-MS(Method 4): Rₜ = 5.17 min.; MS(ESIpos): m/z = 609(M + Na)⁺ |
| 6 | 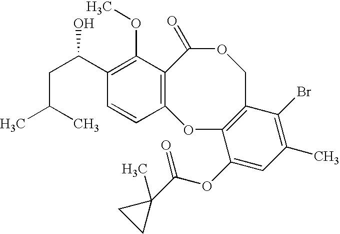 | ¹H-NMR(300 MHz, CDCl₃): δ =0.84-0.89(m, 2H), 0.97(d, 3H), 0.99 (d, 3H), 1.45(s, 3H), 1.48-1.52(m, 2H), 1.63-1.86(m, 2H), 1.89-1.95(m, 1H), 2.37(s, 3H), 3.99(s, 3H), 5.05-5.12(m, 1H), 5.36-5.50(m, 2H), 6.90 (d, 2H), 7.06(s, 1H), 7.59(d, 1H); LC-MS(Method 4): Rₜ = 4.75 min.; MS(ESIpos): m/z = 555(M + Na)⁺ |
| 7 | 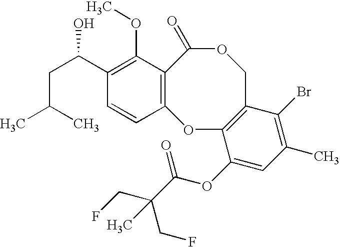 | ¹H-NMR(200 MHz, CDCl₃): δ =0.97(d, 3H), 0.99(d, 3H), 1.43(t, 3H), 1.44-1.53(m, 1H), 1.63-1.73(m, 1H), 1.75-1.86(m, 1H), 1.91(bd, 1H), 2.38(s, 3H), 3.99(s, 3H), 4.59 (dd, 1H), 4.73(dd, 1H), 4.75(dd, 1H), 4.89(dd, 1H), 5.05-5.12(m, 1H), 5.36-5.51(m, 2H), 6.84(d, 1H), 7.06(s, 1H), 7.60(d, 1H); LC-MS(Method 4): Rₜ = 4.60 min.; MS(ESIpos): m/z = 593(M + Na)⁺ |
| 8 | 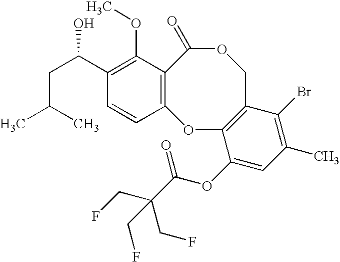 | ¹H-NMR(200 MHz, CDCl₃): δ =0.97(d, 3H), 0.99(d, 3H), 1.43-1.52 (m, 1H), 1.63-1.75(m, 1H), 1.75-1.87 (m, 1H), 1.91(bd, 1H), 2.39(s, 3H), 3.99(s, 3H), 4.76-4.79(m, 3H), 4.91-4.94(m, 3H), 5.04-5.12(m, 1H), 5.37-5.5 1(m, 2H), 6.83(d, 1H), 7.08 (s, 1H), 7.59(d, 1H); LC-MS(Method 4): Rₜ = 4.57 min.; MS(ESIpos): m/z = 611(M + Na)⁺ |
| 9 | 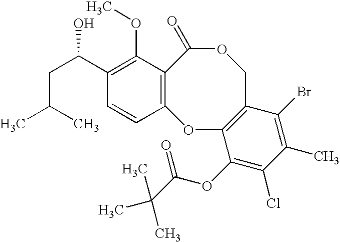 | ¹H-NMR(300 MHz, CDCl₃): δ =0.97(d, 3H), 0.99(d, 3H), 1.43(s, 9H), 1.44-1.54(m, 1H), 1.61-1.89(m, 2H), 1.95(bd, 1H), 2.54(s, 3H), 3.99 (s, 3H), 5.02-5.15(m, 1H), 5.34-5.54 (m, 2H), 6.83-6.92(m, 1H), 7.61(d, 1H); LC-MS(Method 6): Rₜ = 4.80 min.; MS(ESIpos): m/z = 591(M + Na)⁺ |
| 10 | 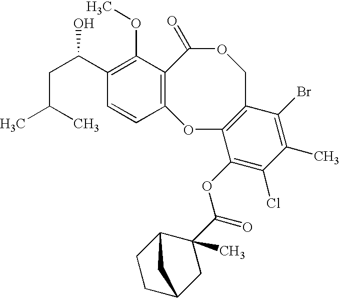 | ¹H-NMR(300 MHz, CDCl₃): δ =0.97(d, 3H), 0.99(d, 3H), 1.27-1.34 (m, 2H), 1.37-1.52(m, 4H), 1.51(s, 3H), 1.63-1.87(m, 4H), 1.90-1.95(m, 1H), 2.20(dd, 1H), 2.28-2.33(m, 1H), 2.42-2.46(m, 1H), 2.54(s, 3H), 3.99(s, 3H), 5.04-5.12(m, 1H), 5.32-5.52(m, 2H), 6.90-6.97(m, 1H), 7.60 (dd, 1H); LC-MS(Method 4): Rₜ = 4.79 min.; MS(ESIpos): m/z = 603 (M + H − H₂O)⁺ |
| 11 | 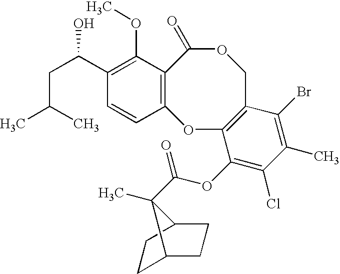 | ¹H-NMR(200 MHz, CDCl₃): δ =0.97(d, 3H), 0.99(d, 3H), 1.24-1.37 (m, 4H), 1.44-1.52(m, 1H), 1.49(s, 3H), 1.64-1.74(m, 1H), 1.76-1.95(m, 4H), 1.98-2.08(m, 2H), 2.33-2.38(m, 2H), 2.54(s, 3H), 3.99(s, 3H), 5.04-m, 1H), 5.28-5.58(m, 2H), 6.93 (d, 1H), 7.60(d, 1H); LC-MS(Method 4): Rₜ = 4.81 min.; MS(ESIpos): m/z = 603 (M + H − H₂O)⁺ |
| 12 | 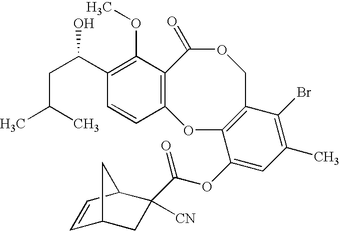 | ¹H-NMR(200 MHz, CDCl₃): δ =0.97(d, 3H), 1.00(d, 3H), 1.43-1.52 (m, 1H), 1.63-1.97(m, 5H), 2.17(dd, 0.7H), 2.35-2.42(m, 1H), 2.39(s, 2H), 2.40(s, 1H), 2.70(dd, 0.3H), 3.13-3.19(m, 1H), 3.61-3.65(m, 0.3H), 3.74-3.79(m, 0.7H), 3.99(s, 3H), 5.09(dd, 1H), 5.36-5.53(m, 2H), 6.01(dd, 0.7H), 6.32(dd, 0.7H), 6.38(dd, 0.3H), 6.50(dd, 0.3H), 6.91 (dd, 0.7H), 6.95(d, 0.3H), 7.09(s, 0.7H), 7.16(s, 0.3H), 7.61(d, 1H); |
| # LC-MS(Method 6): Rₜ = 4.60 min.; MS(ESIpos): m/z = 618(M + Na)⁺ | | |
| 13 | 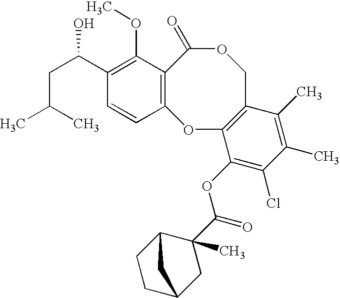 | ¹H-NMR(300 MHz, CDCl₃): δ =0.97(d, 3H), 0.99(d, 3H), 1.25-1.33 (m, 2H), 1.37-1.49(m, 1H), 1.51(s, 3H), 1.59-1.85(m, 5H), 1.91-1.96(m, 1H), 2.11(dd, 1H), 2.18(s, 3H), 2.18-2.23(m, 1H), 2.24-2.32(m, 2H), 2.34(s, 3H), 2.42-2.47(m, 1H), 3.99 (s, 3H), 5.04-5.12(m, 1H), 5.15-5.30 (m, 2H), 6.92-6.98(m, 1H), 7.57(dd, 1H); LC-MS(Method 4): Rₜ = 4.56 min.; MS(ESIpos): m/z = 539 |
| # (M + H − H₂O)⁺ | | |
| 14 | 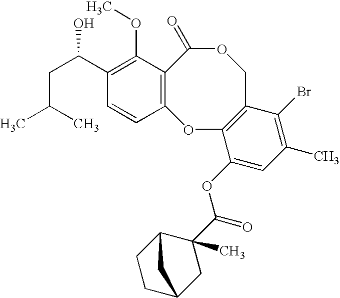 | ¹H-NMR(400 MHz, CDCl₃): δ =0.90(dt, 1H), 0.97(d, 3H), 0.99(d, 3H), 1.22-1.40(m, 4H), 1.45(s, 3H), 1.47-1.57(m, 2H), 1.64-1.73(m, 2H), 1.75-1.86(m, 1H), 1.93-2.05(m, 1H), 2.19(dd, 1H), 2.28-2.33(m, 1H), 2.38(s, 3H), 2.40-2.43(m, 1H), 3.99 (s, 3H), 5.08(dd, 1H), 5.40(d, 1H), 5.49(d, 1H), 6.92(d, 1H), 7.02(s, 1H), 7.59(d, 1H); LC-MS(Method 4): Rₜ = 5.09 min.; MS(ESIpos): m/z = |
| # 609(M + Na)⁺ | | |
| 15 | 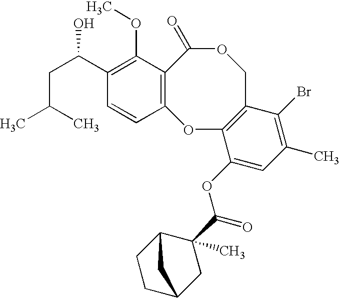 | ¹H-NMR(200 MHz, CDCl₃): δ =0.90(m, 1H), 0.97(d, 3H), 0.99(d, 3H), 1.05(dd, 1H), 1.24-1.34(m, 2H), 1.45(s, 3H), 1.46-1.61(m, 3H), 1.64-1.75(m, 2H), 1.76-1.85(m, 1H), 1.94-2.05(m, 1H), 2.25-2.30(m, 1H), 2.37(s, 3H), 2.50(ddd, 1H), 2.71(d, 1H), 3.98(s, 3H), 5.08(dd, 1H), 5.40 (d, 1H), 5.47(d, 1H), 6.91(d, 1H), 7.01 (s, 1H), 7.59(d, 1H); LC-MS(Method 4): Rₜ = 5.09 min.; MS(ESIpos): m/z = 609 |
| # (M + Na)⁺ | | |
| 16 | 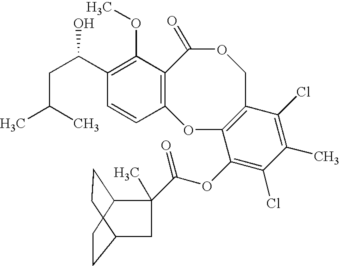 | ¹H-NMR(300 MHz, CDCl₃): δ =0.97(d, 3H), 0.99(d, 3H), 1.29-1.40 (m, 1H), 1.43-1.58(m, 7H), 1.53(s, 3H), 1.65-1.75(m, 2H), 1.76-1.96(m, 4H), 2.03-2.09(m, 1H), 2.46(s, 3H), 2.59(bd, 1H), 3.99(s, 31-i), 5.03-5.12 (m, 1H), 5.26-5.5 1(m, 2H), 6.91(d, 1H), 7.59(d, 1H); LC-MS(Method 5): Rₜ = 4.70 min.; MS(ESIpos): m/z = 613(M + Na)⁺ |
| 17 | 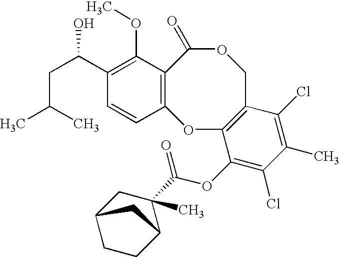 | ¹H-NMR(200 MHz, CDCl₃): δ =0.97(d, 3H), 0.99(d, 3H), 1.22-1.40 (m, 4H), 1.43-1.52(m, 1H), 1.61-2.10 (m, 6H), 2.25-2.46(m, 2H), 2.48(s, 2.96-3.03(m, 1H), 3.99(s, 3H), 5.03-5.15(m, 1H), 5.26-5.53(m, 2H), 6.90(d, 1H), 7.61(d, 1H); LC-MS(Method 4): Rₜ = 4.45 min.; MS(ESIpos): m/z = 648(M + NH₄)⁺ |
| 18 | 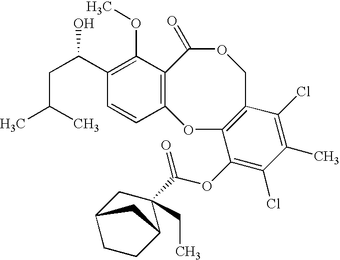 | ¹H-NMR(200 MHz, CDCl₃): δ =0.93(t, 3H), 0.97(d, 3H), 0.99(d, 3H), 1.17-1.34(m, 3H), 1.41-1.57(m, 3H), 1.60-1.88(m, 5H), 1.95(dd, 1H), 2.06(bd, 1H), 2.17(dd, 1H), 2.25-2.34(m, 1H), 2.43-2.46(m, 1H), 2.47(s, 3H), 3.99(s, 3H), 5.03-5.14 1H), 7.60(d, 1H); LC-MS(Method 4): Rₜ = 2.43 min.; MS(ESIpos): m/z = 608(M + NH₄)⁺ |
| 19 | 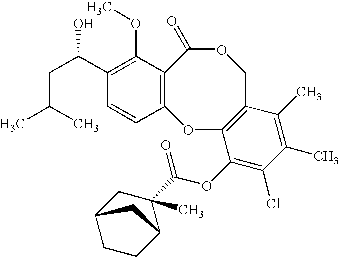 | ¹H-NMR(300 MHz, CDCl₃): δ =0.97(d, 3H), 0.99(d, 3H), 1.21-1.34 (m, 2H), 1.36-1.53(m, 4H), 1.51(s, 3H), 1.64-1.75(m, 2H), 1.76-1.87(m, 2H), 1.94(bs, 1H), 2.18(s, 3H), 2.22 (dd, 1H), 2.27-2.32(m, 1H), 2.34(s, 3H), 2.43-2.46(m, 1H), 3.99(s, 3H), 5.04-5.11(m, 1H), 5.23(bs, 2H), 6.95(d, 1H), 7.57(d, 1H); LC-MS(Method 6): Rₜ = 3.61 min.; MS(ESIpos): m/z = 557(M + H)⁺ |
| 20 | 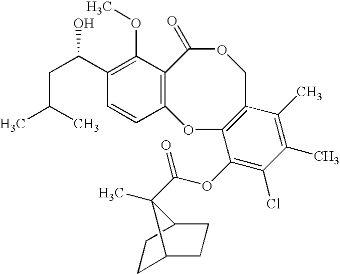 | ¹H-NMR(300 MHz, CDCl₃): δ =0.97(d, 3H), 0.99(d, 3H), 1.25-1.36 (m, 3H), 1A4-1.52(m, 1H), 1.49(s, 3H), 1.64-1.74(m, 1H), 1.75-1.90(m, 4H), 1.91-1.97(m, 1H), 2.01-2.09(m, 2H), 2.17(s, 3H), 2.34(s, 3H), 2.34-2.38(m, 1H), 4.00(s, 3H), 5.04-5.11 (m, 1H), 5.13-5.29(m, 2H), 6.95(d, LC-MS(Method 6): Rₜ = 3.62 min.; MS(ESIpos): m/z = 574(M + NH₄)⁺ |
| 21 | 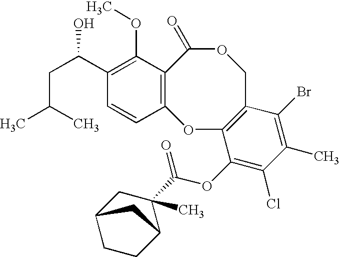 | ¹H-NMR(300 MHz, CDCl₃): δ =0.97(d, 3H), 0.99(d, 3H), 1.19-1.34 (m, 3H), 1.37-1.46(m, 2H), 1.47-1.52 (m, 1H), 1.50(s, 3H), 1.64-1.87(m, 4H), 1.94(d, 1H), 2.20(dd, 1H), 2.28-2.33(m, 1H), 2.42-2.46(m, 1H), 2.54(s, 3H), 3.99(s, 3H), 5.05-5.12 (m, 1H), 5.42(bs, 2H), 6.93(d, 1H), 7.60(d, 1H); LC-MS(Method 6): Rₜ = 3.75 min.; MS(ESIpos): m/z = 638(M + NH₄)⁺ |
| 22 | 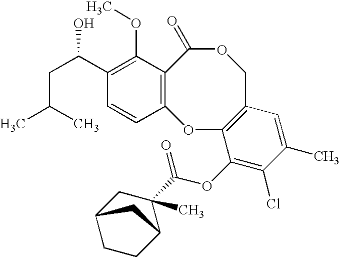 | ¹H-NMR(200 MHz, CDCl₃): δ =0.97(d, 3H), 0.99(d, 3H), 1.16-1.36 (m, 4H), 1.37-1.90(m, 8H), 1.52(s, 3H), 2.22(dd, 1H), 2.34(s, 3H), 2.43-2.49(m, 1H), 3.98(s, 3H), 4.99-5.14(m, 3H), 6.83(s, 1H), 6.98(d, 1H), 7.59(d, 1H); LC-MS(Method 6): Rₜ = 3.55 min.; MS(ESIpos): m/z = 560(M + NH₄)⁺ |
| 23 | 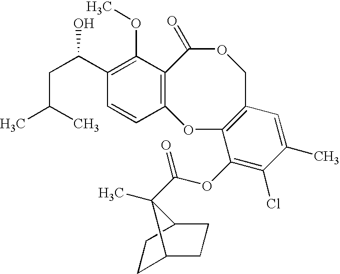 | ¹H-NMR(300 MHz, CDCl₃): δ =0.97(d, 3H), 0.99(d, 3H), 1.25-1.37 (m, 3H), 1.44-1.52(m, 1H), 1.51(s, 3H), 1.62-1.96(m, 6H), 2.02-2.11(m, 2H), 2.33(s, 3H), 2.342.39(m, 2H), 3.98(s, 3H), 4.98-5.12(m, 3H), 6.82 (s, 1H), 6.98(d, 1H), 7.59(d, 1H); LC-MS(Method 5): Rₜ = 4.58 min.; MS(ESIpos): m/z = 565(M + Na)⁺ |
| 24 | 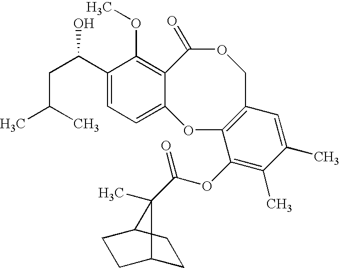 | ¹H-NMR(300 MHz, CDCl₃): δ =0.98(dd, 6H), 1.20-2.08(m, 15H), 2.12(s, 3H), 2.21(s, 3H), 2.35(m, 2H), 3.98(s, 3H), 4.94-5.14(m, 3H), 6.71(s, 1H), 7.00(d, 1H), 7.56(d, 1H); HPLC(Method 1): Rₜ = 5.71 min.; MS(DCI): m/z = 540(M + NH₄)⁺ |
| 25 | 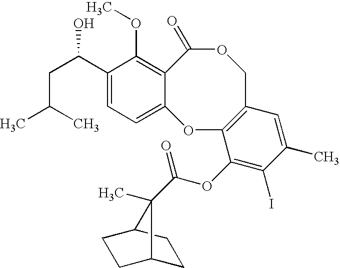 | ¹H-NMR(300 MHz, CDCl₃): δ =0.98(dd, 6H), 1.21-1.90(m, 13H), 2.13(m, 2H), 2.37(m, 2H), 2.42(s, 3H), 3.98(s, 3H), 5.01(m, 2H), 5.08 (m, 1H), 6.85(s, 1H), 7.03(d, 1H), 7.59(d, 1H); HPLC(Method= 5.93 min.; MS(DCl): m/z = 652(M + NH₄)⁺ |
| 26 | 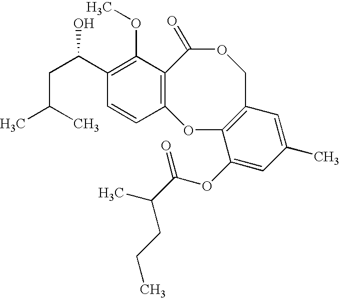 | ¹H-NMR(200 MHz, DMSO-d₆): δ =0.80-1.90(m, 17H), 2.05(d, 1H), 2.24(s, 3H), 2.82(m, 1H), 3.82(s, 3H), 4.90(m, 1H), 5.18(m, 3H), 6.87 (d, 1H), 6.98(m, 1H), 7.10(m, 1H), 7.67(d, 1H); MS(ESI): m/z = 493(M + Na)⁺HPLC(Method 2): Rₜ = 5.30 min. |
| 27 | 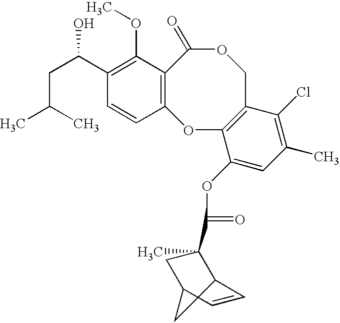 | ¹H-NMR(200 MHz, CDCl₃): δ =0.96(d, 3H), 0.99(d, 3H), 1.18-1.95 (m, 1 1H), 2.32(s, 3H), 2.90(m, 1H), 3.00(m, 1H), 3.98(s, 3H), 5.05(m, 1H), 5.37(d, 1H), 5.45(d, 1H), 6.18 (m, 1H), 6.27(m, 1H), 6.89(d, 1H), 6.91(s, 1H), 7.57(d, 1H); MS(ESI): m/z = 563(M + Na)⁺HPLC(Method 2): Rₜ = 5.70 min. |
| 28 | 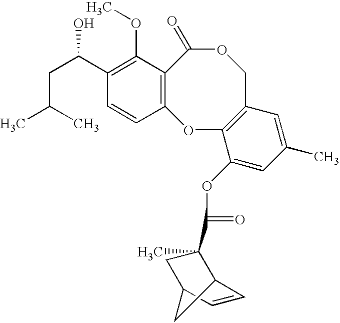 | ¹H-NMR(200 MHz, CDCl₃): δ =0.96(d, 3H), 0.99(d, 3H), 1.18-1.95 (m, 11H), 2.32(s, 3H), 2.90(m, 1H), 3.02(m, 1H), 3.98(s, 3H), 5.05-5.10 (m, 3H), 6.20(m, 1H), 6.30(m, 1H), 6.70(m, 1H), 6.82(m, 1H), 6.90(d, 1H), 7.55(d, 1H); MS(ESI): m/z = 524(M + Na)⁺HPLC(Method 2): Rₜ = 5.50 min. |
| 29 | 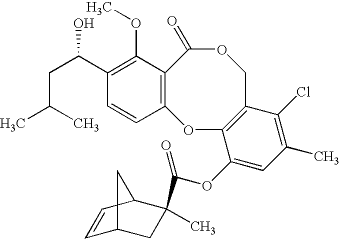 | ¹H-NMR(200 MHz, CDCl₃): δ =0.96(d, 3H), 1.00(d, 3H), 1.18-1.95 (m, 11H), 2.33(s, 3H), 2.60(m, 1H), 2.90(m, 1H), 3.98(s, 3H), 5.00-5.10 (m, 1H), 5.37(d, 1H), 5.44(d, 1H), 6.10(m, 1H), 6.28(m, 1H), 6.90(d, 1H), 7.57(d, 1H); MS(ESI): m/z = 558(M + NH₄)⁺HPLC(Method 2): Rₜ = 5.70 min. |
| 30 | 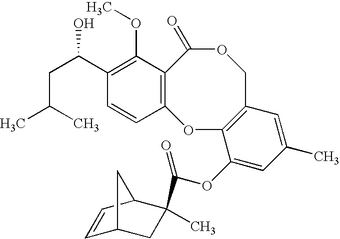 | ¹H-NMR(200 MHz, CDCl₃): δ =0.96(d, 3H), 1.00(d, 3H), 1.18-1.95 (m, 11H), 2.33(s, 3H), 2.60(m, 1H), 2.90(m, 1H), 3.98(s, 3H), 5.00-5.10 (m, 3H), 6.10(m, 1H), 6.28(m, 1H), 6.71(m, 1H), 6.90(m, 1H), 6.91(d, 1H), 7.57(d, 1H); MS(DCl): m/z = 524(M + NH₄)⁺HPLC(Method 2): Rₜ = 5.54 min. |
| 31 | 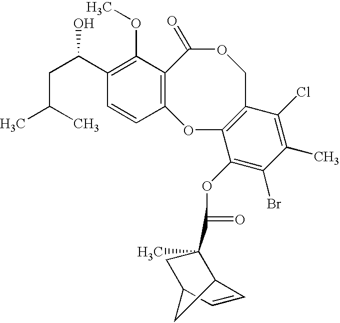 | ¹H-NMR(300 MHz, CDCl₃): δ =0.96(d, 3H), 1.00(d, 3H), 1.18-2.20 (m, 11H), 2.51(s, 3H), 2.90(m, 1H), 3.04(m, 1H), 3.98(s, 3H), 5.05(m, 1H), 5.17-5.50(br. m, 2H), 6.10(m, 1H), 6.32(m, 1H), 6.88(d, 1H), 7.57 (d, 1H); MS(DCI): m/z = 636/638(M + NH₄)⁺HPLC(Method 2): Rₜ = 6.03 min. |
| 32 | 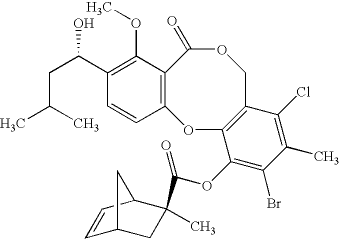 | ¹H-NMR(300 MHz, CDCl₃): δ =0.96(d, 3H), 1.00(d, 3H), 1.18-2.20 (m, 11H), 2.51(s, 3H), 2.90(m, 1H), 3.36(m, 1H), 3.98(s, 3H), 5.05(m, 1H), 5.17-5.50(br. m, 2H), 6.15(m, 1H), 6.30(m, 1H), 6.90(d, 1H), 7.60 (d, 1H); MS(DCI): m/z = 636/638(M + NH₄)⁺HPLC(Method 2): Rₜ = 6.42 min. |
| 33 | 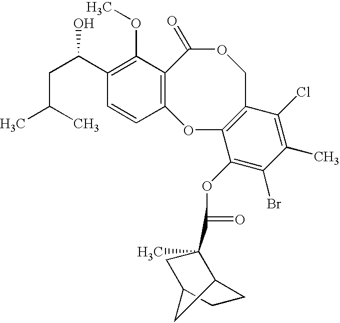 | ¹H-NMR(300 MHz, CDCl₃): δ =0.96(d, 3H), 1.00(d, 3H), 1.18-2.20 (m, 15H), 2.28(m, 1H), 2.51(s, 3H), 2.80(m, 1H), 3.98(s, 3H), 5.08(m, 1H), 5.30-5.50(br. m, 2H), 6.88(d, 1H), 7.57(d, 1H); MS(DCI): m/z = 638/640(M + NH₄)⁺HPLC(Method 2): Rₜ = 6.58 min. |
| 34 | 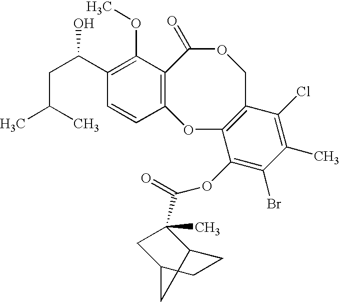 | ¹H-NMR(300 MHz, CDCl₃): δ =0.96(d, 3H), 1.00(d, 3H), 1.18-2.20 (m, 15H), 2.30(m, 1H), 2.45(m, 1H), 2.51(s, 3H), 3.98(s, 3H), 5.08(m, 1H), 5.30-5.50(br. m, 2H), 6.93(d, 1H), 7.60(d, 1H); MS(DCI): m/z = 638/640(M + NH₄)⁺HPLC(Method 2): Rₜ = 6.48 min. |
| 35 | 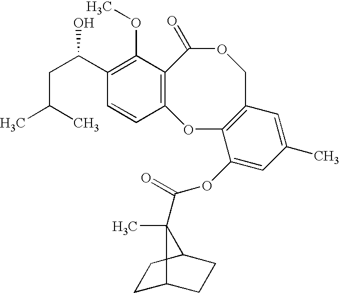 | ¹H-NMR(300 MHz, CDCl₃): δ =0.96(d, 3H), 1.00(d, 3H), 1.18-2.20 (m, 15H), 2.28(s, 3H), 2.32(m, 2H), 3.98(s, 3H), 5.01-5.10(m, 3H), 6.71 (m, 1H), 6.88(m, 1H), 7.00(d, 1H), 7.57(d, 1H); MS(ESI): m/z = 531(M + Na)⁺HPLC(Method 2): Rₜ = 5.67 min. |
| 36 | 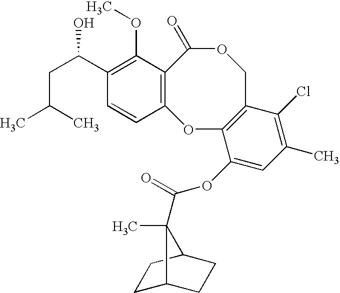 | ¹H-NMR(300 MHz, CDCl₃): δ =0.96(d, 3H), 1.00(d, 3H), 1.18-2.20 (m, 15H), 2.32(m, 2H), 2.33(s, 3H), 3.98(s, 3H), 5.01-5.10(m, 1H), 5.35 (d, 1H), 5.43(d, 1H), 6.90-6.96(m, 2H), 7.57(d, 1H); MS(DCI): m/z = 560(M + NH₄)⁺HPLC(Method 2): Rₜ = 5.93 min. |
| 37 | 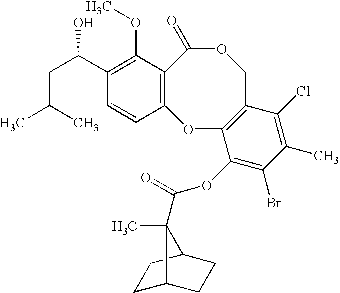 | ¹H-NMR(300 MHz, CDCl₃): δ =0.96(d, 3H), 1.00(d, 3H), 1.18-2.20 (m, 15H), 2.35(m, 2H), 2.55(s, 3H), 3.98(s, 3H), 5.01-5.10(m, 1H), 5.20-5.42(br. m, 2H), 6.93(d, 1H), 7.60 (d, 1H); MS(DCI): m/z = 63 8/640(M + NH₄)⁺HPLC(Method 2): Rₜ = 6.33 min. |
| 38 | 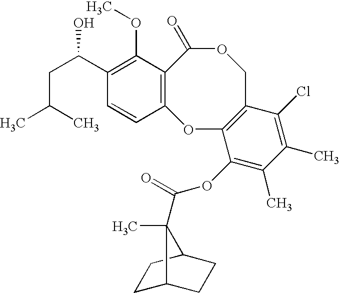 | ¹H-NMR(300 MHz, CDCl₃): δ =0.96(d, 3H), 1.00(d, 3H), 1.18-2.20 (m, 16H), 2.20(s, 3H), 2.35(m, 4H), 3.98(s, 3H), 5.01-5.10(m, 1H), 5.20-5.42(br. m, 2H), 6.93(d, 1H), 7.60 (d, 1H); MS(DCI): m/z = 574(M + NH₄)⁺HPLC(Method 2): Rₜ = 6.10 min. |
| 39 | 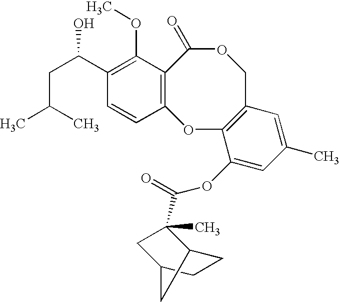 | ¹H-NMR(200 MHz, CDCl₃): δ =0.96(d, 3H), 1.00(d, 3H), 1.18-2.20 (m, 15H), 2.20(dd, 1H), 2.30(s, 3H), 2.44(m, 1H), 3.98(s, 3H), 5.08-5.10 (m, 3H), 6.88(d, 1H), 7.57(d, 1H); MS(DCI): m/z = 526(M + NH₄)⁺HPLC(Method 2): Rₜ = 5.68 min. |
| 40 | 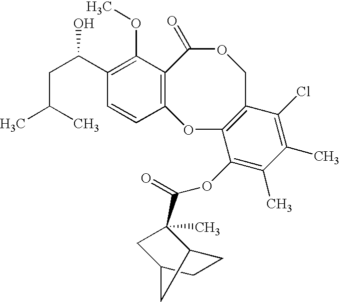 | ¹H-NMR(300 MHz, CDCl₃): δ =0.96(d, 3H), 1.00(d, 3H), 1.18-2.00 (m, 15H), 2.20(s, 3H), 2.25(m, 1H), 2.31(s, 3H), 2.45(m, 1H), 3.98(s, 3H), 5.08(m, 1H), 5.35-5.50(br. m, 2H), 6.93(d, 1H), 7.57(d, 1H); MS(DCI): m/z = 574(M + NH₄)⁺HPLC(Method 2): Rₜ = 6.10 min. |
| 41 | 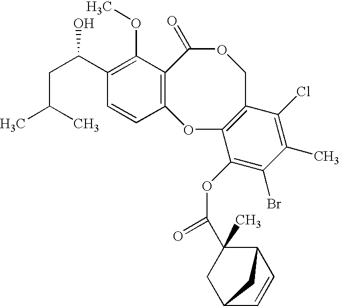 | ¹H-NMR(300 MHz, CDCl₃): δ =0.96(d, 3H), 1.00(d, 3H), 1.18-2.20 (m, 11H), 2.51(s, 3H), 2.90(m, 1H), 3.04(m, 1H), 3.98(s, 3H), 5.05(m, 1H), 5.17-5.50(br. m, 2H), 6.10(m, 1H), 6.32(m, 1H), 6.88(d, 1H), 7.57 (d, 1H); MS(ESI): m/z = 641/643(M + Na)⁺HPLC(Method 2): Rₜ = 6.29 min. |
| 42 | 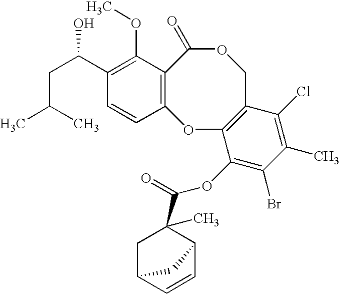 | ¹H-NMR(300 MHz, CDCl₃): δ =0.96(d, 3H), 1.00(d, 3H), 1.18-2.20 (m, 11H), 2.51(s, 3H), 2.90(m, 1H), 3.04(m, 1H), 3.98(s, 3H), 5.05(m, 1H), 5.30(br. m, 1H), 5.50(br. m, 1H), 6.10(m, 1H), 6.32(m, 1H), 6.88 (d, 1H), 7.57(d, 1H); MS(DCI): m/z = 636/638(M + NH₄)⁺HPLC(Method 2): Rₜ = 6.26 min. |
| 43 | 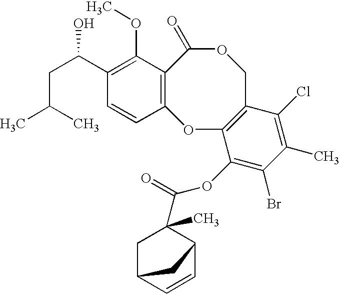 | ¹H-NMR(300 MHz, CDCl₃): δ =0.96(d, 3H), 1.00(d, 3H), 1.18-2.20 (m, 14H), 2.21(dd, 1H), 2.30(m, 1H), 2.49(m, 1H), 2.52(s, 3H), 3.98 (s, 3H), 5.08(m, 1H), 5.4(br. m, 2H), 6.92(d, 1H), 7.57(d, 1H); MS(DCI): m/z = 63 8/640(M + NH₄)⁺HPLC(Method 2): Rₜ = 6.51 min. |
| 44 | 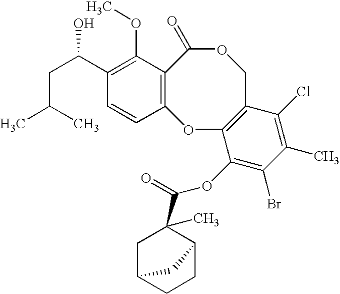 | ¹H-NMR(300 MHz, CDCl₃): δ =0.96(d, 3H), 1.00(d, 3H), 1.18-2.20 (m, 14H), 2.21(dd, 1H), 2.30(m, 1H), 2.47(m, 1H), 2.52(s, 3H), 3.98 (s, 3H), 5.08(m, 1H), 5.3(d, 1H), 5.45(d, 1H), 6.92(d, 1H), 7.57(d, 1H); MS(DCI): m/z = 638/640(M + NH₄)⁺HPLC(Method 2): Rₜ = 6.40 min. |
| 45 | 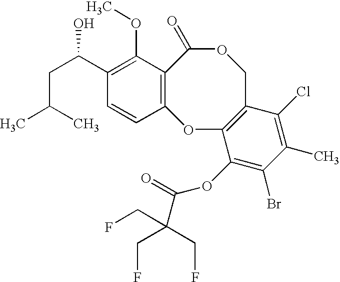 | ¹H-NMR(300 MHz, CDCl₃): δ =0.96(d, 3H), 1.00(d, 3H), 1.18-2.20 (m, 4H), 2.52(s, 3H), 3.98(s, 3H), 4.90(d, 6H), 5.08(m, 1H), 5.4(br. m, 6.82(d, 1H), 7.60(d, 1H); HPLC(Method 2): Rₜ = 5.48 min. |
| 46 | 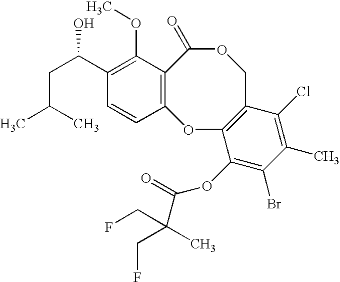 | ¹H-NMR(300 MHz, CDCl₃): δ =0.96(d, 3H), 1.00(d, 3H), 1.18-2.20 (m, 7H), 2.52(s, 3H), 3.98(s, 3H), 4.60-4.93(m, 4H), 5.08(m, 1H), 5.40 (br. m, 2H), 6.82(d, 1H), 7.60(d, 1H); MS(DCI): m/z = 638/640(M + NH₄)⁺HPLC(Method 2): Rₜ = 5.48 min. |
| 47 | 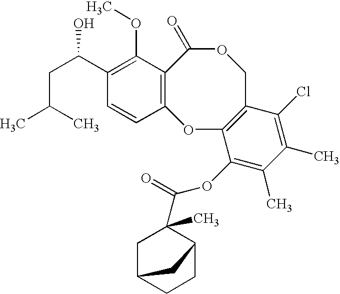 | ¹H-NMR(300 MHz, CDCl₃): δ =0.96(d, 3H), 1.00(d, 3H), 1.18-2.00 (m, 15H), 2.20(s, 3H), 2.25(m, 1H), 2.31(s, 3H), 2.45(m, 1H), 3.98(s, 5.08(m, 1H), 5.35(m, 2H), 6.93 (d, 1H), 7.57(d, 1H); MS(DCI): m/z = 574(M + NH₄)⁺HPLC(Method 2): Rₜ = 6.09 min. |
| 48 | 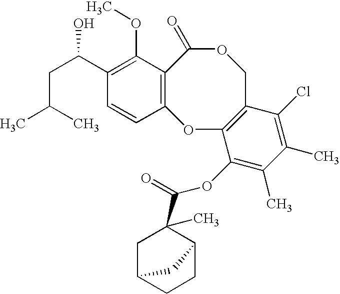 | ¹H-NMR(300 MHz, CDCl₃): δ =0.96(d, 3H), 1.00(d, 3H), 1.18-2.000 (m, 14H), 2.10(dd, 1H), 2.20(s, 3H), 2.25(m, 1H), 2.31(s, 3H), 2.45(m, 1H), 3.98(s, 3H), 5.08(m, 1H), 5.35 (d, 1H), 5.45(d, 1H), 6.93(d, 1H), 7.57(d, 1H); MS(ESI): m/z = 579(M + NH₄)⁺ |
| 49 | 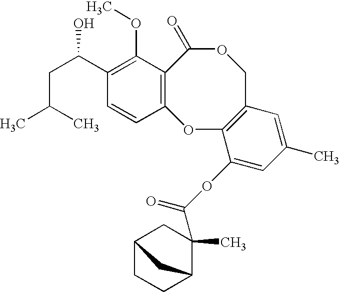 | ¹H-NMR(200 MHz, CDCl₃): δ =0.96(d, 3H), 0.99(d, 3H), 1.18-1.95 (m, 11H), 2.32(s, 3H), 2.90(m, 1H), 3.02(m, 1H), 3.98(s, 3H), 5.05-5.10 (m, 3H), 6.20(m, 1H), 6.30(m, 1H), 6.70(m, 1H), 6.82(m, 1H), 6.90(d, 1H), 7.55(d, 1H); MS(ESI): m/z = 524(M + Na)⁺HPLC(Method 2): Rₜ = 5.50 min. |
| 50 | 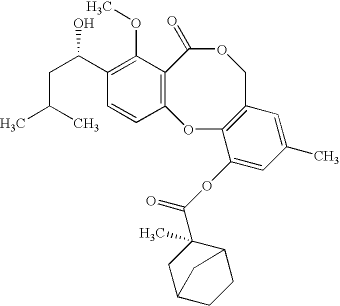 | ¹H-NMR(400 MHz, CDCl₃): δ =0.96(d, 3H), 1.00(d, 3H), 1.18-2.20 (m, 14H), 2.20(dd, 1H), 2.30(s, 3H), 2.31(m, 1H), 2.44(m, 1H), 3.98(s, 3H), 5.00-5.10(m, 3H), 6.72(m, 1H), 6.89(m, 1H), 6.96(d, 1H), 7.57(d, 1H); MS(DCI): m/z = 526(M + NH₄)⁺HPLC(Method 2): Rₜ = 5.60 min. |
| 51 | 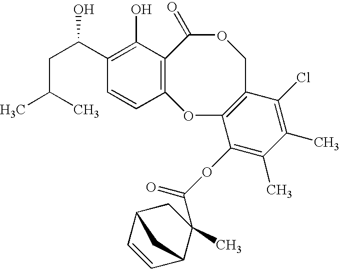 | ¹H-NMR(300 MHz, CDCl₃): δ =0.96(d, 3H), 1.00(d, 3H), 1.18-2.20 (m, 11H), 2.18(s, 3H), 2.31(s, 3H), 2.90(m, 1H), 3.04(m, 1H), 3.98(s, 3H), 5.04(m, 1H), 5.17-5.55(br. m, 2H), 6.10(m, 1H), 6.32(m, 1H), 6.86 (d, 1H), 7.57(d, 1H); MS(DCI): m/z = 572(M + NH₄)⁺HPLC(Method 2): Rₜ = 5.90 min. |
| 52 | 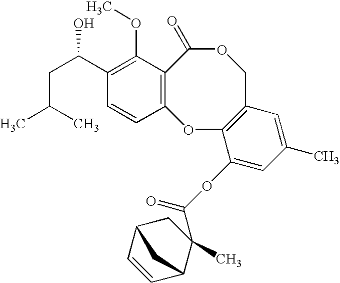 | ¹H-NMR(400 MHz, CDCl₃): δ =0.96.(d, 3H), 0.99(d, 3H), 1.18-1.95 (m, 1OH), 2.08(dd, 1H), 2.28(s, 3H), 2.90(m, 1H), 3.02(m, 1H), 3.98(s, 3H), 5.00-5.10(m, 3H), 6.19(m, 1H), 6.30(m, 1H), 6.70(m, 1H), 6.85(m, 1H), 6.90(d, 1H), 7.57(d, 1H); MS(ESI): m/z = 524(M + Na)⁺HPLC(Method 2): Rₜ = 5.44 min. |
| 53 | 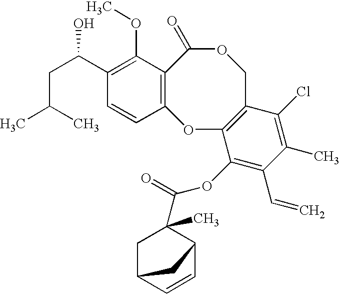 | ¹H-NMR(200 MHz, CDCl₃): δ =0.96(d, 3H), 1.00(d, 3H), 1.18-2.00 (m, 14H), 2.12(dd, 1H), 2.37(m, 1H), 2.31(s, 3H), 2.46(m, 1H), 3.98 (s, 3H), 5.08(m, 1H), 5.40-5.5(m, 3H), 5.62(dd, 1H), 6.51(dd, 1H), 6.95(d, 1H), 7.58(d, 1H); MS(DCI): m/z = 586(M + NH₄)⁺HPLC(Method 2): Rₜ = 6.32 min. |
| 54 | 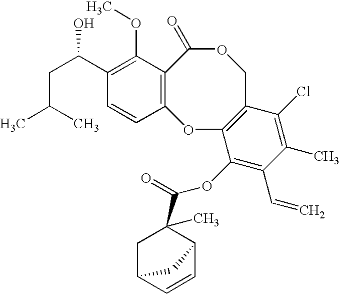 | ¹H-NMR(200 MHz, CDCl₃): δ =0.96(d, 3H), 1.00(d, 3H), 1.18-2.00 (m, 14H), 2.12(dd, 1H), 2.37(m, 1H), 2.31(s, 3H), 2.46(m, 1H), 3.98 (s, 3H), 5.08(m, 1H), 5.40-5.5(m, 3H), 5.62(dd, 1H), 6.51(dd, 1H), 6.95(d, 1H), 7.58(d, 1H); MS(DCI): m/z = 586(M + NH₄)⁺HPLC(Method 2): Rₜ = 6.32 min. |
| 55 | 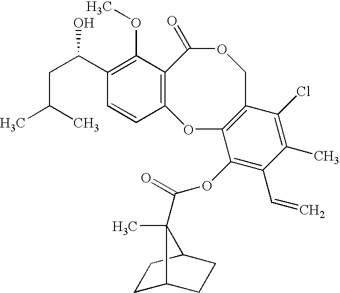 | ¹H-NMR(200 MHz, CDCl₃): δ =0.96(d, 3H), 1.00(d, 3H), 1.18-2.20 (m, 16H), 2.29(m, 1H), 2.32(s, 3H), 3.98(s, 3H), 5.08(m, 1H), 5.40-5.5 (m, 3H), 5.62(dd, 1H), 6.51(dd, 1H), 6.96(d, 1H), 7.58(d, 1H); MS(DCI): m/z = 586(M + NH₄)⁺HPLC(Method 2): Rₜ = 6.33 min. |
| 56 | 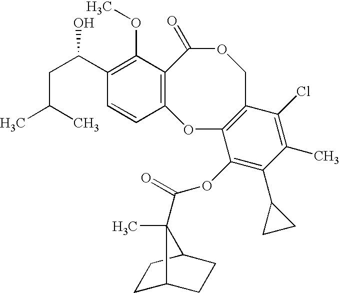 | ¹H-NMR(300 MHz, CDCl₃): δ =0.96(d, 3H), 1.00(d, 3H), 1.00-2.20 (m, 20H), 2.33(m, 2H), 2.46(s, 3H), 3.98(s, 3H), 5.01(m, 1H), 5.20-5.50 (br. m, 2H), 6.98(d, 1H), 7.57(d, 1H); MS(DCI): m/z = 60(M + NH₄)⁺HPLC(Method 2): Rₜ = 6.41 min. |
| 57 | 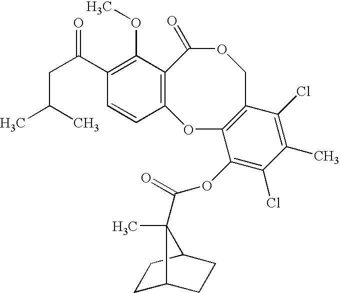 | ¹H-NMR(200 MHz, CDCl₃): δ =0.95(d, 6H), 1 20-2 30(m, 12H), 2.35(m, 2H), 2.50(s, 3H), 2.85(d, 2H), 3.98(s, 3H), 5.40(m, 2H), 6.98 (d, 1H), 7.48(d, 1H); MS(DCI): m/z = 592(M + NH₄)⁺ |
| 58 | 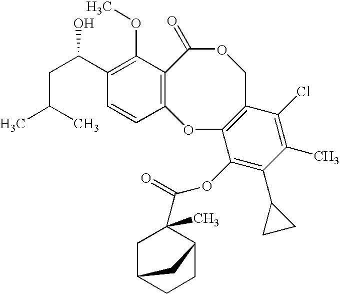 | ¹H-NMR(300 MHz, CDCl₃): δ =0.96(d, 3H), 1.00(d, 3H), 1.00-2.00 (m, 20H), 2.21(m, 1H), 2.30(m, 1H), 2.47(s, 3H), 3.98(s, 3H), 5.08(m, 1H), 5.29-5.45(m, 2H), 6.93(d, 1H), 7.57(d, 1H); MS(DCI): m/z = 60(M + NH₄)⁺HPLC(Method 2): Rₜ = 6.22 min. |
| 59 | 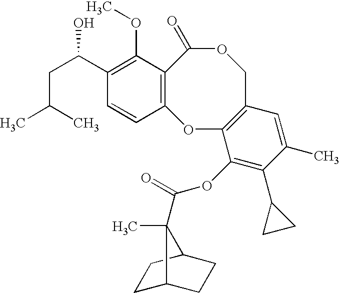 | ¹H-NMR(300 MHz, CDCl₃): δ =0.96(d, 3H), 1.00(d, 3H), 1.00-2.20 (m, 22H), 2.33(s, 3H), 3.98(s, 3H), 4.90-5.10(m, 1H), 6.19(s, 1H), 6.98 (d, 1H), 7.57(d, 1H) MS(DCl): m/z = 60(M + NH₄)⁺HPLC(Method 2): Rₜ = 6.41 min. |
| 60 | 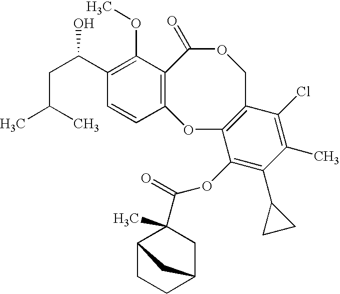 | ¹H-NMR(300 MHz, CDCl₃): δ =0.96(d, 3H), 1.00(d, 3H), 1.00-2.00 (m, 20H), 2.21(m, 1H), 2.30(m, 1H), 2.47(s, 3H), 3.98(s, 3H), 5.08(m, 1H), 5.40(m, 2H), 6.93(d, 1H), 7.57 (d, 1H); LC-MS(Method 5): m/z = 600 (M + Na)⁺, Rₜ = 4.84 min. |
| 61 | 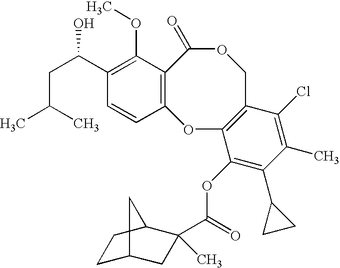 | ¹H-NMR(300 MHz, CDCl₃): δ =0.96(d, 3H), 1.00(d, 3H), 1.00-2.00 (m, 20H), 2.30(m, 1H), 2.47(s, 3H), 2.79(m, 1H), 3.98(s, 3H), 5.06(m, 1H), 5.20-5.50(m, 2H), 6.90(d, 1H), 7.57(d, 1H); LC-MS(Method 5): m/z = 600 (M + Na)⁺ |
| 62 | 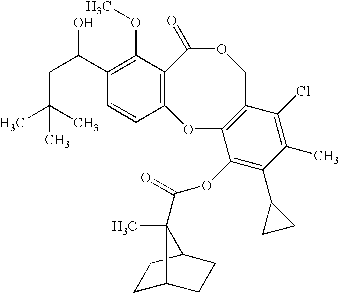 | ¹H-NMR(300 MHz, CDCl₃): δ =0.42-0.88(m, 4H), 1.01(s, 9H), 1.20-2.10(m, 14H), 2.35(m, 2H), 2.47(s, 3H), 3.98(s, 3H), 5.15(m, 1H), 5.20-5.50(br. m, 2H), 6.95(d, 1H), 7.58 (d, 1H); MS(DCl): m/z = 614(M + NH₄)⁺HPLC(Method 2): Rₜ = 6.54 min. |
| 63 | 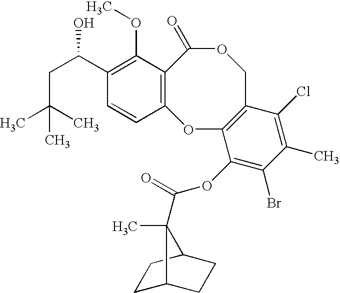 | ¹H-NMR(300 MHz, CDCl₃): δ =1.03(s, 9H), 1.20-2.20(m, 14H), 2.35(m, 2H) 2.55(s, 3H), 3.98(s, 3H), 5.15(m, 1H), 5.30-5.50(br. m, 2H), 6.93(d, 1H), 7.61(d, 1H); MS(DCI): m/z = 652/654(M + NH₄)⁺HPLC(Method 2): Rₜ = 6.66 min. |
| 64 | 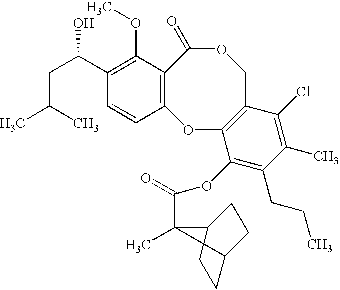 | ¹H-NMR(200 MHz, CDCl₃): δ =0.93-1.06(m, 9H), 1.20-2.08(m, 2.58(br. s, 2H), 3.98(s, 3H), 5.04-5.13(m, 1H), 5.20-5.60(br. s, 2H), 7.00(d, 1H), 7.58(d, 1H); MS(ESIpos): m/z = 607(M + Na)⁺ |
| 65 | 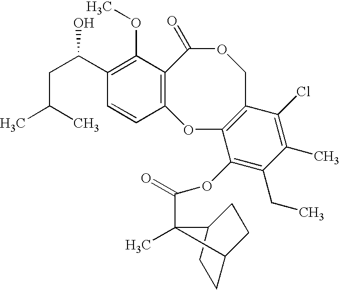 | ¹H-NMR(300 MHz, CDCl₃): δ =0.99(dd, 6H), 1.12(t, 3H), 1.23-2.10 (m, 15H), 2.28(s, 3H), 2.35(s, 2H), 2.59(br. s, 2H), 3.98(s, 3H), 4.90-5.15(m, 3H), 6.71(s, 1H), 7.06(d, 1H), 7.57(d, 1H). |
| 66 | 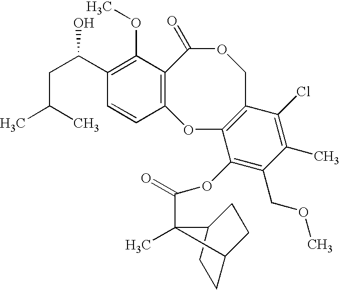 | ¹H-NMR(300 MHz, CDCl₃): δ =0.90(dd, 6H), 1.20-1.98(m, 15H), 2.28(br. s, 1H), 2.34(s, 3H), 3.28(s, 3H), 3.91(s, 3H), 4.36(br. s, 2H), 5.01(dd, 1H), 5.20-5.42(m, 2H), 6.91(d, 1H), 7.50(d, 1H); MS(DCI): rn/z = 604(M + NH₄)⁺ |
| 67 | 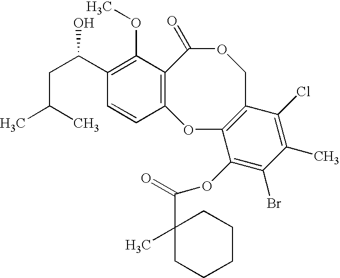 | ¹H-NMR(300 MHz, CDCl₃): δ =0.99(dd, 6H), 1.25-1.90(m, 15H), 2.20-2.32(m, 2H), 2.54(s, 3H), 3.98 (s, 3H), 5.09(dd, 1H), 5.22-5.52(br. s, 2H), 6.92(d, 1H), 7.60(d, 1H); MS(DCI): m/z = 626(M + NH₄)⁺ |
| 68 | 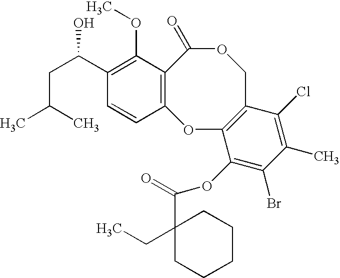 | ¹H-NMR(200 MHz, CDCl₃): δ =0.90-1.08(m, 9H), 1.36-2.08(m, 14H), 2.16-2.3 1(m, 2H), 2.54(s, 3H), 3.98(s, 3H), 5.03-5.13(m, 1H), 5.22-5.52(br. s, 2H), 6.95(d, 1H), 7.60(d, 1H); MS(DCI): m/z = 640(M + NH₄)⁺ |
| 69 | 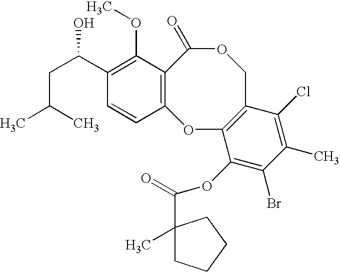 | ¹H-NMR(200 MHz, CDCl₃): δ =0.99(m, 9H), 1.40-1.88(m, 13H), 1.92(d, 1H), 2.35-2.50(m, 2H), 2.54 (s, 3H), 3.98(s, 3H), 5.02-5.13(m, 1H), 5.22-5.60(br. s, 2H), 6.89(d, 1H), 7.60(d, 1H); MS(DCI): m/z = 612(M + NH₄)⁺ |
| 70 | 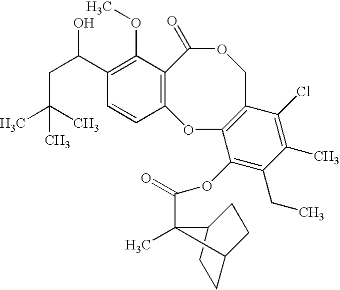 | ¹H-NMR(200 MHz, CDCl₃): δ =1.05(s, 9H), 1.12(t, 3H), 1.22-2.08 (m, 14H), 2.33(s, 5H), 2.63(br. s, 2H), 3.98(s, 3H), 5.09-5.21(m, 1H), 5.21-5.60(br. s, 2H), 6.98(d, 1H), 7.59(d, 1H); MS(DCI): m/z = 602(M + NH₄)⁺ |
| 71 | 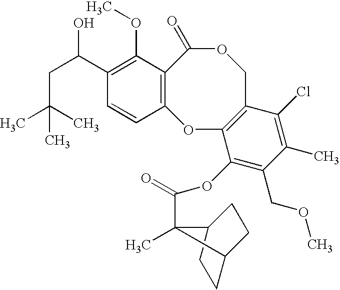 | ¹H-NMR(200 MHz, CDCl₃): δ =1.03(s, 9H), 1.20-2.08(m, 14H), 2.36(br. s, 1H), 2.40(s, 3H), 3.31(s, 3H), 3.98(s, 3H), 4.41(br. s, 2H), 5.09-5.21(m, 1H), 5.22-5.58(m, 2H), 6.97(d, 1H), 7.60(d, 1H); MS(ESIpos): m/z = 600(M)⁺ |
| 72 | 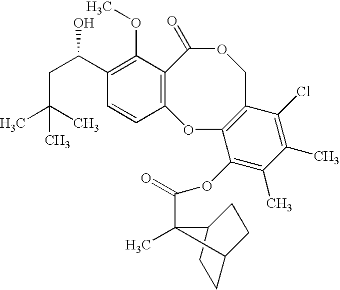 | ¹H-NMR(200 MHz, CDCl₃): δ =1.03(s, 9H), 1.25-1.37(m, 4H), 1.45-1.70(m, 5H), 1.80-1.90(m, 3H), 1.92-2:05(m, 2H), 2.19(s, 3H), 2.32 (s, 3H), 2.35(m, 2H), 3.99(s, 3H), 5.16(m, 1H), 5.21-5.59(m, 2H), 6.94 (d, 1H), 7.59(d, 1H); MS(DCI): m/z = 588(M + NH₄)⁺ |
| 73 | 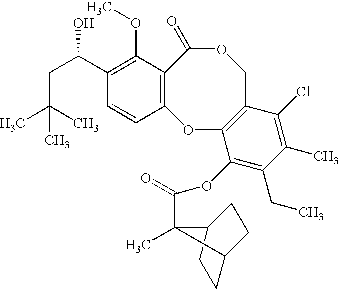 | ¹H-NMR(400 MHz, CDCl₃): δ =1.04(s, 9H), 1.12(t, 3H), 1.24-2.08 (m, 14H), 2.33(s, 5H), 2.63(br. s, 2H), 3.98(s, 3H), 5.13-5.19(m, 1H), 5.21-5.60(br. s, 2H), 6.98(d, 1H), 7.59(d, 1H). |

The examples listed in the table below are prepared analogously to Example B-51, from the corresponding starting materials:

| Example | | |
|---|---|---|
| C- | Structure | Analytical data |
| 74 | 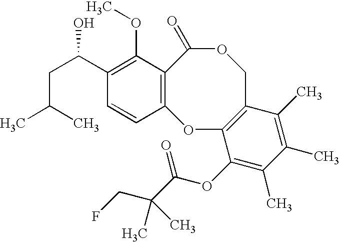 | ¹H-NMR(300 MHz, CDCl₃): δ =0.99(dd, 6H), 1.28(s, 3H), 1.31(s, 3H), 1.45-1.87(m, 4H), 2.12(s, 3H), 2.13(s, 3H), 2.17(s, 3H), 3.99(s, 3H), 4.40(d, 2H), 5.07(dd, 1H), 5.24 (m, 2H), 6.88(d, 1H), 7.56(d; 1H); MS(ESIpos): m/z = 503(M + H)⁺, 525(M + Na)⁺HPLC(Method 1): Rₜ = 5.21 min. |
| 75 | 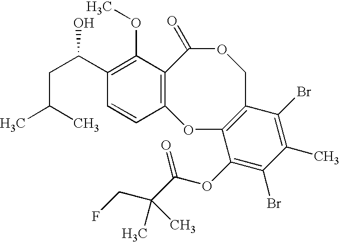 | ¹H-NMR(300 MHz, CDCl₃): δ =0.98(dd, 6H), 1.47(2s, 6H), 1.45-1.87(m, 4H), 2.62(s, 3H), 3.99(s, 3H), 4.59(d, 2H), 5.08(da, 1H), 5.42 (m, 2H), 6.90(d, 1H), 7.60(d, 1H); MS(ESIpos): m/z = 653(M + Na)⁺HPLC(Method 1): Rₜ = 5.69 min. |
| 76 | 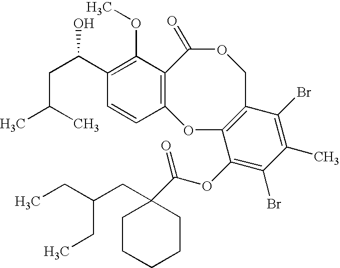 | ¹H-NMR(300 MHz, CDCl₃): δ =0.77(t, 6H), 0.98(dd, 6H), 1.20-1.85 (m, 18H), 1.96(d, 1H), 2.19(d, 2H), 2.62(s, 3H), 3.99(s, 3H), 5.08(m, 1H), 5.40(br. m, 2H), 6.99(d, 1H), 7.60(d, 1H); MS(DCI): m/z = 740(M + NH₄)⁺HPLC(Method 2): Rₜ = 8.14 min. |
| 77 | 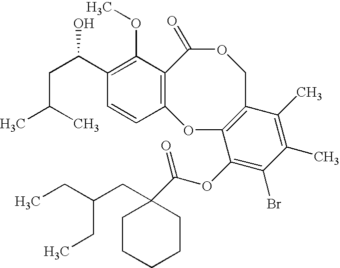 | ¹H-NMR(200 MHz, CDCl₃): δ =0.77(t, 6H), 0.98(dd, 6H), 1.19-1.85 (m, 15H), 1.98(d, 1H), 2.13(d, 2H), 2.09-2.25(m, 10H), 3.99(s, 3H), 5.07(m, 1H), 5.23(br. m, 2H), 7.01 (d, 1H), 7.54(d, 1H); MS(ESIpos): m/z = 595(M + H)⁺, 617(M + Na)⁺HPLC(Method 1): Rₜ = 6.95 min. |
| 78 | 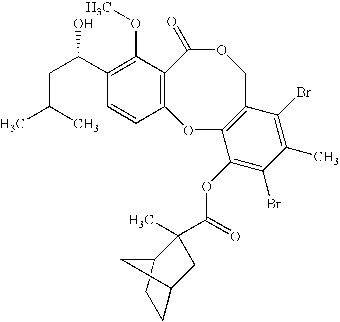 | ¹H-NMR(200 MHz, CDCl₃): δ =0.98(dd, 6H), 1.14-2.50(m, 17H), 2.62(s, 3H), 3.99(s, 3H), 5.09(m, 1H), 5.33-5.48(m, 2H), 6.95(d, 1H), 7.60(d, 1H); MS(ESIpos): m/z = 687(M + Na)⁺HPLC(Method 2): Rₜ = 6.57 min. |
| 79 | 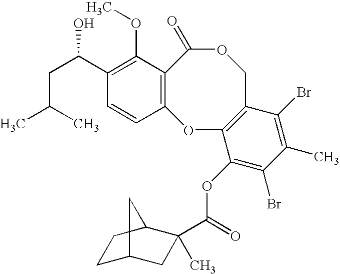 | ¹H-NMR(200 MHz, CDCl₃): δ =0.99(dd, 6H), 1.07-2.00(m, 14H), 2.29(m, 1H), 2.53(m, 1H), 2.61(s, 3H), 2.80(m, 1H), 3.99(s, 3H), 5.09 (m, 1H), 5.42(m, 2H), 6.90(d, 1H), MS(ESIpos): m/z = 687(M + Na)⁺HPLC(Method 2): Rₜ = 6.67. min. |
| 80 | 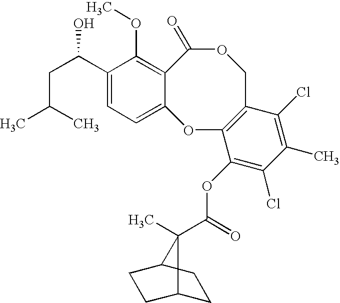 | ¹H-NMR(300 MHz, CDCl₃): δ =0.98(dd, 6H), 1.22-1.95(m, 13H), 2.03(m, 2H), 2.36(m, 2H), 2.49(s, 3H), 3.99(s, 3H), 5.09(m, 1H), 5.38 (br. m, 2H), 6.94(d, 1H), 7.60(d, 1H); MS(DCI): m/z = 594(M + NH₄)⁺HPLC(Method 2): Rₜ = 6.38 min. |
| 81 | 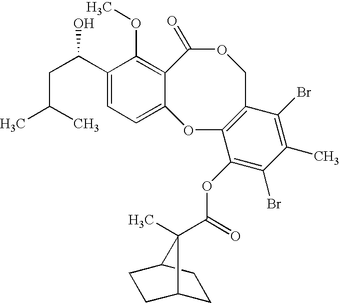 | ¹H-NMR(300 MHz, CDCl₃): δ =0.99(dd, 6H), 1.20-1.97(m, 13H), 2.05(m, 2H), 2.35(m, 2H), 2.62(s, 3H), 3.99(s, 3H), 5.09(m, 1H), 5.40 (br. m, 2H), 6.95(d, 1H), 7.60(d, 1H); MS (DCI): m/z = 681(M + NH₄)⁺HPLC(Method 2): Rₜ = 6.44 min. |
| 82 | 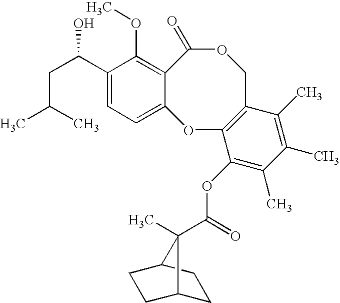 | ¹H-NMR(200 MHz, CDCl₃): δ =0.98(dd, 6H), 1.18-2.07(m, 15H), 2.13(s, 3H), 2.16(s, 3H), 2.17(s, 3H), 2.35(m, 2H), 3.99(s, 3H), 5.07 (m, 1H), 5.23(br. m, 2H), 6.97(d, 1H), 7.55(d, 1H); MS(ESIpos): m/z = 537(M + H)⁺, 559(M + Na)⁺HPLC(Method 1): Rₜ = 5.78 min. |
| 83 | 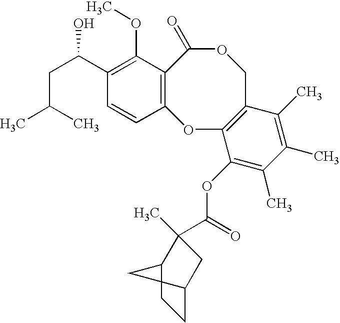 | ¹H-NMR(300 MHz, CDCl₃): δ =0.98(dd,6H),1.15-1.98(m, 13H), 2.13(s, 3H), 2.15(s, 3H), 2.17(s, 3H), 2.18-2.46(m, 3H), 3.99(s, 3H), 5.07(m, 1H), 5.15-5.3 1(m, 2H), 6.96 (d, 1H), 7.54(d, 1H); MS(ESIpos): m/z = 537(M + H)⁺, 559(M + Na)⁺HPLC(Method 2): Rₜ = 5.96 min. |
| 84 | 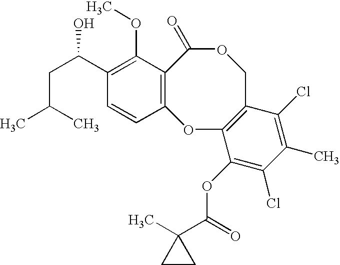 | ¹H-NMR(200 MHz, CDCl₃): δ =0.83-1.05(m, 4H), 0.98(dd, 6H), 1.40-1.85(m, 6H), 1.94(d, 1H), 2.46 (s, 3H), 3.99(s, 3H), 5.09(m, 1H), 5.40(m, 2H), 6.90(d, 1H), 7.61(d, 1H); MS(DCI): m/z = 540(M + NH₄)⁺HPLC(Method 2): Rₜ = 5.70 min. |
| 85 | 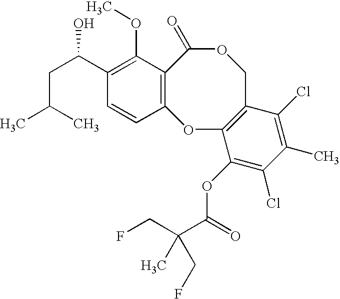 | ¹H-NMR(300 MHz, CDCl₃): δ =0.98(dd, 6H), 1.40-1.88(m, 6H), 1.93(d, 1H), 2.48(s, 3H), 3.99(s, 3H), 4.78(m, 4H), 5.09(m, 1H), 5.40 (m, 2H), 6.83(d, 1H), 7.61(d, 1H); MS(DCI): m/z = 578(M + NH₄)⁺HPLC(Method 1): Rₜ = 5.49 min. |
| 86 | 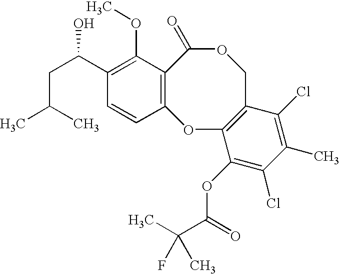 | ¹H-NMR(300 MHz, CDCl₃): δ =0.98(dd, 6H), 1.40-1.88(m, 3H), 1.79(s, 3H), 1.86(s, 3H), 1.91(d, 1H), 2.48(s, 3H), 3.99(s, 3H), 5.09 (m, 1H), 5.41(m, 2H), 6.89(d, 1H), 7.61(d, 1H); MS(DCI): m/z = 546(M + NH₄)⁺HPLC(Method 2): Rₜ = 5.51 min. |
| 87 | 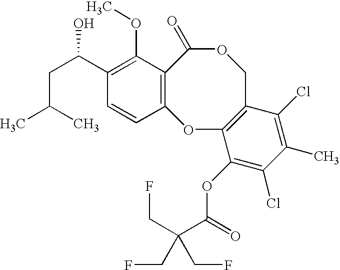 | ¹H-NMR(300 MHz, CDCl₃): δ =0.98(dd, 6H), 1.40-1.87(m, 3H), 1.91(d, 1H), 2.48(s, 3H), 3.98(s, 3H), 4.90(d, 6H), 5.08(m, 1H), 5.40 (m, 2H), 6.82(d, 1H), 7.60(d, 1H); MS(DCI): m/z = 596(M + NH₄)⁺HPLC(Method 2): Rₜ = 5.42 min. |
| 88 | 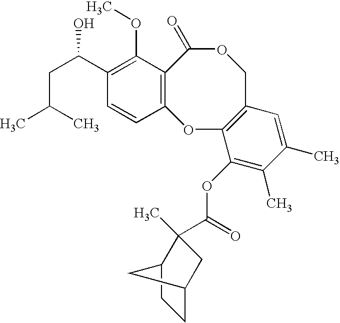 | ¹H-NMR(300 MHz, CDCl₃): δ =0.98(dd, 6H), 1.20-1.96(m, 14H), 2.12(s, 3H), 2.21(s, 3H), 2.18-2.45 (m, 3H), 3.97(s, 3H), 4.98-5.12(m, 3H), 6.71(s, 1H), 6.99(d, 1H), 7.56 (d, 1H); MS(DCI): m/z = 540(M + NH₄)⁺HPLC(Method 1): Rₜ = 5.70 min. |
| 89 | 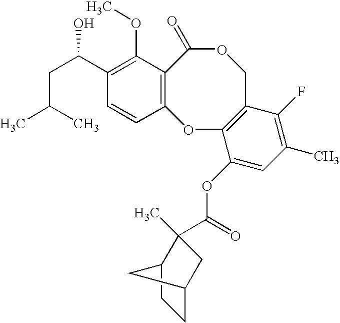 | ¹H-NMR(200 MHz, CDCl₃): δ =0.98(dd, 6H), 1.15-2.04(m, 3H), 1.95 (d, 13H), 2.12-2.46(m, 7H), 3.98(s, 3H), 5.09(m, 1H), 5.24(m, 2H), 6.91(d, 1H), 6.97(dd, 1H), 7.60 (m, 1H); MS(DCI): m/z = 544(M + NH₄)⁺Rf(cyclohexane/ethyl acetate 2:1) =0.39 |
| 90 | 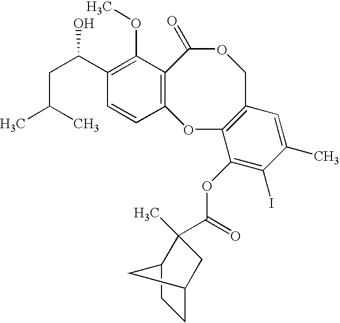 | ¹H-NMR(300 MHz, CDCl₃): δ =0.98(dd, 6H), 1.22-1.95(m, 12H), 2.23-2.48 (m, 5H), 3.97(s, 3H), 5.03 (m, 2H), 5.08(m, 1H), 6.85(s, 1H), 7.01(d, 1H), 7.58(d, 1H); MS(DCl): m/z = 652(M + NH₄)⁺HPLC(Method 1): Rₜ = 5.94 min. |
| 91 | 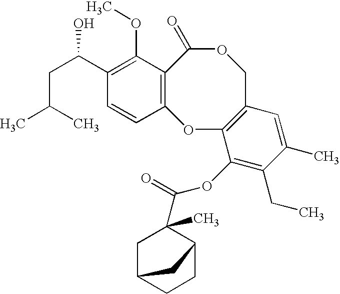 | ¹H-NMR(300 MHz, CDCl₃): δ =0.98(dd, 6H), 1.11(t, 3H), 1.16-1.86 (m, 13H), 1.93(m, 1H), 2.17-2.46 (m, 6H), 2.57(m, 2H), 3.97(s, 3H), 4.95-5.13(m, 3H), 6.72(s, 1H), 7.03 (d, 1H), 7.56(d, 1H); MS(DCI): m/z = 554(M + NH₄)⁺HPLC(Method 1): Rₜ = 5.93 min. |
| 92 | 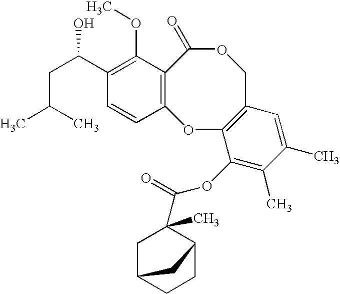 | ¹H-NMR(300 MHz, CDCl₃): δ =0.98(dd, 6H), 1.16-1.88(m, 13H), 1.93(d, 1H), 2.12(s, 3H), 2.21(s, 3H), 2.18-2.49(m, 3H), 3.97(s, 3H), 4.98-5.13(m, 3H), 6.71(s, 1H), 6.99 (d, 1H), 7.56(d, 1H); MS(DCI): m/z = 540(M + NH₄)⁺HPLC(Method 1): Rₜ = 5.77 min. |
| 93 | 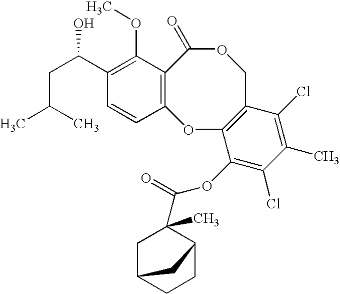 | ¹H-NMR(400 MHz, CDCl₃): δ =0.98(dd, 6H), 1.17-2.02(m, 14H), 2.47(s, 3H), 2.16-2.50(m, 3H), 3.98 (s, 3H), 5.09(m, 1H), 5.40(m, 2H), 6.94(d, 1H), 7.60(d, 1H); MS(DCI): m/z = 594(M + NH₄)⁺HPLC(Method 1): Rₜ = 6.31 min. |
| 94 | 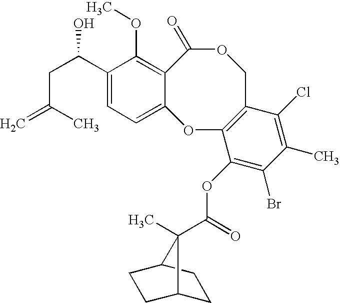 | ¹H-NMR(200 MHz, CDCl₃): δ =1.17-1.61(m, 7H), 1.76-1.94(m, 5H), 1.95-2.49(m, 7H), 2.54(s, 3H), 4.00 (s,3H), 4.81-5.00(m, 2H), 5.12(m, 1H), 5.19-5.54(m, 2H), 6.97(d, 1H), 7.68(d, 1H); MS(DCI): m/z = 636/638(M + NH₄)⁺ |
| 95 | 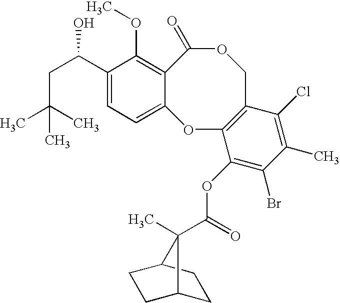 | ¹H-NMR(200 MHz, CDCl₃): δ =1.04(s, 9H), 1.20-1.93(m, 14H), 1.96-2.13(m, 2H), 2.36(m, 2H), 2.54 5.23-5.50(m, 2H), 6.95(d, 1H), 7.62 3H), 3.99(s, 3H), 5.17(m, 1H), MS(DCl): m/z = 652(M + NH₄)⁺ |
| 96 | 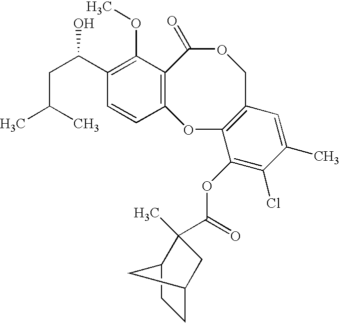 | ¹H-NMR(200 MHz, CDCl₃): δ =0.99(dd, 6H), 1.27-1.88(m, 13H), 1.90-1.97(m, 1H), 2.18-2.38(m, SH), 2.48(br. s, 1H), 3.98(s, 3H), 5.00-5.14(m, 3H), 6.81(s, 1H), 6.98(d, 1H), 7.59(d, 1H); MS(DCI): rn/z = 560(M + NH₄)⁺ |
| 97 | 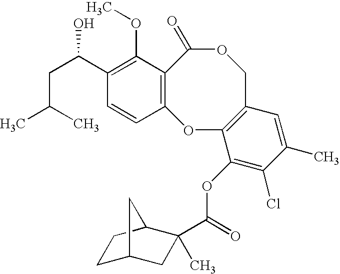 | ¹H-NMR(200 MHz, CDCl₃): δ =0.99(dd, 6H), 1.12-1.88(m, 13H), 1.95(d, 1H), 2.25-2.35(m, 4H), 2.52-2.63(m, 1H), 2.80(br. s, 1H), 3.98(s, 3H), 4.98-5.14(m, 3H), 6.81(s, 1H), 6.92(d, 1H), 7.59(d, 1H); MS(DCI): m/z = 560(M + NH₄)⁺ |
| 98 | 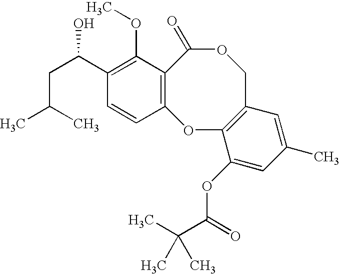 | ¹H-NMR(300 MHz, CDCl₃): δ =0.99(dd, 6H), 1.40(s, 9H), 1.42-1.88 (m, 3H), 1.92(d, 1H), 3.98(s, 3H), 5.05-5.13(m, 3H), 6.91(d, 1H), 7.09 (d, 1H), 7.28(d, 1H), 7.61(d, 1H); MS(DCI): m/z = 538(M + NH₄)⁺ |
| 99 | 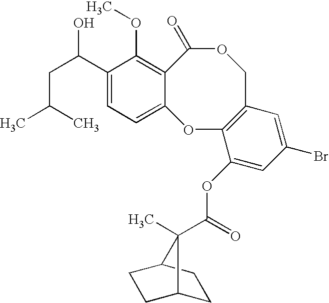 | ¹H-NMR(300 MHz, CDCl₃): δ =0.99(dd, 6H), 1.30-1.99(m, 15H), 2.32(br. s, 2H), 3.98(s, 3H), 5.03-5.14(m, 3H), 6.98(d, 1H), 7.09(d, 1H), 7.22(d, 1H), 7.60(d, 1H); MS(DCI): m/z = 590(M + NH₄)⁺ |
| 100 | 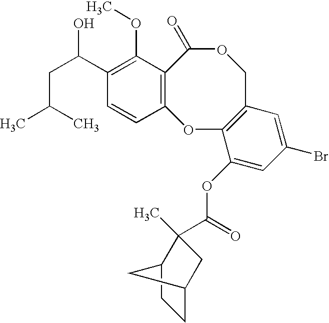 | ¹H-NMR(300 MHz, CDCl₃): δ =0.99(dd, 6H), 1.20-1.88(m, 13H), 1.90(d, 1H), 2.15-2.23(m, 1H), 2.32 (br. s, 1H), 2.41(br. s, 1H), 3.96(s, 3H), 5.03-5.13(m, 3H), 6.96(d, 1H), 7.09(d, 1H), 7.26(d, 1H), 7.60(d, 1H); MS(DCI): m/z = 590(M + NH₄)⁺ |
| 101 | 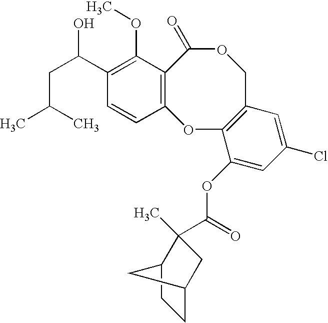 | ¹H-NMR(300 MHz, CDCl₃): δ =0.99(dd, 6H), 1.20-1.88(m, 13H), 1.92(d, 1H), 2.15-2.23(m, 1H), 2.32 (br. s, 1H), 2.41(br. s, 1H), 3.97(s, 3H), 5.03-5.12(m, 3H); 6.92-6.98(m, 2H), 7.11(d, 1H), 7.60(d, 1H); MS(DCI): m/z = 546(M + NH₄)⁺ |
| 102 | 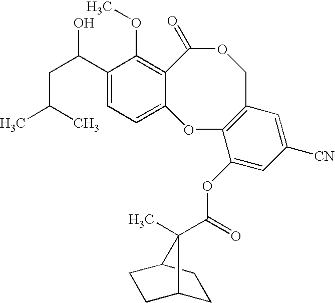 | ¹H-NMR(300 MHz, CDCl₃): δ =0.99(dd, 6H), 1.30-1.98(m, 15H), 2.32(br. s, 2H), 3.98(s, 3H), 5.06-5.12(m, 3H), 6.99(d, 1H), 7.28(d, 1H), 7.40(d, 1H), 7.63(d, 1H); MS(DCI): m/z = 537(M + NH₄)⁺ |
| 103 | 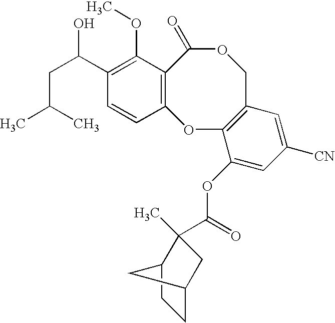 | ¹H-NMR(300 MHz, CDCl₃): δ =0.99(dd, 6H), 1.20-1.88(m, 13H), 1.92(br. s, 1H), 2.15-2.22(m, 1H), 2.32(br. s, 1H), 2.42(br. s, 1H), 3.97 (s, 3H), 5.05-5.14(m, 3H), 6.94-6.99 (m, 1H), 7.29(d, 1H), 7.42(d, 1H), 7.62(d, 1H); MS(DCI): m/z = 537(M + NH₄)⁺ |
| 104 | 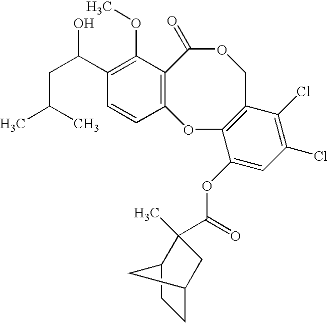 | ¹H-NMR(300 MHz, CDCl₃): δ =0.99(dd, 6H), 1.20-1.88(m, 13H), 1.91(d, 1H), 2.15-2.22(m, 1H), 2.31 (br. s, 1H), 2.41(br. s, 1H), 3.98(s, 3H), 5.05-5.13(m, 1H), 5.32-5.50(m, 2H), 6.88-6.94(m, 1H), 7.27(s, 1H), 7.61(d, 1H); MS(DCI): m/z = 580(M + NH₄)⁺ |
| 105 | 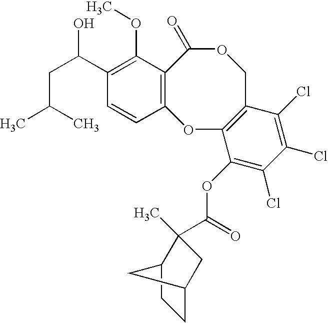 | ¹H-NMR(300 MHz, CDCl₃): δ =0.99(dd, 6H), 1.20-1.88(m, 13H), 1.92(br. s, 1H), 2.15-2.24(m,1H), 2.31(br. s, 1H), 2.44(br. s, 1H), 3.98 (s, 3H), 5.05-5.12(m, 1H), 5.30-5.50 (m, 2H), 6.92(d, 1H), 7.62(d, 1H); MS(DCI): m/z = 614(M + NH₄)⁺ |
| 106 | 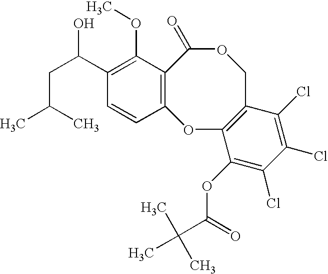 | ¹H-NMR(300 MHz, CDCl₃): δ =0.99(dd, 6H), 1.44(s, 9H), 1.42-1.88 (m, 3H), 1.92(d, 1H), 3.98(s, 3H), 5.05-5.12(m, 1H), 5.31-5.48(m, 2H), 6.88(d, 1H), 7.62(d, 1H); MS(ESIpos): m/z = 487(M + NH₄)⁺ |
| 107 | 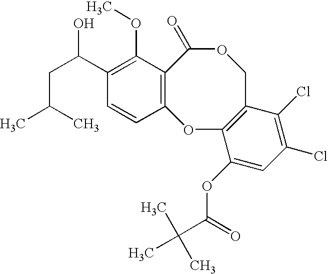 | ¹H-NMR(300 MHz, CDCl₃): δ =0.99(dd, 6H), 1.39(s, 9H), 1.42-1.88 (m, 3H), 1.92(br. s, 1H), 3.98(s, 3H), 5.07-5.13(m, 1H), 5.32-5.48(m, 2H), 6.89(d, 1H), 7.28(s, 1H), 7.61 (d, 1H); MS(DCI): m/z = 528(M + NH₄)⁺ |
| 108 | 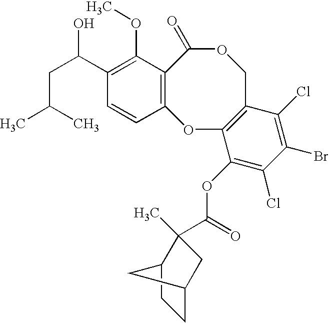 | ¹H-NMR(300 MHz, CDCl₃): δ =0.99(dd, 6H), 1.20-1.88(m, 13H), 1.92(br. s, 1H), 2.15-2.24(m, 1H), 2.32(br. s, 1H), 2.44(br. s, 1H), 3.98 (s, 3H), 5.06-5.13(m, 1H), 5.30-5.50 (m, 2H), 6.92(d, 1H), 7.62(d, 1H); MS(DCI): m/z = 660(M + NH₄)⁺ |
| 109 | 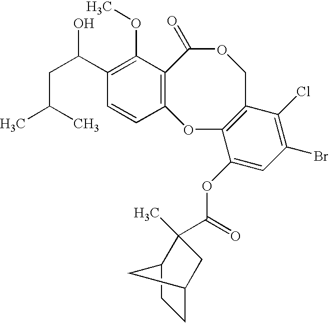 | ¹H-NMR(300 MHz, CDCl₃): δ =0.99(dd, 6H), 1.20-1.88(m, 13H), 1.91(d, 1H), 2.15-2.24(m, 1H), 2.32 (br. s, 1H), 2.42(br. s, 1.H), 3.98(s, 3H), 5.05-5.13(m, 1H), 5.33-5.50(m, 2H), 6.92(d, 1H), 7.41(s, 1H), 7.61 (d, 1H); MS(DCI): m/z = 624(M + NH₄)⁺ |
| 110 | 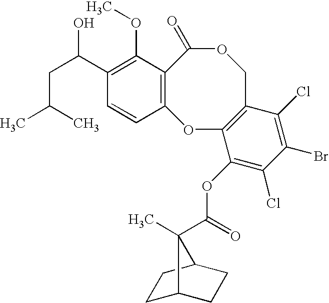 | ¹H-NMR(300 MHz, CDCl₃): δ =0.99(dd, 6H), 1.30-2.08(m, 15H), 2.36(br. s, 2H), 3.99(s, 3H), 5.05-5.13(m, 1H), 5.26-5.50(m, 2H), 6.92 (d, 1H), 7.62(d, 1H); MS(DCI): m/z = 660(M + NH₄)⁺ |
| 111 | 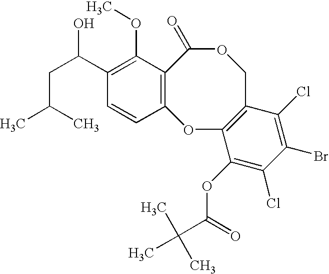 | ¹H-NMR(400 MHz, CDCl₃): δ =0.99(dd, 6H), 1 42(s, 9H), 1.42-1.88 (m, 3H), 1.92(d, 1H), 3.98(s, 3H), 5.06-5.12(m, 1H), 5.32-5.50(m, 2H), 6.88(d, 1H), 7.62(d, 1H); MS(DCI): m/z = 608(M + NH₄)⁺ |
| 112 | 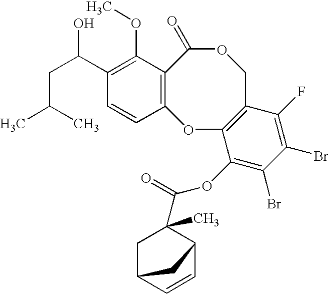 | ¹H-NMR(300 MHz, CDCl₃): δ =0.99(dd, 6H), 1.42-1.88(m, 9H), 1.90(br. s, 1H), 2.08-2. 17(m, 1H), 2.90(br. s, 1H), 3.02(br. s, 1H), 3.98/3.99(s, 3H), 5.07-5.12(m, 1H), 5.13-5.32(m, 3H), 5.07-5.12(m, 1H), s, 1H), 6.90(d, 1H), 7.61(d, 1H); LC-MS(Method 7): Rₜ = 4.56 min. MS(ESIpos): m/z = 649 (M + H − H₂O)⁺ |
| 113 | 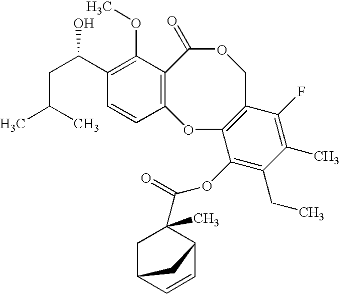 | ¹H-NMR(300 MHz, CDCl₃): δ =0.99(dd, 6H), 1.11(t, 3H), 1.42-1.88 (m, 7H), 1.92(d, 1H), 2.07-2.15(m, 1H), 2.32(s, 3H), 2.59(br. s, 2H), 2.90(br. s, 1H), 3.00(br. s, 1H), 3.88/3.89(2s, 2H), 3.98(s, 3H), 5.03-6.15(br. s, 6.25-6.30 6.90(d, 1H), 7.58(d, 1H). |
| 114 | 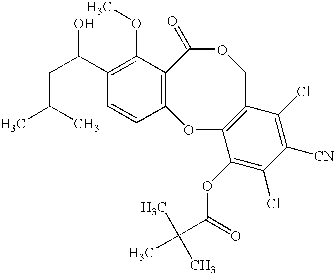 | ¹H-NMR(300 MHz, CDCl₃): δ =0.99(dd, 6H), 1.42(s, 9H), 1.42-1.88 (m, 3H), 1.92(d, 1H), 3.98(s, 3H), 5.06-5.13(m, 1H), 5.28-5.44(m, 2H), 6.87(d; 1H), 7.68(d, 1H); MS(DCI): m/z = 553(M + NH₄)⁺ |
| 115 | 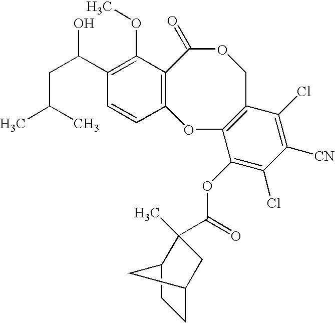 | ¹H-NMR(300 MHz, CDCl₃): δ =0.99(dd, 6H), 1.20-1.88(m, 13H), 1.92(br. s, 1H), 2.15-2.23(m, 1H), 2.32(br. s, 1H), 2.45(br. s, 1H), 3.98 (s, 3H), 5.06-5.13(m, 1H), 5.22-5.47 (m, 2H), 6.92(d, 1H), 7.68(d, 1H); MS(DCI): m/z = 605(M + NH₄)⁺ |
| 116 | 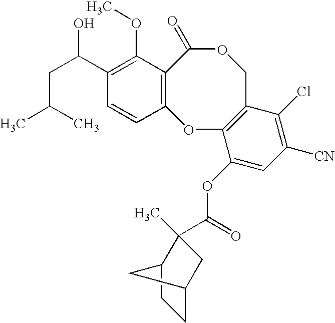 | ¹H-NMR(300 MHz, CDCl₃): δ =0.99(dd, 6H), 1.20-1.88(m, 13H), 1.92(d, 1H), 2. 14-2.22(m, 1H), 2.32 (br. s, 1H), 2.42(br. s, 1H), 3.98(s, 3H), 5.06-5.13(m, 1H), 5.28-5.48(m, 2H), 6.92(d, 1H), 7.44(s, 1H), 7.66 (d, 1H); MS(DCI): m/z = 571(M + NH₄)⁺ |
| 117 | 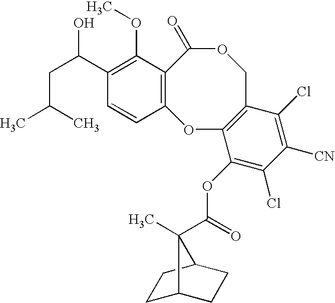 | ¹H-NMR(300 MHz, CDCl₃): δ =0.99(dd, 6H), 1.28-2.06(m, 15H), 2.36(br. s, 2H), 3.99(s, 3H), 5.06-5.14(m, 1H), 5.20-5.48(m, 2H), 6.91 (d, 1H), 7.67(d, 1H); MS(DCI): m/z = 605(M + NH₄)⁺ |
| 118 | 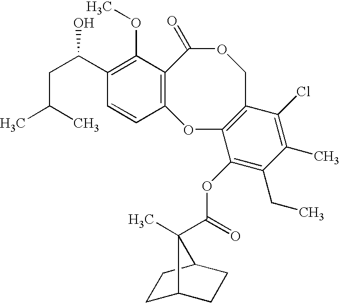 | ¹H-NMR(200 MHz, CDCl₃): δ =0.99(dd, 6H), 1.12(t, 3H), 1.26-2.08 (m, 15H), 2.32(s, 5H), 2.62(br. s, 2H), 3.99(s, 3H), 5.02-5.13(m, 1H), 5.20-5.60(br. s, 2H), 7.00(d, 1H), 7.58(d, 1H); MS(DCI): m/z = 588(M + NH₄)⁺ |
| 119 | 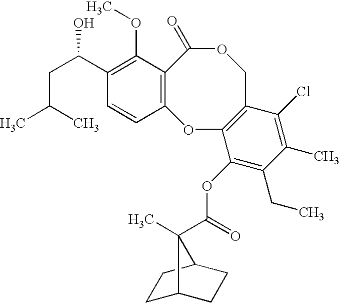 | ¹H-NMR(200 MHz, CDCl₃): δ =0.99(dd, 6H), 1.20-2.08(m, 15H), 2.38(br. s, 2H), 2.59(s, 3H), 3.98(s, 3H), 5.05-5.16(m, 1H), 5.28-5.53(m, 2H), 6.92(d, 1H), 7.63(d, 1H); MS(DCI): m/z = 585(M + NH₄)⁺ |
| 120 | 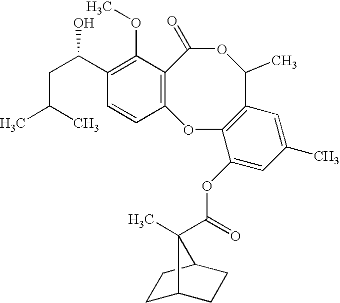 | ¹H-NMR(300 MHz, CDCl₃): δ =0.99(dd, 6H), 1.28-2.03(m, 18H), 2.32(br. s, 5H), 3.97(s, 3H), 5.06-5.12(m, 1H), 5.45(q, 1H), 6.84-6.90 (m, 2H), 6.99(d, 1H), 7.56(d, 1H). |

The examples listed in the table below are prepared analogously to Example B-98, from the corresponding starting materials:

| Example | | |
|---|---|---|
| C- | Structure | Analytical data |
| 121 | 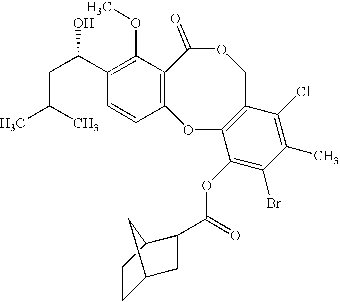 | ¹H-NMR(300 MHz, CDCl₃): δ =0.96(d, 3H), 0.99(d, 3H), 1.18-1.95 (m, 12H), 2.34(m, 1H), 2.52(s, 3H), 2.83(m, 1H), 3.19(m, 1H), 3.98(s, 3H), 5.02(m, 1H), 5.39(m, 2H), 6.91 (d, 1H), 7.47(d, 1H); MS(DCI): m/z = 624/626(M + NH₄)⁺HPLC(Method 2): Rₜ = 5.70 min. |
| 122 | 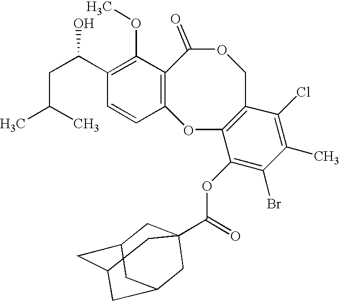 | ¹H-NMR(300 MHz, CDCl₃): δ =0.96(d, 3H), 0.99(d, 3H), 1.18-1.95 (m, 19H), 2.52(s, 3H), 3.98(s, 3H), 5.08(m, 1H), 5.30-5.48(br. m, 2H), 6.88(d, 1H), 7.48(d, 1H); MS(DCI): m/z = 664/666(M + NH₄)⁺HPLC(Method 2): Rₜ = 6.30 min. |
| 123 | 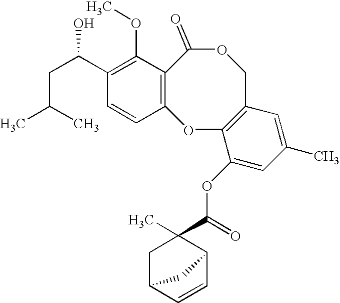 | ¹H-NMR(400 MHz, CDCl₃): δ =0.96(d, 3H), 0.99(d, 3H), 1.18-1.95 (m, 10H), 2.08(dd, 1H), 2.28(s, 3H), 2.90(m, 1H), 3.02(m, 1H), 3.98(s, 3H), 5.00-5.10(m, 3H), 6.19(m, 1H), 6.30(m, 1H), 6.70(m, 1H), 6.85(m, 1H), 6.90(d, 1H), 7.57(d, 1H); MS(ESI): m/z = 524(M + Na)⁺HPLC(Method 2): Rₜ = 5.57 min. |
| 124 | 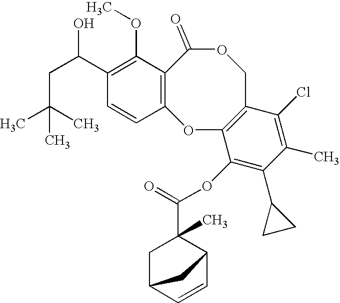 | ¹H-NMR(200 MHz, CDCl₃): δ =0.5-1.0(m, 4H), 1.03(s,9H), 1.20-2.20 (m, 10H), 2.45(s, 3H), 2.88(m, 1H), 3.00(m, 1H), 3.98(s, 3H), 5.15(m, 1H), 5.20-5.50(br. m, 2H), 6.10(m, 1H), 6.28(m, 1H), 6.88(d, 1H), 7.57 (d, 1H); MS(ESI): m/z = 612(M + Na)⁺HPLC(Method 2): Rₜ = 6.25 min. |

The examples listed in the table below are prepared analogously to Example B-168, from the corresponding starting materials:

| Example | | |
|---|---|---|
| C- | Structure | Analytical data |
| 125 | 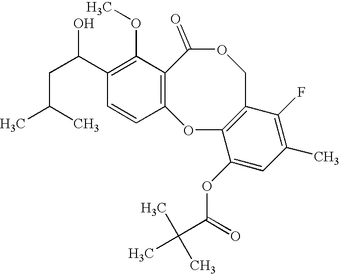 | ¹H-NMR(200 MHz, CDCl₃): δ=0.98(dd, 6H), 1.39(s, 9H), 1.25-1.95 (m, 4H), 2.21(d, 3H), 3.98(s, 3H), 5.09(m, 1H), 5.24(m, 2H), 6.89-6.96 (m, 2H), 7.60(d, 1H); MS(ESIpos): m/z = 475(M + H)⁺, 497(M + Na)⁺ |
| 126 | 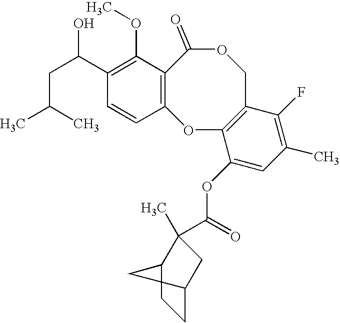 | ¹H-NMR(200 MHz, CDCl₃): δ =0.98(dd, 6H), 1.10-1.90(m, 10H), 1.94(d, 1H), 2.11-2.75(m, 6H), 3.98 (s, 3H), 5.09(m, 1H), 5.24(m, 2H), 6.86-7.01(m, 2H), 7.60(d, 1H); MS(DCI): m/z = 544(M + NH₄)⁺ |
| 127 | 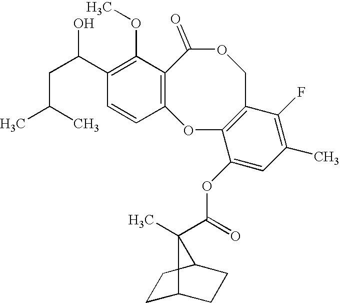 | ¹H-NMR(200 MHz, CDCl₃): δ =0.98(dd, 6H), 1.20-1.99(m, 15H), 2.21(d, 3H), 2.32(m, 2H), 3.98(s, 3H), 5.09(m, 1H), 5.22(m, 2H), 6.88 (d, 1H), 6.98(d, 1H), 7.59(d, 1H); MS(ESIpos): m/z = 527(M + H)⁺HPLC(Method 2): Rₜ = 5.86 min. |
| 128 | 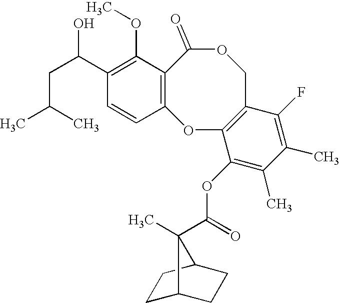 | ¹H-NMR(300 MHz, CDCl₃): δ =0.98(dd, 6H), 1.21-1.91(m, 12H), 1.94-2.05(m, 2H), 2.14(m, 7H), 2.36 (m, 2H), 3.98(s, 3H), 5.08(m, 1H), 5.12-5.25(m, 2H), 6.99(d, 1H), 7.58 (d, 1H); MS(DCI): m/z = 558(M + NH₄)⁺R_{f}(toluene/ethyl acetate 9:1) = 0.24 |
| 129 | 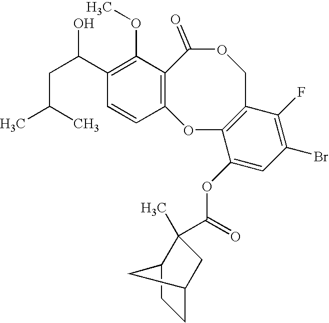 | ¹H-NMR(200 MHz, CDCl₃): δ =0.99(dd, 6H), 1.20-1.88(m, 13H), 1.91(d, 1H), 2.13-2.23(m, 1H), 2.31 (br. s, 1H), 2.41(br. s, 1H), 3.98(s, 3H), 5.05-5.14(m, 1H), 5.16-5.38(m, 2H), 6.95(d, 1H), 7.31(d, 1H), 7.62 (d, 1H); MS(ESIpos): m/z = 613(M + Na)⁺ |

The examples listed in the table below are prepared analogously to Example B-136, from the corresponding starting materials:

| Example | | |
|---|---|---|
| C- | Structure | Analytical data |
| 130 | 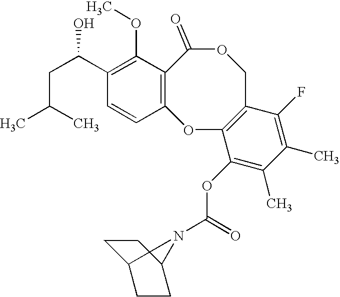 | ¹H-NMR(300 MHz, CDCl₃): δ =0.97(dd, 6H), 1.25-1.95(m, 12H), 2.12(s, 3H), 2.18(s, 3H), 2.22(s, 3H), 3.99(s, 3H), 4.50(br. m, 2H), 5.06(m, 1H), 5.22(m, 2H), 7.11(d, 1H), 7.54(d, 1H); MS(DCI): m/z = 541(M + NH₄)⁺HPLC(Method 2): Rₜ = 5.33 min. |
| 131 | 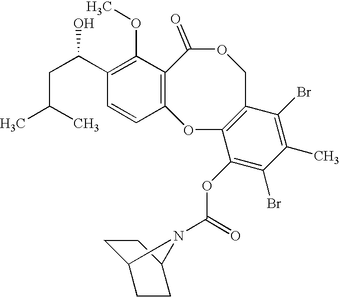 | ¹H-NMR(300 MHz, CDCl₃): δ =0.98(dd, 6H), 1.25-1.95(m, 12H), 2.61(s, 3H), 3.98(s, 3H), 4.50(br. m, 2H), 5.08(m, 1H), 5.38(br. m, 2H), 7.13(d, 1H), 7.60(d, 1H); MS(DCI): m/z = 669(M + NH₄)⁺HPLC(Method 2): Rₜ = 5.81 min. |
| 132 | 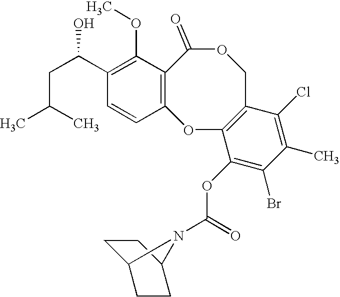 | ¹H-NMR(300 MHz, CDCl₃): δ =0.96(d, 3H), 0.99(d, 3H), 1.18-1.30 (m,4H), 1.40-1.50(m,4H), 1.61-1.99 (m, 4H), 2.52(s, 3H), 3.98(s, 3H), 4.44-4.56(m, 2H), 5.04-5.12(m, 1H), 5.21-5.55(br. m, 2H), 7.12(d, 1H), 7.59(d, 1H); MS(DCI): m/z = 625/627(M + NH₄)⁺HPLC(Method 2): Rₜ = 5.60 min. |

The examples listed in the table below are prepared analogously to Example A-42 using 1,8-diazabicyclo[5.4.0]undec-7-ene instead of sodium hydride, from the corresponding starting materials:

| Example | | |
|---|---|---|
| C- | Structure | Analytical data |
| 133 | 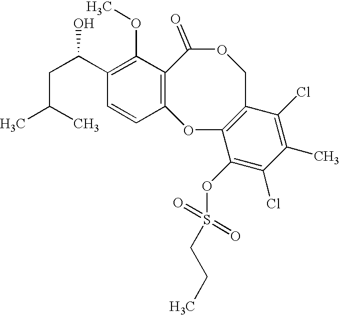 | ¹H-NMR(400 MHz, CDCl₃): δ =0.99(dd, 6H), 1.12(t, 3H), 1.42-1.88 (m, 3H), 1.98(br. s, 1H), 2.05-2.18 (m, 2H), 2.50(s, 3H), 3.51(t, 3H), 3.98(s, 3H), 5.06-5.14(m, 1H), 5.32-5.50(m, 2H), 7.11(d, 1H), 7.65(d, 1H); MS(DCI): m/z = 564(M + NH₄)⁺ |
| 134 | 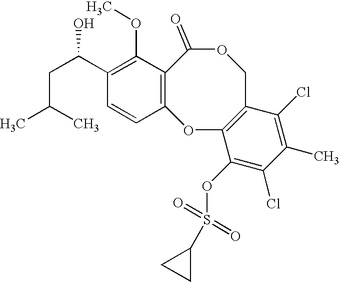 | ¹H-NMR(400 MHz, CDCl₃): δ =0.99(dd, 6H), 1.15-1.22(m, 2H), 1.39-1.88(m, 5H), 1.92(d, 1H), 2.50 (s, 3H), 2.88-2.98(m, 1H), 3.98(s, 3H), 5.06-5.14(m, 1H), 5.38-5.50(m, 2H), 7.12(d, 1H), 7.64(d, 1H); MS(DCI): m/z = 562(M + NH₄)⁺ |
| 135 | 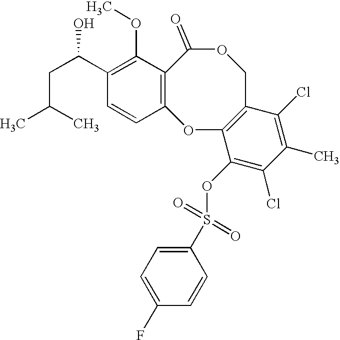 | ¹H-NMR(300 MHz, CDCl₃): δ =0.99(dd, 6H), 1.42-1.88(m, 3H), 1.91(d, 1H), 2.48(s, 3H), 3.95(s, 3H), 5.03-5.10(m, 1H), 2H), 6.83(d, 1H), 7.14-7.21(m, 2H), 7.61(d, 1H), 7.98-8.06(m, 2H); MS(ESIpos): m/z = 621(M + Na)⁺. |

### EXAMPLE C-136

### 4-Cyano-9-[(1S)-1-hydroxy-3-methylbutyl]-8-methoxy-3-methyl-7-oxo-5H,7H-dibenzo[b,g][1,5]dioxocin-1-yl pivalate

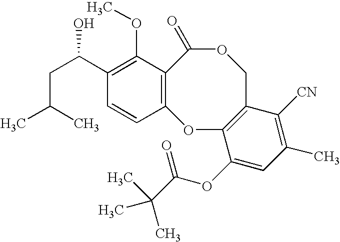

Example C-1 (95 mg, 0.18 mmol) is dissolved in DMF (3.8 ml), copper(I) cyanide (79 mg, 0.89 mmol) is added and the mixture is heated at 140° C. for 6 h. After cooling, the reaction mixture is purified directly by preparative RP-HPLC. This gives 24 mg (28% of theory) of the desired product.

¹H-NMR (200 MHz, CDCl₃): δ=0.97 (d, 3H), 1.00 (d, 3H), 1.40 (s, 9H), 1.45-1.86 (m, 3H), 1.94 (d, 1H), 2.52 (s, 3H), 3.98 (s, 3H), 5.03-5.16 (m, 1H), 5.32-5.50 (m, 2H), 6.89 (d, 1H), 7.10 (s, 1H), 7.63 (d, 1H); LC-MS (Method 4): Rₜ=4.52 min.;

MS (ESIpos): m/z=504 (M+Na)⁺.

### EXAMPLE C-137

### 4-Cyano-9-[(1S)-1-hydroxy-3-methylbutyl]-8-methoxy-3-methyl-7-oxo-5H,7H-dibenzo[b,g][1,5]dioxocin-1-yl 2-methylbicyclo[2.2.1]heptane-2-carboxylate

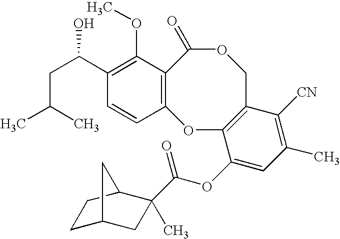

Example C-2 (50 mg, 0.09 mmol) is reacted analogously to Example C-1. This gives 14 mg (30% of theory) of the title compound.

¹H-NMR (200 MHz, CDCl₃): δ=0.97 (d, 3H), 1.00 (d, 3H), 1.15-1.86 (m, 10.5H), 1.46 (s, 3H), 1.96 (d, 1H), 2.17 (dd, 0.5H), 2.26-2.35 (m, 1H), 2.39-2.44 (m, 0.5H), 2.52 (s, 3H), 2.67-2.73 (m, 0.5H), 3.98 (s, 3H), 5.03-5.15 (m, 1H), 5.32-5.50 (m, 2H), 6.92 (dd, 1H), 7.05-7.10 (1H), 7.63 (1H);

LC-MS (Method 4): Rₜ=4.78 min.;

MS (ESIpos): m/z=556 (M+Na)⁺.

The examples listed in the table below are prepared analogously to Example B-51, from the corresponding starting materials:

| Example | |
|---|---|
| C- | Structure |
| 138 | 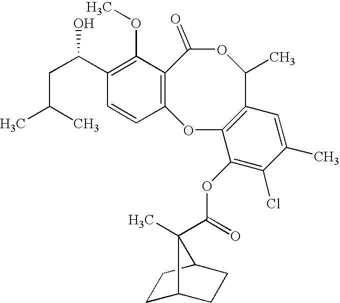 |
| 139 | 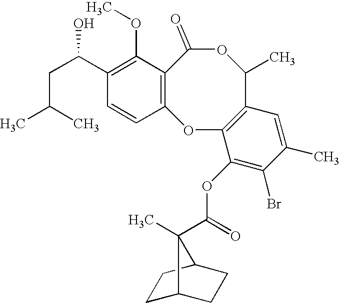 |
| 140 | 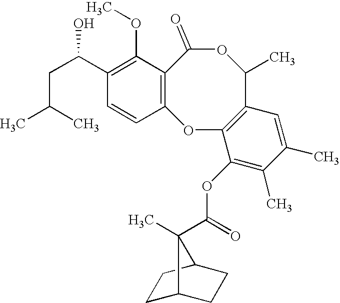 |
| 141 | 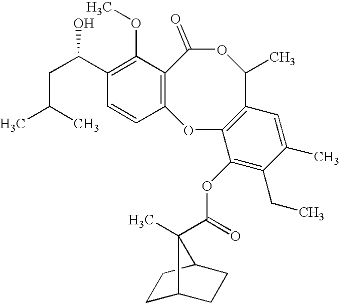 |
| 142 | 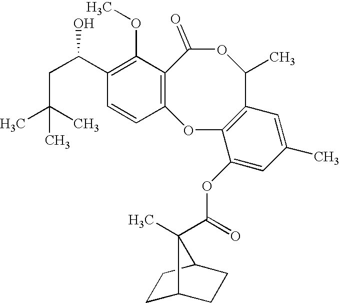 |
| 143 | 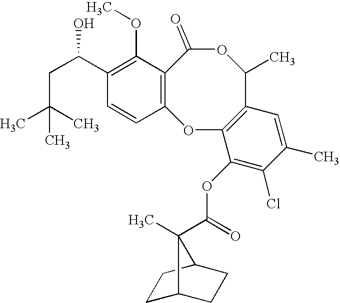 |
| 144 | 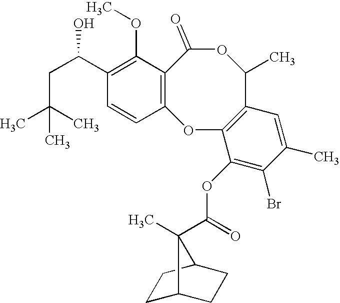 |
| 145 | 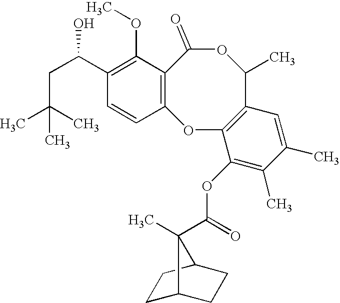 |
| 146 | 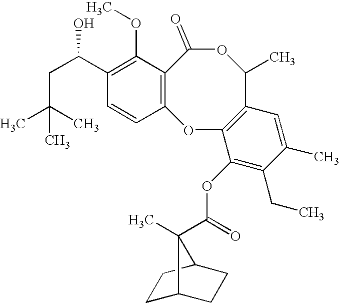 |
| 147 | 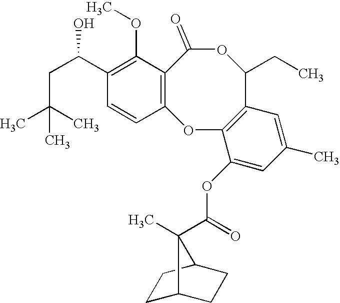 |
| 148 | 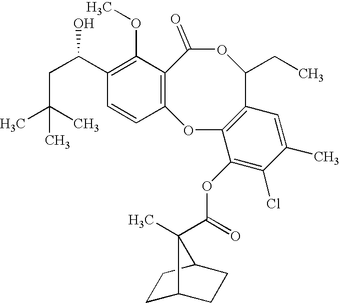 |
| 149 | 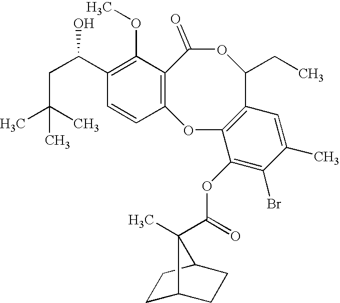 |
| 150 | 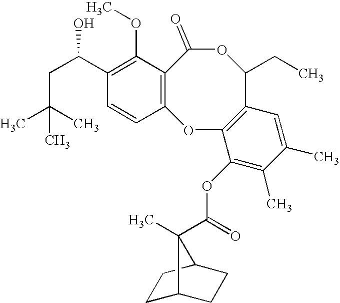 |
| 151 | 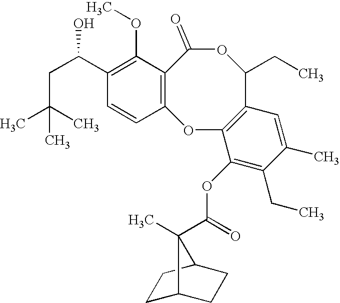 |

A. CETP-Inhibition Testing In Vitro
A1. Obtainment of CETP

CETP is obtained in partially purified form from human plasma by differential centrifugation and column chromatography and used for the test. To this end, human plasma is adjusted to a density of 1.21 g per ml using NaBr and centrifuged at 4° C. at 50 000 rpm for 18 h. The bottom fraction (d>1.21 g/ml) is applied to a Sephadex® Phenyl-Sepharose 4B (Pharmacia) column, washed with 0.15 M NaCl/0.001 M tris HCl pH 7.4 and then eluted with distilled water. The CETP-active fractions are pooled, dialysed against 50 mM sodium acetate pH 4.5 and applied to a CM-Sepharose® column (Pharmacia). The mixture is then eluted using a linear gradient (0-1 M NaCl). The pooled CETP fractions are dialysed against 10 mM Tris HCl pH 7.4 and then further purified by chromatography on a Mono Q® column (Pharmacia).

A2. CETP Fluorescence Test

Measurement of the CETP-catalysed transfer of a fluorescent cholesterol ester between liposomes—modified according to the procedure of Bisgaier et al., J. Lipid Res. 34, 1625 (1993):

For the production of the donor liposomes, 1 mg of cholesteryl 4,4-difluoro-5,7-dimethyl-4-bora-3a,4a-diaza-s-indacene-3-dodecanoate (cholesteryl BODIPY® FL C₁₂, Molecular Probes) is dissolved in 600 μl of dioxane with 5.35 mg of triolein and 6.67 mg of phosphatidylcholine with gentle warming in an ultrasonic bath and this solution is added very slowly with ultrasonication to 63 ml of 50 mM tris/HCl, 150 mM NaCl, 2 mM EDTA buffer pH 7.3 at RT.

The suspension is then ultrasonicated under an N₂ atmosphere for 30 minutes in the Braukson ultrasonic bath at about 50 watts, the temperature being kept at about 20° C.

The acceptor liposomes are obtained analogously from 86 mg of cholesteryl oleate, 20 mg of triolein and 100 mg of phosphatidylcholine dissolved in 1.2 ml of dioxane and 114 ml of above buffer by ultrasonication at 50 watts (20° C.) for 30 minutes.

For testing, a test mix consisting of 1 part of above buffer, 1 part of donor liposomes and 2 parts of acceptor liposomes is used.

80 μl of test mix are treated with 1-3 μg of enriched CETP fraction, obtained from human plasma by means of hydrophobic chromatography, and 2 μl of the substance to be investigated in DMSO and incubated at 37° C. for 4 hours.

The change in the fluorescence at 485/535 nm is a measure of the CE transfer; the inhibition of the transfer in comparison to the control batch without substance is determined.

The following Table 1 gives representative results for the working examples:

TABLE 1

| Example | IC₅₀ [nM] |
|---|---|
| No. | Fluor. test |
| A-23 | 250 |
| A-35 | 200 |
| A-42 | 700 |
| A-47 | 200 |
| A-55 | 1500 |
| A-63 | 2000 |
| A-64 | 500 |
| A-70 | 2000 |
| A-78 | 3000 |
| A-80 | 5000 |
| B-1 | 1000 |
| B-5 | 1500 |
| B-8 | 1000 |
| B-22 | 700 |
| B-42 | 300 |
| B-45 | 70 |
| B-50 | 250 |
| B-69 | 100 |
| B-70 | 150 |
| B-81 | 60 |
| B-84 | 100 |
| B-147 | 200 |
| B-165 | 300 |
| B-176 | 5000 |
| C-21 | 70 |
| C-22 | 75 |
| C-37 | 60 |
| C-47 | 50 |
| C-56 | 57 |
| C-62 | 60 |
| C-63 | 32 |
| C-65 | 50 |
| C-66 | 50 |
| C-67 | 90 |
| C-71 | 40 |
| C-72 | 30 |
| C-73 | 15 |
| C-82 | 60 |
| C-91 | 40 |
| C-118 | 58 |
| C-120 | 100 |
| C-128 | 55 |

A3. Obtainment of Radiolabelled HDL

50 ml of fresh human EDTA plasma is adjusted to a density of 1.12 using NaBr and centrifuged at 4° C. in a Ty 65 rotor at 50 000 rpm for 18 h. The upper phase is used for the obtainment of cold LDL. The lower phase is dialysed against 3×41 of PDB buffer (10 mM tris/HCl pH 7.4, 0.15 mM NaCl, 1 mM EDTA, 0.02% NaN₃). Per 10 ml of retentate volume, 20 μl of ³H-cholesterol (Dupont NET-725; 1 μC/μl dissolved in ethanol) is then added and the mixture is incubated at 37° C. under N₂ for 72 h.

The batch is then adjusted to the density 1.21 using NaBr and centrifuged at 20° C. in a Ty 65 rotor at 50 000 rpm for 18 h. The upper phase is recovered and the lipoprotein fractions are purified by gradient centrifugation. To this end, the isolated, labelled lipoprotein fraction is adjusted to a density of 1.26 using NaBr. 4 ml each of this solution are covered in centrifuge tubes (SW 40 rotor) with a layer of 4 ml of a solution of density 1.21 and 4.5 ml of a solution of density 1.063 (density solutions of PDB buffer and NaBr) and then centrifuged for 24 h at 38 000 rpm and 20° C. in the SW 40 rotor. The intermediate layer lying between the density 1.063 and 1.21, containing the labelled HDL, is dialysed against 3×100 volumes of PDB buffer at 4° C.

The retentate contains radiolabelled ³H-CE-HDL, which, adjusted to about 5×10⁶ cmp per ml, is used for the test.

A4. CETP-SPA Test

For testing of the CETP activity, the transfer of ³H-cholesterol ester from human HD lipoproteins to biotinylated LD lipoproteins is measured.

The reaction is ended by addition of streptavidin-SPA® beads (Amersham) and the transferred radioactivity is determined directly in a liquid scintillation counter.

In the test batch, 10 μl of HDL-3H-cholesterol ester (˜50 000 cpm) are incubated at 37° C. for 18 h with 10 μl of biotin-LDL (Amersham) in 50 mM Hepes/0.15 M NaCl/0.1% bovine serum albumin/0.05% NaN₃ pH 7.4 containing 10 μl of CETP (1 mg/ml) and 3 μl of solution of the substance to be tested (dissolved in 10% DMSO/1% RSA). 200 μl of the SPA-streptavidin bead solution (TRKQ 7005) are then added, incubated further with shaking for 1 h and then measured in a scintillation counter. Corresponding incubations with 10 μl of buffer, 10 μl of CETP at 4° C. and 10 μl of CETP at 37° C. serve as controls.

The activity transferred in the control batches with CETP at 37° C. is rated as 100% transfer. The substance concentration at which this transfer is reduced to half is specified as the IC₅₀ value.

B. CETP Inhibition Test Ex Vivo/In Vivo

B1. Measurement of the Ex Vivo Activities on Transgenic hCETP Mice

To test for CETP-inhibitory activity, the substances are administered orally using a stomach tube to transgenic hCETP mice bred in-house (Dinchuk et al. BBA (1995) 1295-301). To this end, male animals are randomly assigned to groups having an equal number of animals, as a rule n=3, one day before the start of the experiment. Before administration of the substance, blood is taken from each mouse by puncture of the retro-orbital venous plexus for the determination of its basal CETP activity in the serum (T1). The test substance is then administered to the animals using the stomach tube. At specific times after administration of the test substance, blood is taken from the animals by puncture a second time (T2), as a rule 0.5, or 1 and 2 h after substance administration, but if appropriate this can also be carried out at another time.

In order to be able to assess the inhibitory activity of a substance, for each time, i.e. 0.5 or 1 or 2 h, a corresponding control group is employed whose animals only receive the formulating agent without substance. In the control animals, the second blood sampling per animal is carried out as in the substance-treated animals in order to be able to determine the change in the CETP activity without inhibitor over the corresponding experimental time interval (0.5, 1 or 2 h).

After termination of the clotting, the blood samples are centrifuged and the serum is removed by pipette.

For the determination of the CETP activity, the cholesteryl ester transport over 4 h is determined. To this end, as a rule 2 μl of serum are employed in the test batch and the test is carried out as described under “CETP fluorescence test”.

The differences in the cholesteryl ester transport [pM CE*/h (T2)−pM CE*/h (T1)] are calculated for each animal and averaged in the groups. A substance which at one of the times reduces the cholesteryl ester transport by >30% is regarded as active.

The following Table 2 gives representative results for the working examples:

TABLE 2

| | % inhibition at | | | | | % inhibition at | | |
|---|---|---|---|---|---|---|---|---|
| Example | 30 mg/kg | | | | | 100 mg/kg | | |
| No. | 0.5 h | 1 h | 2 h | 3 h | 6 h | 0.5 h | 1 h | 2 h |
| A-23 | | | | | | 59 | 52 | 31 |
| B-45 | 70 | 71 | 63 | | | | | |
| B-69 | 52 | 55 | 27 | | | | | |
| B-70 | 48 | 30 | 44 | | | | | |
| B-81 | 54 | 59 | 55 | | | | | |
| B-84 | 31 | 28 | 35 | | | | | |
| B-147 | | | | | | 76 | 54 | 33 |
| C-31 | 83 | 76 | 64 | | | | | |
| C-56 | | 66 | | 53 | 14 | | | |
| C-59 | | 52 | | 32 | 26 | | | |
| C-63 | | 83 | | 65 | 48 | | | |
| C-71 | | 60 | | 29 | 7 | | | |
| C-73 | | 91 | | 81 | 56 | | | |
| C-118 | | 75 | | 50 | 20 | | | |

B2. Measurement of the In Vivo Activity in Syrian Golden Hamsters

In experiments for the determination of the oral action on lipoproteins and triglycerides, test substance dissolved in DMSO and 0.5% suspended in Tylose are administered perorally by means of a stomach tube to Syrian golden hamsters bred in-house. For the determination of the CETP activity, before the start of the experiment blood is taken by retro-orbital puncture (about 250 μl). The test substances are then administered perorally by means of a stomach tube. The control animals receive identical volumes of solvent without test substance. The feed is then withdrawn from the animals and blood is taken at various times—up to 24 hours after substance administration—by puncture of the retro-orbital venous plexus.

Clotting is terminated by incubation of 4° C. overnight, then centrifugation is carried out for 10 minutes at 6000×g. The content of cholesterol and triglycerides in the serum thus obtained is determined with the aid of modified commercially obtainable enzymatic tests (Ecolin 25 Cholesterol, 1.14830.0001 Merck Diagnostica; Ecoline 25 Triglycerides, 1.14856.0001 Merck Diagnostica). Serum is suitably diluted using physiological saline solution.

10 μl of serum dilution are treated with 200 μl of Ecoline 25 reagent in 96-hole plates and incubated for 10 minutes at room temperature. The optical density is then determined at a wavelength of 490 nm using an automatic plate reader. The triglyceride or cholesterol concentration contained in the samples is determined with the aid of a standard curve measured in parallel.

The determination of the content of HDL cholesterol is carried out after precipitation of the ApoB-containing lipoproteins (Sigma 3524 HDL cholesterol reagent) according to the manufacturer's instructions.

The following Table 3 gives representative results for the working examples:

TABLE 3

| Example | % increase of HDL after |
|---|---|
| No. | 24 h (dose: 2 × 30 mg/kg) |
| C-63 | 17 |

B3. Measurement of the In Vivo Activity in Transgenic hCETP Mice

In experiments for the determination of the oral action on lipoproteins and triglycerides, test substance is administered to transgenic mice [Dinchuck et al., BBA, 1295-301 (1995)] using a stomach tube. Before the start of the experiment, blood is taken from the mice retro-orbitally in order to determine cholesterol and triglycerides in the serum. The serum is obtained as described above for hamsters by incubation at 4° C. overnight and subsequent centrifugation at 6000×g. After a week, blood is again taken from the mice in order to determine lipoproteins and triglycerides. The change in the parameters measured are expressed as the percentage change compared with the starting value.

The following Table 4 gives representative results for the working examples:

TABLE 4

| Example | % increase of HDL after |
|---|---|
| No. | 4 d (dose: 4 × 30 mg/kg) |
| C-72 | 68 |
| C-73 | 56 |

1. A method of treating or preventing a disorder controlled by inhibition of the cholesterol ester transfer protein (CETP), comprising administering to a patient a therapeutically effective amount of a compound of the general formula (I) 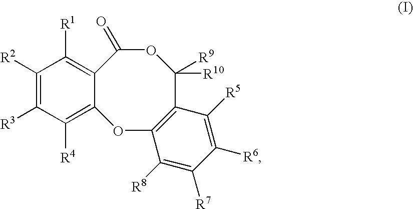
in which
R¹ represents hydrogen, halogen, cyano, (C₁-C₄)-alkyl, (C₁-C₄)-alkoxy, mono- or di-(C₁-C₄)-alkylamino, trifluoromethyl, trifluoromethoxy, hydroxy, vinyl or ethynyl,
R² represents a group of the formula 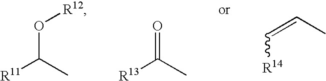
where
R¹¹ represents (C₁-C₆)-alkyl or (C₂-C₆)-alkenyl, each of which may be mono- or polysubstituted by substituents selected from the group consisting of (C₃-C₆)-cycloalkyl, phenyl, (C₁-C₄)-alkoxy and fluorine, or represents (C₆-C₁₀)-aryl which may be mono- or disubstituted by identical or different substituents from the group consisting of halogen, (C₁-C₄)-alkyl, (C₁-C₄)-alkoxy, trifluoromethyl and trifluoromethoxy,
R¹² represents hydrogen or formyl,
R¹³ and R¹⁴ each represent (C₁-C₆)-alkyl,
R³ and R⁴ independently of one another represent hydrogen, halogen, trifluoromethyl, trifluoromethoxy, (C₁-C₄)-alkyl, (C₁-C₄)-alkoxy, (C₂-C₄)-alkenyl or (C₃-C₆)-cycloalkyl,
R⁵, R⁶ and R⁷ independently of one another represent hydrogen, halogen, cyano, nitro, hydroxy, trifluoromethoxy, formyl, (C₁-C₄)-alkoxy, (C₂-C₄)-alkenyl, (C₃-C₆)-cycloalkyl or represent (C₁-C₄)-alkyl which may be substituted by hydroxy, trifluoromethoxy, (C₁-C₄)-alkoxy or up to three times by fluorine,
R⁸ represents (C₁-C₈)-alkyl, (C₂-C₈)-alkenyl or (C₂-C₈)-alkynyl, each of which may be substituted by (C₃-C₈)-cycloalkyl, (C₁-C₄)-alkoxy, pyrrolyl, imidazolyl, triazolyl, tetrazolyl or phenyl which for its part is optionally substituted by (C₁-C₄)-alkyl,
represents (C₆-C₁₀)-aryl which may be mono- or disubstituted by identical or different substituents from the group consisting of halogen, (C₁-C₄)-alkyl, (C₁-C₄)-alkoxy, trifluoromethyl, trifluoromethoxy, cyano and nitro,
represents (C₁-C₈)-alkoxy or (C₂-C₈)-alkenyloxy, each of which may be substituted by (C₃-C₈)-cycloalkyl, (C₃-C₈)-cycloalkenyl or phenyl, (which for its part is optionally substituted by halogen, nitro or cyano) or up to five times by fluorine and/or chlorine,
represents (C₃-C₈)-cycloalkoxy or represents (C₆-C₁₀)-aryloxy which may be substituted by halogen, nitro or cyano,
represents mono- or di-(C₁-C₈)-alkylamino, (C₁-C₈)-alkylsulphonylamino or N-[(C₁-C₈)-alkyl]-(C₁-C₈)-alkylsulphonylamino, or
represents a group of the formula —O—SO₂—R¹⁵, —O—C(O)—R⁶, —O—C(O)—NR¹⁷R¹⁸, —C(O)—OR¹⁹, —NR²⁰—C(O)—R²¹ or —NR²²—C(O)—NR²³R²⁴, where
R¹⁵ represents (C₁-C₈)-alkyl which may be substituted up to five times by fluorine, represents (C₃-C₈)-cycloalkyl or represents phenyl which may be substituted by halogen or (C₁-C₄)-alkyl,
R¹⁶ represents (C₁-C₁₀)-alkyl which may be substituted by phenyl or phenoxy (which for their part may each be mono- or disubstituted by halogen), by (C₃-C₈)-cycloalkyl, (C₃-C₈)-cycloalkenyl, (C₁-C₆)-alkoxy, (C₁-C₆)-alkylthio, (C₂-C₆)-alkenylthio or up to six times by fluorine,
represents (C₃-C₁₂)-cycloalkyl which may be mono- or polysubstituted by substituents selected from the group consisting of phenyl, (C₂-C₆)-alkenyl, trifluoromethyl, (C₁-C₆)-alkyl, cyano and fluorine, where phenyl for its part may be mono- or disubstituted by identical or different substituents from the group consisting of halogen, (C₁-C₄)-alkyl and (C₁-C₄)-alkoxy,
represents (C₃-C₁₂)-cycloalkenyl which may be substituted up to three times by (C₁-C₄)-alkyl, trifluoromethyl or fluorine,
represents a 5- to 7-membered mono- or bicyclic saturated or partially unsaturated heterocycle which has up to two heteroatoms from the group consisting of N, O and S and which may be substituted up to two times by (C₁-C₄)-alkyl, or
represents (C₆-C₁₀)-aryl which may be mono- or disubstituted by identical or different substituents from the group consisting of halogen, nitro, cyano, trifluoromethyl, trifluoromethoxy, (C₁-C₄)-alkyl and (C₁-C₄)-alkoxy,
R¹⁷ and R¹⁸ independently of one another represent hydrogen, (C₁-C₆)-alkyl which may be substituted by (C₁-C₄)-alkoxycarbonyl or up to three times by fluorine, represent (C₂-C₆)-alkenyl, (C₃-C₈)-cycloalkyl, (C₁-C₄)-alkylsulphonyl or represent phenyl which may be mono- or disubstituted by identical or different substituents from the group consisting of halogen and trifluoromethyl, or
together with the nitrogen atom to which they are attached form a 4- to 12-membered mono-, bi- or tricyclic saturated or partially unsaturated heterocycle which may contain up to two further heteroatoms from the group consisting of N, O and S and which may be substituted by phenyl or up to four times by (C₁-C₄)-alkyl,
R¹⁹ represents (C₁-C₆)-alkyl which may be substituted by (C₃-C₈)-cycloalkyl, represents (C₃-C₁₀)-cycloalkyl which may be substituted up to two times by (C₁-C₄)-alkyl or represents (C₂-C₆)-alkenyl,
R²⁰ represents hydrogen or (C₁-C₆)-alkyl,
R²¹ represents (C₁-C₈)-alkoxy, (C₁-C₈)-alkyl, (C₆-C₁₀)-aryl or represents (C₃-C₁₀)-cycloalkyl which may be substituted up to two times by (C₁-C₄)-alkyl,
R²² represents hydrogen or (C₁-C₆)-alkyl, and
R²³ and R²⁴ independently of one another represent hydrogen, (C₁-C₆)-alkyl or (C₃-C₁₀)-cycloalkyl, and
R⁹ and R¹⁰ independently of one another represent hydrogen or (C₁-C₄)-alkyl,
or a pharmaceutically acceptable salt thereof.

2. (canceled)

3. (canceled)

4. The method of claim 1, wherein the disorder controlled by inhibition of the cholesterol ester transfer protein (CETP) is a cardiovascular disorder.

5. The method of claim 1, wherein the disorder controlled by inhibition of the cholesterol ester transfer protein (CETP) is selected from hypolipoproteinaemia, dyslipidaemias, hypertriglyceridaemias, hyperlipidaemias and arteriosclerosis.

6. A compound of the formula (I) as defined in claim 1 in which
R⁸ represents a group of the formula —O—C(O)—R¹⁶ where
R¹⁶ represents (C₁-C₁₀)-alkyl which may be substituted by phenyl or phenoxy (which for their part may each be mono- or disubstituted by halogen), by (C₃-C₈)-cycloalkyl, (C₃-C₈)-cycloalkenyl, (C₁-C₆)-alkoxy, (C₁-C₆)-alkylthio, (C₂-C₆)-alkenylthio or up to six times by fluorine,
represents (C₃-C₁₂)-cycloalkyl which may be mono- or polysubstituted by substituents selected from the group consisting of phenyl, (C₂-C₆)-alkenyl, trifluoromethyl, (C₁-C₆)-alkyl, cyano and fluorine, where phenyl for its part may be mono- or disubstituted by identical or different substituents from the group consisting of halogen, (C₁-C₄)-alkyl and (C₁-C₄)-alkoxy,
represents (C₃-C₁₂)-cycloalkenyl which may be substituted up to three times by (C₁-C₄)-alkyl, trifluoromethyl or fluorine,
represents a 5- to 7-membered mono- or bicyclic saturated or partially unsaturated heterocycle which has up to two heteroatoms from the group consisting of N, O and S and which may be substituted up to two times by (C₁-C₄)-alkyl, or
represents (C₆-C₁₀)-aryl which may be mono- or disubstituted by identical or different substituents from the group consisting of halogen, nitro, cyano, trifluoromethyl, trifluoromethoxy, (C₁-C₄)-alkyl and (C₁-C₄)-alkoxy,
and R¹, R², R³, R⁴, R⁵, R⁶, R⁷, R⁹ and R¹⁰ are each as defined in claim 1.

7. A compound of the general formula (I) as defined in claim 1 in which
R⁸ represents a group of the formula —O—C(O)—NR¹⁷R¹⁸ where
R¹⁷ and R¹⁸ independently of one another represent hydrogen, (C₁-C₆)-alkyl which may be substituted by (C₁-C₄)-alkoxycarbonyl or up to three times by fluorine, represent (C₂-C₆)-alkenyl, (C₃-C₈)-cycloalkyl, (C₁-C₄)-alkylsulphonyl or represent phenyl which may be mono- or disubstituted by identical or different substituents from the group consisting of halogen and trifluoromethyl or
together with the nitrogen atom to which they are attached form a 4- to 12-membered mono-, bi- or tricyclic saturated or partially unsaturated heterocycle which may contain up to two further heteroatoms from the group consisting of N, O and S and which may be substituted by phenyl or up to four times by (C₁-C₄)-alkyl,
and R¹, R², R³, R⁴, R⁵, R⁶, R⁷, R⁹ and R¹⁰ are each as defined in claim 1.

8. A compound of the formula (I) as defined in claim 1 in which
R⁸ represents a group of the formula —C(O)—OR¹⁹ where
R¹⁹ represents (C₁-C₆)-alkyl which is substituted by (C₃-C₈)-cycloalkyl or represents (C₃-C₁₀)-cycloalkyl which may be substituted up to two times by (C₁-C₄)-alkyl,
and R¹, R², R³, R⁴, R⁵, R⁶, R⁷, R⁹ and R¹⁰ are each as defined in claim 1.

9. A compound of the formula (I) as defined in claim 1 in which
R⁸ represents a group of the formula —NR²⁰—C(O)—R²¹ where
R²⁰ represents hydrogen or (C₁-C₆)-alkyl, and
R² represents (C₁-C₈)-alkoxy, (C₁-C₈)-alkyl, (C₆-C₁₀)-aryl or represents (C₃-C₁₀)-cycloalkyl which may be substituted up to two times by (C₁-C₄)-alkyl,
and R¹, R², R³, R⁴, R⁵, R⁶, R⁷, R⁹ and R¹⁰ are each as defined in claim 1.

10. A compound of the formula (I) as defined in claim 1 in which
R⁸ represents a group of the formula —NR²²—C(O)—NR²³R²⁴ where
R²² represents hydrogen or (C₁-C₆)-alkyl, and
R²³ and R²⁴ independently of one another represent hydrogen, (C₁-C₆)-alkyl or (C₃-C₁₀)-cycloalkyl,
and R¹, R², R³, R⁴, R⁵, R⁶, R⁷, R⁹ and R¹⁰ are each as defined in claim 1.

11. A compound of the formula (I-A) 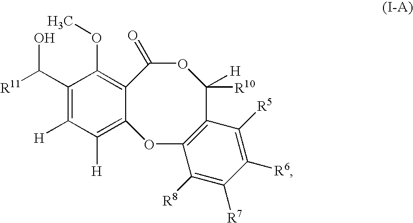
in which
R⁵, R⁶ and R⁷ independently of one another represent hydrogen, fluorine, chlorine, bromine, cyano or represent methyl or ethyl which may be substituted by methoxy or up to three times by fluorine,
R⁸ represents a group of the formula 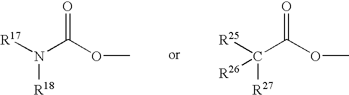
where
R¹⁷ and R¹⁸ independently of one another represent hydrogen, (C₁-C₆)-alkyl which may be substituted up to three times by fluorine, represent (C₃-C₆)-alkenyl or represent (C₃-C₆)-cycloalkyl, or
together with the nitrogen atom to which they are attached form a 4- to 10-membered mono-, bi- or tricyclic saturated or partially unsaturated heterocycle which may contain an oxygen atom as further heteroatom and which may be substituted up to four times by methyl,
R²⁵ and R²⁶ together with the carbon atom to which they are attached represent (C₃-C₁₀)-cycloalkyl which may be substituted up to four times by substituents selected from the group consisting of fluorine, methyl and trifluoromethyl, represent (C₅-C₁₀)-cycloalkenyl which may be substituted up to two times by methyl or represent a 5- to 7-membered saturated or partially saturated mono- or bicyclic heterocycle having a ring oxygen atom, and
R²⁷ represents hydrogen, (C₁-C₄)-alkyl, cyano or trifluoromethyl,
R¹⁰ represents hydrogen, methyl or ethyl, and
R¹¹ represents (C₁-C₆)-alkyl or (C₂-C₆)-alkenyl, each of which may be mono- to trisubstituted by substituents selected from the group consisting of cyclopropyl, cyclobutyl, methoxy and fluorine.

12. A compound of the formula (I-B) 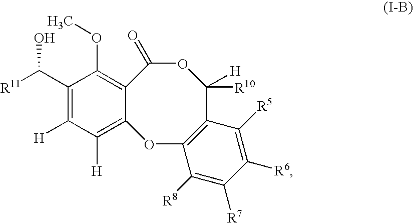
in which
R⁵, R⁶ and R⁷ independently of one another represent hydrogen, fluorine, chlorine, bromine, cyano or represent methyl or ethyl which may be substituted by methoxy or up to three times by fluorine,
R⁸ represents a group of the formula 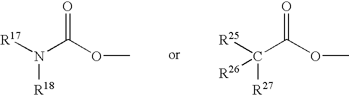
where
R¹⁷ and R¹⁸ independently of one another represent (C₁-C₆)-alkyl which may be substituted up to three times by fluorine, represent (C₃-C₆)-alkenyl or represent (C₃-C₆)-cycloalkyl, or
together with the nitrogen atom to which they are attached form a 4- to 10-membered saturated mono- or bicyclic heterocycle which may contain an oxygen atom as further heteroatom and which may be substituted up to two times by methyl,
R²⁵ and R²⁶ together with the carbon atom to which they are attached represent (C₃-C₁₀)-cycloalkyl which may be substituted up to four times by substituents selected from the group consisting of fluorine, methyl and trifluoromethyl, represent (C₅-C₇)-cycloalkenyl, 7-oxabicyclo[2.2.1]heptanyl or represent 7-oxabicyclo[2.2.1]hept-5-enyl, and
R²⁷ represents methyl, ethyl, propyl, cyano or trifluoromethyl,
R¹⁰ represents hydrogen, methyl or ethyl and
R¹¹ represents (C₁-C₆)-alkyl or (C₂-C₆)-alkenyl, each of which may be mono- to trisubstituted by substituents selected from the group consisting of cyclopropyl, cyclobutyl, methoxy and fluorine.

13. A method of treating or preventing a disorder controlled by inhibition of the cholesterol ester transfer protein (CETP), comprising administering to a patient a therapeutically effective amount of a compound of claim 11 or 12.

14. (canceled)

15. (canceled)

16. The method of claim 13, wherein the disorder controlled by inhibition of the cholesterol ester transfer protein (CETP) is a cardiovascular disorder.

17. The method of claim 16 wherein the cardiovascular disorder is selected from hypolipoproteinaemia, dyslipidaemias, hypertriglyceridaemias, hyperlipidaemias and arteriosclerosis.

18. A method of treating or preventing a disorder controlled by inhibition of the cholesterol ester transfer protein (CETP), comprising administering to a patient a therapeutically effective amount of a pharmaceutical composition, comprising a compound of the formula (I), as defined in claim 1, a compound of claim 11 or a compound of claim 12.
